SEQUENCE LISTING (76 sequences)

SEQ ID NO: 1 - DNA, 414 bp, Camelus dromedarius
gaagttcagctgcaggaatctggtggtggtctggttcagccgggtggttctctgcgtctgtcttgcgcggcgtctggtttcatcttctctaacgacgcgatgacctgggttcgtcaggcgccgggtaaaggtctggaatgggtttcttctatcaactggtctggtacccacaccaactacgcggactctgttaaaggtcgtttcaccatctctcgtgacaacgcgaaacgtaccctgtacctgcagatgaactctctgaaagacgaagacaccgcgctgtactactgcgttaccggttacggtgttaccaaaaccccgaccggtcagggtacccaggttaccgtttcttctcaccaccaccaccaccactctccgtctaccccgccgaccccgtctccgtctaccccgccgtgc

SEQ ID NO: 2 - protein, 138 aa, Camelus dromedarius
EVQLQESGGGLVQPGGSLRLSCAASGFIFSNDAMTWVRQAPGKGLEWVSSINWSGTHTNYADSVKGRFTISRDNAKRTLYLQMNSLKDEDTALYYCVTGYGVTKTPTGQGTQVTVSSHHHHHHSPSTPPTPSPSTPPC

SEQ ID NO: 3 - DNA, 375 bp, Camelus dromedarius
atggaagttcagctggttgaatctggtggtggtctggttcaggcgggtggttctctgcgtctgtcttgcgcggcgtctggtatcaccttctctatcaacaccatgggttggtaccgtcaggcgccgggtaaacagcgtgaactggttgcgctgatctcttctatcggtgacacctactacgcggactctgttaaaggtcgtttcaccatctctcgtgacaacgcgaaaaacaccgtttacctgcagatgaactctctgaaaccggaagacaccgcggtttactactgcaaacgtttccgtaccgcggcgcagggtaccgactactggggtcagggtacccaggttaccgtttcttctcaccaccaccaccaccac

SEQ ID NO: 4 - protein, 125 aa, Camelus dromedarius
MEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSHHHHHH

SEQ ID NO: 5 - DNA, 375 bp, Artificial Sequence
  Amino-acid coding DNA sequence of Anti-HER2 dsAb pcNB2
atggaagttcagctggttgaaaaaggtggtggtcgtgttcaggcgggtggttctctgcgtctgcgttgcgcggcgtctggtatcaccttctctatcaacaccatgggttggtaccgtcaggcgccgggtaaacagcgtgaactggttgcgctgatctcttctatcggtgacacctactacgcggactctgttaaaggtcgtttccgtatccgtcgtgacaacgcgaaaaacaccgtttacctgcgtatgcgtcgtctgaaaccggaagacaccgcggtttactactgcaaacgtttccgtaccgcggcgcagggtaccgactactggggtcagggtacccgtgttaccgtttctaaacaccaccaccaccaccac

SEQ ID NO: 6 - protein, 125 aa, Artificial Sequence
  Amino-acid sequence of Anti-HER2 dsAb pcNB2
MEVQLVEKGGGRVQAGGSLRLRCAASGITFSINTMGWYRQAPGKQRELVALISSIGDTYYADSVKGRFRIRRDNAKNTVYLRMRRLKPEDTAVYYCKRFRTAAQGTDYWGQGTRVTVSKHHHHHH

SEQ ID NO: 7 - DNA, 378 bp, Camelus dromedarius
gcggcgcaggttaaactggaagaatctggtggtggttctgttcagaccggtggttctctgcgtctgacctgcgcggcgtctggtcgtacctctcgttcttacggtatgggttggttccgtcaggcgccgggtaaagaacgtgaattcgtttctggtatctcttggcgtggtgactctaccggttacgcggactctgttaaaggtcgtttcaccatctctcgtgacaacgcgaaaaacaccgttgacctgcagatgaactctctgaaaccggaagacaccgcgatctactactgcgcggcggcggcgggttctgcgtggtacggtaccctgtacgaatacgactactggggtcagggtacccaggttaccgtttcttct

SEQ ID NO: 8 - protein, 126 aa, Camelus dromedarius
AAQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 9 - DNA, 381 bp, Camelus dromedarius
gaagttcagctggttgaatctggtggtggtctggttcaggcgggtggttctctgcgtctgtcttgcgcggcgtctggtcgtaccttctcttcttacgcgatgggttggttccgtcaggcgccgggtaaagaacgtgaattcgttgttgcgatcaactggtcttctggttctacctactacgcggactctgttaaaggtcgtttcaccatctctcgtgacaacgcgaaaaacaccatgtacctgcagatgaactctctgaaaccggaagacaccgcggtttactactgcgcggcgggttaccagatcaactctggtaactacaacttcaaagactacgaatacgactactggggtcagggtacccaggttaccgtttcttct

SEQ ID NO: 10 - protein, 127 aa, Camelus dromedarius
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSS

SEQ ID NO: 11 - DNA, 378 bp, Artificial Sequence
  Amino-acid coding DNA sequence of Anti-VGFR2 dsAb NTV1
atggcgcaggttcagctgctggaatctggtggtggtctggttcagccgggtggttctctgcgtctgtcttgcgcggcgtctggttactctgttatcaacgacttcatgacctgggttcgtcaggcgccgggtaaaggtctggaatgggtttcttctatctctgttgcggacggttctacctactacgcggactctgttaaaggtcgtttcaccatctctcgtgacaactctaaaaacaccctgtacctgcagatgaactctctgcgtgcggaagacaccgcggtttactactgcgcggcgcgtgttggtggtcgtgacctgggttggccgtacgaactggactactggggtcagggtaccctggttaccgtttcttct

SEQ ID NO: 12 - protein, 126 aa, Artificial Sequence
  Amino-acid sequence of Anti-VGFR2 dsAb NTV1
MAQVQLLESGGGLVQPGGSLRLSCAASGYSVINDFMTWVRQAPGKGLEWVSSISVADGSTYYADSVKGRFTISRDNSKNTLYLQMNSLRAEDTAVYYCAARVGGRDLGWPYELDYWGQGTLVTVSS

SEQ ID NO: 13 - DNA, 372 bp, Artificial Sequence
  Amino-acid coding DNA sequence of Anti-VGFR2 dsAb NTV2
atggcgcaggttcagctgctggaatctggtggtggtctggttcagccgggtggttctctgcgtctgtcttgcgcggcgtctggtttcaaaatcaccaacaaaaccatggcgtgggttcgtcaggcgccgggtaaaggtctggaatgggtttcttctatcggttcttcttctggttctacctactacgcggactctgttaaaggtcgtttcaccatctctcgtgacaactctaaaaacaccctgtacctgcagatgaactctctgcgtgcggaagacaccgcggtttactactgcgcgcgtcgtaaaggtaaccgtctgggtccggcggcgctgcgttcttggggtcagggtaccctggttaccgtttcttct

SEQ ID NO: 14 - protein, 124 aa, Artificial Sequence
  Amino-acid sequence of Anti-VGFR2 dsAb NTV2
MAQVQLLESGGGLVQPGGSLRLSCAASGFKITNKTMAWVRQAPGKGLEWVSSIGSSSGSTYYADSVKGRFTISRDNSKNTLYLQMNSLRAEDTAVYYCARRKGNRLGPAALRSWGQGTLVTVSS

SEQ ID NO: 15 - DNA, 363 bp, Artificial Sequence
  Amino-acid coding DNA sequence of Anti-VGFR2 dsAb NTV3
atggcgcaggttcagctgctggaatctggtggtggtctggttcagccgggtggttctctgcgtctgtcttgcgcggcgtctggtgttcgtgttaactacaaatctatgtcttgggttcgtcaggcgccgggtaaaggtctggaatgggtttctaccatcacctctcgtaacggttctacctactacgcggactctgttaaaggtcgtttcaccatctctcgtgacaactctaaaaacaccctgtacctgcagatgaactctctgcgtgcggaagacaccgcggtttactactgcgcgaccggtcgtgcgcaccacgcgccggttcgttactggggtcagggtaccctggttaccgtttcttct

SEQ ID NO: 16 - protein, 121 aa, Artificial Sequence
  Amino-acid sequence of Anti-VGFR2 dsAb NTV3
MAQVQLLESGGGLVQPGGSLRLSCAASGVRVNYKSMSWVRQAPGKGLEWVSTITSRNGSTYYADSVKGRFTISRDNSKNTLYLQMNSLRAEDTAVYYCATGRAHHAPVRYWGQGTLVTVSS

SEQ ID NO: 17 - DNA, 363 bp, Artificial Sequence
  Amino-acid coding DNA sequence of Anti-VGFR2 dsAb NTV4
atggcgcaggttcagctgctggaatctggtggtggtctggttcagccgggtggttctctgcgtctgtcttgcgcggcgtctggtgttaccatcaccgacgaagacatgacccgtgttcgtcaggcgccgggtaaaggtctggaatgggtttcttctatcctgaacaccggtggttctacctactacgcggactctgttaaaggtcgtttcaccatctctcgtgacaactctaaaaacaccctgtacctgcagatgaactctctgcgtgcggaagacaccgcggtttactactgcgcggcggttcacgaaaaagcggcggacatgaacttctggggtcagggtaccctggttaccgtttcttct

SEQ ID NO: 18 - protein, 120 aa, Artificial Sequence
  Amino-acid sequence of Anti-VGFR2 dsAb NTV4
AQVQLLESGGGLVQPGGSLRLSCAASGVTITDEDMTRVRQAPGKGLEWVSSILNTGGSTYYADSVKGRFTISRDNSKNTLYLQMNSLRAEDTAVYYCAAVHEKAADMNFWGQGTLVTVSS

SEQ ID NO: 19 - DNA, 327 bp, Camelus bactrianus
gaagttcagctgctggaatctggtggtggtctggttcagccgggtggttctctgcgttcttgcgaagcgtctggtttcaacgcgatgacctctatcgactcttggaccgacgcggttaaaggttgggttcgtcagccgccgggtaaaggtctggaatgggtttctcgtttcgcgatctctcaggacaacgcgaaaaacaccgtttacctgcagatgaactctctgaaaccggaagacaccgcgatgtactactgcgcgctgtctaaatgctacacccgtgtttacgactactggggtcagggtacccaggttaccgtttcttctggt

SEQ ID NO: 20 - protein, 108 aa, Camelus bactrianus
EVQLLESGGGLVQPGGSLRSCEASGFNAMTSIDSWTDAVKGWVRQPPGKGLEWVSRFAISQDNAKNTVYLQMNSLKPEDTAMYYCALSKCYTRVYDYWGQGTQVTVSS

SEQ ID NO: 21 - DNA, 312 bp, Camelus bactrianus
gaagttcagctgcaggaatctggtggtggtctggttcagccgggtggttctctgcgtctgtcttgcgcggcgtctggtttcatctacatggttggtatcaaaaccgaacgtgacggtgttaaaggttgggttcgtcaggcgccgggtaaaggtctggaatggctgtctcgtttcaccatcccgcgtgacaacgcgaaaaacaccctgtacctgcagatgaacaacctgaaatctgaagacaccgcgctgtactactgcgcgaccgaagaaaacgactggggtcagggtacccaggttaccgtttcttctggt

SEQ ID NO: 22 - protein, 103 aa, Camelus bactrianus
EVQLQESGGGLVQPGGSLRLSCAASGFIYMVGIKTERDGVKGWVRQAPGKGLEWLSRFTIPRDNAKNTLYLQMNNLKSEDTALYYCATEENDWGQGTQVTVSS

SEQ ID NO: 23 - DNA, 351 bp, Camelus dromedarius
caggttcagctgcaggaatctggtggtggttctgttcaggcgggtggttctctgcgtctgtcttgcaccgcgtctggtttcggtgttgacggtaccgacatgggttggtaccgtcaggcgccgggtaacgaatgcgaactggtttcttctatctcttctatcggtatcggttactactctgaatctgttaaaggtcgtttcaccatctctcgtgacaacgcgaaaaacaccgtttacctgcagatgaactctctgcgtccggacgacaccgcggtttactactgcggtcgtcgttggatcggttaccgttgcggtaactggggtcgtggtacccaggttaccgtttcttct

SEQ ID NO: 24 - protein, 117 aa, Camelus dromedarius
QVQLQESGGGSVQAGGSLRLSCTASGFGVDGTDMGWYRQAPGNECELVSSISSIGIGYYSESVKGRFTISRDNAKNTVYLQMNSLRPDDTAVYYCGRRWIGYRCGNWGRGTQVTVSS

SEQ ID NO: 25 - DNA, 369 bp, Camelus dromedarius
caggttcagctgcaggaatctggtggtggttctgttcaggcgggtggttctctgcgtctgtcttgcaccggttctcgttacacctacaccatgggttggttccgtcaggcgccgggtaaagaacgtgaaggtgttgttgcgatcaccgcgttcggttctccgttctacgcggactctgttaaaggtcgtttcaccatctctcgtgacaacgcgaacaacaccatcttcctgcagatgaactctctgaaaccggaagactctgcgatgtactactgcgcggcgcgtggttcttctggtacctcttacaaatggaacgaatacggttcttacaactactggggtcagggtacccaggttaccgtttcttct

SEQ ID NO: 26 - protein, 123 aa, Camelus dromedarius
QVQLQESGGGSVQAGGSLRLSCTGSRYTYTMGWFRQAPGKEREGVVAITAFGSPFYADSVKGRFTISRDNANNTIFLQMNSLKPEDSAMYYCAARGSSGTSYKWNEYGSYNYWGQGTQVTVSS

SEQ ID NO: 27 - DNA, 351 bp, Camelus dromedarius
caggttcagctgcaggaatctggtggtggttctgttcaggcgggtggttctctgcgtctgtcttgcgcggcgtctaaatacacctcttgcatgggttggttccgtcaggcgccgggtaaagaacgtgaagttgttgcgcacatcgactctggtccgcgtaccctgtacgcggactctgttaaaggtcgtttcaccatctctaaagacaacgcgaaaaacaccctgtacctggaaatgtctaccctgaaaccggacgacaccgcgatgtactactgcgcggcgggtccgatgtactctggttcttgcaactacaactactggggtcagggtacccaggttaccgtttcttct

SEQ ID NO: 28 - protein, 117 aa, Camelus dromedarius
QVQLQESGGGSVQAGGSLRLSCAASKYTSCMGWFRQAPGKEREVVAHIDSGPRTLYADSVKGRFTISKDNAKNTLYLEMSTLKPDDTAMYYCAAGPMYSGSCNYNYWGQGTQVTVSS

SEQ ID NO: 29 - DNA, 366 bp, Camelus dromedarius
caggttcagctgcaggaatctggtggtggtctggttcagccgggtggttctctgcgtctgtcttgcgcggcgtctggtttcaccctggactactacgcgatcggttggttccgtcaggcgccgggtaaagaacgtgaaggtgtttcttgcatctcttcttctgacggttctacctactacgcggactctgttaaaggtcgtttcaccatctctcgtgacaacgcgaaaaacaccgtttacctgcagatgtcttctctgaaaccggaagacaccgcggtttactactgcggtatctctggttcttgcctgctggaagactacggtatggactactggggtaaaggtacccaggttaccgtttcttct

SEQ ID NO: 30 - protein, 122 aa, Camelus dromedarius
QVQLQESGGGLVQPGGSLRLSCAASGFTLDYYAIGWFRQAPGKEREGVSCISSSDGSTYYADSVKGRFTISRDNAKNTVYLQMSSLKPEDTAVYYCGISGSCLLEDYGMDYWGKGTQVTVSS

SEQ ID NO: 31 - DNA, 378 bp, Camelus dromedarius
caggttcagctgcaggaatctggtggtggtctggttcacccgggtggttctctgcgtctgtcttgcgcggcgtctggtttctctctggacaactacgcgatcggttggttccgtcaggcgccgggtaaagaacgtgaaggtgtttcttgcatctcttctggttctgaaggtcgtcgttactacgcggacttcgttaaaggtcgtttcaccatctctcgtgacaacgcgaaaaacaccgcgttcctgcagatgaactctctgaaaccggaagacaccgcggactactactgcgcgaccgttggtttctgctcttctcagtacggtatggaattcgttggtgactactggggtcagggtacccaggttaccgtttcttct

SEQ ID NO: 32 - protein, 126 aa, Camelus dromedarius
QVQLQESGGGLVHPGGSLRLSCAASGFSLDNYAIGWFRQAPGKEREGVSCISSGSEGRRYYADFVKGRFTISRDNAKNTAFLQMNSLKPEDTADYYCATVGFCSSQYGMEFVGDYWGQGTQVTVSS

SEQ ID NO: 33 - protein, 115 aa, Artificial Sequence
  Amino-acid sequence of Anti-mouse serum albumin dsAb MSA21
QVQLQESGGGLVQPGGSLRLSCEASGFTFSRFGMTWVRQAPGKGVEWVSGISSLGDSTLYADSVKGRFTISRDNAKNTLYLQMNSLKPEDTAVYYCTIGGSLNPGGQGTQVTVSS

SEQ ID NO: 34 - protein, 115 aa, Artificial Sequence [sequence omitted]

SEQ ID NO: 35 - protein, 114 aa, Artificial Sequence
  Amino-acid sequence of Anti-human serum albumin dsAb Alb23
EVQLLESGGGLVQPGGSLRLSCAASGFTFRSFGMSWVRQAPGKGPEWVSSISGSGSDTLYADSVKGRFTISRDNSKTLYLQMNSLRPEDTAVYYCTIGGSLSRSSQGTLVTVSS

SEQ ID NO: 36 - protein, 125 aa, Artificial Sequence
  Amino-acid sequence of Anti-EGFR VHH 7A5
EVQLVESGGGLVQAGGSLRLSCAASDRTFSSNNMGWFRQAPGKEREFVAAIGWGGLETHYSDSVKGRFTISRDNAKNTVYLQMNSLKPEDTARYYCAVSSTRTVIYTLPRMYNYWGQGTQVTVSS

SEQ ID NO: 37 - protein, 124 aa, Artificial Sequence
  Amino-acid sequence of Anti-EGFR VHH 7D12
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 38 - protein, 124 aa, Artificial Sequence
  Amino-acid sequence of Anti-EGFR VHH 7C12
AVQLVESGGGSVQAGGSLRLTCAASGRTSRSYGMGWFRQAPGKEREFVSGISWRGDSTGYADSVKGRFTISRDNAKNTVDLQMNSLKPEDTAIYYCAAAAGSTWYGTLYEYDYWGQGTQVTVSS

SEQ ID NO: 39 - protein, 114 aa, Artificial Sequence
  Amino-acid sequence of Anti-insulin-like growth factor 1 receptor VHH 4B11
EVQLVESGGGLVQPGGSLRLSCAASGSIFTFNAMGWYRQAPGKQRELVAVIISGGSTHYVDSVKGRFTISRDNAKKMVYLQMNSLKPEDTAVYYCNVKKFGDYWGQGTQVTVSS

SEQ ID NO: 40 - protein, 116 aa, Artificial Sequence
  Amino-acid sequence of Anti-insulin-like growth factor 1 receptor VHH 3G7
DVQLVESGGGLVQAGGSLRLSCAASESISTINVMAWYRQAPGKQRELVAEITRSGRTNYVDSVKGRFTISRDNAKNTMYLQMNSLNLEDTAVYYCRTIDGSWREYWGQGTQVTVSS

SEQ ID NO: 41 - protein, 127 aa, Artificial Sequence
  Amino-acid sequence of Anti-insulin-like growth factor 1 receptor VHH 2C7
QVKLEESGGGLVQPGGSLRLSCVASGRTFSNYAIVIGWFRQAPGQEREFVAAINWNSRSTYYADSVKGRFTISRLNARNTVYLQMNRLKPEDTAVYDCAASHDSDYGGTNANLYDYWGQGTQVTVSS

SEQ ID NO: 42 - protein, 124 aa, Artificial Sequence
  Amino-acid sequence of Anti-insulin-like growth factor 1 receptor VHH 1C7
QVKLEESGGGLVQAGGSLRLSCVASGRTFSRTANAWFRQAPGKEREFVATITWNSGTTRYADSVKGRFFISKDSAKNTIYLEMNSLEPEDTAVYYCAATAAAVITPTRGYYNYWGQGTQVTVSS

SEQ ID NO: 43 - protein, 123 aa, Artificial Sequence
  Amino-acid sequence of Anti-CEACAM VHH NbCEA5
EVQLVESGGGSVQAGGSLRLSCAASGDTYGSYWMGWFRQAPGKEREGVAAINRGGGYTVYADSVKGRFTISRDTAKNTVYLQMNSLRPDDTADYYCAASGVLGGLHEDWFNYWGQGTLVTVSS

SEQ ID NO: 44 - protein, 123 aa, Artificial Sequence
  Amino-acid sequence of Anti-CEACAM VHH CEA5
EVQLVESGGGSVQAGGSLRLSCAASGDTYGSYWMGWFRQAPGQEREAVAAINRGGGYTVYADSVKGRFTISRDNAKNTLYLQMNSLRPDDTADYYCAASGVLGGLHEDWFNYWGQGTLVTVSS

SEQ ID NO: 45 - protein, 116 aa, Artificial Sequence
  Amino-acid sequence of Anti-CD123 VHH 57A07
EVQLVESGGGLVQAGGSLRLSCAASGSIFSGNVMGWYRRQAPGKEREWVAAIASGGSIYYRDSVKGRFTISRDNAKNTVYLQMNSLKPEDTAVYYCNSHPPTLPYWGLGTQVTVSS

SEQ ID NO: 46 - protein, 116 aa, Artificial Sequence
  Amino-acid sequence of Anti-CD123 VHH 57B04
EVQLVESGGGLVQPGGSLRLSCAASGINFRFNSMGWWRRRAPGKEREWVAAITSGDITNYRDSVRGRFTISRDNVKNTVYLQMNTLKLEDTAVYYCNTFPPIADYWGLGTQVTVSS

SEQ ID NO: 47 - protein, 123 aa, Artificial Sequence
  Amino-acid sequence of Anti-CD123 VHH 51D09
EVQLVESGGGLVQPGGSLRLSCAASGSIFSGNTMGWYRQAPGKQRELVAAISSGGSTDYADSVKGRFTISRDNSKNTVYLQMNSLRPEDTAVYYCNAAILLYRLYGYEEGDYWGLGTLVTVSS

SEQ ID NO: 48 - protein, 125 aa, Artificial Sequence
  Amino-acid sequence of Anti-CD123 VHH 55C05
EVQLVESGGGLVPAGDSLRLSCVASGRSLNTYTMGWFRQAPGKECEEVAAINWNGVYRDYADSAKGRETASRDNAMNTVFLQMNSLKPEDTAVYFCATATQGWDRHTEPSDFGSWGLGTQVTVSS

SEQ ID NO: 49 - protein, 124 aa, Artificial Sequence
  Amino-acid sequence of Anti-CD123 VHH 50F07
EVQLVESGGGLVQPGGSLRLSCTGSGSTFSINAMGWYRQAPGKQRELVAAITSGGRTNYADSVKGRFTISRDNSKNTVYLQMNSLRPEDTAVYYCNARISAGTAFWLWSDYEYWGLGTLVTVSS

SEQ ID NO: 50 - protein, 125 aa, Artificial Sequence
  Amino-acid sequence of Anti-CD123 VHH 55F03
EVQLVESGGGLVQAGGPLRLSCAASGRTFSSYVMGWFRQAPGKEREFVAAIYWSNGKTQYTDSVKGRFTISGDNAKNTVYLQMNSLNPEDTAVYYCVADKDETGFRTLPIAYDYWGLGTQVTVSS

SEQ ID NO: 51 - protein, 126 aa, Artificial Sequence
  Amino-acid sequence of Anti-CD123 VHH 55A01
EVQLVESGGGSVQAGGSLRLSCTTSGRALNMYVMGWFRQAPGNEREFVAATSSSGGSTSYPDSVKGRFTISRDNAKNTVYLQMNSLKPEDTAAYRCAASPYVSTPTMNILEEYRYWGLGTQVTVSS

SEQ ID NO: 52 - protein, 125 aa, Artificial Sequence
  Amino-acid sequence of Anti-c-MET VHH 04E09
EVQLVESGGGLVQPGGSLRLSCAASGFILDYYAIGWFRQAPGKEREGVLCIDASDDITYYADSVKGRFTISRDNAKNTVYLQMNSLKPEDTGVYYCATPIGLSSSCLLEYDYDYWGQGTLVTVSS

SEQ ID NO: 53 - protein, 130 aa, Artificial Sequence
  Amino-acid sequence of Anti-c-MET VHH 06B08
EVQLVESGGGLVQAGGSLRLSCAASGRTISRYTMGWFRQAPGKEREFVAAISWSGDNTNYADSVKGRFTISRPNTKNTMYLQMNSLKPEDTAVYYCAADYRSGSYYQASEWTRPSGYDYWGQGTLVTVSS

SEQ ID NO: 54 - protein, 125 aa, Artificial Sequence
  Amino-acid sequence of Anti-c-MET VHH 06C12
EVQLVESGGGLVQPGGSLRLSCAASGFSLDYFAIGWFRQAPGKEREEISCISNSDGSTYYANSVKGRFTISIDNAKNTVYLQMTSLKPEDTAVYYCATPVGLGPFCKTTNDYDYSGQGTLVTVSS

SEQ ID NO: 55 - protein, 124 aa, Artificial Sequence
  Amino-acid sequence of Anti-c-MET VHH 06F10
EVQLVESGGGLVQPGGSLRLSCAASGFTLDYYAINWFRQAPGKEREGVSCISGGDGSTYYADSVKGRFTISRDNAKNTVYLQMNSLKPEDTAVYYCATALGLSSSCHGDGYDYWGQGTLVTVSS

SEQ ID NO: 56 - protein, 125 aa, Artificial Sequence
  Amino-acid sequence of Anti-Her3 VHH 21F6
EVQLVESGGGLVQAGGSLRLSCAASGRTYYLNAMGWFRQGPGKDREFVAAIDWSDGNKDYADSVKGRFTISRDNAKNTVYLQMNSLKPEDTAVYYCAADTPPWGPMIYIESYDSWGQGTLVTVSS

SEQ ID NO: 57 - protein, 113 aa, Artificial Sequence
  Amino-acid sequence of Anti-Her3 VHH 4C7
EVQLVESGGGLVQAGGSLRLSCAASGFTFSSYPMSWVRQAPGKGPAWVSTVSPGGITTSYADSVKGRFTISRDNAKNTLYLQMNSLKPEDTAVYYCLRDLNNRGQGTLVTVSS

SEQ ID NO: 58 - protein, 118 aa, Artificial Sequence
  Amino-acid sequence of Anti-Her3 VHH 17B5
EVQLVESGGGLVQPGGSLRLSCAASGSIGGLNAMAWYRQAPGKERELVAGIFGVGSTRYADSVKGRFTISRDIAKNTVFLQMNSLNSEDTAVYYCRMSSVTRGSSDYWGQGTQVTVSS

SEQ ID NO: 59 - protein, 122 aa, Artificial Sequence
  Amino-acid sequence of Anti-Her3 VHH 18G11
EVQLVESGGGLVQPGGSLRLSCAASGTLFKINAMGWYRQAPGKRRELVALITSSDTTDYAESVEGRFTISRDNTWNAVYLQMNSLKPEDTAVYYCHSDHYSMGVPEKRVIMYGQGTQVTVSS

SEQ ID NO: 60 - protein, 120 aa, Artificial Sequence
  Amino-acid sequence of Anti-Her3 VHH 34C7
EVQLVESGGGLVQPGGSLGLSCVASGSIFRINAMAWYRQAPGKQRELVAEITAGGSTNYADSVKGRFTISVDNAWNTLYLQMNSLKVEDTAVYYCNLDHYTTWDRRSAYWGQGTQVTVSS

SEQ ID NO: 61 - protein, 119 aa, Lama glama
KVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 62 - protein, 124 aa, Lama glama
EVQLVESGGSLVQPGGSLRLSCAASGFTFDDYAMSWVRQVPGKGLEWVSSINWSGTHTDYADSVKGRFTISRNNANNTLYLQMNSLKSEDTAVYYCAKNWRDAGTTWFEKSGSAGQGTQVTVSS

SEQ ID NO: 63 - protein, 118 aa, Lama glama
EVQLVESGGGLVQPGGSLRLSCAASGFTFSINTMGWYRQAPGKQRELVALISSIGDTYYADSVKGRFTISRDNAKNTVYLQMNSLKPEDTAVYYCKRFRTAAQGTDYWGQGTQVTVSS

SEQ ID NO: 64 - protein, 118 aa, Lama glama
EVQLVESGGGLVHPGGSLRLSCVGSGFSLDDYGMTWVRRAPGKGLEWVSSINWSGTHTDYADSVKGRFTISRDNAKNTLFLQMNSLNPEDTAVYYCGQGWKIVPTNPRGHGTQVTVSS

SEQ ID NO: 65 - protein, 118 aa, Lama glama
EVQLVESGGGLVQPGGSLRLSCVGSGFSLDDYAMTWVRQAPGKGLEWVSSINWSGTHTDYADSVKGRFTISRDNAKNTLFLQMNSLSPEDTAVYYCNQGWKIRPTIPMGHGTQVTVSS

SEQ ID NO: 66 - protein, 118 aa, Lama glama
EVQLVESGGGLVQPGGSLRLSCVASGFSLDDYGMTWVRQAPGKGLEWVSSINWSGTHTDYTDPVKGRFTISRDNAKNTLFLQMNNLTPEDTAVYYCNRGWKIVPTDLGGHGTQVTVSS

SEQ ID NO: 67 - protein, 122 aa, Lama glama
EVQLVESGGGLVQAGGSLRLSCAASGRTFISNYAMGWFRQAPGKEREFVATINWSGSHSDYADSVKGRFTISRDNAKNTVYLQMNNLKSEDTAVYYCAPGWGTAPLSTSVYWGQGTQVTVSS

SEQ ID NO: 68 - protein, 119 aa, Lama glama
EVQLVESGGGMVQAGGSLRLSCAASGLTLSNYGMGWFRQAPGKEREFVSSINWSGTHTYDADFVKGRFIISRDNAKNTVYLQINSLKPEDTAVYYCAAGGWGTGRYNYWGQGTQVTVSS

SEQ ID NO: 69 - protein, 119 aa, Lama glama; identical to SEQ ID NO: 68

SEQ ID NO: 70 - protein, 120 aa, Lama glama
EVQLVESGGGLVQAGGSLRLSCAASGRTFNAMGWFRQAPGKEREFVAAISRSPGVTYYADSVKGRFTTSRDNAKNTVYLQMNDLKPEDTAVYYCAADFYLATLAHEYDYWGQGTQVTVSS

SEQ ID NO: 71 - protein, 122 aa, Lama glama
EVQLVESGGGLVQAGGSLRLSCAASGRTFSSYAMAWFRQAPGTEREFIAGIRWSDGSTYYADSVKGRFTISRDNAKNTVYLQMNSLKPEDTAVYYCAADFYVSTLAHEYDYWGQGTQVTVSS

SEQ ID NO: 72 - protein, 122 aa, Lama glama
KVQLVESGGGLVQAGGSLRLSCAASGRTFSSYAMAWFRQAPGTEREFIAGIRWSDGSTYYADSVKGRFTISRANAKNTVYLQMNGLKPEDTAVYYCAADFYVSTLAHEYDYWGQGTQVTVSS

SEQ ID NO: 73 - protein, 576 aa, Artificial Sequence
  Bivalent VHHEGFR-dianthin-Cys
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSEVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGGSAAATAYTLNLANPSASQYSSFLDQIRNNVRDTSLIYGGTDVAVIGAPSTTDKFLRLNFQGPRGTVSLGLRRENLYVVAYLAMDNANVNRAYYFKNQITSAELTALFPEVVVANQKQLEYGEDYQAIEKNAKITTGDQSRKELGLGINLLITMIDGVNKKVRVVKDEARFLLIAIQMTAEAARFRYIQNLVTKNFPNKFDSENKVIQFQVSWSKISTAIFGDCKNGVFNKDYDFGFGKVRQAKDLQMGLLKYLGRPKGGGGSGGGGSHRWCCPGCCKTFGGGGSHHHHHHHHHHHRWCCPGCCKTF

SEQ ID NO: 74 - protein, 283 aa, Artificial Sequence
  Bivalent VHHEGFR-Cys
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSEVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSHHHHHHHHHHHRWCCPGCCKTF

SEQ ID NO: 75 - protein, 124 aa, Artificial Sequence; identical to SEQ ID NO: 37
  VHH 7D12

SEQ ID NO: 76 - protein, 127 aa, Artificial Sequence; identical to SEQ ID NO: 10
  VHH 9G8

(19) United States
(12) Patent Application Publication
(10) Pub. No.: US 2023/0293708 A1
(43) Pub. Date: Sep. 21, 2023

(54) CONJUGATE OF SAPONIN AND SINGLE-DOMAIN ANTIBODY, PHARMACEUTICAL COMPOSITION COMPRISING SAID CONJUGATE, THERAPEUTIC USE THEREOF

(71) Applicants: SAPREME TECHNOLOGIES B.V., Bilthoven (NL); CHARITÉ - UNIVERSITÄTSMEDIZIN BERLIN, Berlin (DE)

(72) Inventors: Ruben POSTEL, Bilthoven (NL); Guy HERMANS, Bilthoven (NL); Hendrik FUCHS, Berlin (DE)

(21) Appl. No.: 18/012,715

(22) PCT Filed: Jun. 22, 2021
(86) PCT No.: PCT/EP2021/066985

§ 371 Date: Dec. 23, 2022

(30) Foreign Application Priority Data
Jun. 24, 2020 (NL) 2025898
Jul. 9, 2020 (WO) PCT/EP2020/069341

(51) Int. Cl. A61K 47/68; A61K 45/06; A61P 35/00

(52) U.S. Cl. CPC A61K 47/6809; A61K 47/6889; A61K 47/6851; A61K 47/6849; A61K 45/06; A61P 35/00

(57) ABSTRACT

The invention relates to a conjugate for transferring a saponin of the monodesmosidic triterpene glycoside type or of the bidesmosidic triterpene glycoside type from outside a cell into said cell, the conjugate comprising a single-domain antibody, capable of binding to said cell, covalently bound to a saponin. The invention also relates to a pharmaceutical combination comprising a first pharmaceutical composition comprising the conjugate of the invention and a second pharmaceutical composition comprising an active pharmaceutical ingredient, or to a pharmaceutical composition comprising the conjugate of the invention and an active pharmaceutical ingredient. In addition, the invention relates to the pharmaceutical combination of the invention or the pharmaceutical composition of the invention, for use as a medicament, or for use in the treatment or the prophylaxis of a cancer, an auto-immune disease, an infection, an enzyme deficiency, a gene defect. Furthermore, the invention relates to an in vitro or ex vivo method for transferring a molecule from outside a cell to inside said cell, comprising the application of the conjugate of the invention. Finally, the invention relates to a kit of parts, comprising the pharmaceutical combination of the invention or the pharmaceutical composition of the invention, or the conjugate of the invention, and instructions for use thereof.

### TECHNOLOGICAL FIELD

The invention relates to a conjugate for transferring a saponin of the monodesmosidic triterpene glycoside type or of the bidesmosidic triterpene glycoside type from outside a cell into said cell, the conjugate comprising a single-domain antibody, capable of binding to said cell, covalently bound to a saponin. The invention also relates to a pharmaceutical combination comprising a first pharmaceutical composition comprising the conjugate of the invention and a second pharmaceutical composition comprising an active pharmaceutical ingredient, or to a pharmaceutical composition comprising the conjugate of the invention and an active pharmaceutical ingredient. In addition, the invention relates to the pharmaceutical combination of the invention or the pharmaceutical composition of the invention, for use as a medicament, or for use in the treatment or the prophylaxis of a cancer, an auto-immune disease, an infection, an enzyme deficiency, a gene defect. Furthermore, the invention relates to an in vitro or ex vivo method for transferring a molecule from outside a cell to inside said cell, comprising the application of the conjugate of the invention. Finally, the invention relates to a kit of parts, comprising the pharmaceutical combination of the invention or the pharmaceutical composition of the invention, or the conjugate of the invention, and instructions for use thereof.

### BACKGROUND

Molecules with a therapeutic biological activity are in many occasions in theory suitable for application as an effective therapeutic drug for the treatment of a disease such as a cancer in human patients in need thereof. A typical example are small-molecule biologically active moieties. However, many if not all potential drug-like molecules and therapeutics currently used in the clinic suffer from at least one of a plethora of shortcomings and drawbacks. When administered to a human body, therapeutically active molecules may exert off-target effects, in addition to the biologically activity directed to an aspect underlying a to-be-treated disease or health problem. Such off-target effects are undesired and bear a risk for induction of health- or even life-threatening side effects of the administered molecule. It is the occurrence of such adverse events that cause many drug-like compounds and therapeutic moieties to fail phase III clinical trials or even phase IV clinical trials (post-market entry follow-up). Therefore, there is a strong desire to provide drug molecules such as small-molecule therapeutics, wherein the therapeutic effect of the drug molecule should, e.g., (1) be highly specific for a biological factor or biological process driving the disease, (2) be sufficiently safe, (3) be sufficiently efficacious, (4) be sufficiently directed to the diseased cell with little to no off-target activity on non-diseased cells, (5) have a sufficiently timely mode of action (e.g. the administered drug molecule should reach the targeted site in the human patient within a certain time frame and should remain at the targeted site for a certain time frame), and/or (6) have sufficiently long lasting therapeutic activity in the patient's body, amongst others. Unfortunately, to date, ‘ideal’ therapeutics with many or even all of the beneficial characteristics here above outlined, are not available to the patients, despite already long-lasting and intensive research and despite the impressive progress made in several areas of the individually addressed encountered difficulties and drawbacks.

Chemotherapy is one of the most important therapeutic options for cancer treatment. However, it is often associated with a low therapeutic window because it has no specificity towards cancer cells over dividing cells in healthy tissue. The invention of monoclonal antibodies offered the possibility of exploiting their specific binding properties as a mechanism for the targeted delivery of cytotoxic agents to cancer cells, while sparing normal cells. This can be achieved by chemical conjugation of cytotoxic effectors (also known as payloads or warheads) to antibodies, to create antibody—drug conjugates (ADCs). Typically, very potent payloads such as emtansine (DM1) are used which have a limited therapeutic index (a ratio that compares toxic dose to efficacious dose) in their unconjugated forms. The conjugation of DM1 to trastuzumab (ado-trastuzumab emtansine), also known as Kadcycla, enhances the tolerable dose of DM1 at least two-fold in monkeys. In the past few decades tremendous efforts and investments have been made to develop therapeutic ADCs. However, it remains challenging to bring ADCs into the clinic, despite promising preclinical data. The first ADC approved for clinical use was gemtuzumab ozogamicin (Mylotarg, CD33 targeted, Pfizer/Wyeth) for relapsed acute myelogenous leukemia (AML) in 2000. Mylotarg was however, withdrawn from the market at the request of the Federal Drug Administration (FDA) due to a number of concerns including its safety profile. Patients treated with Mylotarg were more often found to die than patients treated with conventional chemotherapy. Mylotarg was admitted to the market again in 2017 with a lower recommended dose, a different schedule in combination with chemotherapy or on its own, and a new patient population. To date, only five ADCs have been approved for clinical use, and meanwhile clinical development of approximately fifty-five ADCs has been halted. However, interest remains high and approximately eighty ADCs are still in clinical development in nearly six-hundred clinical trials at present.

Despite the potential to use toxic payloads that are normally not tolerated by patients, a low therapeutic index (a ratio that compares toxic dose to efficacious dose) is a major problem accounting for the discontinuance of many ADCs in clinical development, which can be caused by several mechanisms such as off-target toxicity on normal cells, development of resistance against the cytotoxic agents and premature release of drugs in the circulation. A systematic review by the FDA of ADCs found that the toxicity profiles of most ADCs could be categorized according to the payload used, but not the antibody used, suggesting that toxicity is mostly determined by premature release of the payload. Of the approximately fifty-five ADCs that were discontinued, it is estimated that at least twenty-three were due to a poor therapeutic index. For example, development of a trastuzumab tesirine conjugate (ADCT-502, HER-2 targeted, ADC therapeutics) was recently discontinued due to a narrow therapeutic index, possibly due to an on-target, off-tissue effect in pulmonary tissue which expresses considerable levels of HER-2. In addition, several ADCs in phase 3 trials have been discontinued due to missing primary endpoint. For example, phase 3 trials of a depatuxizumab mafodotin conjugate (ABT-414, EGFR targeted, AbbVie) tested in patients with newly diagnosed glioblastoma, and a mirvetuximab soravtansine conjugate (IMGN853, folate receptor alpha (FRα) targeted, ImmunoGen) tested in patients with platinum-resistant ovarian cancer, were recently stopped, showing no survival benefit. It is important to note that the clinically used dose of some ADCs may not be sufficient for its full anticancer activity. For example, ado-trastuzumab emtansine has an MTD of 3.6 mg/kg in humans. In preclinical models of breast cancer, ado-trastuzumab emtansine induced tumor regression at dose levels at or above 3 mg/kg, but more potent efficacy was observed at 15 mg/kg. This suggests that at the clinically administered dose, ado-trastuzumab emtansine may not exert its maximal potential anti-tumor effect.

ADCs are mainly composed of an antibody, a cytotoxic moiety such as a payload, and a linker. Several novel strategies have been proposed and carried out in the design and development of new ADCs to overcome the existing problems, targeting each of the components of ADCs. For example, by identification and validation of adequate antigenic targets for the antibody component, by selecting antigens which have high expression levels in tumor and little or no expression in normal tissues, antigens which are present on the cell surface to be accessible to the circulating ADCs, and antigens which allows internalizing of ADCs into the cell after binding; and alternative mechanisms of activity; design and optimize linkers which enhance the solubility and the drug-to-antibody ratio (DAR) of ADCs and overcome resistance induced by proteins that can transport the chemotherapeutic agent out of the cells; enhance the DAR ratio by inclusion of more payloads, select and optimize antibodies to improve antibody homogeneity and developability. In addition to the technological development of ADCs, new clinical and translational strategies are also being deployed to maximize the therapeutic index, such as, change dosing schedules through fractionated dosing; perform biodistribution studies; include biomarkers to optimize patient selection, to capture response signals early and monitor the duration and depth of response, and to inform combination studies.

An example of ADCs with clinical potential are those ADCs such as brentuximab vedotin, inotuzumab ozogamicin, moxetumomab pasudotox, and polatuzumab vedotin, which are evaluated as a treatment option for lymphoid malignancies and multiple myeloma. Polatuzumab vedotin, binding to CD79b on (malignant) B-cells, and pinatuzumab vedotin, binding to CD22, are tested in clinical trials wherein the ADCs each were combined with co-administered rituximab, a monoclonal antibody binding to CD20 and not provided with a payload [B. Yu and D. Liu, Antibody-drug conjugates in clinical trials for lymphoid malignancies and multiple myeloma; Journal of Hematology & Oncology (2019) 12:94]. Combinations of monoclonal antibodies such as these examples are yet a further approach and attempt to arrive at the ‘magic bullet’ which combines many or even all of the aforementioned desired characteristics of ADCs.

Meanwhile in the past few decades, nucleic acid-based therapeutics are under development. Therapeutic nucleic acids can be based on deoxyribonucleic acid (DNA) or ribonucleic acid (RNA), Anti-sense oligonucleotides (ASOs, AONs), and short interfering RNAs (siRNAs), MicroRNAs, and DNA and RNA aptamers, for approaches such as gene therapy, RNA interference (RNAi). Many of them share the same fundamental basis of action by inhibition of either DNA or RNA expression, thereby preventing expression of disease-related abnormal proteins. The largest number of clinical trials is being carried out in the field of gene therapy, with almost 2600 ongoing or completed clinical trials worldwide but with only about 4% entering phase 3. This is followed by clinical trials with ASOs. Similarly to ADCs, despite the large number of techniques being explored, therapeutic nucleic acids share two major issues during clinical development: delivery into cells and off-target effects. For instance, ASOs such as peptide nucleic acid (PNA), phosphoramidate morpholino oligomer (PMO), locked nucleic acid (LNA) and bridged nucleic acid (BNA), are being investigated as an attractive strategy to inhibit specifically target genes and especially those genes that are difficult to target with small molecules inhibitors or neutralizing antibodies. Currently, the efficacy of different ASOs is being studied in many neurodegenerative diseases such as Huntington's disease, Parkinson's disease, Alzheimer's disease, and amyotrophic lateral sclerosis and also in several cancer stages. The application of ASOs as potential therapeutic agents requires safe and effective methods for their delivery to the cytoplasm and/or nucleus of the target cells and tissues. Although the clinical relevance of ASOs has been demonstrated, inefficient cellular uptake, both in vitro and in vivo, limit the efficacy of ASOs and has been a barrier to therapeutic development. Cellular uptake can be <2% of the dose resulting in too low ASO concentration at the active site for an effective and sustained outcome. This consequently requires an increase of the administered dose which induces off-target effects. Most common side-effects are activation of the complement cascade, the inhibition of the clotting cascade and toll-like receptor mediated stimulation of the immune system.

Chemotherapeutics are most commonly small molecules, however, their efficacy is hampered by the severe off-target side toxicity, as well as their poor solubility, rapid clearance and limited tumor exposure. Scaffold-small-molecule drug conjugates such as polymer-drug conjugates (PDCs) are macromolecular constructs with pharmacologically activity, which comprises one or more molecules of a small-molecule drug bound to a carrier scaffold (e.g. polyethylene glycol (PEG)).

Such conjugate principle has attracted much attention and has been under investigation for several decades. The majority of conjugates of small-molecule drugs under pre-clinical or clinical development are for oncological indications. However, up-to-date only one drug not related to cancer has been approved (Movantik, a PEG oligomer conjugate of opioid antagonist naloxone, AstraZeneca) for opioid-induced constipation in patients with chronic pain in 2014, which is a non-oncology indication. Translating application of drug-scaffold conjugates into treatment of human subjects provided little clinical success so far. For example, PK1 (N-(2-hydroxypropyl)methacrylamide (HPMA) copolymer doxorubicin; development by Pharmacia, Pfizer) showed great anti-cancer activity in both solid tumors and leukemia in murine models, and was under clinical investigation for oncological indications. Despite that it demonstrated significant reduction of nonspecific toxicity and improved pharmacokinetics in man, improvements in anticancer efficacy turned out to be marginal in patients, and as a consequence further development of PK1 was discontinued.

The failure of scaffold-small-molecule drug conjugates is at least partially attributed to its poor accumulation at the tumor site. For example, while in murine models PK1 showed 45-250 times higher accumulation in the tumor than in healthy tissues (liver, kidney, lung, spleen, and heart), accumulation in tumor was only observed in a small subset of patients in the clinical trial.

A potential solution to the aforementioned problems is application of nanoparticle systems for drug delivery such as liposomes. Liposomes are sphere-shaped vesicles consisting of one or more phospholipid bilayers, which are spontaneously formed when phospholipids are dispersed in water. The amphiphilicity characteristics of the phospholipids provide it with the properties of self-assembly, emulsifying and wetting characteristics, and these properties can be employed in the design of new drugs and new drug delivery systems. Drug encapsulated in a liposomal delivery system may convey several advantages over a direct administration of the drug, such as an improvement and control over pharmacokinetics and pharmacodynamics, tissue targeting property, decreased toxicity and enhanced drug activity. An example of such success is liposome-encapsulated form of a small molecule chemotherapy agent doxorubicin (Doxil: a pegylated liposome-encapsulated form of doxorubicin; Myocet: a non-pegylated liposomal doxorubicin), which have been approved for clinical use.

Therefore, a solution still needs to be found that allows for drug therapies such as anti-tumor therapies, applicable for non-systemic use when desired, wherein the drug has for example an acceptable safety profile, little off-target activity, sufficient efficacy, sufficiently low clearance rate from the patient's body, a sufficiently wide therapeutic window, etc.

### SUMMARY OF THE INVENTION

A first aspect of the current invention relates to a conjugate comprising a single-domain antibody (sdAb) covalently bound to at least one saponin, directly or via a linker. The at least one saponin is typically a monodesmosidic triterpene glycoside or a bidesmosidic triterpene glycoside. Preferably, the conjugate is suitable for transferring a saponin from outside a cell into said cell, wherein the conjugate comprises the sdAb, which sdAb is capable of binding to said cell, and which sdAb is covalently bound to the at least one saponin, directly or via a linker, wherein the at least one saponin is a monodesmosidic triterpene glycoside or a bidesmosidic triterpene glycoside.

A second aspect of the invention relates to a pharmaceutical combination comprising:
a first pharmaceutical composition comprising the conjugate of the invention and optionally comprising a pharmaceutically acceptable excipient and/or a pharmaceutically acceptable diluent; and
a second pharmaceutical composition comprising an active pharmaceutical ingredient (API), and optionally comprising a pharmaceutically acceptable excipient and/or a pharmaceutically acceptable diluent. The API comprised by the second pharmaceutical composition is for example an API such as selected from any one or more of: a drug molecule, an oligonucleotide such as an mRNA, an anti-sense oligonucleotide, a ligand-drug conjugate such as EGF-dianthin or EGF-saporin, an antibody-drug conjugate (ADC) such as OKT9 monoclonal anti-CD71 antibody, trastuzumab or cetuximab conjugated with saporin, dianthin, a BNA, an antibody-oligonucleotide conjugate (AOC) such as an antibody-BNA conjugate or an antibody-siRNA conjugate, wherein the antibody of the ADC or the AOC optionally comprises or consists of at least one sdAb, wherein the at least one sdAb comprised by the ADC or AOC is/are different from or the same as the sdAb of the conjugate of the invention. It is part of the invention that the sdAb of the sdAb-saponin conjugate comprises a binding region for binding to a binding site on a cell surface. The sdAb can bind to a cell. When the API is any one of a ligand-drug conjugate, an ADC and an AOC, such an API can bind to the same cell via the ligand or antibody, as the cell to which the conjugate of sdAb and saponin can bind. Thus, the sdAb-saponin conjugate and the API bind to the same cell. The binding site on the same cell can be the same or can be different for the saponin-comprising conjugate and the ligand- or antibody comprising API. Typically, the binding site(s) on the same cell is/are (an) epitope on a cell-surface molecule such as a cell-surface receptor. Typical cell-surface receptors are CD71, EGFR, Her2, HIVgp41. For example, the sdAb comprised by the sdAb-saponin conjugate binds to a first epitope on a first cell-surface molecule such as a receptor and the API binds to the same first epitope or binds to a second epitope on the same first cell-surface molecule or on a second cell-surface molecule exposed on the same cell surface. Preferred API's are an ADC and an AOC, wherein such an ADC or AOC comprises preferably an antibody such as an IgG or an sdAb such as one or more V_{HH}'s.

A third aspect of the invention relates to a pharmaceutical composition comprising:
the conjugate of the invention;
one or more APIs, and
   optionally comprising a pharmaceutically acceptable excipient and/or a pharmaceutically acceptable diluent. The API comprised by the pharmaceutical composition is for example an API such as selected from: a drug molecule, an oligonucleotide such as an mRNA, an ASO, a ligand-drug conjugate such as EGF-dianthin or EGF-saporin, an ADC such as OKT9 monoclonal anti-CD71 antibody, trastuzumab or cetuximab conjugated with saporin, dianthin, a BNA, an AOC such as an antibody-BNA conjugate or an antibody-siRNA conjugate, wherein optionally the antibody of the ADC or the AOC comprises or consists of at least one sdAb, wherein the at least one sdAb comprised by the ADC or AOC is/are different from or the same as the sdAb of the conjugate of the invention. Again, if the API comprises a cell-binding moiety, domain, molecule, such as a ligand for a cell-surface molecule (receptor) such as for example EGF or a cytokine, or such as an antibody or a binding domain or binding fragment thereof, such as an IgG, a Fab. An scFv. a sdAb such as a Vh domain or a V_{HH}, the saponin-comprising conjugate and the API can bind to the same cell, wherein the saponin-comprising conjugate binds to the cell via the sdAb. Typically, the conjugate and the API bind to the same cell-surface molecule such as a receptor exposed at the cell surface, or bind to a first and second cell-surface molecule exposed on the same cell, for example a first and second cell-surface receptor which are different receptors on the same cell. Preferred API's are an ADC and an AOC, wherein such an ADC or AOC comprises preferably an antibody such as an IgG or an sdAb such as one or more V_{HH}'s.

A fourth aspect of the invention relates to the pharmaceutical combination of the invention, comprising the first pharmaceutical composition and comprising the second pharmaceutical composition, or to the pharmaceutical composition of the invention, for use as a medicament.

A fifth aspect of the invention relates to the pharmaceutical combination of the invention, comprising the first pharmaceutical composition and comprising the second pharmaceutical composition, or to the pharmaceutical composition of the invention, for use in the treatment or the prophylaxis of a cancer, an auto-immune disease, an infection such as a viral infection, an enzyme deficiency, a disorder or disease relating to an enzyme deficiency, a gene defect, a disorder or disease relating to a gene defect. Such treatment involves the binding of the sdAb comprised by the saponin-comprising conjugate to a first cell-surface molecule presented at the cell surface of the target cell involved in the disease or health condition to be treated, and involves the binding of an API comprising a ligand or an antibody such as an IgG or an sdAb for binding to the same first cell-surface molecule of the same cell or for binding to a second cell-surface molecule present on the same cell as at which the first cell-surface molecule is present, such that the saponin-comprising conjugate and the API can bind to the same cell such as a disease-related cell such as a tumor cell, etc. Thus, combinations of the saponin-comprising conjugate and the API are selected for their (simultaneous) binding to the same cell, either by binding to the same cell-surface molecule such as a receptor, or by binding to a first and second cell-surface molecule such as a first receptor and a second receptor, which are different but present at the same cell.

A sixth aspect of the invention relates to an in vitro or ex vivo method for transferring a molecule from outside a cell to inside said cell, preferably to the cytosol of said cell, comprising the steps of:
a) providing a cell which expresses a binding site for the sdAb comprised by the conjugate of the invention on its surface, according to embodiments of the invention, preferably selected from a liver cell, an aberrant cell such as a virally infected cell, an auto-immune cell and a tumor cell;
b) providing the molecule for transferring into the cell provided in step a), wherein the molecule is any one or more of (an) API(s) of embodiments of the invention;
c) providing the conjugate of the invention;
d) contacting the cell of step a) in vitro or ex vivo with the molecule of step b) and the conjugate of step c),
   therewith establishing the transfer of the molecule from outside the cell into said cell, preferably into the cytosol of said cell. It is to be understood that when the API comprises a ligand or antibody for binding to a cell-surface molecule, said cell-surface molecule is present at the same cell as at which the binding site for the saponin-comprising conjugate is present. The saponin-comprising conjugate and the API bind to the same cell. Either to the same cell-surface molecule exposed on the target cell, or to two different cell-surface molecules exposed on the same target cell in which the molecule comprised by the API (for example an effector moiety such as an oligonucleotide such as a BNA or a (protein) toxin) is to be transferred.

A seventh aspect of the invention relates to a kit of parts, comprising the pharmaceutical combination of the invention, comprising the first pharmaceutical composition and comprising the second pharmaceutical composition, or comprising the pharmaceutical composition of the invention, or comprising the conjugate of the invention, and optionally instructions for use of said pharmaceutical combination according to the invention, or optionally instructions for use of said pharmaceutical composition according to the invention, or optionally instructions for use of said conjugate according to the invention. The kit comprises the saponin-comprising conjugate for binding to a first cell-surface molecule of a selected target cell such as a selected tumor cell, and comprises for example an API comprising a ligand such as EGF or a cytokine, or an antibody such as an IgG or an sdAb, such as an ADC or an AOC, wherein the ligand or antibody can bind to the same first cell-surface molecule present on the target cell, or can bind to a second cell-surface molecule present on the same target cell as at which the first cell-surface molecule is present.

### Definitions

The term “proteinaceous” has its regular scientific meaning and here refers to a molecule that is protein-like, meaning that the molecule possesses, to some degree, the physicochemical properties characteristic of a protein, is of protein, relating to protein, containing protein, pertaining to protein, consisting of protein, resembling protein, or being a protein. The term “proteinaceous” as used in for example ‘proteinaceous molecule’ refers to the presence of at least a part of the molecule that resembles or is a protein, wherein ‘protein’ is to be understood to include a chain of amino-acid residues at least two residues long, thus including a peptide, a polypeptide and a protein and an assembly of proteins or protein domains. In the proteinaceous molecule, the at least two amino-acid residues are for example linked via (an) amide bond(s), such as (a) peptide bond(s). In the proteinaceous molecule, the amino-acid residues are natural amino-acid residues and/or artificial amino-acid residues such as modified natural amino-acid residues. In a preferred embodiment, a proteinaceous molecule is a molecule comprising at least two amino-acid residues, preferably between two and about 2.000 amino-acid residues. In one embodiment, a proteinaceous molecule is a molecule comprising from 2 to 20 (typical for a peptide) amino acids. In one embodiment, a proteinaceous molecule is a molecule comprising from 21 to 1.000 amino acids (typical for a polypeptide, a protein, a protein domain, such as an antibody, a Fab, an scFv, a ligand for a receptor such as EGF). Preferably, the amino-acid residues are (typically) linked via (a) peptide bond(s). According to the invention, said amino-acid residues are or comprise (modified) (non-)natural amino acid residues.

The term “effector molecule”, or “effector moiety” when referring to the effector molecule as part of e.g. a covalent conjugate, has its regular scientific meaning and here refers to a molecule that can selectively bind to for example any one or more of the target molecules: a protein, a peptide, a carbohydrate, a saccharide such as a glycan, a (phospho)lipid, a nucleic acid such as DNA, RNA, an enzyme, and regulates the biological activity of such one or more target molecule(s). In the conjugate of the invention the effector moiety for example exerts its effect in the cytosol, in the cell nucleus, is delivered intracellularly in the endosome and/or lysosome, and/or is active after exiting or escaping the endosomal-lysosomal pathway. The effector molecule is for example a molecule selected from any one or more of a small molecule such as a drug molecule, a toxin such as a protein toxin, an oligonucleotide such as a BNA, a xeno nucleic acid or an siRNA, an enzyme, a peptide, a protein, or an active fragment or an active domain thereof, or any combination thereof. Thus, for example, an effector molecule or an effector moiety is a molecule or moiety selected from any one or more of a small molecule such as a drug molecule, a toxin such as a protein toxin, an oligonucleotide such as a BNA, a xeno nucleic acid or an siRNA, an enzyme, a peptide, a protein, or any combination thereof, that can selectively bind to any one or more of the target molecules: a protein, a peptide, a carbohydrate, a saccharide such as a glycan, a (phospho)lipid, a nucleic acid such as DNA, RNA, an enzyme, and that upon binding to the target molecule regulates the biological activity of such one or more target molecule(s). For example, an effector moiety is a toxin or an active toxic fragment thereof or an active toxic derivative or an active toxic domain thereof. Typically, an effector molecule can exert a biological effect inside a cell such as a mammalian cell such as a human cell, such as in the cytosol of said cell. An effector molecule or moiety of the invention is thus any substance that affects the metabolism of a cell by interaction with an intracellular effector molecule target, wherein this effector molecule target is any molecule or structure inside cells excluding the lumen of compartments and vesicles of the endocytic and recycling pathway but including the membranes of these compartments and vesicles. Said structures inside cells thus include the nucleus, mitochondria, chloroplasts, endoplasmic reticulum, Golgi apparatus, other transport vesicles, the inner part of the plasma membrane and the cytosol. Typical effector molecules are thus drug molecules, an enzyme, plasmid DNA, toxins such as toxins comprised by antibody-drug conjugates (ADCs), oligonucleotides such as siRNA, BNA, nucleic acids comprised by an antibody-oligonucleotide conjugate (AOC). For example, an effector molecule is a molecule which can act as a ligand that can increase or decrease (intracellular) enzyme activity, gene expression, or cell signalling. In the context of the invention, an effector molecule or effector moiety when the effector molecule is part of a conjugate, is not a saponin, and is not a cell-surface molecule binding molecule such as an antibody such as an sdAb. Typically, an effector moiety comprised by the conjugate exerts its therapeutic (for example toxic, enzymatic, inhibitory, gene silencing, etc.) effect in the cytosol and/or in the cell nucleus. Typically, the effector moiety is delivered intracellularly in the endosome and/or in the lysosome, and typically the effector moiety is active after exiting or escaping the endosomal-lysosomal pathway.

The term “saponin” has its regular scientific meaning and here refers to a group of amphipatic glycosides which comprise one or more hydrophilic glycone moieties combined with a lipophilic aglycone core which is a sapogenin. The saponin may be naturally occurring or synthetic (i.e. non-naturally occurring). The term “saponin” includes naturally-occurring saponins, functional derivatives of naturally-occurring saponins as well as saponins synthesized de novo through chemical and/or biotechnological synthesis routes. Saponin has a triterpene backbone, which is a pentacyclic C30 terpene skeleton, also referred to as sapogenin or aglycone. Within the context of the invention saponin is not considered an effector molecule nor an effector moiety, for example when being part of a conjugate. Thus, in the conjugates comprising a saponin, the saponin is not an effector moiety.

The term “modified saponin” has its regular scientific meaning and here refers to a saponin, i.e. a saponin functional derivative, which has one or more chemical modifications at positions where previously any of an aldehyde group, a carboxyl group, an acetate group and/or an acetyl group was present in the non-derivatised saponin before being subjected to chemical modification for provision of the modified saponin. For example, the modified saponin is provided by chemical modification of any one or more of an aldehyde group, a carboxyl group, an acetate group and/or an acetyl group in a saponin upon which the modified saponin is based, i.e. the saponin is provided and any of an aldehyde group, a carboxyl group, an acetate group and/or an acetyl group is chemically modified therewith providing the modified saponin. For example, the saponin that is modified for provision of the modified saponin is a naturally occurring saponin. Typically, the modified saponin is a synthetic saponin, typically the modified saponin is a modification of a natural saponin, and is thus derived from a natural saponin, although a modified saponin can also be derived from a synthetic saponin which may or may not have a natural counterpart. Typically, the modified saponin has not a natural counterpart, i.e. the modified saponin is not produced naturally by e.g. plants or trees.

The term “saponin derivative” (also known as “modified saponin” and saponin functional derivative) has its regular scientific meaning and here refers to a compound corresponding to a naturally-occurring saponin which has been derivatised by one or more chemical modifications, such as the oxidation of a functional group, the reduction of a functional group and/or the formation of a covalent bond with another molecule (also referred to as “conjugation” or as “covalent conjugation”). Preferred modifications include derivatisation of an aldehyde group of the aglycone core; of a carboxyl group of a saccharide chain or of an acetoxy group of a saccharide chain. Typically, the saponin derivative is a functional saponin and does not have a natural counterpart, i.e. the saponin derivative is not produced naturally by e.g. plants or trees. The term “saponin derivative” includes derivatives obtained by derivatisation of naturally-occurring saponins as well as derivatives synthesized de novo through chemical and/or biotechnological synthesis routes resulting in a compound corresponding to a naturally-occurring saponin which has been derivatised by one or more chemical modifications. The term “derivative” in the context of a saponin should be understood as a functional derivative. “Functional” in the context of the saponin derivative is understood as the capacity or activity of the saponin or the saponin derivative to enhance the endosomal escape of an effector molecule which is contacted with a cell together with the saponin or the saponin derivative.

The term “aglycone core structure” has its regular scientific meaning and here refers to the aglycone core of a saponin without the one or two carbohydrate antenna or saccharide chains (glycans) bound thereto. For example, quillaic acid is the aglycone core structure for SO1861, QS-7 and QS21. Typically, the glycans of a saponin are mono-saccharides or oligo-saccharides, such as linear or branched glycans.

The term “saccharide chain” has its regular scientific meaning and here refers to any of a glycan, a carbohydrate antenna, a single saccharide moiety (mono saccharide) or a chain comprising multiple saccharide moieties (oligosaccharide, polysaccharide). The saccharide chain can consist of only saccharide moieties or may also comprise further moieties such as any one of 4E-Methoxycinnamic acid, 4Z-Methoxycinnamic acid, and 5-O-[5-O-Ara/Api-3,5-dihydroxy-6-methyl-octanoyl]-3,5-dihydroxy-6-methyl-octanoic acid), such as for example present in QS-21.

The term “chemically modified” has its regular scientific meaning and here refers to the chemical modification of a first chemical group or first chemical moiety such that a second chemical group or second chemical moiety is provided. Examples are the chemical modification of a carbonyl group into a —(H)C—OH group, the chemical modification of an acetate group into a hydroxyl group, the provision of a saponin conjugated at its aldehyde group with an N-ε-maleimidocaproic acid hydrazide (EMCH) moiety via a chemical reaction, etc.

The term “chemically modified aldehyde group” has its regular scientific meaning and here refers to the chemical reaction product obtained by the chemical reaction involving the aldehyde group of a saponin resulting in replacement of the initial aldehyde group by a new chemical group. For example, the formation of a —(H)C—OH group from the initial aldehyde group of a saponin.

The term “chemically modified carboxyl group” has its regular scientific meaning and here refers to the chemical reaction product obtained by the chemical reaction involving the carboxyl group of a saponin, such as the carboxyl group of a glucuronic acid moiety, and a further molecule, resulting in replacement of the initial carboxyl group by a new chemical group. For example, the formation of the conjugate between a saponin and any one of 2-amino-2-methyl-1,3-propanediol (AMPD), N-(2-aminoethyl)maleimide (AEM) or 1-[Bis(dimethylamino)methylene]-1H-1,2,3-triazolo[4,5-b]pyridinium 3-oxid hexafluorophosphate (HATU), involving the carboxyl group of the glucuronic acid of the saponin.

The term “Api/Xyl-” or “Api- or Xyl-” in the context of the name of a saccharide chain has its regular scientific meaning and here refers to the saccharide chain either comprising an apiose (Api) moiety, or comprising a xylose (Xyl) moiety.

The term “modified saponin based on a saponin” has its regular scientific meaning and here refers to a saponin that has been subjected to a chemical modification step such that the modified saponin is provided, wherein the saponin from which the modified saponin has been made is typically a naturally occurring saponin.

The term “oligonucleotide” has its regular scientific meaning and here refers to amongst others any natural or synthetic string of nucleic acids encompassing DNA, modified DNA, RNA, mRNA, modified RNA, synthetic nucleic acids, presented as a single-stranded molecule or a double-stranded molecule, such as a BNA, an antisense oligonucleotide (ASO, AON), a short or small interfering RNA (siRNA; silencing RNA), an anti-sense DNA, anti-sense RNA, etc. The term “oligonucleotide” here also refers to a string of two or more nucleotides, i.e., an oligonucleotide is a short oligomer composed of ribonucleotides or deoxyribonucleotides. Examples are RNA and DNA, and any modified RNA or DNA, such as a string of nucleic acids comprising a nucleotide analogue such as a bridged nucleic acid (BNA), also known as locked nucleic acid (LNA) or a 2′-O,4′-C-aminoethylene or a 2′-O,4′-C-aminomethylene bridged nucleic acid (BNA^{NC}), wherein the nucleotide is a ribonucleotide or a deoxyribonucleotide. The term “antibody-drug conjugate” or “ADC” has its regular scientific meaning and here refers to any conjugate of an antibody such as an IgG, a Fab, an scFv, an immunoglobulin, an immunoglobulin fragment, one or multiple V_{H} domains, single-domain antibodies, a V_{HH}, a camelid V_{H}, etc., and any molecule that can exert a therapeutic effect when contacted with cells of a subject such as a human patient, such as an active pharmaceutical ingredient, a toxin, an oligonucleotide, an enzyme, a small molecule drug compound, etc.

The term “antibody-oligonucleotide conjugate” or “AOC” has its regular scientific meaning and here refers to any conjugate of an antibody such as an IgG, a Fab, an scFv, an immunoglobulin, an immunoglobulin fragment, one or multiple V_{H} domains, single-domain antibodies, a V_{HH}, a camelid V_{H}, etc., and any oligonucleotide molecule that can exert a therapeutic effect when contacted with cells of a subject such as a human patient, such as an oligonucleotide selected from a natural or synthetic string of nucleic acids encompassing DNA, modified DNA, RNA, mRNA, modified RNA, synthetic nucleic acids, presented as a single-stranded molecule or a double-stranded molecule, such as a BNA, an antisense oligonucleotide (ASO), a short or small interfering RNA (siRNA; silencing RNA), an anti-sense DNA, anti-sense RNA, etc.

The term “bridged nucleic acid”, or “BNA” also known as locked nucleic acid (LNA) or a 2′-O,4′-C-aminoethylene or a 2′-O,4′-C-aminomethylene bridged nucleic acid (BNA^{NC}), has its regular scientific meaning and here refers to a modified RNA nucleotide. A BNA is also referred to as ‘constrained RNA molecule’ or ‘inaccessible RNA molecule’. A BNA monomer can contain a five-membered, six-membered or even a seven-membered bridged structure with a “fixed” C3′-endo sugar puckering. The bridge is synthetically incorporated at the 2′, 4′-position of the ribose to afford a 2′, 4′-BNA monomer. A BNA monomer can be incorporated into an oligonucleotide polymeric structure using standard phosphoramidite chemistry known in the art. A BNA is a structurally rigid oligonucleotide with increased binding affinity and stability.

The term “single domain antibody”, or “sdAb”, in short, has its regular scientific meaning and here refers to an antibody fragment consisting of a single monomeric variable antibody domain. In the conjugates of the invention, more than one sdAb can be present, which sdAbs can be the same (multivalent and mono-specific) or can be different (multivalent and/or for example multi-paratope, bi-paratope, multi-specific, bi-specific). In addition, the more than two sdAbs can for example be a combination of mono-specific and multivalent sdAbs and at least one further sdAb that binds to a different epitope (e.g. multi-specific or bi-paratope).

The term ‘S’ as used such as in an antibody-saponin conjugate comprising a linker, represents ‘stable linker’ which remains intact in the endosome, endolysosome and in the lysosome of mammalian cells, such as human cells, such as a human tumor cell, thus under slightly acid conditions (pH<6.6, such as pH 4.0-5.5).

The term ‘L’ as used such as in an antibody-saponin conjugate comprising a linker, represents ‘labile linker’ which is cleaved under slightly acid conditions (pH<6.6, such as pH 4.0-5.5) in the endosome, endolysosome and in the lysosome of mammalian cells, such as human cells, such as a human tumor cell.

The terms first, second, third and the like in the description and in the claims, are used for distinguishing between for example similar elements, compositions, constituents in a composition, or separate method steps, and not necessarily for describing a sequential or chronological order. The terms are interchangeable under appropriate circumstances and the embodiments of the invention can operate in other sequences than described or illustrated herein, unless specified otherwise.

The embodiments of the invention described herein can operate in combination and cooperation, unless specified otherwise.

Furthermore, the various embodiments, although referred to as “preferred” or “e.g.” or “for example” or “in particular” and the like are to be construed as exemplary manners in which the invention may be implemented rather than as limiting the scope of the invention.

The term “comprising”, used in the claims, should not be interpreted as being restricted to for example the elements or the method steps or the constituents of a compositions listed thereafter; it does not exclude other elements or method steps or constituents in a certain composition. It needs to be interpreted as specifying the presence of the stated features, integers, (method) steps or components as referred to, but does not preclude the presence or addition of one or more other features, integers, steps or components, or groups thereof. Thus, the scope of the expression “a method comprising steps A and B” should not be limited to a method consisting only of steps A and B, rather with respect to the present invention, the only enumerated steps of the method are A and B, and further the claim should be interpreted as including equivalents of those method steps. Thus, the scope of the expression “a composition comprising components A and B” should not be limited to a composition consisting only of components A and B, rather with respect to the present invention, the only enumerated components of the composition are A and B, and further the claim should be interpreted as including equivalents of those components.

In addition, reference to an element or a component by the indefinite article “a” or “an” does not exclude the possibility that more than one of the element or component are present, unless the context clearly requires that there is one and only one of the elements or components. The indefinite article “a” or “an” thus usually means “at least one”.

The term “Saponinum album” has its normal meaning and here refers to a mixture of saponins produced by Merck KGaA (Darmstadt, Germany) containing saponins from Gypsophila paniculata and Gypsophila arostii, containing SA1657 and mainly SA1641.

The term “Quillaja saponin” has its normal meaning and here refers to the saponin fraction of Quillaja saponaria and thus the source for all other QS saponins, mainly containing QS-18 and QS-21.

“QS-21” or “QS21” has its regular scientific meaning and here refers to a mixture of QS-21 A-apio (˜63%), QS-21 A-xylo (˜32%), QS-21 B-apio (˜3.3%), and QS-21 B-xylo (˜1.7%).

Similarly, “QS-21A” has its regular scientific meaning and here refers to a mixture of QS-21 A-apio (˜65%) and QS-21 A-xylo (˜35%).

Similarly, “QS-21 B” has its regular scientific meaning and here refers to a mixture of QS-21 B-apio (˜65%) and QS-21 B-xylo (˜35%).

The term “Quil-A” refers to a commercially available semi-purified extract from Quillaja saponaria and contains variable quantities of more than 50 distinct saponins, many of which incorporate the triterpene-trisaccharide substructure Gal-(1→2)-[Xyl-(1→3)]-GlcA- at the C-3beta-OH group found in QS-7, QS-17, QS18, and QS-21. The saponins found in Quil-A are listed in van Setten (1995), Table 2 [Dirk C. van Setten, Gerrit van de Werken, Gijsbert Zomer and Gideon F. A. Kersten, Glycosyl Compositions and Structural Characteristics of the Potential Immuno-adjuvant Active Saponins in the Quillaja saponaria Molina Extract Quil A, RAPID COMMUNICATIONS IN MASS SPECTROMETRY, VOL. 9,660-666 (1995)]. Quil-A and also Quillaja saponin are fractions of saponins from Quillaja saponaria and both contain a large variety of different saponins with largely overlapping content. The two fractions differ in their specific composition as the two fractions are gained by different purification procedures.

The term “QS1861” and the term “QS1862” refer to QS-7 and QS-7 api. QS1861 has a molecular mass of 1861 Dalton, QS1862 has a molecular mass of 1862 Dalton. QS1862 is described in Fleck et al. (2019) in Table 1, row no. 28 [Juliane Deise Fleck, Andresa Heemann Betti, Francini Pereira da Silva, Eduardo Artur Troian, Cristina Olivaro, Fernando Ferreira and Simone Gasparin Verza, Saponins from Quillaja saponaria and Quillaja brasiliensis: Particular Chemical Characteristics and Biological Activities, Molecules 2019, 24, 171; doi:10.3390/molecules24010171]. The described structure is the api-variant QS1862 of QS-7. The molecular mass is 1862 Dalton as this mass is the formal mass including proton at the glucuronic acid. At neutral pH, the molecule is deprotonated. When measuring in mass spectrometry in negative ion mode, the measured mass is 1861 Dalton.

### BRIEF DESCRIPTION OF THE DRAWINGS

FIG. 1A (FIG. 1A): The targeted 2-component approach (1 target). SO1861 and toxin (ribosomal inactivating protein) are each, separately, conjugated to a V_{HH} or antibody (mAb) for delivery and internalization into target cells. 1) mAb-toxin and V_{HH}-SO1861 bind to the cell surface receptor, 2) receptor-mediated endocytosis of both conjugates occurs (binding of conjugates to the receptor is followed by internalization of the conjugate/receptor complex), 3) at low endolysosomal pH and appropriate concentration, SO1861 becomes active to enable endolysosomal escape, 4) release of toxin into cytoplasm occurs and induces cell death.

FIG. 1B (FIG. 1B): The targeted 2-component approach (2 targets). SO1861 and toxin (ribosomal inactivating protein) are each, separately, conjugated to a V_{HH} or antibody (mAb) for delivery and internalization into target cells. 1) mAb-toxin and V_{HH}-SO1861 bind to their corresponding cell surface receptor, 2) receptor-mediated endocytosis of both conjugates occurs (binding of conjugates to the receptor is followed by internalization of the conjugate/receptor complex), 3) at low endolysosomal pH and appropriate concentration, SO1861 becomes active to enable endolysosomal escape, 4) release of toxin into cytoplasm occurs and induces cell death.

FIG. 1C (FIG. 1C): The targeted 2-component approach (2 targets). SO1861 and toxin (ribosomal inactivating protein) are each, separately, conjugated to a V_{HH} for delivery and internalization into target cells. 1) V_{HH}1-toxin and V_{HH}2-SO1861 bind to their corresponding cell surface receptor, 2) receptor-mediated endocytosis of both conjugates occurs (binding of conjugates to the receptor is followed by internalization of the conjugate/receptor complex), 3) at low endolysosomal pH and appropriate concentration, SO1861 becomes active to enable endolysosomal escape, 4) release of toxin into cytoplasm occurs and induces cell death.

FIG. 10 (FIG. 10): The targeted 2-component approach (2 targets). SO1861 and toxin (ribosomal inactivating protein) are each, separately, conjugated to a V_{HH} or mAb for delivery and internalization into target cells. 1) V_{HH}-toxin and mAb-SO1861 bind to their corresponding cell surface receptor, 2) receptor-mediated endocytosis of both conjugates occurs (binding of conjugates to the receptor is followed by internalization of the conjugate/receptor complex), 3) at low endolysosomal pH and appropriate concentration, SO1861 becomes active to enable endolysosomal escape, 4) release of toxin into cytoplasm occurs and induces cell death.

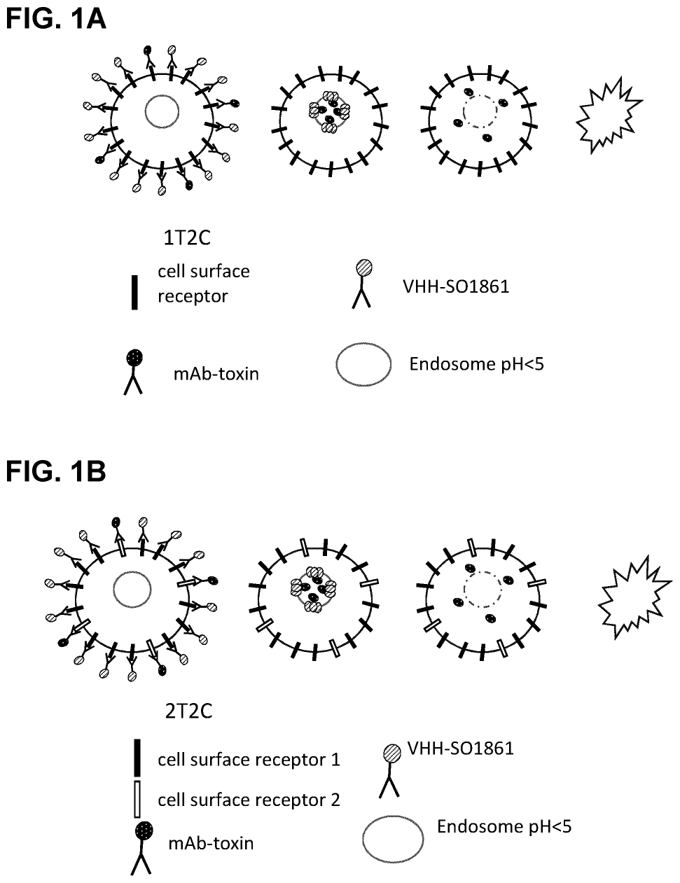

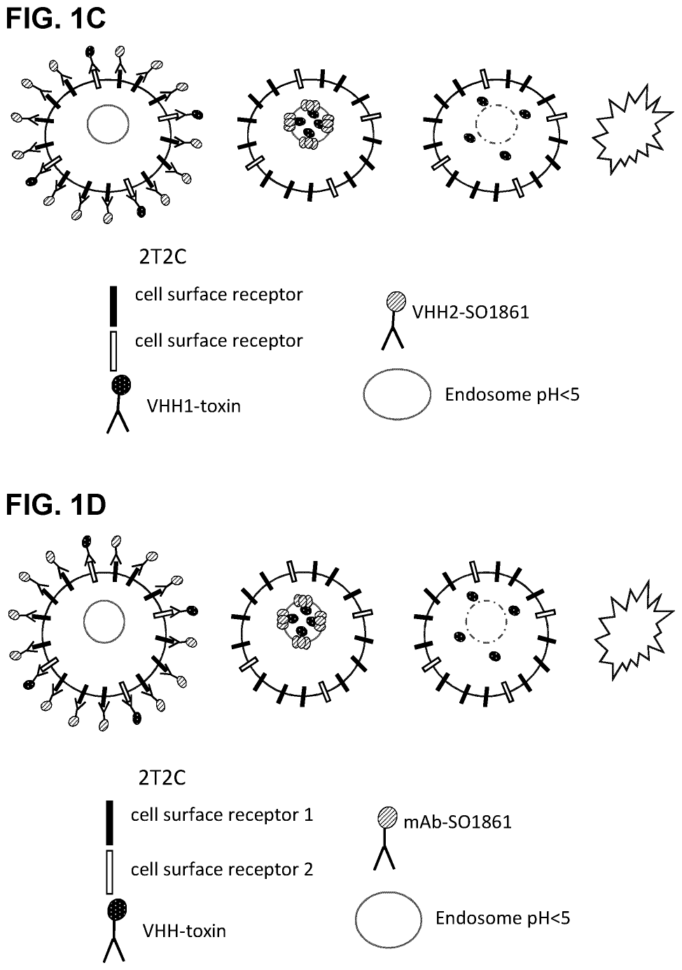

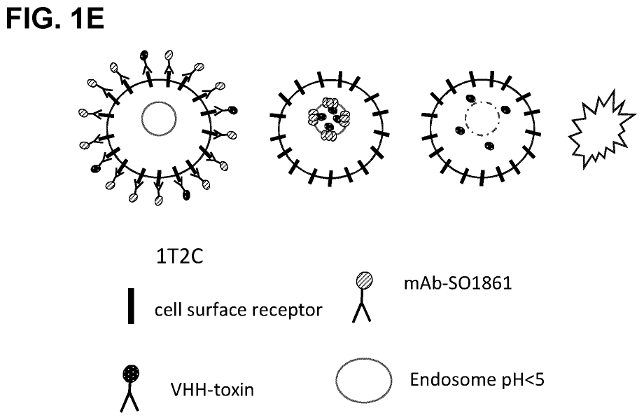

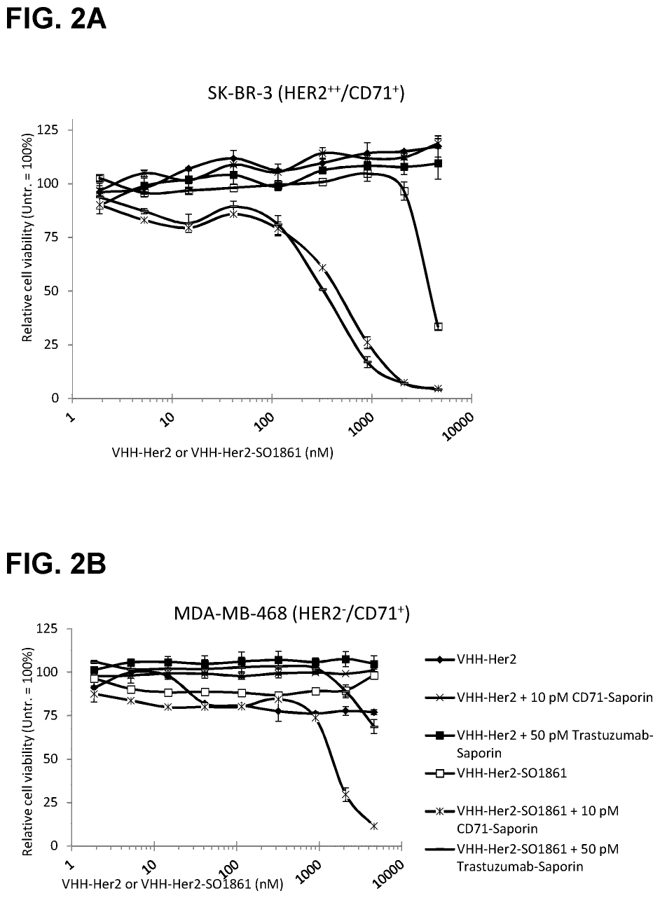

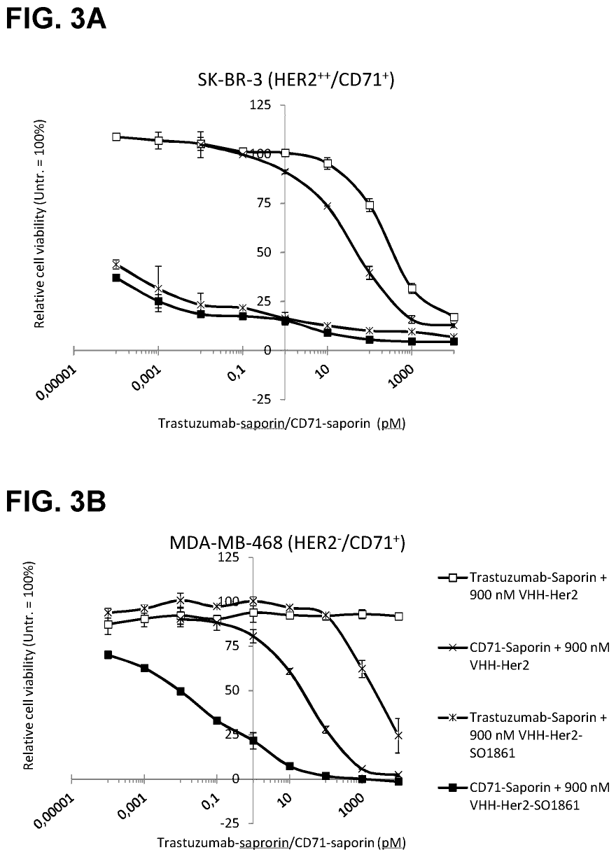

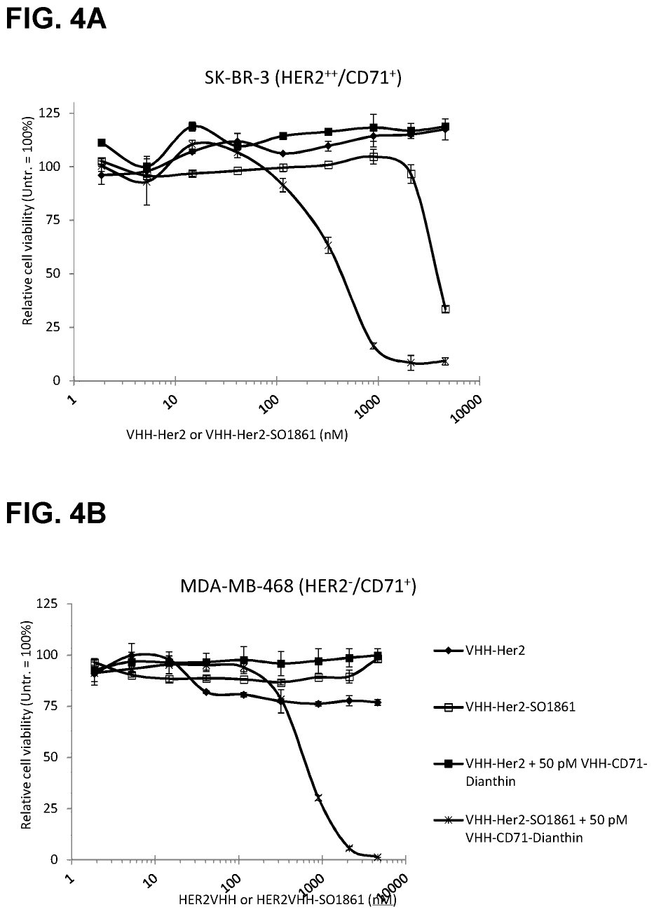

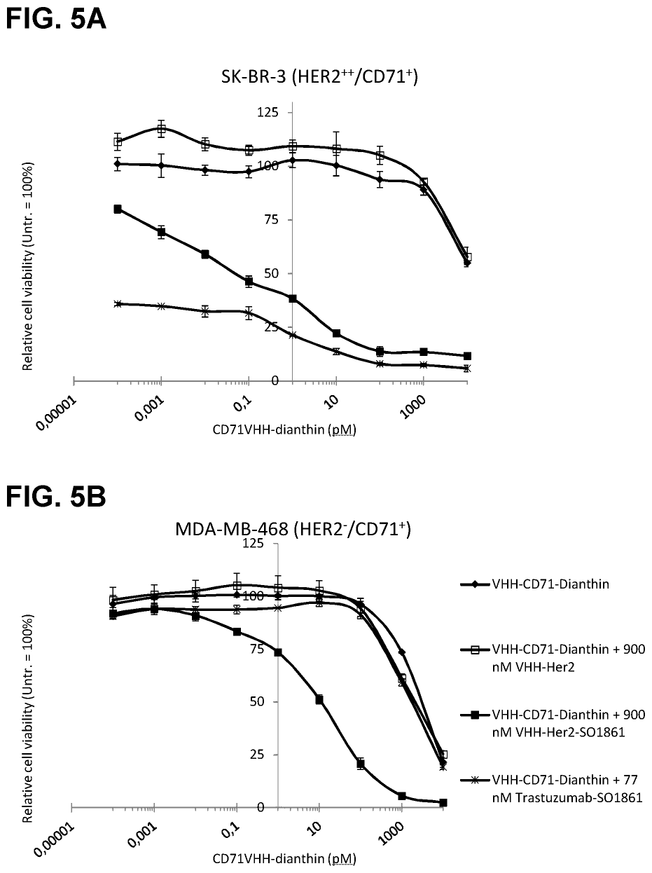

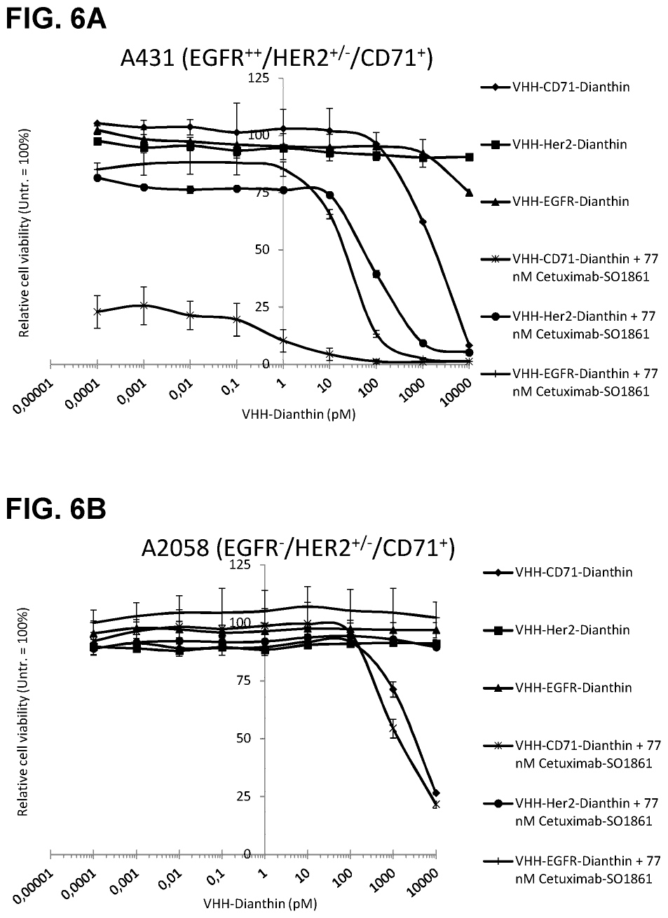

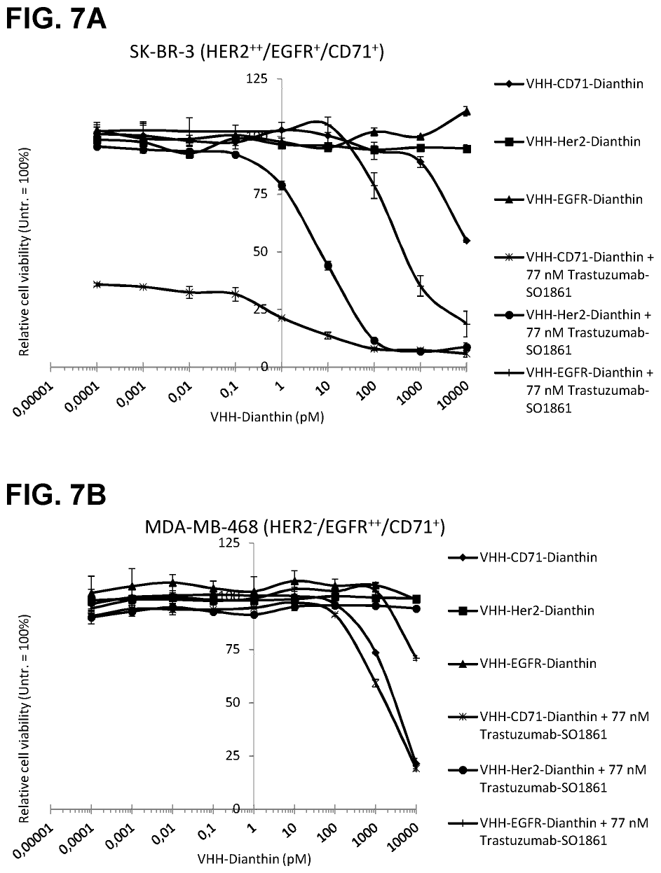

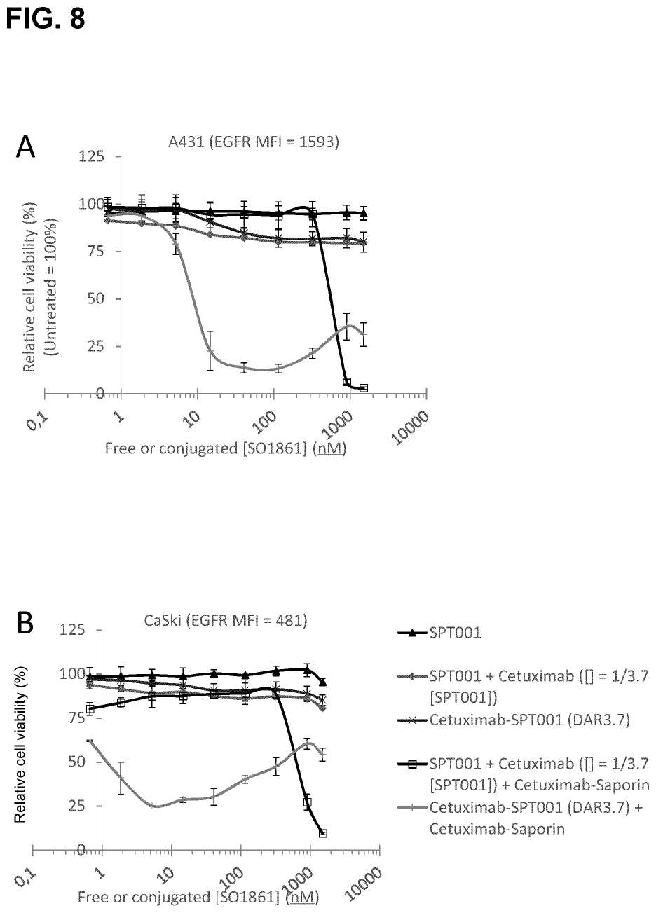

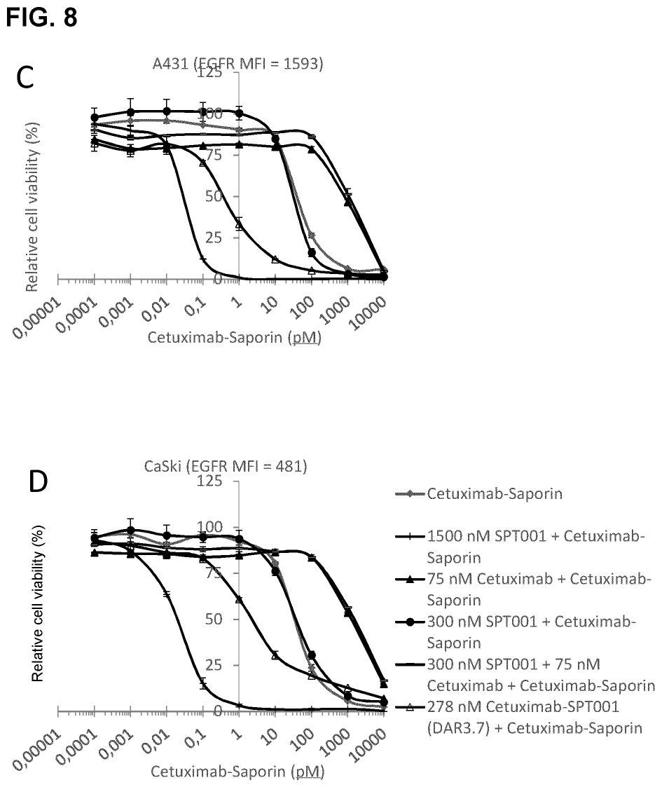

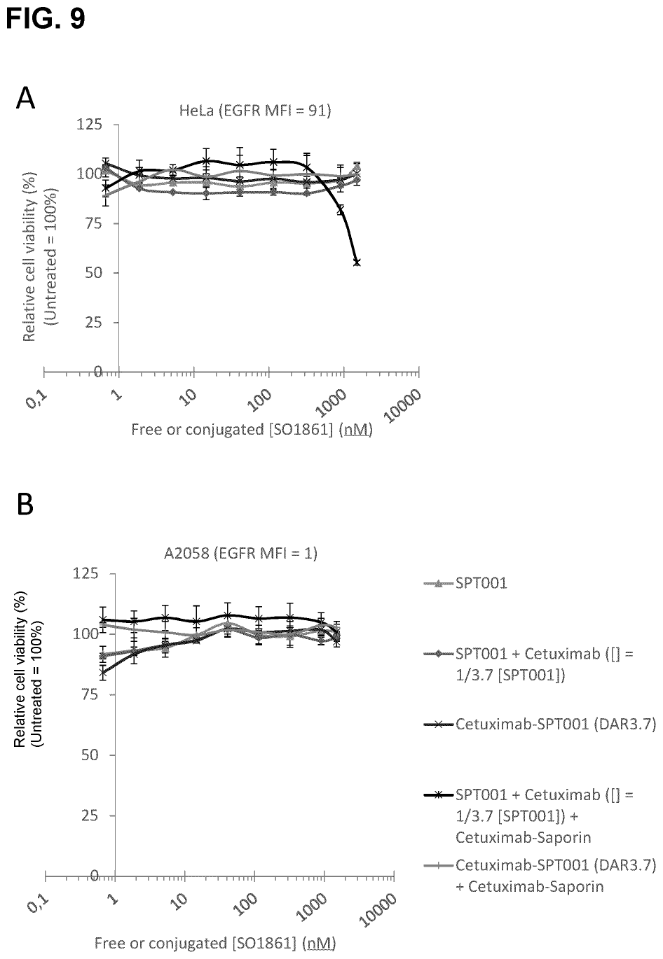

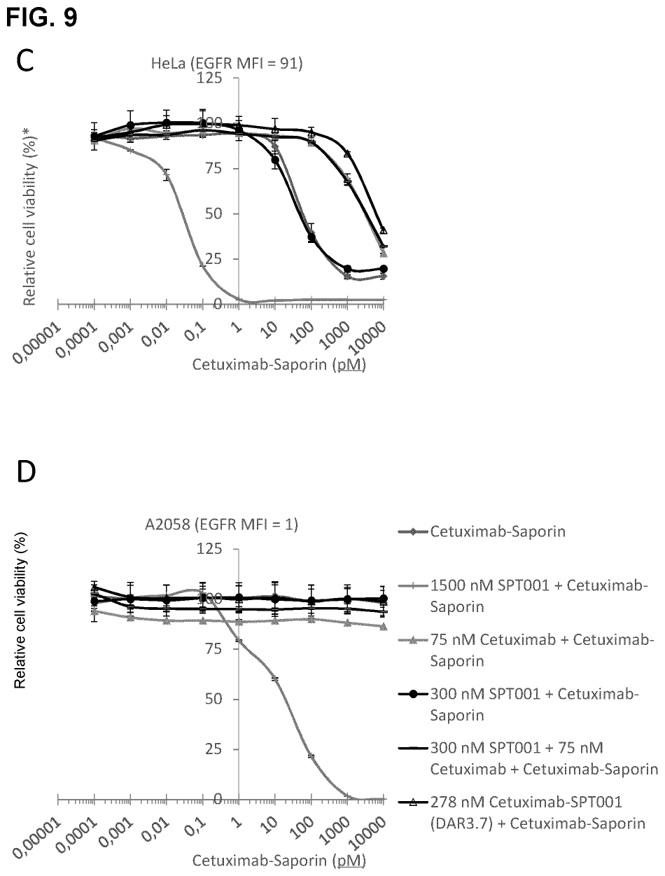

FIG. 1E (FIG. 1E): The targeted 2-component approach (1 target). SO1861 and toxin (ribosomal inactivating protein) are each, separately, conjugated to a V_{HH} or antibody (mAb) for delivery and internalization into target cells. 1) V_{HH}-toxin and mAb-SO1861 bind to the cell surface receptor, 2) receptor-mediated endocytosis of both conjugates occurs (binding of conjugates to the receptor is followed by internalization of the conjugate/receptor complex), 3) at low endolysosomal pH and appropriate concentration, SO1861 becomes active to enable endolysosomal escape, 4) release of toxin into cytoplasm occurs and induces cell death.

FIG. 2 (FIG. 2): Cell killing (MTS) assay) with the combination treatment according to the invention of V_{HH}(HER2)-SO1861 (DAR1)+50 pM trastuzumab-saporin (DAR4) or 10 pM CD71mab-saporin (DAR4) on SK-BR-3 cells (HER2⁺⁺/CD71⁺) (A) and MD-MB-468 cells (HER2⁻/CD71⁺) (B).

FIG. 3 (FIG. 3): Cell killing (MTS) assay) with the combination treatment according to the invention of Trastuzumab-saporin (DAR4) or CD71-saporin (DAR4)+900 nM HER2V_{HH}-SO1861 (DAR1) on SK-BR-3 cells (HER2++/CD71-9 (A) and MD-MB-468 cells (HER2⁻/CD71⁺) (B).

FIG. 4 (FIG. 4): Cell killing (MTS) assay) with the combination treatment according to the invention of V_{HH}(HER2)-SO1861 (DAR1)+50 pM CD71V_{HH}-dianthin (DAR1) on SK-BR-3 cells (HER2⁺⁺/CD71-9 (A) and MD-MB-468 cells (HER2⁻/CD71⁺) (B).

FIG. 5 (FIG. 5): Cell killing (MTS) assay) with the combination treatment according to the invention of CD71V_{HH}-dianthin (DAR1)+900 nM HER2V_{HH}-SO1861 (DAR1) on SK-BR-3 cells (HER2++/CD71-9 (A) and MD-MB-468 cells (HER2⁻/CD71⁺) (B).

FIG. 6 (FIG. 6): Cell killing (MTS) assay) with the combination treatment according to the invention of CD71V_{HH}-dianthin (DAR1)+cetuximab-SO1861 (DAR4) or HER2V_{HH}-dianthin (DAR1)+cetuximab-SO1861 (DAR4) or EGFRV_{HH}-dianthin (DAR1)+cetuximab-SO1861 (DAR4) on A431 cells (EGFR⁺⁼/HER^{+/-}/CD71-9 (A) and A2058 cells (EGFR⁻/HER^{+/-}/CD71⁺) (B).

FIG. 7 (FIG. 7): Cell killing (MTS) assay) with the combination treatment according to the invention of CD71V_{HH}-dianthin (DAR1)+77 nM cetuximab-SO1861 (DAR4) or HER2V_{HH}-dianthin (DAR1)+77 nM cetuximab-SO1861 (DAR4) or EGFRV_{HH}-dianthin (DAR1)+77 nM cetuximab-SO1861 (DAR4) on A431 cells (EGFR⁺⁼/HER^{+/-}/CD71-9 (A) and MDA-MB-468 cells (HER2⁻/EGFR⁺⁺/CD71⁺) (B).

FIG. 8. (FIG. 8): 1-target 2-component (EGFR high expression). EGFR targeted cell killing in A431 (EGFR⁺⁺⁺) and CaSKi (EGFR⁺⁺), by a therapeutic combination. A, B) cetuximab-SO1861 titration in combination with a fixed concentration of 10 pM cetuximab-saporin shows that a 100-400 fold reduced concentration of conjugated SO1861 is required, versus unconjugated SO1861, to induce cell killing by cetuximab-saporin. C, D), Cetuximab-saporin titration in combination with 278 nM cetuximab-SO1861 kills cells in contrast to 300 nM unconjugated SO1861+cetuximab-saporin. 1500 nM unconjugated SO1861+cetuximab-saporin is more efficient compared to the therapeutic combination, since both cetuximab conjugates compete for the same EGFR receptor. Only simultaneous targeted delivery of both cetuximab conjugates leads to efficient cell-killing, in contrast to monotherapy with either conjugate alone. REMARK: the legend displayed in FIG. 8B also relates to the graphs in FIG. 8A. REMARK: the legend displayed in FIG. 8D also relates to the graphs in FIG. 8C.

FIG. 9 (FIG. 9): 1-target 2-component (EGFR no/low expression). EGFR targeted cell killing in HeLa (EGFR⁺) and A2058 (EGFR⁻) cells, by a therapeutic combination. A, B) cetuximab-SO1861 titration in combination with a fixed concentration of 10 pM cetuximab-saporin does not induce cell killing by cetuximab-saporin. C, D), Cetuximab-saporin titration in combination with 278 nM cetuximab-SO1861 cannot induce cell killing. Low EGFR receptor expression is prohibitive for sufficient SO1861 to be delivered via antibody-mediated delivery, while 1500 nM of unconjugated SO1861 induces cell killing. REMARK: the legend displayed in FIG. 9B also relates to the graphs in FIG. 9A. REMARK: the legend displayed in FIG. 9D also relates to the graphs in FIG. 9C.

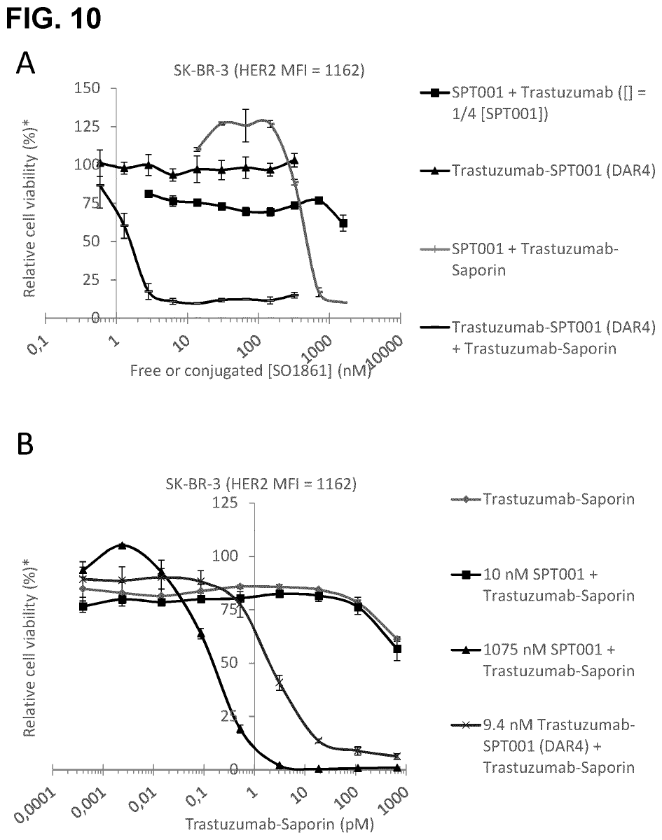

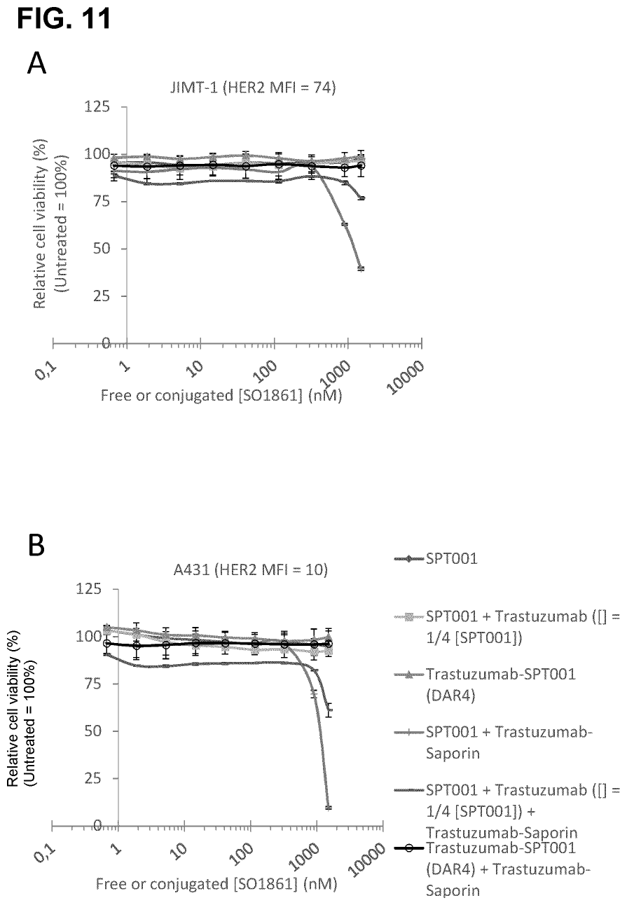

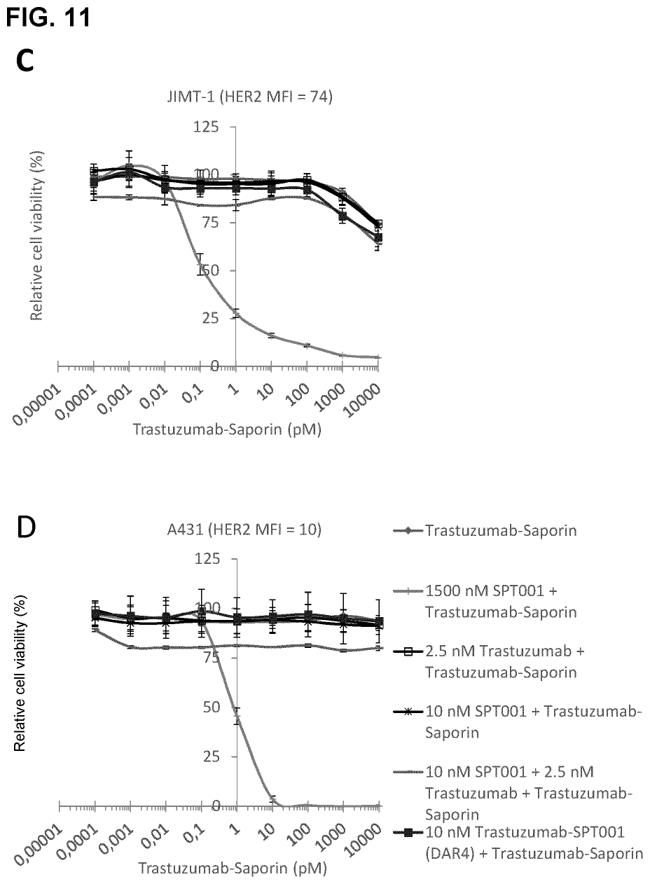

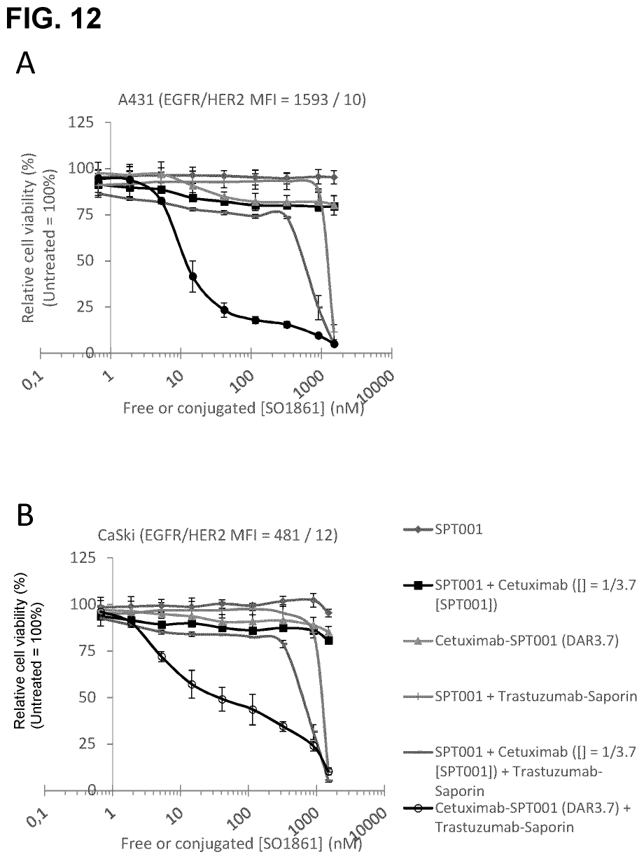

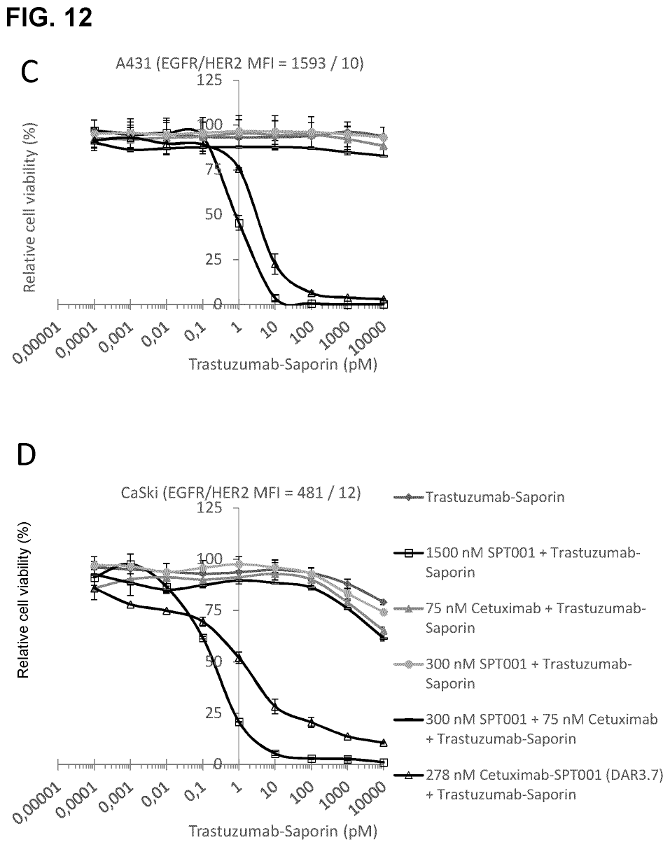

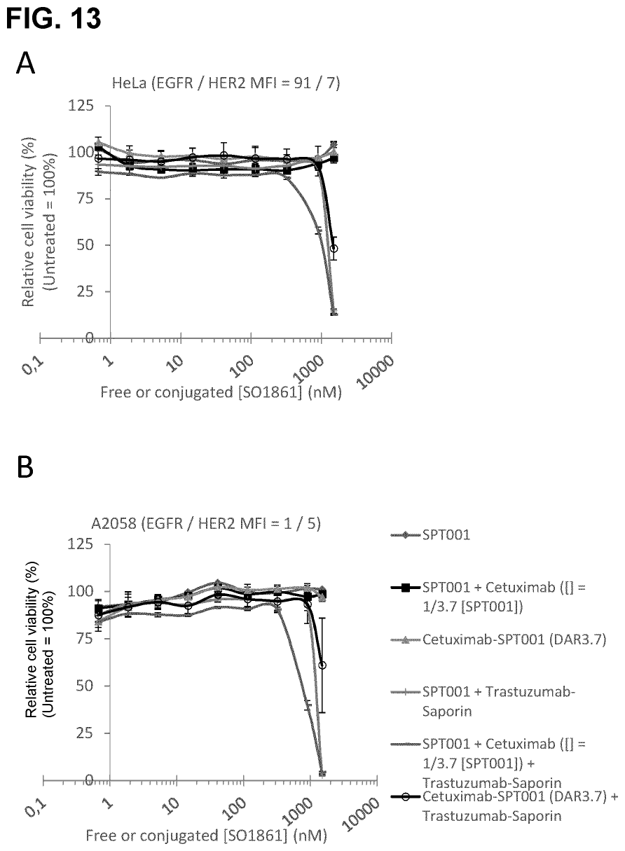

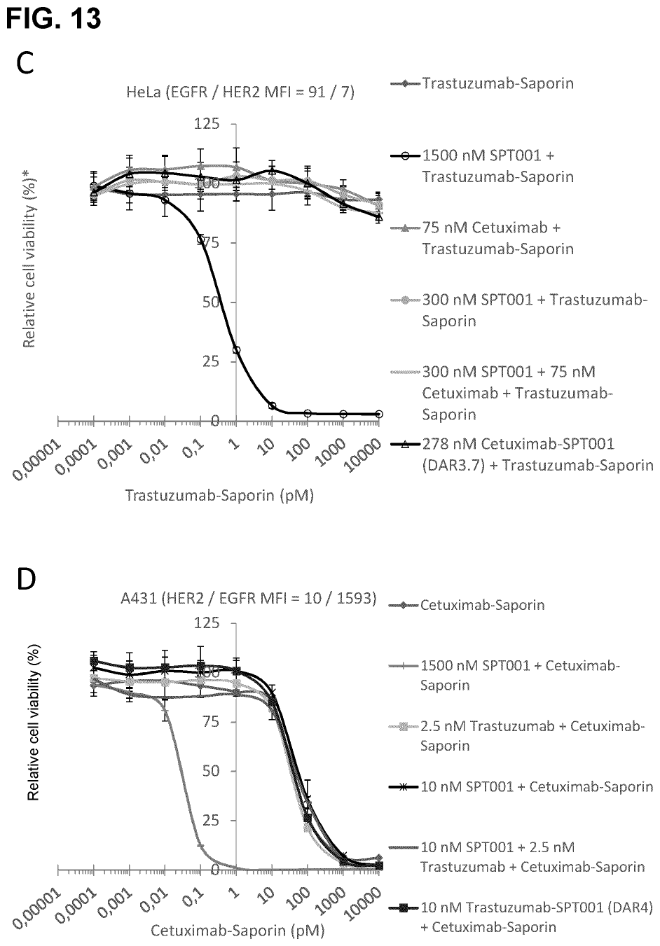

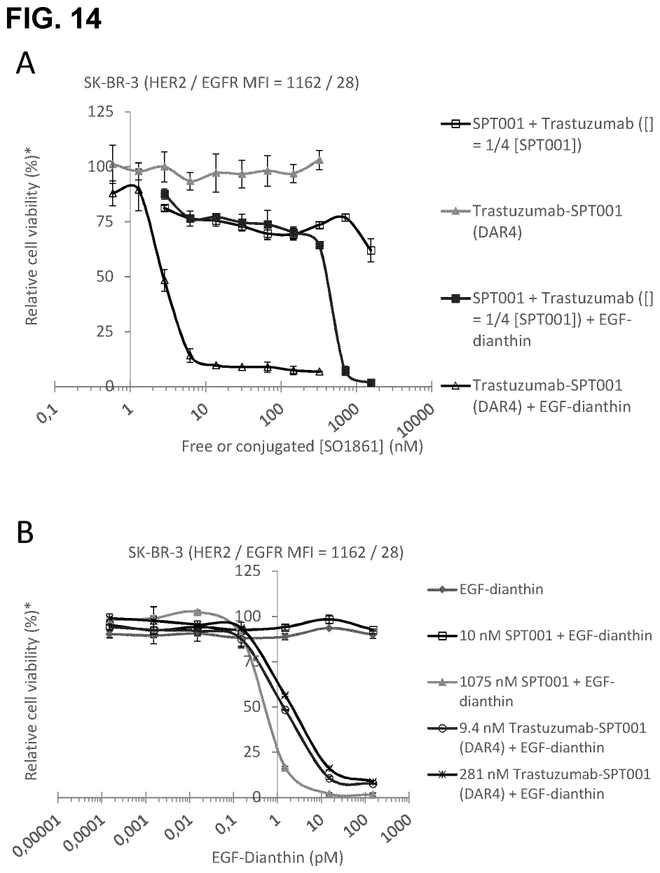

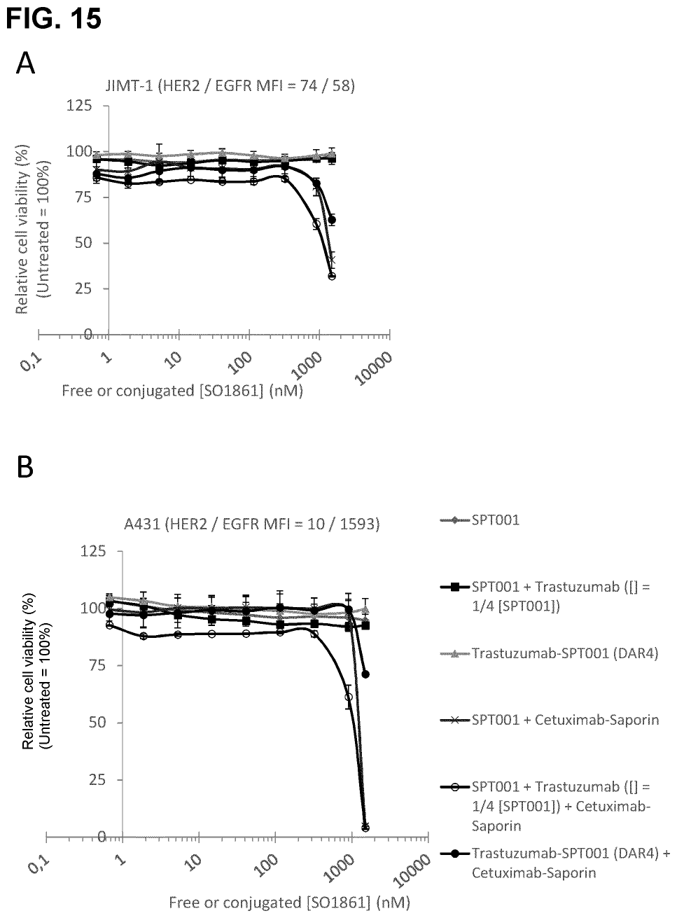

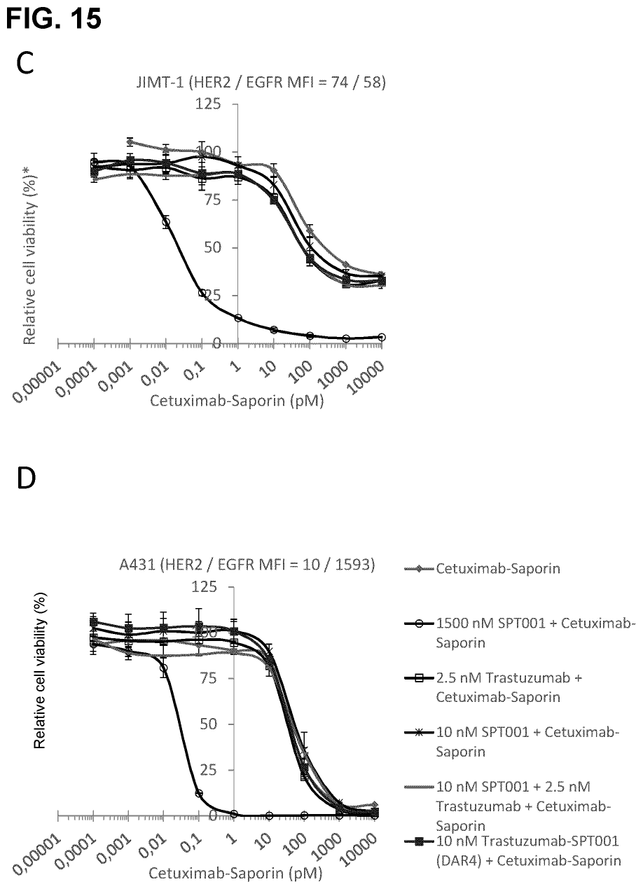

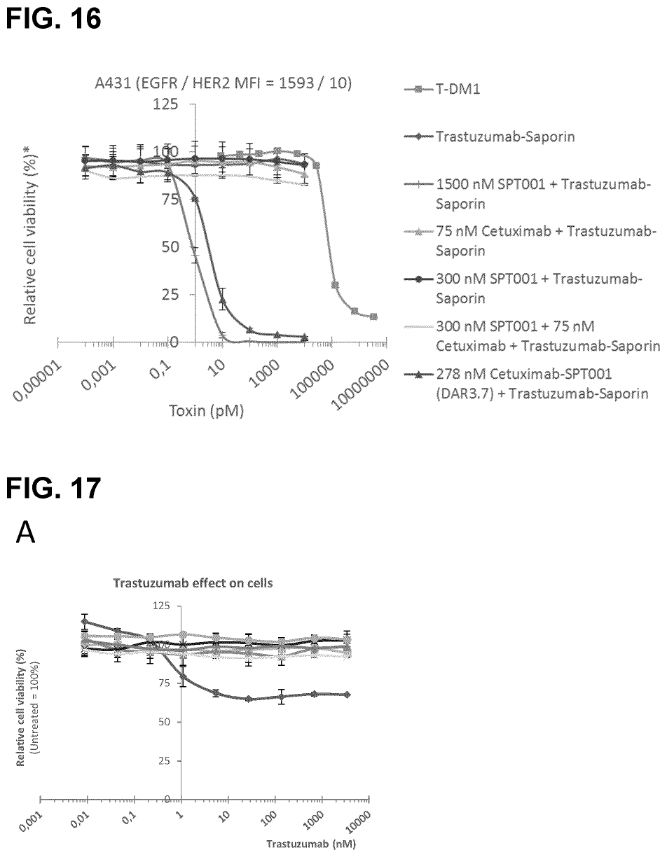

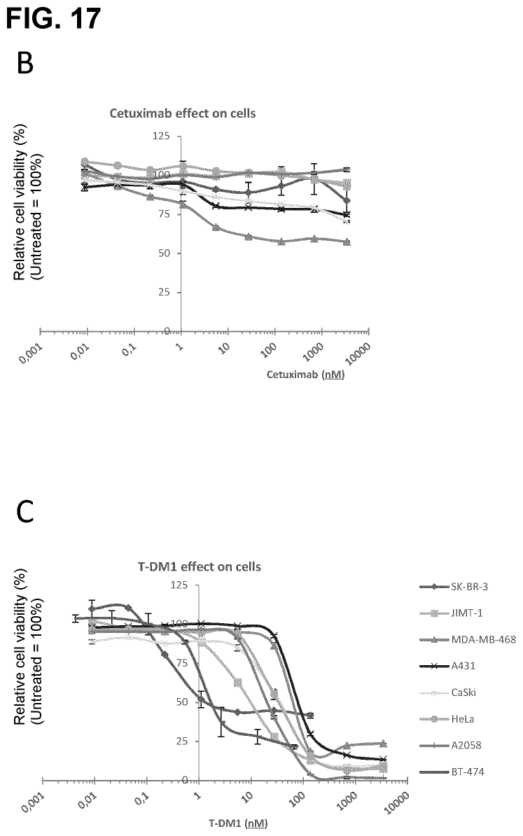

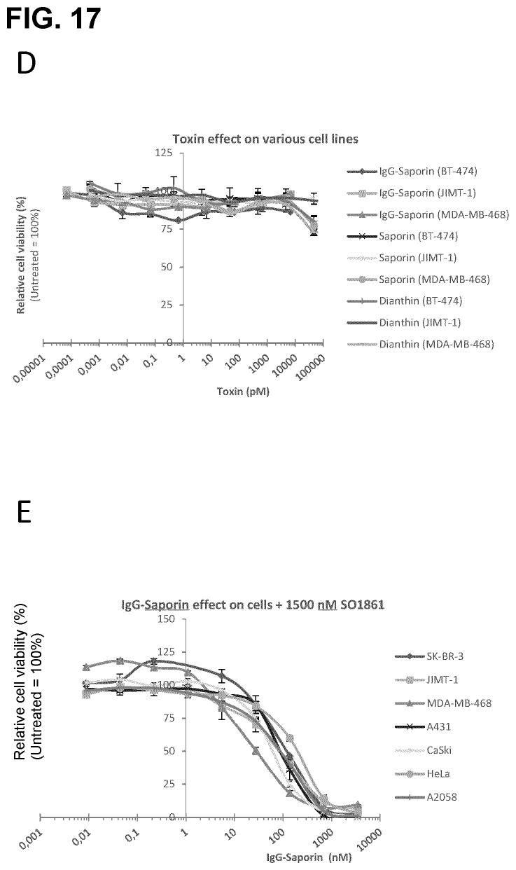

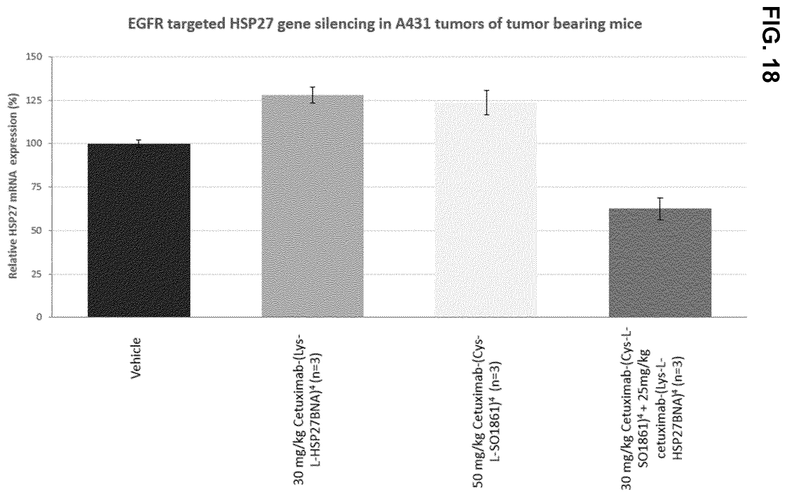

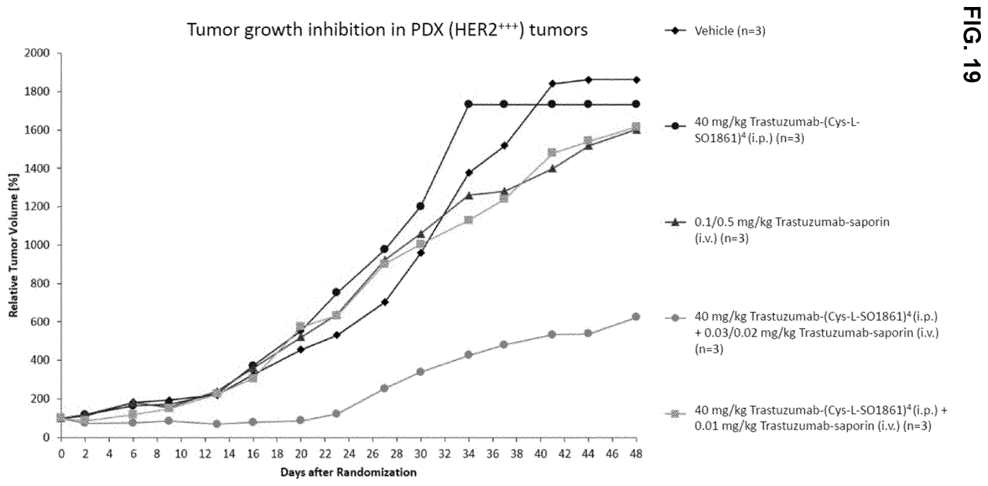

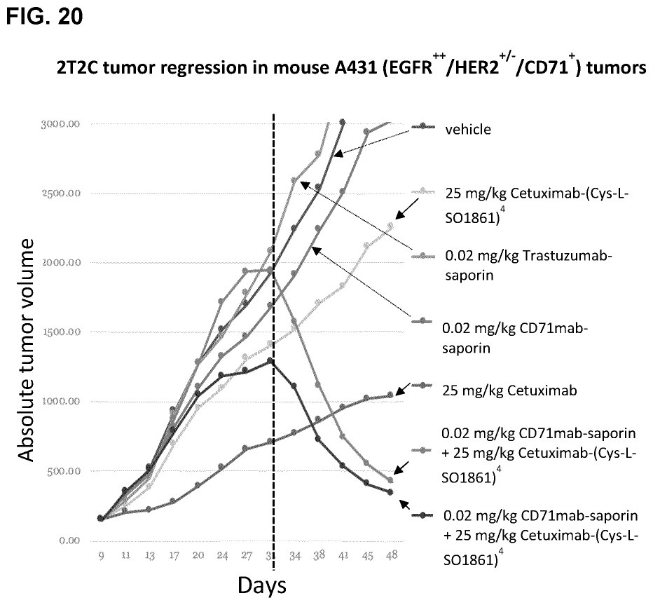

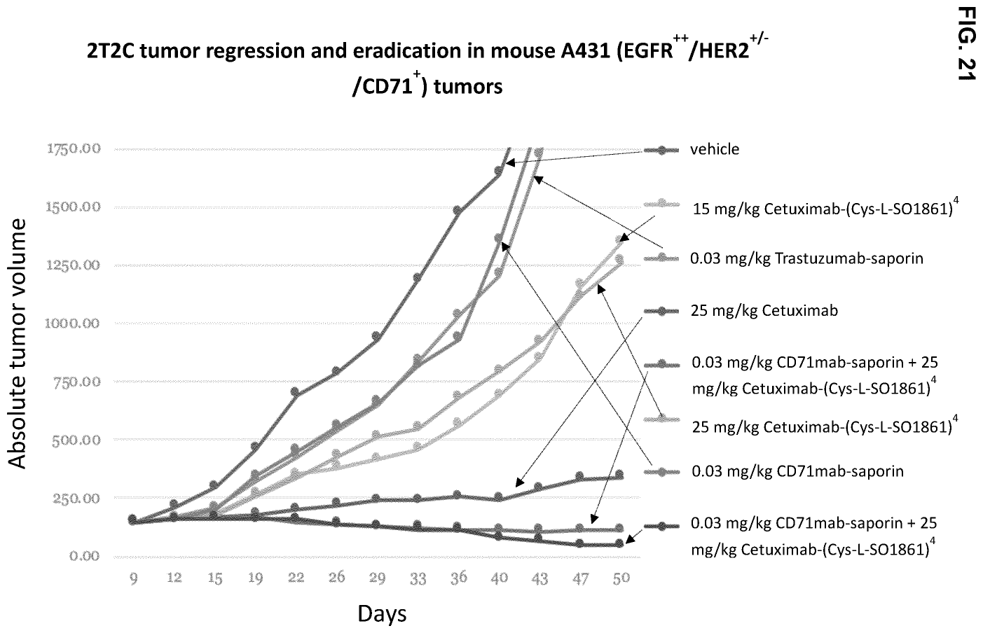

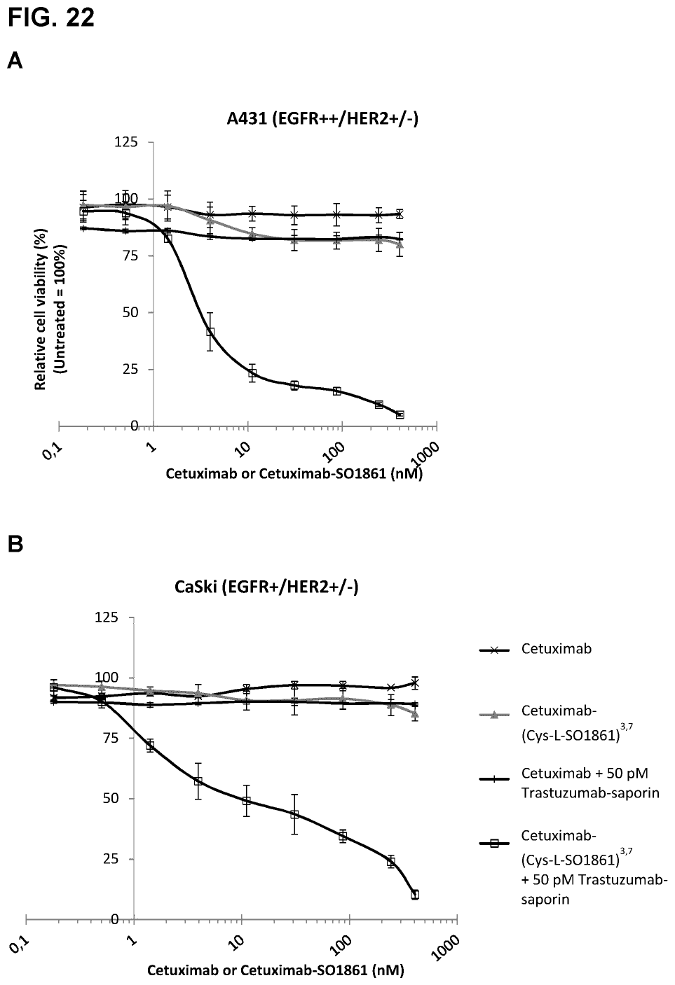

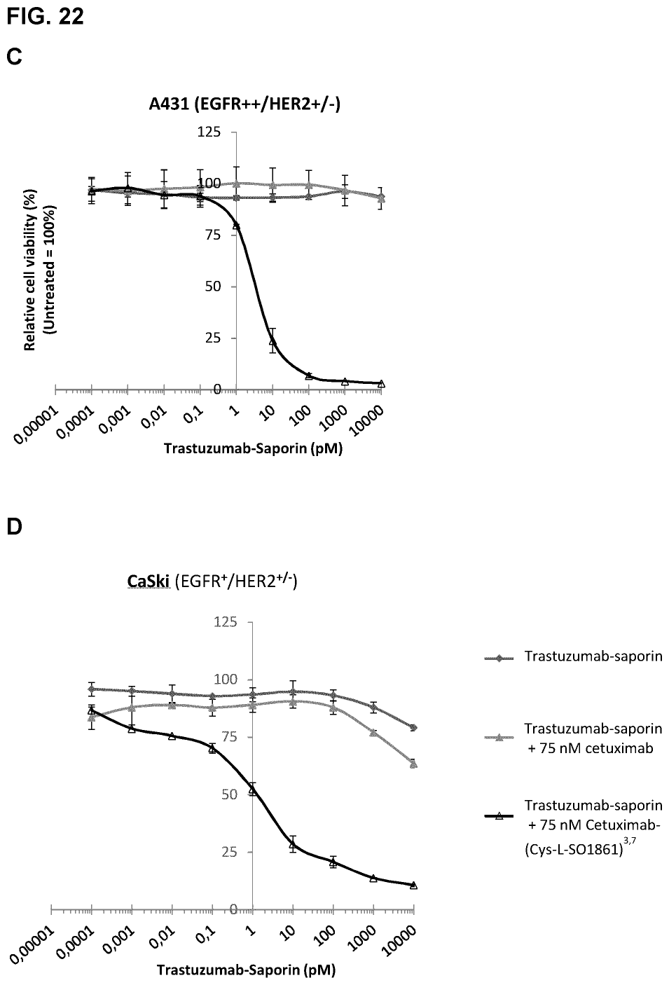

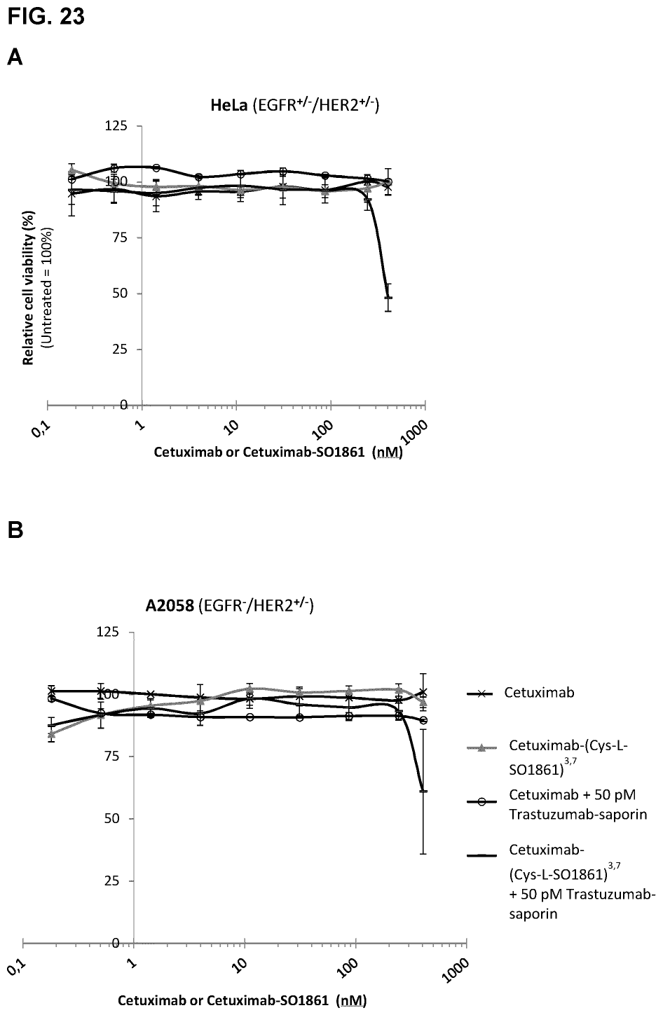

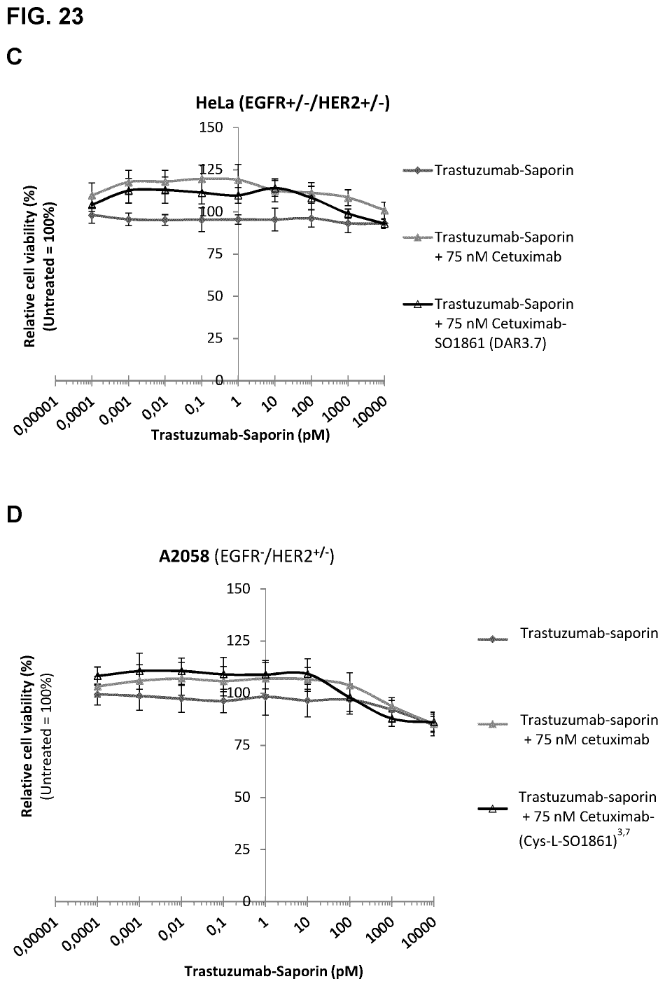

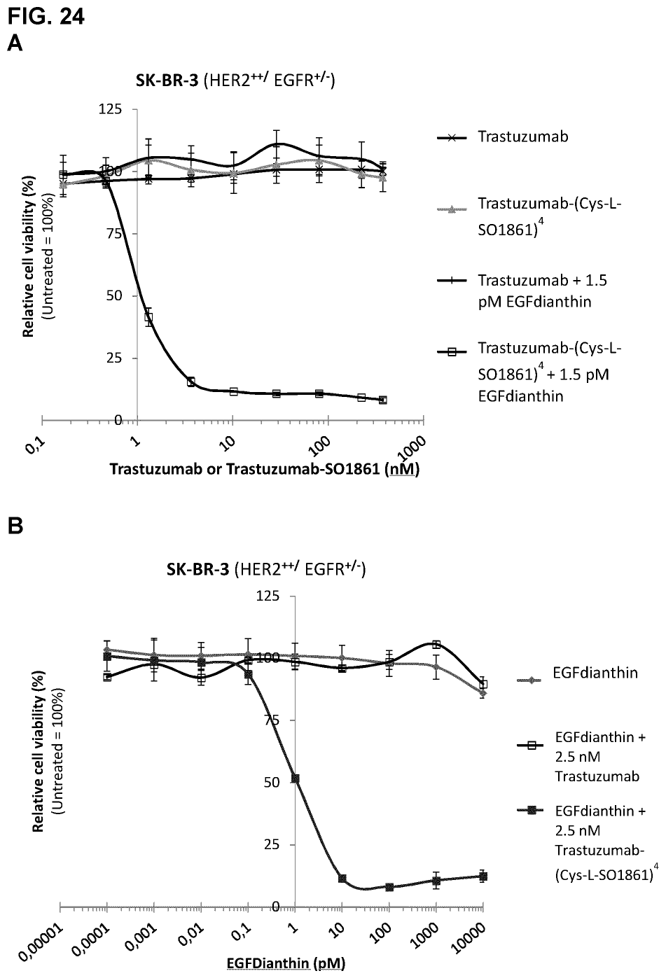

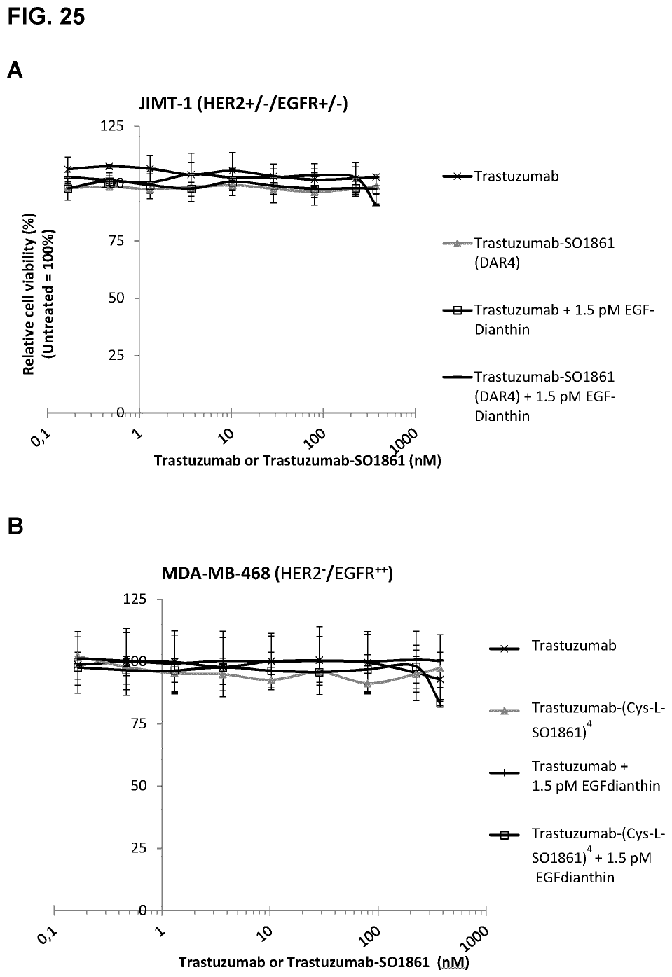

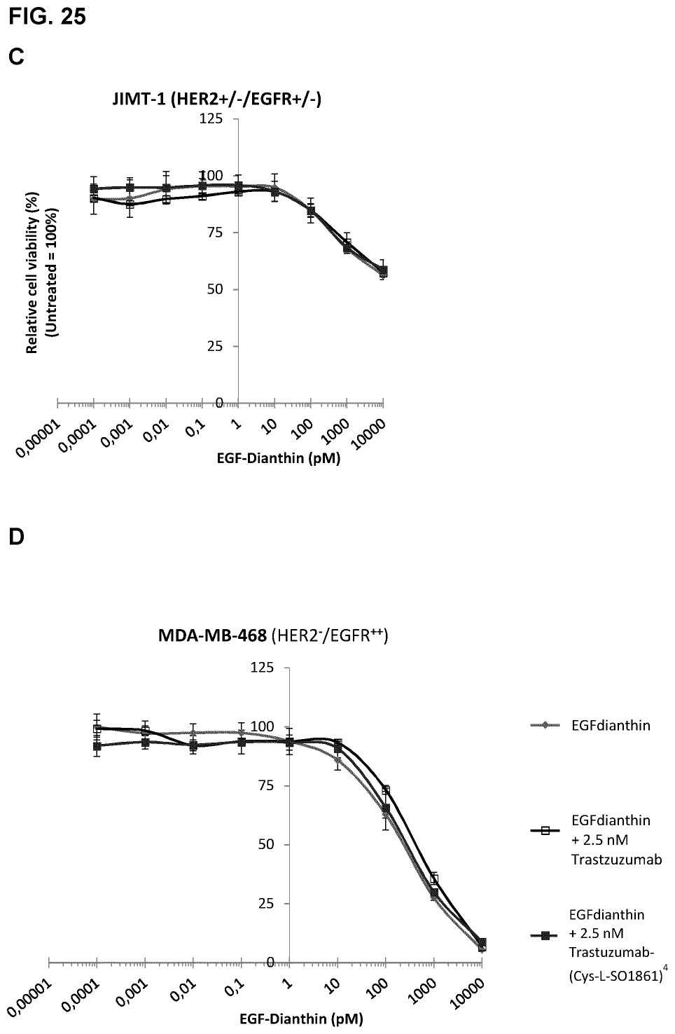

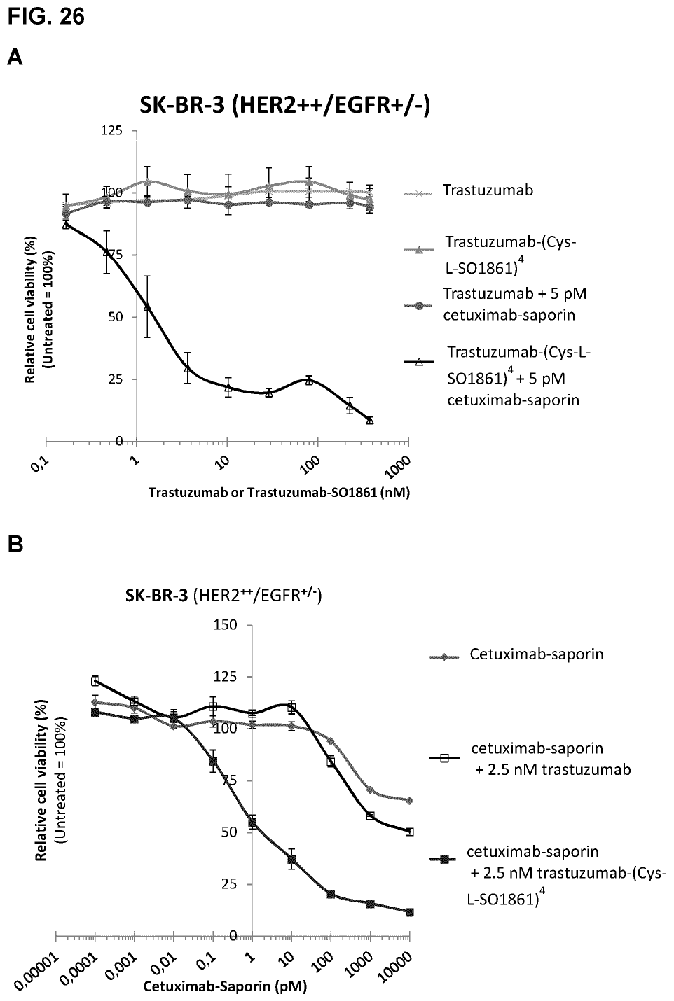

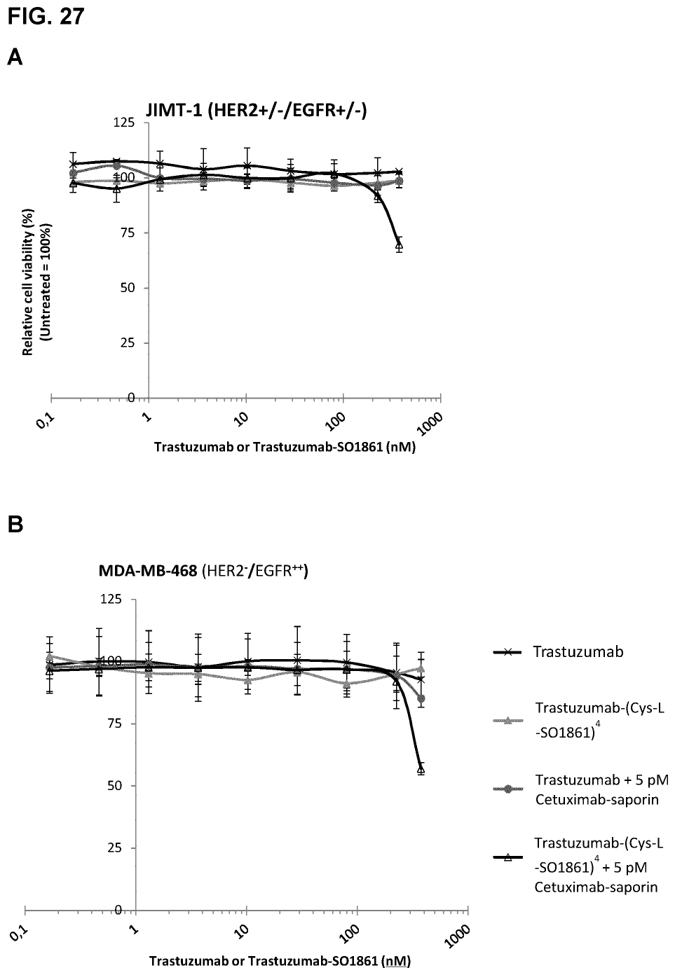

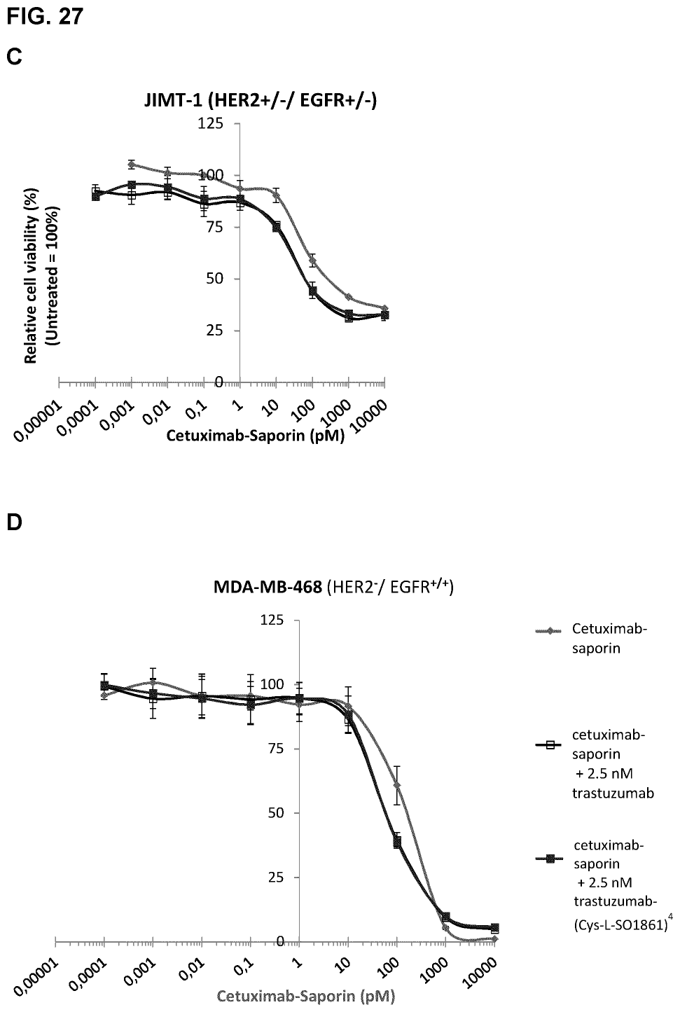

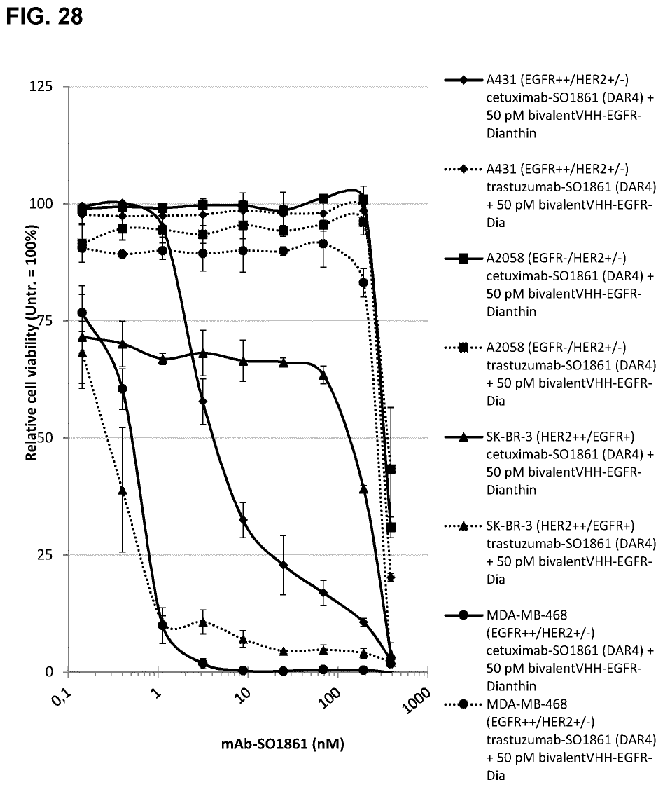

FIG. 10 (FIG. 10): 1-target 2-component (HER2 high expression). HER2 targeted cell killing in SKBR3 (HER2⁺⁺⁺) cells by a therapeutic combination. A) Trastuzumab-SO1861 titration in combination with a fixed concentration of 673 pM trastuzumab-saporin shows that a 1000 fold reduced concentration of conjugated SO1861 is required, versus unconjugated SO1861, to induce cell killing by trastuzumab-saporin. B), Trastuzumab-saporin titration in combination with 9.4 nM Trastuzumab-SO1861 kills cells in contrast to 10 nM unconjugated SO1861+trastuzumab-saporin. 1075 nM unconjugated SO1861+trastuzumab-saporin is more efficient compared to the therapeutic combination, since both trastuzumab conjugates compete for the same HER2 receptor. Only simultaneous targeted delivery of both trastuzumab conjugates leads to efficient cell-killing, in contrast to monotherapy with either conjugate alone. SPT001 is SO1861.

FIG. 11 (FIG. 11): 1-target 2-component (HER2 no/low expression). HER2 targeted cell killing in JIMT1 (HER2⁺) and A431 (HER2^{+/-}) cells, by a therapeutic combination. A, B) trastuzumab-SO1861 titration in combination with a fixed concentration of 50 pM trastuzumab-saporin does not induce cell killing by trastuzumab-saporin. C, D), Trastuzumab-saporin titration in combination with 10 nM trastuzumab-SO1861 can not induce cell killing. Low HER2 receptor expression is prohibitive for sufficient SO1861 to be delivered via antibody-mediated delivery, while 1500 nM of unconjugated SO1861 induces efficient cell killing. REMARK: the legend displayed in FIG. 11B also relates to the graphs in FIG. 11A. REMARK: the legend displayed in FIG. 11D also relates to the graphs in FIG. 11C. SPT001 is SO1861.

FIG. 12 (FIG. 12): 2-target 2-component (EGFR high expression and HER2 low expression). EGFR/HER2 targeted cell killing in A431 (EGFR⁺⁺⁺/HER2^{+/-}) and Caski (EGFR⁺⁺/HER2^{+/-}) cells by a therapeutic combination. A, B) Cetuximab-SO1861 titration in combination with a fixed concentration of 50 pM trastuzumab-saporin shows a 100 fold reduced concentration of conjugated SO1861 is required, versus unconjugated SO1861, to induce cell killing by trastuzumab-saporin. C, D), Trastuzumab-saporin titration in combination with 278 nM cetuximab-SO1861 kills cells in contrast to 300 nM unconjugated SO1861+trastuzumab-saporin. 1500 nM unconjugated SO1861+trastuzumab-saporin has comparable cell killing efficiency compared to the therapeutic combination, 278 nM cetuximab-SO1861+trastuzumab-saporin, since both conjugates do not compete for the same receptor. Only simultaneous targeted delivery of both conjugates leads to efficient cell-killing, in contrast to monotherapy with either conjugate alone. REMARK: the legend displayed in FIG. 12B also relates to the graphs in FIG. 12A. REMARK: the legend displayed in FIG. 12D also relates to the graphs in FIG. 12C. SPT001 is SO1861.

FIG. 13. (FIG. 13): 2-target 2-component (EGFR low expression and HER2 no/low expression). EGFR/HER2 targeted cell killing in HeLa (EGFR⁺/HER2^{+/-}) and A2058 (EGFR⁻/HER2^{+/-}) cells by a therapeutic combination. A, B) Cetuximab-SO1861 titration in combination with a fixed concentration of 50 pM trastuzumab-saporin does not induce cell killing by trastuzumab-saporin. C, D), Trastuzumab-saporin titration in combination with 278 nM cetuximab-SO1861 does not potentiate cell killing, while 1500 nM of unconjugated SO1861 induces efficient cell killing. Low EGFR receptor expression is prohibitive for sufficient SO1861 to be delivered via antibody-mediated delivery. REMARK: the legend displayed in FIG. 13B also relates to the graphs in FIG. 13A. REMARK: the legend displayed in FIG. 13D also relates to the graphs in FIG. 13C. SPT001 is SO1861.

FIG. 14. (FIG. 14): 2-target 2-component (HER2 high expression and EGFR low expression). HER2 targeted cell killing in SKBR3 (HER2⁺⁺⁺/EGFR^{+/-}) cells by a therapeutic combination. A) Trastuzumab-SO1861 titration in combination with a fixed concentration of 1.5 pM EGFdianthin shows that a 400 fold reduced concentration of conjugated SO1861 is required, versus unconjugated SO1861, to induce cell killing by EGFdianthin. B), EGFdianthin titration in combination with 9.4 nM trastuzumab-SO1861 can kill cells in contrast to 10 nM unconjugated SO1861+EGFdianthin. 1075 nM unconjugated SO1861+EGFdianthin has comparable cell killing efficiency compared to the therapeutic combination, 9.4 nM trastuzumab-SO1861+EGFdianthin, since both conjugates do not compete for the same receptor. Only simultaneous targeted delivery of both conjugates leads to efficient cell-killing, in contrast to monotherapy with either conjugate alone. SPT001 is SO1861.

FIG. 15. (FIG. 15): 2-target 2-component (HER2 low expression and EGFR low or high expression). HER2 targeted cell killing in JIMT1 (HER2′) and A431 (HER2+/−) cells, by a therapeutic combination according to the invention. A, B) trastuzumab-SO1861 titration in combination with a fixed concentration of 5 pM cetuximab-saporin does not induce cell killing by cetuximab-saporin. C, D), Cetuximab-saporin titration in combination with 10 nM trastuzumab-SO1861 can not induce cell killing. Low HER2 receptor expression is prohibitive for sufficient SO1861 to be delivered via antibody-mediated delivery, while 1500 nM of unconjugated SO1861 induces efficient cell killing. Even a high EGFR receptor expression level (D) for delivery of cetuximab-saporin does not change its potency in the presence of trastuzumab-SO1861, indicating that the bottleneck for cell-killing activity is a too low HER2 expression level, leading to insufficient SO1861 inside target cells to switch on endosomal escape. REMARK: the legend displayed in FIG. 15B also relates to the graphs in FIG. 15A. REMARK: the legend displayed in FIG. 15D also relates to the graphs in FIG. 15C. SPT001 is SO1861.

FIG. 16. (FIG. 16): 2-target 2-component versus T-DM1. Cells with high EGFR expression and low HER2 expression (A431) are efficiently killed with the therapeutic combination, however T-DM1 is not effective at such low toxin concentrations. T-DM1 is Trastuzumab-emtansine (Kadcyla®), carrying −3.5 DM1 toxin molecules per antibody.

FIG. 17A-E. (FIG. 17A-E): displays the relative cell viability when trastuzumab (FIG. 17A), cetuximab (FIG. 17B) or T-DM1 (FIG. 17C), free toxins saporin (FIG. 17D) and dianthin (FIG. 17D), saporin coupled to a non-cell binding IgG (FIG. 17D), and saporin coupled to a non-cell binding IgG combined with free saponin SO1861 (FIG. 17E) are contacted with the indicated cell lines SK-BR-3, JIMT-1, MDA-MB-468, A431, CaSki, HeLa, A2058, BT-474. REMARK: the legend displayed in FIG. 17C also relates to the curves in FIGS. 17A and 17B.

FIG. 18. (FIG. 18): 1T2C in vivo activity. The 1T2C combination of 50 mg/kg cetuximab-(Cys-L-SO1861)⁴+25 mg/kg cetuximab-(-L-HSP27BNA)⁴ in A431 tumor bearing mice reveals strong tumor targeted gene silencing, compared to the controls.

FIG. 19. (FIG. 19): 1T2C in vivo activity. The 1T2C combination of 40 mg/kg trastuzumab-(Cys-L-SO1861)⁴+0.02/0.03 mg/kg trastuzumab-saporin in a PDX tumor mouse model (high HER2 expression) shows effective tumor growth inhibition.

FIG. 20. (FIG. 20): The 2T2 component system tested in A431 tumor bearing mice model reveals tumor regression.

FIG. 21. (FIG. 21): The 2T2 component system tested in A431 tumor bearing mice model reveals tumor regression and eradication.

FIG. 22 (FIG. 22): 2-target 2-component. EGFR/HER2 targeted cell killing in A431 cells (EGFR⁺⁺/HER2^{+/-}) (A, C) and CaSKi cells (EGFR⁺⁺/HER2^{+/-}) (B, D) by a therapeutic combination according to the invention. A, B) Cetuximab-(Cys-L-SO1861)^{3,7} titration+fixed concentration 50 pM trastuzumab-saporin and controls on A431 cells. C, D) Trastuzumab-saporin titration+fixed concentration of 75 nM cetuximab-(Cys-L-SO1861)^{3,7} and controls on Caski cells. The legends and/or axes are the same for A, B, and the legends are the same for figures C and D.

FIG. 23. (FIG. 23): 2-target 2-component. EGFR/HER2 targeted cell killing in HeLa cells (EGFR^{+/-}/HER2^{+/-}) (A, C) and A2058 cells (EGFRIHER2^{+/-}) (B, D) by a therapeutic combination according to the invention. A, B) Cetuximab-(Cys-L-SO1861)^{3,7} titration+fixed concentration 50 pM trastuzumab-saporin and controls on HeLa cells. C, D) Trastuzumab-saporin titration+fixed concentration of 75 nM cetuximab-(Cys-L-SO1861)^{3,7} and controls on A2058 cells. The legends and/or axes are the same for C and D.

FIG. 24. (FIG. 24): 2-target 2-component. HER2/EGFR targeted cell killing in SKBR3 cells (HER2⁺⁺/EGFR^{+/-}) (A, B) by a therapeutic combination according to the invention. A Trastuzumab-(Cys-L-SO1861)4 titration+fixed concentration 1.5 pM EGFdianthin and controls on SKBR3 cells. B) EGFdianthin titration+fixed concentration of 2.5 nM trastuzumab-(Cys-L-SO1861)⁴ and controls on SKBR3 cells.

FIG. 25. (FIG. 25): 2-target 2-component. HER2/EGFR targeted cell killing in JIMT-1 cells (HER2^{+/-}EGFR^{+/-}) (A, C) and MDA-MB-468 cells (HER2⁻/EGFR⁺⁺) (B, D) by a therapeutic combination according to the invention. A, B) Trastuzumab-(Cys-L-SO1861)⁴ titration+fixed concentration 1.5 pM EGFdianthin and controls on JIMT-1 cells. C, D) EGFdianthin titration+fixed concentration of 2.5 nM trastuzumab-(Cys-L-SO1861)⁴ and controls on MDA-MB-468 cells. The legends and/or axes are the same for C and D.

FIG. 26. (FIG. 26): 2-target 2-component. HER2/EGFR targeted cell killing in SKBR3 cells (HER2⁺⁺/EGFR^{+/-}) (A, B) by a therapeutic combination according to the invention. A) Trastuzumab-(Cys-L-SO1861)⁴ titration+fixed concentration 10 pM cetuximab-saporin and controls on SKBR3 cells. B) Cetuximab-saporin titration+fixed concentration of 2.5 nM trastuzumab-(Cys-L-SO1861)⁴ and controls on SKBR3 cells.

FIG. 27. (FIG. 27): 2-target 2-component. HER2/EGFR targeted cell killing in JIMT-1 cells (HER2^{+/-}EGFR^{+/-}) (A, C) and MDA-MB-468 cells (HER2⁻/EGFR⁺⁺) (B, D) by a therapeutic combination according to the invention. A, B) Trastuzumab-(Cys-L-SO1861)⁴ titration+fixed concentration 10 pM cetuximab-saporin and controls on JIMT-1 cells. C, D) Cetuximab-saporin titration+fixed concentration of 2.5 nM trastuzumab-(Cys-L-SO1861)⁴ and controls on MDA-MB-468 cells. The legends and/or axes are the same for A and B, and the legends are the same for C and D.

FIG. 28 (FIG. 28): Cell killing (MTS) assay) with the combination treatment according to the invention of cetuximab-SO1861 (DAR4)+50 pM bivalent VHHEGFR-dianthin (recombinant fusion protein) or trastuzumab-SO1861 (DAR4)+50 pM bivalent VHHEGFR-dianthin (recombinant fusion protein) on A431 cells (EGFR⁺⁺/HER^{+/-}), A2058 cells (EGFR⁻/HER^{+/-}), SK-BR-3 cells (EGFR⁻/HER2⁺⁺) and MDA-MB-468 cells (EGFR⁺⁺/HER2⁻).

### DETAILED DESCRIPTION

In order for a bioactive molecule (e.g. an effector molecule) to work, the molecule must be able to engage with its target, e.g. in the blood serum, on the outside of the cell surface or inside a cell or an organelle. The active moiety of almost all protein-based targeted toxins, e.g., must enter the cytosol of the target cell to mediate its target modulatory effect. In many constellations the toxin remains ineffective since (1) the targeting moiety is poorly internalized and remains bound to the outside of the cells, (2) is recycled back to the cell surface after internalization or (3) transported to the endolysosomes where it is degraded. Although these fundamental issues are known for decades and more than 500 targeted toxins have been investigated in the past decades, the problems have not been solved yet and only a couple of antibody-targeted protein toxin have been admitted to the market, albeit with warning labels for severe toxicity. Moxetumomab pasudotox-tdfk (LUMOXITI®, AstraZeneca Pharmaceuticals LP), has been approved for relapsed or refractory hairy cell leukemia by the FDA to date. Other of such approved ADCs are Elzonris, Ontak.

To overcome these problems, many strategies have been described including approaches to redirect the toxins to endogenous cellular membrane transport complexes of the biosynthetic pathway in the endoplasmic reticulum and techniques to disrupt or weaken the membrane integrity of endosomes, i.e. the compartments of the endocytic pathway in a cell, and thus facilitating the endosomal escape. This comprises the use of lysosomotropic amines, carboxylic ionophores, calcium channel antagonists, various cell-penetrating peptides of viral, bacterial, plant, animal, human and synthetic origin, other organic molecules and light-induced techniques. Although the efficacy of the targeted toxins was typically augmented in cell culture hundred- or thousand-fold, in exceptional cases more than million-fold, the requirement to co-administer endosomal escape enhancers with other substances harbors new problems including additional side effects, loss of target specificity, difficulties to determine the therapeutic window and cell type-dependent variations.

All strategies, including physicochemical techniques, require enhancer molecules that interact more or less directly with membranes and comprise essentially small chemical molecules, secondary metabolites, peptides and proteins. A common feature of all these substances is that they are per se not target cell-specific and distribute with other kinetics than the targeted toxins. This is one major drawback of the current approaches.

It is a first goal of the present invention to provide improved enhancers for ADCs and AOCs, and to provide an improved therapeutic combination or an improved pharmaceutical composition for delivery of an effective amount of an effector molecule, when for example the delivery from outside a target cell into said cell, is considered, or more in particular when the delivery of the effector molecule in the cytosol of said target cell is considered. It is a second goal of the present invention to provide an improved method of treatment of a (human) patient suffering from a disease to be treated with a conjugate comprising an effector molecule and a ligand, such as EGF, a cytokine, Her2 targeting V_{HH} or IgG, EGFR targeting IgG, etc, or an active domain, active derivative or an active fragment thereof, preferably an sdAb or multiple sdAb's, such as one or more V_{HH}s, for e.g. a target tumor cell, i.e. to improve the therapeutic window of the ADC or the AOC comprising the effector molecule to be delivered in the cytosol of target tumor cells.

It is an objective of the current invention to provide effector-molecule enhancing molecules for use in therapy such as anti-cancer therapy, a therapeutic composition or a therapeutic combination of e.g. two therapeutic compositions, comprising such a molecule that is capable of improving a biological effect of an effector molecule once delivered inside a target cell which comprises the molecular target for the effector molecule, which provided molecule, when administered to a (human) patient in need thereof, bearing the target cell, experience an improved therapeutic effect or a sufficient effect at a lower dose of the effector molecule than the currently required dose for reaching an effective dose of the effector molecule. This way, the therapeutic window of the effector molecule, such as an effector molecule which is part of a conjugate, such as an ADC or an AOC, is widened effectively.

At least one of the above objectives is achieved by providing the conjugates of the invention.

The present invention will be described with respect to particular embodiments but the invention is not limited thereto but only by the claims. While the invention has been described in terms of several embodiments, it is contemplated that alternatives, modifications, permutations and equivalents thereof will become apparent to one having ordinary skill in the art upon reading the specification and upon study of the drawings and graphs. The invention is not limited in any way to the illustrated embodiments. Changes can be made without departing from the scope which is defined by the appended claims.

The inventors invented and established that the therapeutic window of a conjugate such as an antibody drug conjugate or an antibody-oligonucleotide conjugate, increases when to the same subject in need thereof such as a human cancer patient or a tumor bearing mammal, is administered a second conjugate comprising at least one covalently bound saponin (see for example the examples in Tables A5-A7, and see for example FIGS. 2-5 for conjugates comprising V_{HH} and FIGS. 5-16 and 18-27 for conjugates comprising a monoclonal antibody (mAb), in the Examples section for a series of in vitro and in vivo tumor cell-model examples of the effect of saponin covalently bound to an antibody such as an IgG or a V_{HH}, when combined with an ADC or an AOC). The saponin is conjugated with an antibody such as an IgG or a V_{HH}, and an effector molecule such as protein toxins or an oligonucleotide such as a BNA is conjugated with a second antibody such as an IgG or an sdAb. The inventors were the first who established and determined that conjugating a saponin of the invention with a ligand for binding to a cell-surface molecule, such as an antibody such as a full-length intact IgG, or such as an sdAb such as a V_{HH}, provides a conjugate for cell-specific delivery of the saponin at the cell surface of a target cell exposing the cell-surface molecule at its cell surface, and subsequent delivery of the saponin inside the cell, such as the cell endosome, endolysosome, lysosome and ultimately in the cell cytosol. Examples of such cell-targeting saponin conjugates are for example provided in FIGS. 2-5 for saponin-V_{HH} conjugates and FIGS. 5-16 and 18-27, as outlined in the Examples section here below. Saponin is conjugated to ligands such as EGF, Her2 targeting V_{HH} or IgG, EGFR targeting IgG, EGFR binding V_{HH} 7D12 with amino-acid sequence as depicted as SEQ ID NO: 75, EGFR binding V_{HH} 9G8 with amino acid sequence as depicted as SEQ ID NO: 76, EGFR binding covalently linked tandem of biparatopic V_{HH}'s 7D12-9G8 with amino-acid sequence as depicted as SEQ ID NO: 74, etc. The conjugate of the invention, comprising an sdAb or a full-length antibody or a different immunoglobulin (Ig) format, as the cell-surface molecule binding molecule in the conjugate of the invention, has at least one glycoside such as a saponin of the invention bound thereto, preferably, and in all exemplary examples, covalently, more preferably via a (cleavable) linker. The saponin augments the therapeutic efficacy of the effector moiety covalently bound to a second cell-surface molecule targeting molecule (antibody, sdAb), i.e. a second conjugate of for example an effector molecule and an antibody such as an IgG or an sdAb, without wishing to be bound by any theory, likely by enhancing the endosomal escape of the effector moiety into the cytosol where the activity of the effector moiety is desired. This way, already at a lower dose of the effector molecule than the conventional dose of the ADC or the AOC, under influence of a certain dose of the saponin-comprising conjugate of the invention, therapeutic effect is established under influence of the presence of the conjugate comprising the saponin(s) thereby bringing the saponin(s) near, at and/or inside the targeted cell. The targeted cell is for example a diseased cell such as a tumor cell or an auto-immune cell or a B-cell disease related B-cell, etc. The effector moiety is for example a toxin as part of an ADC or an oligonucleotide such as an antisense BNA as part of an AOC according to the invention. The second cell-surface molecule targeting molecule can for example bind to the same cell-surface molecule as the conjugate of the invention, wherein the second conjugate comprises a copy of the same cell-surface molecule binding molecule as the conjugate of the invention. Alternatively, the second conjugate comprises a second cell-surface molecule targeting molecule that can specifically bind to a cell-surface molecule different from the cell-surface molecule to which the conjugate of the invention, comprising the saponin, can bind.

A first aspect of the invention relates to a conjugate for transferring a saponin from outside a cell into said cell, comprising a single-domain antibody (sdAb), capable of binding to said cell, wherein the sdAb is covalently bound to at least one saponin, optionally directly or optionally via a linker, wherein the at least one saponin is a monodesmosidic triterpene glycoside or a bidesmosidic triterpene glycoside. The conjugate of the invention is capable of transporting the at least one saponin comprised by the conjugate from outside a cell, preferably a mammalian cell such as a human cell, to inside said cell. Preferably, the cell is a target tumor cell or a target auto-immune cell.

The conjugate can migrate through the cell membrane from outside the cell to inside the cell, therewith delivering the saponin in the cell. Typically, the conjugate migrates from the cell membrane to the endosome and/or the lysosome of the cell, therewith delivering the saponin inside the endosome and lysosome. Typically, such migration is due to receptor-mediated endocytosis of the conjugate upon binding of the cell-surface molecule binding molecule to such a receptor involved in endocytosis of the receptor with bound conjugate thereto (binding of conjugates to the receptor is followed by internalization of the conjugate/receptor complex). The sdAb is capable of binding to a binding site exposed on the outer surface of the cell, wherein the binding site is typically present on a cell-surface molecule such as a proteinaceous molecule, and preferably a cell-surface receptor. Transferring the conjugate over the cell membrane and into the cell typically involves the binding of the conjugate to a cell-surface receptor via the sdAb with specificity for binding to said cell-surface receptor, followed by endocytocis of the conjugate such that the saponin in the conjugate is delivered inside the cell, and typically delivered in the endosome of the cell. The conjugate can comprise a single sdAb for binding to a cell-surface molecule, or can comprise more than one sdAb, such as 2-10, or 3-6, sdAbs, for binding to a single type of cell-surface molecule.

An embodiment is the conjugate of the invention, wherein the sdAb is a V_{H} domain derived from a heavy chain of an antibody, preferably of immunoglobulin G origin, preferably of human origin, a V_{L} domain derived from a light chain of an antibody, preferably of immunoglobulin G origin, preferably of human origin, a V_{HH} domain such as derived from a heavy-chain only antibody (HCAb) such as from Camelidae origin or Ig-NAR origin such as a variable heavy chain new antigen receptor (V_{NAR}) domain, preferably the HCAb is from Camelidae origin, preferably the sdAb is a V_{HH} domain derived from an HCAb from Camelidae origin (camelid V_{H}) such as derived from an HCAb from camel, lama, alpaca, dromedary, vicuna, guanaco and Bactrian camel.

To explain the invention in more detail, the process of cellular uptake of substances and the used terminology in this invention is described first. The uptake of extracellular substances into a cell by vesicle budding is called endocytosis. Said vesicle budding can be characterized by (1) receptor-dependent ligand uptake mediated by the cytosolic protein clathrin, (2) lipid-raft uptake mediated by the cholesterol-binding protein caveolin, (3) unspecific fluid uptake (pinocytosis), or (4) unspecific particle uptake (phagocytosis). All types of endocytosis run into the following cellular processes of vesicle transport and substance sorting called the endocytic pathways. The endocytic pathways are complex and not fully understood. Earlier, it was thought that organelles are formed de novo and mature into the next organelle along the endocytic pathway. Nowadays, it is hypothesized that the endocytic pathways involve stable compartments that are connected by vesicular traffic. A compartment is a complex, multifunctional membrane organelle that is specialized for a particular set of essential functions for the cell. Vesicles are considered to be transient organelles, simpler in composition, and are defined as membrane-enclosed containers that form de novo by budding from a preexisting compartment. In contrast to compartments, vesicles can undergo maturation, which is a physiologically irreversible series of biochemical changes. Early endosomes and late endosomes represent stable compartments in the endocytic pathway while primary endocytic vesicles, phagosomes, multivesicular bodies (also called endosome carrier vesicles), secretory granules, and even lysosomes represent vesicles. The endocytic vesicle, which arises at the plasma membrane, most prominently from clathrin-coated pits, first fuses with the early endosome, which is a major sorting compartment of approximately pH 6.5. A large part of the cargo and membranes internalized are recycled back to the plasma membrane through recycling vesicles (recycling pathway). Components that should be degraded are transported to the acidic late endosome (pH lower than 6) via multivesicular bodies. Lysosomes are vesicles that can store mature lysosomal enzymes and deliver them to a late endosomal compartment when needed. The resulting organelle is called the hybrid organelle or endolysosome. Lysosomes bud off the hybrid organelle in a process referred to as lysosome reformation. Late endosomes, lysosomes, and hybrid organelles are extremely dynamic organelles, and distinction between them is often difficult. Degradation of the endocytosed molecules occurs inside the endolysosomes. Endosomal escape is the active or passive release of a substance from the inner lumen of any kind of compartment or vesicle from the endocytic pathway, preferably from clathrin-mediated endocytosis, or recycling pathway into the cytosol. Endosomal escape thus includes but is not limited to release from endosomes, endolysosomes or lysosomes, including their intermediate and hybrid organelles. After entering the cytosol, said substance might move to other cell units such as the nucleus. Glycoside molecules (saponins) in the context of the invention are compounds that are able to enhance the effect of an effector molecule, in particular by facilitating the endosomal escape. The glycoside molecules interact with the membranes of compartments and vesicles of the endocytic and recycling pathway and make them leaky for said effector molecules resulting in augmented endosomal escape.

With the term “improving an effect of an effector molecule” is meant that a saponin increases the functional efficacy of the effector molecule (e.g. the therapeutic index of a toxin or a drug; the metabolic efficacy of a modifier in biotechnological processes; the transfection efficacy of genes in cell culture research experiments), preferably by enabling or improving its target engagement. Acceleration, prolongation, or enhancement of antigen-specific immune responses are preferably not included. Therapeutic efficacy includes but is not limited to a stronger therapeutic effect with lower dosing and/or less side effects. “Improving an effect of an effector molecule” can also mean that an effector molecule, which could not be used because of lack of effect (and was e.g. not known as being an effector molecule), becomes effective when used in combination with a conjugate of the present invention. Any other effect, which is beneficial or desired and can be attributed to the combination of an effector moiety, e.g. as part of an ADC or an AOC, and a saponin comprised by a conjugate of the invention, as provided by the invention, is considered to be “an improved effect”. In the context of the invention, a saponin comprised by the conjugate of the invention is an “enhancer” of the functional efficacy of an effector molecule, which can be comprised by a second, separate conjugate such as an ADC or an AOC, according to the invention.

One major drawback of targeted toxin enhancement by glycosides, such as for instance saponins, up to the present invention is that the targeted toxins are internalized by receptor-mediated endocytosis (binding of targeted toxins to the receptor is followed by internalization of the targeted toxin/receptor complex) while glycosides passively diffuse through the plasma membrane and reach the endosomal membranes presumably via interaction with cholesterol. In principal, glycosides such as the saponins in the conjugates of the invention, when in free non-conjugated form, can enter any cell, also non-target cells (off-target cells), resulting in inefficient enhancer availability in the target cells for effective release of the targeted toxin and possible side effects in non-target cells. One major problem is that entry of the targeted toxin and the glycosides proceed with different kinetics and that these kinetics are different from cell (line) to cell (line) and from tissue to tissue, so that the correct time difference for the application of the two substances (e.g. ADC, with a free saponin) can widely vary from tumor (cell (line)) to tumor (cell (line)). Moreover, in living organisms, liberation, absorption, distribution, metabolism and excretion of these substances is also different. Furthermore, the a-specific uptake of glycosides by non-targeted cells may induce unwanted effects in these cells. This can, e.g., be cytosolic delivery of compounds that should have been delivered to the lysosomes, disturbed antigen presentation, etc. Non-targeted administration of the glycoside and the targeted drug may also be problematic in drug development and may hinder or at least postpone marketing authorization by the relevant authorities (e.g. FDA or EMA). With targeted toxin or targeted drug in the context of the present invention is meant that a toxin or drug is specifically targeted to a membrane bound molecule on a target cell (cell-surface molecule), e.g. a toxin or drug bound to a ligand of a membrane receptor or bound to an antibody such as an sdAb that specifically recognizes a structure (binding site, epitope) on the cell membrane of a target cell.

It is thus very useful to direct the glycoside (a saponin of the invention) via the same route as the effector molecule, e.g., via a targeting ligand to the target cell in order for the enhancer to be available at effective concentration inside the acidic compartments of the endocytic pathway of the target cell and in order to exhibit a synergistic effect with the toxin. The present invention, therefore, provides novel approaches to redirect both the effector molecule and the endosomal escape enhancer (i.e. a saponin of the invention) via a targeting ligand (binding molecule) to the acidic compartments of the endocytic pathway of the target cell. The targeting ligand comprising the saponin in the conjugate of the invention can be the same as, or can be different from the targeting ligand comprised by the conjugate comprising the effector molecule. When the targeting ligands are different, it is appreciated that both targeting ligands bind to a first and second cell-surface molecule that are different but that are present on the same target cell such as a tumor cell. For example, the conjugate of the invention, comprising a saponin moiety, can bind to EGFR or CD71 or HER2, and for example the second conjugate comprising an effector moiety can bind to EGFR or CD71 or HER2, wherein the conjugate of the invention and the second conjugate bind to the same cell receptor or to a different cell receptor.

The inventors established that an effector molecule which is part of the second conjugate comprising a targeting ligand such as an antibody or an sdAb is delivered inside a cell with high efficiency under influence of a saponin which is comprised by the conjugate of the invention, when the effect of the effector molecule inside the cell is considered. Surprisingly, despite the relative small size of an sdAb such as a V_{HH}, binding of the conjugate of the invention comprising the saponin and comprising such sdAb, to the target cell surface receptor is still occurring when a saponin and an sdAb are comprised by the conjugate, the sdAb preferably being a V_{HH}. The binding of a saponin to the sdAb such as a V_{HH}, forming the conjugate of the invention, does not result in e.g. steric hindrance when the capacity of the V_{HH} to bind to the cell surface molecule is considered. That is to say, contacting e.g. tumor cells with a sub-optimal dose of e.g. an ADC or an AOC does not result in intracellular effector molecule activity (the target cell is not efficiently killed upon biological activity of the effector molecule), in the absence of the conjugate of the invention comprising the saponin covalently coupled to the sdAb. However, when the target tumor cell is contacted with the conjugate of the invention comprising the saponin, and with the ADC or the AOC, efficient tumor cell killing is achieved.

By targeting a single cell-surface molecule with the conjugate of the invention and an ADC or an AOC, or by targeting a first cell-surface molecule with the conjugate of the invention and targeting a second cell-surface molecule with an ADC or an AOC, wherein the first and second cell-surface molecules are different and are present at the same target cell, the delivery of the saponin bound to the cell-surface molecule targeting antibody such as an sdAb in the conjugate of the invention, and the delivery of the ADC or AOC, at and inside the cytosol of the targeted cell, exposing the cell-surface molecule(s) on the cell surface, is improved and more specific, compared to for example contacting the cell with only a regular ADC without contacting the cell simultaneously with the conjugate comprising the saponin of the invention, thus without the presence of the cell-targeted saponin (conjugate of the invention). An aberrant cell selected for targeting by the cell-surface molecule targeting sdAb of the conjugate, ideally bears the epitope on the cell-surface molecule to which the cell-surface molecule targeting molecule can bind, to a high extent (i.e. relatively higher expression of the targeted cell-surface molecule on the targeted cell such as for example a tumor cell or an auto-immune cell, than the expression on a non-targeted cell such as for example a healthy cell) and/or expose the epitope in the targeted cell-surface molecule for binding of the cell-surface molecule targeting sdAb of the conjugate, specifically, when (neighboring) healthy cells in a patient are considered. Preferably, the cell-surface molecule targeted by the cell-surface molecule targeting sdAb of the conjugate of the invention is relatively highly and/or specifically expressed on the targeted (diseased, tumor) cell compared to healthy cells. An embodiment is the conjugate of the invention, wherein the target cell-surface molecule for the cell-surface molecule targeting sdAb of the conjugate such as a tumor-cell receptor, is expressed specifically or to a relatively higher extent when compared to expression of the cell-surface molecule on the surface of a healthy (neighboring) cell. Thus, the epitope on the targeted cell-surface molecule is ideally unique to the targeted diseased cells, and is at least specifically present and exposed at the surface of the targeted cells. Binding of the conjugate of the invention to the epitope on the cell-surface molecule on a targeted cell is followed by endocytosis of the complex of the conjugate and the target cell-surface molecule (binding of conjugates to the receptor (the cell-surface molecule) is followed by internalization of the conjugate/receptor complex). Since the conjugate only can enter the target cell through binding interaction with a cell-surface molecules specifically expressed to a sufficient extent or uniquely expressed on the targeted cell when compared to healthy cells that should not be targeted, accumulation of a therapeutically active amount of effector moiety comprised by an ADC or an AOC which targets the same cell-surface molecule as the conjugate of the invention or which targets a different cell-surface molecule though present at the same target cell as the cell-surface molecule targeted by the conjugate of the invention, and saponin comprised by the conjugate of the invention, inside the target cells is only possible and occurring if expression levels of the targeted cell-surface molecule(s) is above a certain minimal expression threshold. At the same time, the fact that the effector moiety bound to a cell-surface molecule targeting ligand such as an antibody such as an sdAb, of the second conjugate is only capable of exerting its intracellular (e.g. cytotoxic or gene silencing) activity in the presence of the conjugate bearing the covalently bound saponin of the invention, also provides a safeguard against negative and undesired side effects of the effector moiety towards e.g. healthy cells and healthy tissue not meant to be targeted and affected by the effector moiety, when compared to exposure of cells to an ADC without the presence of the conjugate of the invention comprising the covalently bound saponin(s). That is to say, sufficiently low expression or even absence of exposed cell-surface molecule(s), to which a conjugate of the invention and a second conjugate such as an ADC or an AOC could bind, does ideally not allow entrance into (non-targeted) healthy cells of the conjugate of the invention and the second conjugate comprising the effector molecule, to amounts that would result in endosomal escape of the effector moiety of the ADC or the AOC under influence of the saponin comprised by the conjugate of the invention. Since the ADC or the AOC can be used at lower dose in the presence of the saponin comprising conjugate of the invention, compared to when the ADC or AOC was applied in the therapeutic regimen in the absence of the conjugate of the invention thus in the absence of saponin, entrance of ADC or entrance of AOC in healthy cells to low extent already bears a lower risk for occurrence of unwanted side effects when for example the targeting and killing of target diseased cells such as tumor cells and auto-immune cells is considered.

Inclusion of an sdAb in the conjugate has thus manifold advantages compared to inclusion of an antibody such as an IgG, or of a binding fragment or binding domain thereof. Importantly, since sdAbs do not comprise the Fc tail present in IgGs, risk for off-target side effects due to binding of the conjugate to Fc receptors on cells such as endothelial cells of a host to whom the conjugate is administered, is absent. Thus, the risk profile of the conjugate of the invention is improved compared to IgG-based conjugates comprising saponin, or compared to conjugates comprising an Fc tail and saponin. In addition, since the conjugate of the invention cannot be bound by Fc receptors, the conjugate is already effective at a dose which is lower than the dose required for reaching the same effector molecule activity with full-length antibody-based saponin comprising conjugates, due to less or no undesired capturing of the conjugate by cell-surface receptors, different from the aimed target cell-surface molecule. Furthermore, due to the relatively small size of sdAbs compared to e.g. Fab, scFv, IgG, tissue penetration is improved, which is beneficial for reaching the target cells once the conjugate is administered to a patient in need of therapy. All these advantages of the application of a sdAb in the conjugate of the invention, when compared to the application of larger antibodies or fragments thereof, such as IgGs comprising an Fc tail, in similar ADCs or AOCs, result in an improved therapeutic window for the effector molecule, when comprised by e.g. an ADC or an AOC and when combined in a therapeutic regimen together with the conjugate of the invention comprising saponin. For example, an ADC or AOC combined with a conjugate of the invention based on an sdAb may achieve improved target cell killing in case of a targeted tumor cell when the effector molecule is for example a toxin or a BNA, at the same dose at which a conjugate comprising saponin of the invention and based on an IgG and combined with the same effector molecule (ADC, AOC), is not or only sub-optimally effective. Thanks to the aspects of the invention, it is now possible to treat patients with a lower dose of effector molecule when the saponin is part of a conjugate comprising the sdAb, i.e. the conjugate of the invention, therewith reaching the same or improved effector molecule mediated effect in the target cells, compared to a higher dose required when an antibody-based saponin comprising conjugate is combined with an ADC or AOC, which comprises the same effector molecule. Administering such conjugate of the invention at lower dose lowers the risk for the patient for occurrence of side effects, e.g. by non-specific entrance of non-targeted, healthy cells. This is for example important when the cell-surface molecule that is targeted by the sdAb comprised by the conjugate is expressed to a higher extent on target (tumor) cells, but is not uniquely expressed on such target cells. A lower dose of the conjugate lowers the risk for binding of the conjugate to such low expressors, such as non-tumor healthy cells.

The inventors also found that the therapeutic window of ADCs and AOCs in the presence of the conjugate of the invention is widened due to the presence of covalently bound saponin in the conjugate of the invention. That is to say, when the ADC or the AOC provided with (combined with) a conjugate comprising saponin (i.e. the conjugate of the invention) is contacted with target cells, upon binding of the ADC or AOC, and the sdAb to its binding partner(s) (cell-surface molecules targeted by the ADC or the AOC and targeted by the sdAb of the conjugate of the invention are the same or are different) at the surface of the target cell, the saponin that is comprised by the conjugate of the invention is also brought in close proximity, i.e. at the surface of the target cell, together with the effector molecule of the second conjugate (i.e. the ADC or the AOC). When target cells that bear the cell-surface molecule(s), i.e. the target for the sdAb comprised by the conjugate of the invention and the target for the ligand binding molecule in the ADC or in the AOC, are contacted with the conjugate of the invention and with the ADC or AOC, both the effective dose of the effector molecule and the effective dose of the saponin is lower than when the target cells are contacted with an ADC or AOC in the absence of saponin or when the target cells are contacted with an ADC or AOC in the presence of free (untargeted) saponin. The presence of the targeted saponin as part of the conjugate of the invention potentiates the activity of the effector molecule in the target cells, such that the therapeutic window of the second conjugate (i.e. the ADC or the AOC), and therewith the therapeutic window of the effector molecule is widened. Sufficient effector molecule efficiency is achieved at lower ADC or AOC dose when target cells are contacted with the conjugate of the invention together with the ADC or the AOC. The similar effect is found by the inventors when an ADC or an AOC is contacted with the target cells in the presence of saponin or a functional derivative thereof, when effector-molecule potentiating activity of the saponin is considered, however, at a 100-fold to 1000-fold higher concentration of the free saponin (functional derivative) compared to the effective dose established when the conjugate of the invention is applied, which now comprises the effector molecule activity enhancing saponin, together with the sdAb for targeted binding of the conjugate to the target (tumor) cell. Thus, providing the saponin or the functional derivative thereof with a binding molecule (i.e. the sdAb comprised by the conjugate of the invention) and combining the conjugate with a second conjugate comprising an effector molecule (i.e. the effector molecule comprised by the ADC or the AOC) results in an improved effector-molecule activity potentiating effect, when the conjugate of the invention is contacted with the target cell that expresses the cell-surface molecule for binding the sdAb in the conjugate of the invention and that expresses the cell-surface molecule for binding the ADC or the AOC, on its surface, i.e. the binding target for the sdAb and the binding target for the ADC or AOC, which can be the same or which can be different as long as they are exposed on the same target (tumor) cell. Targeted saponin is already effective at lower dose than free saponin, in delivery of the effector molecule inside the target cell, and in delivery from the endosome or lysosome of said cell into the cytosol, where the effector molecule should bind its target binding partner and should exerts its biological activity (e.g. cell killing in case of the target cell being a tumor cell and the effector molecule being e.g. a toxin), however the present inventors have found that the combination of the conjugate of the invention comprising saponin and a cell targeting moiety (sdAb) and the second conjugate comprising the effector molecule (e.g. toxin for an ADC, e.g. BNA for an AON) is even more effective.

Hence, the inventors provide a pharmaceutical composition comprising the conjugate comprising the saponin (functional derivative) of the invention, and a second conjugate comprising an effector molecule and an antibody such as an sdAb, for targeted delivery of the two conjugates at the same target cells, which pharmaceutical composition has an improved therapeutic window, less risk for inducing side effects when an effective dose of the effector molecule comprised by the second conjugate is administered to a patient in need of effector molecule based therapy, and improved effector molecule activity due to improved delivery of the second conjugate inside target cells under influence of the targeted saponin as part of the conjugate of the invention, more specifically inside the cytosol of such target cells, when compared to current ADCs based on full-length antibodies or Fc comprising constructs thereof, which are not applied in combination with a covalently linked saponin as part of the conjugate of the invention. It is part of the invention that such conjugates of the invention are administered to patients in need of effector molecule based therapy together with an ADC or an AOC, and optionally together with a dose of free saponin (functional derivative), although the application of the conjugate of the invention alone is preferred as the source of saponin for delivery into the endosome/endolysosome/lysosome of a target cell.

An example of a saponin suitable for application in the conjugate of the invention is a monodesmosidic or bi-desmosidic triterpene saponin belonging to the type of a 12,13-dehydrooleanane with the aldehyde group in position C-23 and optionally comprising a glucuronic acid group in a carbohydrate substituent at the C-3beta-OH group of the saponin, preferably a bi-desmosidic triterpene saponin belonging to the type of a 12,13-dehydrooleanane with the aldehyde group in position C-23 and comprising a glucuronic acid group in a carbohydrate substituent at the C-3beta-OH group of the saponin. An exemplary saponin according to the invention comprises one, several, or all of the features of the saponin depicted as SAPONIN A and illustrated by the following structure:

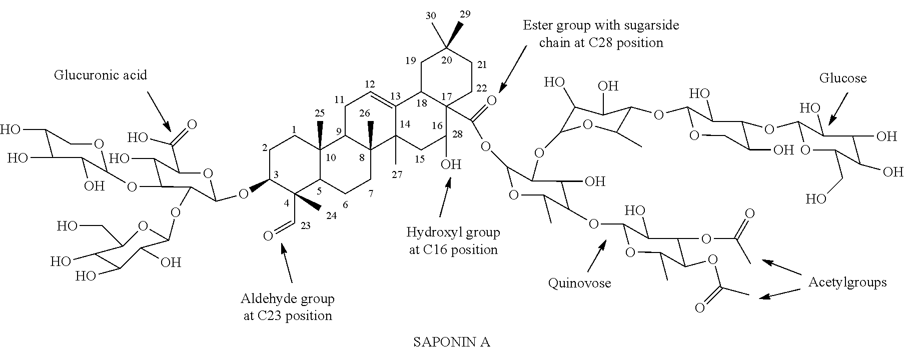

This group of saponins has demonstrated endosomal escape enhancing activity towards an effector moiety when the saponin and the effector moiety were present in the endosome of a cell. Typically, the saponins suitable for application in the conjugates according to the invention are saponins with a triterpene backbone wherein the structure of the triterpene backbone is a pentacyclic C30 terpene skeleton (also referred to as sapogenin or aglycone). Table A1 lists saponins suitable for synthesizing a conjugate comprising at least one sdAb such as V_{HH} and comprising at least one saponin, such as 1-16 saponin moieties, 1-8 such as 2, 4 or 8 saponin moieties, wherein the saponin is for example SO1861 or QS-21, preferably SO1861.

It is preferred that the one or more sdAbs in the conjugate is/are camelid VHS. Such VHS can readily be obtained using an immunization approach and/or by applying phage display techniques with either a naïve library, or a library obtained using an immunization strategy, including immunization of a Camelidae species such as a lama with at least the portion of a cell-surface molecule exposed on the outer surface of the cell selected for shuttling a saponin from outside said cell inside said cell. Application of such camelid V_{H} in the conjugate of the invention is beneficial since such binding domain is relatively stable, and smaller than a conventional immunoglobulin G type of antibody, or common fragments thereof such as scFv, Fab, commonly resulting in improved tissue penetration when administered to e.g. a human subject. It will be appreciated that any sdAb capable of inducing endocytosis (receptor mediated internalization) once bound to a cell-surface molecule is suitable for application in the conjugate of the invention.

An embodiment is the conjugate of the invention, wherein the conjugate comprises at least two sdAbs with a single first sdAb covalently linked to the at least one saponin, or with two or more sdAbs linked to at least one saponin, or with all of the at least two sdAbs linked to at least one saponin.

An embodiment is the conjugate of the invention, comprising one—eight sdAbs, capable of binding to the same binding site on a cell-surface molecule of the cell, wherein the at least one saponin is linked to a single first sdAb of the one—eight sdAbs or wherein the at least one saponin is linked to two or more of the sdAbs, if present. It is part of the invention that more than one sdAb of the same type, which are the same and which are capable of binding to the same binding site on the cell surface, are linked together in the conjugate of the invention. Multiple sdAbs in the conjugate contribute to improved binding affinity and avidity when more than one of these multiple sdAbs can bind simultaneously to binding sites present on the (same) cell surface. Such multiple binding events at the cell surface may facilitate uptake of the conjugate by the cell, e.g. via receptor-mediated endocytosis when the binding site on the cell surface is part of a cell-surface receptor capable of endocytosing the conjugate once bound to such receptor. If the conjugate of the invention comprises more than one copy of the sdAb for binding to the binding site on the target cell, a single copy of those sdAbs is bound to at least one saponin, or more than one copy of the sdAbs, or all of the sdAbs comprised by the conjugate, are bound to at least one saponin, such as one saponin bound to each copy of the more than one sdAb present in the conjugate. An embodiment is the conjugate of the invention wherein the conjugate comprises a single sdAb and comprises a single saponin or multiple saponins covalently bound thereto. Thus a conjugate of the invention comprises in some embodiment a single sdAb moiety and a single or multiple saponin moieties. An embodiment is a conjugate of the invention, comprising at least two sdAbs which are biparatopic, preferably comprising two sdAbs which are biparatopic. An example of such a biparatopic tandem of sdAbs is the biparatopic tandem of sdAbs with amino-acid sequence as depicted as SEQ ID NO: 74. The bivalent tandem of sdAb having amino-acid sequence of SEQ ID NO: 74 binds to EGFR. A first sdAb, 7D12 with amino-acid sequence as depicted as SEQ ID NO: 75, binds to a first epitope on the EGFR and a second sdAb, 9G8, with amino-acid sequence as depicted as SEQ ID NO: 76, binds to a second epitope on the EGFR. Of course, the sdAb 7D12 or the sdAb 9G8 are also suitable for application in a conjugate of the invention which comprises a single sdAb, or which comprises at least one further sdAb different from 7D12 and 9G8, which at least one further sdAb binds to EGFR or to a further cell-surface molecule present on the same cell surface as at which the EGFR is exposed.

An embodiment is the conjugate of the invention, wherein the sdAb-saponin conjugate comprises 1-100 saponin moieties of the at least one saponin, preferably 2-64 saponin moieties, more preferably 4-32 saponin moieties, most preferably 8-16 saponin moieties, or any number therein between. Typically, the sdAb-saponin conjugate of the invention comprises 1, 2, 4, 8 or 16 saponin moieties (copies of the saponin molecule). For example, 4 saponin molecules are covalently linked to a G2 dendron and at least one, such as one or two, of the dendron-(saponin)⁴ conjugate (saponin conjugate) is covalently linked to an sdAb. For example, 8 saponin molecules are covalently linked to a G3 dendron and at least one, such as one or two, of the dendron-(saponin)₈ conjugate (saponin conjugate) is covalently linked to an sdAb. An embodiment is the conjugate of the invention wherein the sdAb-saponin conjugate comprises more than one saponin moieties wherein the saponin moieties are the same or different. That is to say, if more than one saponins are covalently linked to the sdAb(s) in the sdAb-saponin conjugate of the inventions, these saponins can all be copies of the same saponin, or the saponin are different saponins. Preferred is the conjugate comprising multiple saponin moieties, wherein the saponins that are bound to the sdAb(s) are the same. For example, 2, 4, 8, 16 saponin molecules covalently linked to the sdAb's in the sdAb-saponin conjugate, for example 2-16 SO1861 copies or QS-21 copies, preferably SO1861.

Synchronization is the missing link between a successful delivery strategy for mice and its application in humans, when the application of the endosomal escape enhancing effect of saponin towards effector molecules is considered. Indeed, the inventors established in a series of in vivo mouse tumor models that separately administering to the mice a dose of free saponin and a dose of e.g. ADC without the presence of a conjugate comprising saponin according to the invention, did not result in any desired anti-tumor activity such as delayed tumor growth, tumor regression, diminished and slower tumor growth, compared to control animals not treated with the ADC in the presence of free saponin.

See also the FIGS. 18-21 for in vivo tumor models and the Examples described in the Examples section, here below. The free saponin was administered using various routes of administration and using various time points of administering the free saponin compared to the moment of administering the ADC (administering free saponin before, during and after administering the ADC). The ADC tested in in vivo tumor models was cetuximab-dianthin (with free SO1861), or trastuzumab-saporin (with free SO1861). Varying the dose of free saponin did not provide for an efficacious anti-tumor activity. The ADCs referred to were administered at a dose that in itself did not inflict any beneficial anti-tumor effect on the tumor-bearing animals. Surprisingly, the inventors now established that beneficial anti-tumor activity in various in vitro mammalian cell-based bioassays using human tumor cells and/or in various in vivo animal tumor models can be achieved by treating the cells or animals with conjugates according to the invention in combination with an ADC or an AOC. The conjugates of the invention optionally comprising a scaffold according to the invention (see below; a scaffold such as a covalent saponin conjugate comprising an oligomeric or polymeric structure with one or multiple saponin moieties covalently bound thereto). The scaffold for example being a tri-functional linker with a covalently bound saponin (e.g. SO1861, QS-21) via a cleavable or non-cleavable linkage, the scaffold linked with a covalent bond to the cell-surface molecule targeting molecule of the conjugate such as a monoclonal antibody such as cetuximab, trastuzumab, OKT-9, but preferably an sdAb such as a V_{HH}, or the scaffold being a dendron, such as a dendron, for example G4-dendron or G5-dendron, to which for example four moieties can bind such as four saponin molecules, or a dendron for binding for example eight saponins, the dendron comprising a chemical group for (covalent) coupling to the cell-surface molecule targeting antibody such as an sdAb, of the conjugate. Reference is made to the further embodiments and the Examples section, exemplifying several of these scaffolds according to the invention, showing in vivo and/or in vitro anti-tumor cell activity when cell toxicity exerted by e.g. a proteinaceous toxin is considered or when gene silencing in the tumor cell is considered.

Without wishing to be bound by any theory, in view of the failures observed when treatment of tumor-bearing animals with an ADC together with free saponin is considered, it is preferred to synchronize the presence of both, the at least one saponin, and the effector moiety, preferably a toxin or an oligonucleotide, in compartments or vesicles of the endocytic pathway of the target cell, e.g. a tumor cell or an auto-immune cell. With ADC and free saponin, synchronizing the presence of the molecules in the late endosomes, in order to obtain the synergistic effects in vivo was not beneficially obtainable according to attempts of the inventors. In one aspect, the invention preferably solves at least the following problem with respect to combining a conjugate comprising the effector moiety (e.g. an ADC or an AOC) with a conjugate of the invention comprising the saponin(s): without wishing to be bound by any theory the only reasonable chemical group within, e.g., the saponins that can be used for (covalent), in particular single and cleavable, retainable coupling is required for the endosomal escape activity.

Known restrictions are most likely the reason why saponins have not been used in combination with pharmaceutically active substances in clinical investigations other than the application of saponins in vaccination regimes wherein the use of an immune-potentiating adjuvant substance was implied, although the striking endosomal escape enhancer effect of, e.g., saponins of the invention and exemplified herein, is known for more than 10 years. For example providing a conjugate of the invention with a covalently bound saponin, for example in the context of a scaffold carrying several saponins, solves these difficulties, at least in part. Surprisingly, the saponins previously applied for their immune-potentiating activity in the vaccination context involving saponins as adjuvant component, are now also suitably for (covalent) coupling to the cell-surface molecule targeting antibody, such as an sdAb, comprised by the conjugate of the invention, for anti-tumor activity in vitro and in vivo when used in combination with an ADC or an AOC.

An embodiment is the conjugate of the invention, wherein the at least one saponin comprises an aglycone core structure selected from any one of:
2alpha-hydroxy oleanolic acid;
16alpha-hydroxy oleanolic acid;
hederagenin (23-hydroxy oleanolic acid);
16alpha,23-dihydroxy oleanolic acid;
gypsogenin;
quillaic acid;
protoaescige n in-21(2-m ethylbut-2-enoate)-22-acetate;
23-oxo-barringtogenol C-21,22-bis(2-methylbut-2-enoate);
23-oxo-barri ngtogenol C-21 (2-methylbut-2-enoate)-16,22-diacetate;
digitogenin;
3,16,28-trihydroxy oleanan-12-en; and
gypsogenic acid,
   preferably, the at least one saponin comprises an aglycone core structure selected from quillaic acid and gypsogenin, more preferably the aglycone core structure of the at least one saponin is quillaic acid.

Without wishing to be bound by any theory, presence of an aldehyde group (or derivative thereof) in the aglycone core structure of the saponin (here, also referred to as ‘aglycone’) is beneficial for the capacity of the saponin to stimulate and/or potentiate the endosomal escape of the effector molecule comprised by a second conjugate such as an ADC or AOC, according to the invention, when such a saponin co-localizes in a cell, in the endosome of said cell, with these effector molecules, as part of the conjugate of the invention or when in free form inside the endosome (e.g. split off from the conjugate once the conjugate is delivered inside the target cell endosome or lysosome), and when exposed to the cells in the presence of the second conjugate bearing the effector molecule. Therefore, the conjugates of the invention comprising saponin which has an aglycone with an aldehyde group is preferred. In quillaic acid and in gypsogenin the aldehyde group is at the C23 atom of the aglycone.

An embodiment is the conjugate of the invention, wherein the at least one saponin comprises a first saccharide chain, which is bound to the 03 atom or the 028 atom of the aglycone core structure of the at least one saponin, preferably to the 03 atom, and/or wherein the at least one saponin comprises a second saccharide chain, which is bound to the 028 atom of the aglycone core structure of the at least one saponin, preferably, the at least one saponin comprises the first and second saccharide chain. Thus, when the saponin comprised by the conjugate of the invention bears two glycans (saccharide chains), the first saccharide chain is bound at position C3 of the aglycone core structure and the second saccharide chain is bound at position 028 of the aglycone core structure.

An embodiment is the conjugate of the invention, wherein the at least one saponin comprises a first saccharide chain selected from:
GlcA-,
Glc-,
Gal-,
Rha-(1→2)-Ara-,
Gal-(1→2)-[Xyl-(1→3)]-GlcA-,
Glc-(1→2)-[Glc-(1-4)]-GlcA-,
Glc-(1→2)-Ara-(1→3)-[Gal-(1→2)]-GlcA-,
Xyl-(1→2)-Ara-(1→3)-[Gal-(1→2)]-GlcA-,
Glc-(1→3)-Gal-(1→2)-[Xyl-(1→3)]-Glc-(1-4)-Gal-,
Rha-(1→2)-Gal-(1→3)-[Glc-(1→2)]-GlcA-,
Ara-(1→4)-Rha-(1→2)-Glc-(1→2)-Rha-(1→2)-GlcA-,
Ara-(1→4)-Fuc-(1→2)-Glc-(1→2)-Rha-(1→2)-GlcA-,
Ara-(1→4)-Rha-(1→2)-Gal-(1→2)-Rha-(1→2)-GlcA-,
Ara-(1→4)-Fuc-(1→2)-Gal-(1→2)-Rha-(1→2)-GlcA-,
Ara-(1→4)-Rha-(1→2)-Glc-(1→2)-Fuc-(1→2)-GlcA-,
Ara-(1→4)-Fuc-(1→2)-Glc-(1→2)-Fuc-(1→2)-GlcA-,
Ara-(1→4)-Rha-(1→2)-Gal-(1→2)-Fuc-(1→2)-GlcA-,
Ara-(1→4)-Fuc-(1→2)-Gal-(1→2)-Fuc-(1→2)-GlcA-,
Xyl-(1→4)-Rha-(1→2)-Glc-(1→2)-Rha-(1→2)-GlcA-,
Xyl-(1→4)-Fuc-(1→2)-Glc-(1→2)-Rha-(1→2)-GlcA-,
Xyl-(1→4)-Rha-(1→2)-Gal-(1→2)-Rha-(1→2)-GlcA-,
Xyl-(1→4)-Fuc-(1→2)-Gal-(1→2)-Rha-(1→2)-GlcA-,
Xyl-(1→4)-Rha-(1→2)-Glc-(1→2)-Fuc-(1→2)-GlcA-,
Xyl-(1→4)-Fuc-(1→2)-Glc-(1→2)-Fuc-(1→2)-GlcA-,
Xyl-(1→4)-Rha-(1→2)-Gal-(1→2)-Fuc-(1→2)-GlcA-, and
Xyl-(1→4)-Fuc-(1→2)-Gal-(1→2)-Fuc-(1→2)-GlcA-,
   and/or wherein the at least one saponin comprises a second saccharide chain is selected from:
Glc-,
Gal-,
Rha-(1→2)-[Xyl-(1-4)]-Rha-,
Rha-(1→2)-[Ara-(1→3)-Xyl-(1→4)]-Rha-,
Ara-,
Xyl-,
Xyl-(1→4)-Rha-(1→2)-[R1-(→4)]-Fuc- wherein R1 is 4E-Methoxycinnamic acid,
Xyl-(1→4)-Rha-(1→2)-[R2-(→4)]-Fuc- wherein R2 is 4Z-Methoxycinnamic acid,
Xyl-(1→4)-[Gal-(1→3)]-Rha-(1→2)-4-OAc-Fuc-,
Xyl-(1→4)-[Glc-(1→3)]-Rha-(1→2)-3,4-di-OAc-Fuc-,
Xyl-(1→4)-[Glc-(1→3)]-Rha-(1→2)-[R3-(→4)]-3-OAc-Fuc- wherein R3 is 4E-Methoxycinnamic acid,
Glc-(1→3)-Xyl-(1→4)-[Glc-(1→3)]-Rha-(1→2)-4-OAc-Fuc-,
Glc-(1→3)-Xyl-(1→4)-Rha-(1→2)-4-OAc-Fuc-,
(Ara- or Xyl-)(1→3)-(Ara- or Xyl-)(1→4)-(Rha- or Fuc-)(1→2)-[4-OAc-(Rha- or Fuc-)(1-4)]-(Rha- or Fuc-),
Xyl-(1→3)-Xyl-(1-4)-Rha-(1→2)-[Qui-(1-4)]-Fuc-,
Api-(1→3)-Xyl-(1→4)-[Glc-(1→3)]-Rha-(1→2)-Fuc-,
Xyl-(1→4)-[Gal-(1→3)]-Rha-(1→2)-Fuc-,
Xyl-(1→4)-[Glc-(1→3)]-Rha-(1→2)-Fuc-,
Ara/Xyl-(1-4)-Rha/Fuc-(1-4)-[Glc/Gal-(1→2)]-Fuc-,
Api-(1→3)-Xyl-(1→4)-[Glc-(1→3)]-Rha-(1→2)-[R4-(→4)]-Fuc- wherein R4 is 5-O-[5-O-Ara/Api-3,5-dihydroxy-6-methyl-octanoyl]-3,5-dihydroxy-6-methyl-octanoic acid),
Api-(1→3)-Xyl-(1→4)-Rha-(1→2)-[R5-(→4)]-Fuc- wherein R5 is 5-O-[5-O-Ara/Api-3,5-dihydroxy-6-methyl-octanoyl]-3,5-dihydroxy-6-methyl-octanoic acid),
Api-(1→3)-Xyl-(1→4)-Rha-(1→2)-[Rha-(1→3)]-4-OAc-Fuc-,
Api-(1→3)-Xyl-(1→4)-[Glc-(1→3)]-Rha-(1→2)-[Rha-(1→3)]-4-OAc-Fuc-,
6-OAc-Glc-(1→3)-Xyl-(1→4)-Rha-(1→2)-[3-OAc-Rha-(1→3)]-Fuc-,
Glc-(1→3)-Xyl-(1→4)-Rha-(1→2)-[3-OAc-Rha-(1→3)]-Fuc-,
Xyl-(1→3)-Xyl-(1→4)-Rha-(1→2)-[Qui-(1→4)]-Fuc-,
Glc-(1→3)-[Xyl-(1→4)]-Rha-(1→2)-[Qui-(1-4)]-Fuc-,
Glc-(1→3)-Xyl-(1-4)-Rha-(1→2)-[Xyl-(1→3)-4-OAc-Qui-(1-4)]-Fuc-,
Xyl-(1→3)-Xyl-(1→4)-Rha-(1→2)-[3,4-di-OAc-Qui-(1→4)]-Fuc-,
Glc-(1→3)-[Xyl-(1→4)]-Rha-(1→2)-Fuc-, 6-OAc-Glc-(1→3)-[Xyl-(1→4)]-Rha-(1→2)-Fuc-,
Glc-(1→3)-[Xyl-(1→3)-Xyl-(1-4)]-Rha-(1→2)-Fuc-,
Xyl-(1→3)-Xyl-(1→4)-Rha-(1→2)-[Xyl-(1→3)-4-OAc-Qui-(1→4)]-Fuc-,
Api/Xyl-(1→3)-Xyl-(1→4)-[Glc-(1→3)]-Rha-(1→2)-[Rha-(1→3)]-40Ac-Fuc-,
Api-(1→3)-Xyl-(1→4)-[Glc-(1→3)]-Rha-(1→2)-[Rha-(1→3)]-40Ac-Fuc-,
Api/Xyl-(1→3)-Xyl-(1→4)-[Glc-(1→3)]-Rha-(1→2)-[R6-(→4)]-Fuc- wherein R6 is 5-O-[5-O-Rha-(1→2)-Ara/Api-3,5-dihydroxy-6-methyl-octanoyl]-3,5-dihydroxy-6-methyl-octanoic acid),
Api/Xyl-(1→3)-Xyl-(1→4)-[Glc-(1→3)]-Rha-(1→2)-[R7-(→4)]-Fuc- wherein R7 is 5-O-[5-O-Ara/Api-3,5-dihydroxy-6-methyl-octanoyl]-3,5-dihydroxy-6-methyl-octanoic acid),
Api/Xyl-(1→3)-Xyl-(1→4)-[Glc-(1→3)]-Rha-(1→2)-[R8-(→4)]-Fuc- wherein R8 is 5-O-[5-O-Ara/Api-3,5-dihydroxy-6-methyl-octanoyl]-3,5-dihydroxy-6-methyl-octanoic acid),
Api-(1→3)-Xyl-(1→4)-Rha-(1→2)-[R9-(→4)]-Fuc- wherein R9 is 5-O-[5-O-Ara/Api-3,5-dihydroxy-6-methyl-octanoyl]-3,5-dihydroxy-6-methyl-octanoic acid),
Xyl-(1→3)-Xyl-(1→4)-Rha-(1→2)-[R10-(→4)]-Fuc- wherein R10 is 5-O-[5-O-Ara/Api-3,5-dihydroxy-6-methyl-octanoyl]-3,5-dihydroxy-6-methyl-octanoic acid),
Api-(1→3)-Xyl-(1→4)-Rha-(1→2)-[R11-(→3)]-Fuc- wherein R11 is 5-O-[5-O-Ara/Api-3,5-dihydroxy-6-methyl-octanoyl]-3,5-dihydroxy-6-methyl-octanoic acid),
Xyl-(1→3)-Xyl-(1→4)-Rha-(1→2)-[R12-(→3)]-Fuc- wherein R12 is 5-O-[5-O-Ara/Api-3,5-dihydroxy-6-methyl-octanoyl]-3,5-dihydroxy-6-methyl-octanoic acid) and
Glc-(1→3)-[Glc-(1→6)]-Gal-,
   preferably the at least one saponin comprises such a selected first saccharide chain and such a selected second saccharide chain. Thus, when the saponin comprised by the conjugate of the invention bears two glycans (saccharide chains), the first saccharide chain is bound at position C₃ of the aglycone core structure and the second saccharide chain is bound at position C₂₈ of the aglycone core structure.

An embodiment is the conjugate of the invention, wherein the at least one saponin is selected from: 3-O-beta-D-galactopyranosyl-(1→2)-[beta-D-xylopyranosyl-(1→3)]-beta-D-glucuronopyranosyl quillaic acid 28-O-beta-D-glucopyranosyl-(1→3)-beta-D-xylopyranosyl-(1→4)-alpha-L-rhamnopyranosyl-(1→2)-[beta-D-xylopyranosyl-(1→3)-4OAc-beta-D-quinovopyranosyl-(1→4)]-beta-D-fucopyranoside, Quillaja bark saponin, dipsacoside B, saikosaponin A, saikosaponin D, macranthoidin A, esculentoside A, phytolaccagenin, aescinate, AS6.2, NP-005236, AMA-1, AMR, alpha-Hederin, NP-012672, NP-017777, NP-017778, NP-017774, NP-018110, NP-017772, NP-018109, NP-017888, NP-017889, NP-018108, SA1641, AE X55, NP-017674, NP-017810, AG1, NP-003881, NP-017676, NP-017677, NP-017706, NP-017705, NP-017773, NP-017775, SA1657, AG2, SO1861, SO1862, SO1904, GE1741, SO1542, SO1584, SO1658, SO1674, SO1832, QS-7, QS1861, QS-7 api, QS1862, QS-17, QS-18, QS-21 A-apio, QS-21 A-xylo, QS-21 B-apio, QS-21 B-xylo, beta-Aescin, Aescin Ia, Teaseed saponin I, Teaseedsaponin J, Assamsaponin F, Digitonin, Primula acid 1 and AS64R, and/or a functional derivative thereof, and/or a stereoisomer thereof, and/or any combination thereof, the functional derivative optionally without an aldehyde group in the aglycone core structure of the at least one saponin, and/or without a glucuronic acid moiety comprising a carboxyl group in a first saccharide chain of the at least one saponin, and/or without an acetyl group in a second saccharide chain of the at least one saponin, preferably the at least one saponin is selected from: QS-21, GE1741, SA1641 and SO1861, and/or a functional derivative thereof, the functional derivative preferably without an aldehyde group in the aglycone core structure of the at least one saponin, and/or without a glucuronic acid moiety comprising a carboxyl group in a first saccharide chain of the at least one saponin, and/or without an acetyl group in a second saccharide chain of the at least one saponin. Preferred is the saponin-comprising conjugate of the invention, wherein the at least one saponin is selected from: 3-O-beta-D-galactopyranosyl-(1→2)-[beta-D-xylopyranosyl-(1→3)]-beta-D-glucuronopyranosyl quillaic acid 28-O-beta-D-glucopyranosyl-(1→3)-beta-D-xylopyranosyl-(1→4)-alpha-L-rhamnopyranosyl-(1→2)-[beta-D-xylopyranosyl-(1→3)-40Ac-beta-D-quinovopyranosyl-(1→4)]-beta-D-fucopyranoside, Quillaja bark saponin, dipsacoside B, saikosaponin A, saikosaponin D, macranthoidin A, esculentoside A, phytolaccagenin, aescinate, AS6.2, NP-005236, AMA-1, AMR, alpha-Hederin, NP-012672, NP-017777, NP-017778, NP-017774, NP-018110, NP-017772, NP-018109, NP-017888, NP-017889, NP-018108, SA1641, AE X55, NP-017674, NP-017810, AG1, NP-003881, NP-017676, NP-017677, NP-017706, NP-017705, NP-017773, NP-017775, SA1657, AG2, SO1861, S01862, S01904, GE1741, S01542, S01584, S01658, S01674, S01832, QS-7, QS1861, QS-7 api, QS1862, QS-17, QS-18, QS-21 A-apio, QS-21 A-xylo, QS-21 B-apio, QS-21 B-xylo, beta-Aescin, Aescin Ia, Teaseed saponin I, Teaseedsaponin J, Assamsaponin F, Digitonin, Primula acid 1 and AS64R, preferably the at least one saponin is selected from: QS-21, GE1741, SA1641 and SO1861, and/or a functional derivative thereof, the functional derivative lacking an aldehyde group in the aglycone core structure of the at least one saponin, and/or without a glucuronic acid moiety comprising a carboxyl group in a first saccharide chain of the at least one saponin, more preferably QS-21, SO1861, most preferably, SO1861. Typically, the saponin is a saponin selected from Table A1, known for the endosomal escape enhancing activity towards an effector molecule when contacted with a selected cell together with the effector molecule (such as part as an effector moiety of an ADC or an AOC).

Such saponins of the triterpene glycoside type are capable of enhancing the endosomal escape of (effector) molecules that are present in the endosome (or lysosome) of a cell, when the saponin co-localizes with such (effector) molecule inside the cell. The inventors established that the endosomal escape enhancing activity of these saponins is about 100 to 1000 times more potent when the saponin is contacted with a cell as part of a conjugate of the invention. The free saponin is capable of stimulating the delivery of (effector) molecules in the cytosol of cells, when such cells are contacted with the (effector) molecules and the saponin, at 100-1000 times higher saponin concentration, compared to the concentration of the same saponin which is comprised by the conjugate of the invention, required to achieve the same extent of delivery of the (effector) molecule from outside the cell to inside the endosome and finally in the cytosol of said cell. Saponins which display such endosomal escape enhancing activity are listed in Table A1, as well as saponins with high structural similarity with saponins for which the ability to potentiate the cytosolic delivery of (effector) molecules has been established. When the saponin is part of the conjugate of the invention, the targeted delivery of the saponin upon binding of the sdAb to the cell-surface binding site on the target cell, on said cell, and after endocytosis, into the endosome of said cell, is thus about 100 to 1000 times more effective compared to contacting the same cell with free, untargeted saponin which is not provided with a binding molecule such as an antibody or an sdAb for binding to a cell-surface molecule of a target cell. The small size of the sdAb of the conjugates of the invention, compared to e.g. IgG type of antibodies, or fragments thereof such as Fab, scFv, contribute to efficient uptake by the target cell that exposes the binding site for binding of the sdAb, e.g. uptake by endocytosis. Typically, the sdAbs in the conjugates of the invention are capable of binding to a cell-surface receptor of a target cell, such as a tumor cell specific cell-surface receptor. This way, the conjugates of the invention are particularly suitable for endocytosis into e.g. tumor cells expressing the cell-surface receptor.

An embodiment is the conjugate of the invention, wherein the at least one saponin is selected from: SO1861, SA1657, GE1741, SA1641, QS-21, QS-21A, QS-21 A-api, QS-21 A-xyl, QS-21B, QS-21 B-api, QS-21 B-xyl, QS-7-xyl, QS-7-api, QS-17-api, QS-17-xyl, QS1861, QS1862, Quillaja saponin, Saponinum album, QS-18, Quil-A, Gyp1, gypsoside A, AG1, AG2, SO1542, SO1584, SO1658, SO1674, SO1832, SO1862, SO1904, and/or a stereoisomer thereof, and/or a functional derivative thereof, and/or any combination thereof, optionally the functional derivative without an aldehyde group in the aglycone core structure of the at least one saponin, and/or without a glucuronic acid moiety comprising a carboxyl group in a first saccharide chain of the at least one saponin, and/or without an acetyl group in a second saccharide chain of the at least one saponin, preferably the at least one saponin is selected from: SO1861, GE1741, SA1641 and QS-21, preferably a (functional) derivative thereof without an aldehyde group in the aglycone core structure of the at least one saponin, and/or without a glucuronic acid moiety comprising a carboxyl group in a first saccharide chain of the at least one saponin, and/or without an acetyl group in a second saccharide chain of the at least one saponin. An embodiment is the saponin-comprising conjugate of the invention, wherein the at least one saponin is selected from: SO1861, SA1657, GE1741, SA1641, QS-21, QS-21A, QS-21 A-api, QS-21 A-xyl, QS-21B, QS-21 B-api, QS-21 B-xyl, QS-7-xyl, QS-7-api, QS-17-api, QS-17-xyl, QS1861, QS1862, Quillaja saponin, Saponinum album, QS-18, Quil-A, Gyp1, gypsoside A, AG1, AG2, SO1542, SO1584, SO1658, SO1674, SO1832, SO1862, SO1904, and preferably the at least one saponin is selected from: SO1861, GE1741, SA1641 and QS-21, preferably a functional derivative thereof without an aldehyde group in the aglycone core structure of the at least one saponin, and/or without a glucuronic acid moiety comprising a carboxyl group in a first saccharide chain of the at least one saponin. Typically, the at least one saponin comprised by the sdAb-saponin conjugate is selected from Table A1. SO1861 is preferred.

An embodiment is the conjugate of the invention, wherein the at least one saponin comprises an aglycone core structure comprising an aldehyde group, and/or comprises a first saccharide chain comprising a glucuronic acid moiety comprising a carboxyl group, and/or comprises a second saccharide chain comprising an acetyl group, preferably, the at least one saponin comprises an aglycone core structure comprising an aldehyde group, a first saccharide chain comprising a glucuronic acid moiety comprising a carboxyl group and a second saccharide chain comprising an acetyl group, more preferably, the at least one saponin comprises one or two of: an aglycone core structure comprising an aldehyde group, a first saccharide chain comprising a glucuronic acid moiety comprising a carboxyl group, a second saccharide chain comprising an acetyl group.

An embodiment is the conjugate of the invention, wherein the at least one saponin is a saponin (functional) derivative comprising none of an aglycone core structure comprising an aldehyde group, a first saccharide chain comprising a glucuronic acid moiety comprising a carboxyl group and a second saccharide chain comprising an acetyl group.

An embodiment is the conjugate of the invention, wherein the at least one saponin is covalently bound to the sdAb via a linker, preferably a cleavable linker. Coupling of the saponin to the sdAb via a linker provides flexibility when the binding site on the sdAb for coupling of the saponin is considered. Furthermore, such a linker may act as a spacer between the sdAb and the saponin, such that both the sdAb maintains its capability to bind to a binding site on a cell surface molecule and the saponin maintains its capability to enhance endosomal escape of a(n) (effector) molecule, when the conjugate co-localizes in the endosome or lysosome of the cell targeted by the conjugate upon binding of the sdAb to the cell-surface molecule, together with the (effector) molecule.

An embodiment is the conjugate of the invention, wherein the cleavable linker is subject to cleavage under acidic conditions, reductive conditions, enzymatic conditions and/or light-induced conditions, and preferably the cleavable linker comprises a cleavable bond selected from a hydrazone bond and a hydrazide bond subject to cleavage under acidic conditions, and/or a bond susceptible to proteolysis, for example proteolysis by Cathepsin B, and/or a bond susceptible for cleavage under reductive conditions such as a disulfide bond.

An embodiment is the conjugate of the invention, wherein the cleavable linker is subject to cleavage in vivo under acidic conditions such as for example present in endosomes and/or lysosomes of mammalian cells, preferably human cells, preferably the cleavable linker is subject to cleavage in vivo at pH 4.0-6.5, and more preferably at pH 5.5. Such cleavable linkers that are cleavable under the conditions as apparent in endosomes and lysosomes facilitates the delivery of free saponin inside the endosome or lysosome, upon cleavage of the saponin from the remainder of the conjugate of the invention. This way, the conjugate of the invention combines the benefits of cell-targeted delivery of the saponin upon specific binding of the sdAb to a cell-surface molecule on the target cell, and of the presence of the free saponin inside the cell, i.e. inside the endosome (or lysosome), which contributes to the ability of the free saponin to stimulate and/or facilitate the delivery of (effector) molecule out of the endosome (or lysosome) and into the cytosol of the target cell.

An embodiment is the conjugate of the invention, wherein the at least one saponin is covalently bound to the sdAb as part of a covalent saponin conjugate comprising an oligomeric molecule or a polymeric molecule, which oligomeric molecule or polymeric molecule is covalently bound to the at least one saponin and is covalently bound to the sdAb. An embodiment is the conjugate of the invention, wherein the at least one saponin is part of a covalent saponin conjugate comprising an oligomeric molecule or a polymeric molecule to which the saponin is covalently bound, and wherein the sdAb is also covalently bound to the same oligomeric molecule or polymeric molecule as to which the saponin is bound. Thus, the oligomeric molecule or the polymeric molecule comprises the one or more covalently bound saponin moieties and is covalently bound to a sdAb. The saponin and the sdAb are covalenty bound to each other via the polymeric molecule or the oligomeric molecule. The oligomeric molecule or the polymeric molecule links the sdAb('s) and the saponin(s) together, providing a saponin-comprising conjugate of the invention. Such a covalent saponin conjugate of saponins bound to the oligomeric or polymeric molecule serves as a carrier (scaffold, support) for multiple saponin moieties, which can be bound to the sdAb via a single bond, preferably via a (cleavable) linker. Since the covalent saponin conjugate can bear any selected number of covalently bound saponin moieties, such as 1-200 saponin moieties, relating to the type of selected oligomeric or polymeric structure comprising binding sites for covalent linking these saponins, application of such covalent saponin conjugate provides freedom when the number of saponin moieties in the sdAb-saponin conjugate of the invention is considered. For example, for cytosolic delivery of a selected effector molecule, the number of saponins present in a sdAb-saponin conjugate of the invention can be adapted by providing the covalent saponin conjugate with a number of saponin moieties sufficient and enough for stimulating the cytosolic delivery of the effector molecule, when the covalent saponin conjugate is part of the sdAb-saponin conjugate of the invention, and when the effector molecule co-localizes with the sdAb-saponin conjugate in the endosome or lysosome of a target cell in which the effector molecule should exert its biological activity.

An embodiment is the conjugate of the invention, wherein 1-8 of the covalent saponin conjugates is/are covalently bound to the sdAb, or 2-4 of the covalent saponin conjugates. An embodiment is the sdAb-saponin conjugate of the invention, wherein the oligomeric molecule or the polymeric molecule of the covalent saponin conjugate is a dendron, such as a G2 dendron, G3 dendron, G4 dendron or G5 dendron, wherein 1-32 saponin moieties, preferably 2, 3, 4 (for example for a G2 dendron), 5, 6, 8 (for example for a G3 dendron), 10, 16 (for example for a G4 dendron), 32 (for example for a G5 dendron) saponin moieties, or any number of saponin moieties therein between, such as 7, 9, 12 saponin moieties, are covalently bound to the oligomeric molecule or to the polymeric molecule of the covalent saponin conjugate.

An embodiment is the saponin-comprising conjugate of the invention, wherein 1-8 of the oligomeric molecules or polymeric molecules comprising the saponin(s) is/are covalently bound to the sdAb.

An embodiment is the saponin-comprising conjugate of the invention, wherein 2-4 of the oligomeric molecules or polymeric molecules comprising the saponin(s) are covalently bound to the sdAb.

An embodiment is the saponin-comprising conjugate of the invention, wherein the oligomeric molecule or the polymeric molecule of the covalent saponin conjugate is a dendron. Preferred is an oligomeric molecule or a polymeric molecule such as a dendron or a polyethylene glycol that is biologically sufficiently inactive or inert, preferably biologically inactive or inert.

An embodiment is the saponin-comprising conjugate of the invention, wherein the oligomeric molecule or the polymeric molecule of the covalent saponin conjugate is a G2 dendron, a G3 dendron, a G4 dendron or a G5 dendron with 4, 8, 16 and 32 binding sites for covalently binding 4, 8, 16 or 32 saponin moieties, respectively.

An embodiment is the saponin-comprising conjugate of the invention, wherein 1-32 saponin moieties, preferably 2, 3, 4, 5, 6, 8, 10, 16, 32 saponin moieties, or any number of saponin moieties therein between, such as 7, 9, 12 saponin moieties, are covalently bound to the oligomeric molecule or to the polymeric molecule of the covalent saponin conjugate.

Preferably, one or two of the covalent saponin conjugates is/are bound to a single sdAb in the sdAb-saponin conjugate of the invention. For many purposes, coupling of a single saponin or coupling of a single covalent saponin conjugate to a single sdAb comprised by the conjugate, suffices for efficient stimulation of effector molecule delivery into a target cell and into the cytosol of said cell. Typically, 4, 8 or 16 saponins are comprised by the conjugate of the invention, such as 4 or 8 saponins comprised by a single covalent saponin conjugate coupled to an sdAb in the conjugate of the invention. Typically, such conjugates of the invention comprise a single sdAb, to which the saponin or saponins or the covalent saponin conjugate(s) is/are bound, preferably a single saponin or a single covalent saponin conjugate. It is to be understood that “sdAb-saponin” in the context of sdAb-saponin conjugate is referring to the conjugate of at least one saponin molecule and at least one sdAb, and is not limited to a conjugate of the invention comprising a single sdAb and a single saponin molecule. For example, the conjugate comprises or consists of one or two sdAb's such as V_{HH}('s), and 1-32 saponin molecules, such as 2, 4, 8, 16 saponin molecules.

An embodiment is the conjugate of the invention, wherein the at least one saponin is covalently bound to the oligomeric molecule or to the polymeric molecule of the covalent saponin conjugate via any one or more of an imine bond, a hydrazone bond, a hydrazide bond, an oxime bond, a 1,3-dioxolane bond, a disulfide bond, a thio-ether bond, an amide bond, a peptide bond or an ester bond, preferably via a linker such as a cleavable linker.

An embodiment is the conjugate of the invention, wherein the at least one saponin comprises an aldehyde function in position 023 of the aglycone core structure of the at least one saponin and optionally a glucuronic acid function in a first saccharide chain at the C3beta-OH group of the aglycone core structure of the at least one saponin, which aldehyde function is involved in the covalent bonding to the sdAb directly or is involved in the covalent bonding to the oligomeric molecule or to the polymeric molecule of the covalent saponin conjugate, and/or, if present, the glucuronic acid function is involved in the covalent bonding to the sdAb directly or is involved in the covalent bonding of the at least one saponin to the oligomeric molecule or to the polymeric molecule of the covalent saponin conjugate, the bonding of the at least one saponin either via a direct covalent bond, or via a linker, wherein the linker is a cleavable linker or a stable linker. Here, stable refers to a bond between the saponin and the sdAb or to a bond between the saponin and the oligomeric or polymeric structure, which bond remains intact (is not cleaved) under the acidic conditions inside a cell, in particular the acidic conditions in the endosome or lysosome of such a cell. In addition, such a stable bond remains intact (i.e. is not cleaved) in e.g. the circulation and in the organs of a human subject to whom the conjugate of the invention comprising the covalent saponin conjugate, is administered. In contrast, a cleavable linker in the context of the binding of a saponin to a sdAb or to an oligomeric structure or a polymeric refers to a bond that is cleaved under the acidic conditions as apparent inside endosomes and lysosomes of mammalian cells such as human cell, e.g. tumor cell, whereas such cleavable linker remains intact (is not cleaved) when a conjugate comprising such cleavable bonds is present in the circulation or in organs, i.e. outside cells, of e.g. a human subject to whom the conjugate of the invention is administered.

An embodiment is the conjugate of the invention, wherein the aldehyde function in position C23 of the aglycone core structure of the at least one saponin is covalently bound to a linker, preferably a cleavable linker, more preferably to linker N-ε-maleimidocaproic acid hydrazide (EMCH), which linker is covalently bound via a thio-ether bond to a sulfhydryl group in the oligomeric molecule or in the polymeric molecule of the covalent saponin conjugate, such as a sulfhydryl group of a cysteine. Binding of the EMCH linker to the aldehyde group of the aglycone of the saponin results in formation of a hydrazone bond. Such a hydrazone bond is a typical example of a cleavable bond under the acidic conditions inside endosomes and lysosomes. A saponin that is coupled to an sdAb in the conjugate of the invention, or to an oligomeric structure or polymeric structure of a covalent saponin conjugate, wherein such a covalent saponin conjugate is coupled to a sdAb in the conjugate of the invention, is releasable from the conjugate of the invention once delivered in the endosome or lysosome of a target cell that exposes the cell-surface molecule to which the sdAb of the conjugate can bind. This way, saponin coupled to sdAb in the conjugate of the invention is transferred from outside the cell into the endosome (or lysosome), and in the endosome (or the lysosome), the saponin is released from the conjugate upon pH driven cleavage of the hydrazone bond. In the endosome (or the lysosome) the free saponin can exert its stimulatory activity when the delivery of an effector molecule co-localized in the endosome (or lysosome) into the cytosol is considered. Surprisingly, the inventors established that for the saponin it is not a prerequisite for endosomal escape enhancing activity of the saponin, that the saponin is present in endosomes or lysosomes in free form. Also saponins comprised by e.g. conjugates of the invention or comprised e.g. by a conjugate of an antibody and the saponin, are potentiating the delivery of effector molecule out of the endosome/lysosome into the cytosol of targeted cells, once the effector molecule and the saponin as part of a conjugate are contacted with the same target cell.

An embodiment is the conjugate of the invention, wherein the glucuronic acid function in the first saccharide chain at the C₃beta-OH group of the aglycone core structure of the at least one saponin is covalently bound to a linker such as linker 1-[Bis(dimethylamino)methylene]-1H-1,2,3-triazolo[4,5-b]pyridinium 3-oxid hexafluorophosphate (HATU), which linker is covalently bound via an amide bond to the sdAb directly or is covalently bound via an amide bond to an amine group in the oligomeric molecule or in the polymeric molecule of the covalent saponin conjugate, such as an amine group of a lysine or an N-terminus of a protein. When the HATU linker is coupled to the saponin and to the sdAb, the saponin is for example bound to the N-terminus of the sdAb or to the amine group of a lysine present in the sdAb.

An embodiment is the conjugate of the invention, wherein the polymeric molecule or the oligomeric molecule of the covalent saponin conjugate is bound to the sdAb, preferably to an amino-acid residue of the sdAb, involving a click chemistry group on the polymeric molecule or the oligomeric molecule of the covalent saponin conjugate, the click chemistry group preferably selected from a tetrazine, an azide, an alkene or an alkyne, or a cyclic derivative of these groups, more preferably the click chemistry group is an azide.

An embodiment is the conjugate of the invention, wherein the polymeric molecule or the oligomeric molecule of the covalent saponin conjugate comprises a polymeric structure and/or an oligomeric structure selected from: a linear polymer, a branched polymer and/or a cyclic polymer, an oligomer, a dendrimer, a dendron, a dendronized polymer, a dendronized oligomer, a DNA, a polypeptide, a poly-lysine, a poly-ethylene glycol, an oligo-ethylene glycol (OEG), such as OEG₃, OEG₄ and OEG₅, or an assembly of these polymeric structures and/or oligomeric structures which assembly is preferably built up by covalent cross-linking, preferably the polymeric molecule or the oligomeric molecule of the covalent saponin conjugate is a dendron such as a poly-amidoamine (PAMAM) dendrimer. An embodiment is the conjugate of the invention, wherein the polymeric molecule or the oligomeric molecule of the covalent saponin conjugate comprises a polymeric structure and/or an oligomeric structure selected from: a dendrimer, a dendron, a dendronized polymer, a dendronized oligomer, a DNA, for example 2-200 nucleic acids, a poly-ethylene glycol, an oligo-ethylene glycol (OEG), such as OEG₃, OEG₄ and OEG₅. The oligomeric molecule or the polymeric molecule is selected for the absence of intrinsic biological activity. Typically, the selected oligomeric molecule or the polymeric molecule is an inert molecule when biological activity is considered that would or could pose a health risk or that would or could result in adverse events in a human subject when the saponin-comprising conjugate of the invention which comprises such an oligomeric molecule or polymeric molecule is administered to said human subject. Driven by the number of selected saponins to be incorporated in the conjugate of the invention, the type and size or length of the oligomeric structure or polymeric structure is selected. That is to say, the number of saponins to be coupled to a sdAb for formation of the conjugate of the invention, can determine the selection of a suitable oligomeric or polymeric structure, bearing the sufficient amount of binding sites for coupling the desired number of saponins, therewith providing a covalent saponin conjugate bearing the selected number of saponin moieties to be coupled to a sdAb for provision of the conjugate of the invention. For example, length of an OEG or size of a Dendron or poly-lysine molecule determines the maximum number of saponins which can be covalently linked to such oligomeric or polymeric structure.

A conjugate according to the invention thus comprises at least one saponin. With “at least one” in this context is meant that the conjugate comprises one saponin molecule but may also comprise a couple (e.g. two, three or four) of saponins or a multitude (e.g. 10, 20 or 100) of saponins. Depending on the application, the conjugate may comprise a covalently bound scaffold (covalent saponin conjugate) with covalently bound saponins, wherein the scaffold may be designed such that it comprises a defined number of saponins. Preferably, a conjugate according to the invention comprises a defined number or range of saponins, rather than a random number. This is especially advantageous for drug development in relation to marketing authorization. A defined number in this respect means that a conjugate preferably comprises a previously defined number of saponins. This is, e.g., achieved by designing a scaffold comprising a polymeric structure with a certain number of possible moieties for the saponin(s) to (covalently) attach. Under ideal circumstances, all of these moieties are coupled to a saponin and the scaffold than comprises the prior defined number of saponins. It is envisaged to offer a standard set of scaffolds, comprising, e.g., two, four, eight, sixteen, thirty-two, sixty-four, etc., saponins so that the optimal number can be easily tested by the user according to his needs. An embodiment is the conjugate of the invention comprising the scaffold of the invention (covalent saponin conjugate of the invention), wherein the saponin is present in a defined range as, e.g., under non-ideal circumstances, not all moieties present in a polymeric structure bind a saponin. Such ranges may for instance be 2-4 saponin molecules per scaffold, 3-6 saponin molecules per scaffold, 4-8 saponin molecules per scaffold, 6-8 saponin molecules per scaffold, 6-12 saponin molecules per scaffold and so on. In such case, a conjugate comprising a scaffold according to the invention (covalent saponin conjugate of the invention), thus comprises 2, 3 or 4 saponins if the range is defined as 2-4.

The scaffold is fundamentally independent of the type of saponin covalently bound to the scaffold, the scaffold subsequently (in sequential order) covalently coupled to the conjugate of the invention. Thus, the conjugate of the invention comprising the scaffold (covalent saponin conjugate of the invention) is the basis product for a platform technology. Since the at least one covalently bound saponin mediates intracellular delivery of the effector moiety bound to the cell-surface molecule targeting ligand such as an antibody such as an sdAb, comprised by the second conjugate such as an ADC or an AOC, according to the invention, the scaffold technology according to the invention is a system that mediates controlled intracellular effector moiety delivery by saponins, by combining an ADC or an AOC with the conjugate of the invention which comprises the saponin and an sdAb. The scaffold provides an optimized and functionally active unit that can be linked to the saponin(s) and to the cell-surface molecule targeting sdAb comprised by the conjugate, at a single and defined position in the sdAb.

An embodiment is the conjugate of the invention comprising a scaffold according to the invention (covalent saponin conjugate of the invention), wherein the number of monomers of the polymeric or oligomeric structure is an exactly defined number or range. Preferably, the polymeric or oligomeric structure comprises structures such as poly(amines), e.g., polyethylenimine and poly(amidoamine), or structures such as polyethylene glycol, poly(esters), such as poly(lactides), poly(lactams), polylactide-co-glycolide copolymers, poly(dextrin), or a peptide or a protein, or structures such as natural and/or artificial polyamino acids, e.g. poly-lysine, DNA polymers, such as DNA comprising 2-100 nucleotides, stabilized RNA polymers or PNA (peptide nucleic acid) polymers, for example comprising 2-200 nucleotides, either appearing as linear, branched or cyclic polymer, oligomer, dendrimer, dendron (for example any of a G2, G3, G4 or G5 dendron, for maximally covalently binding of 4, 8, 16 or 32 saponin moieties, respectively), dendronized polymer, dendronized oligomer or assemblies of these structures, either sheer or mixed. Preferably, the polymeric or oligomeric structures are biocompatible, wherein biocompatible means that the polymeric or oligomeric structure does not show substantial acute or chronic toxicity in organisms and can be either excreted as it is or fully degraded to excretable and/or physiological compounds by the body's metabolism. Assemblies can be built up by covalent cross-linking or non-covalent bonds and/or attraction. They can therefore also form nanogels, microgels, or hydrogels, or they can be attached to carriers such as inorganic nanoparticles, colloids, liposomes, micelles or particle-like structures comprising cholesterol and/or phospholipids. Said polymeric or oligomeric structures preferably bear an exactly defined number or range of coupling moieties (chemical groups) for the coupling of glycoside molecules (and/or carrier molecules such as a ligand, monoclonal antibody or a fragment thereof such as an sdAb, wherein the sdAb is preferred according to the invention). Preferably at least 50%, more preferably at least 75%, more preferably at least 85%, more preferably at least 90%, more preferably at least 95%, more preferably at least 98%, more preferably at least 99%, most preferably (about) 100% of the exactly defined number or range of coupling moieties (chemical groups) in the polymeric or oligomeric structure is occupied by a glycoside molecule (saponin of the invention) in a scaffold according to the invention (covalent saponin conjugate of the invention).

Preferably, a dendron is a branched, clearly defined tree-like polymer with a single chemically addressable group at the origin of the tree, called the focal point. A dendrimer is a connection of two or more dendrons at their focal point. A dendronized polymer is a connection of the focal point of one or more dendrons to a polymer. In a preferred embodiment, a scaffold according to the invention is provided, wherein the polymeric or oligomeric structure comprises a linear, branched or cyclic polymer, oligomer, dendrimer, dendron, dendronized polymer, dendronized oligomer or assemblies of these structures, either sheer or mixed, wherein assemblies can be built up by covalent cross-linking or non-covalent attraction and can form nanogels, microgels, or hydrogels, and wherein, preferably, the polymer is a derivative of a poly(amine), e.g., polyethylenimine and poly(amidoamine), and structures such as polyethylene glycol, poly(esters), such as poly(lactids), poly(lactams), polylactide-co-glycolide copolymers, and poly(dextrin), and structures such as natural and/or artificial polyamino acids such as poly-lysine, or a peptide or a protein or DNA polymers, such as DNA comprising 2-100 nucleotides, stabilized RNA polymers or PNA (peptide nucleic acid) polymers, for example 2-200 nucleotides. Preferably, the polymeric or oligomeric structures are biocompatible.

An embodiment is the conjugate of the invention, wherein the sdAb is a single sdAb or are at least two, preferably two sdAbs, wherein the sdAb(s) can bind to a tumor-cell surface molecule of the cell, preferably a tumor-cell surface receptor such as a tumor-cell specific receptor, more preferably a receptor of the (target) cell selected from any one or more of: CD71, CA125, EpCAM(17-1A), CD52, CEA, CD44v6, FAP, EGF-IR, integrin, syndecan-1, vascular integrin alpha-V beta-3, HER2, EGFR, CD20, CD22, Folate receptor 1, CD146, CD56, CD19, CD138, CD27L receptor, prostate specific membrane antigen (PSMA), CanAg, integrin-alphaV, CA6, CD33, mesothelin, Cripto, CD3, CD30, CD239, CD70, CD123, CD352, DLL3, CD25, ephrinA4, MUC-1, Trop2, CEACAM5, CEACAM6, HER3, CD74, PTK7, Notch3, FGF2, C4.4A, FLT3, CD38, FGFR3, CD7, PD-L1, CTLA-4, CD52, PDGFRA, VEGFR1, VEGFR2, c-Met (HGFR), EGFR1, RANKL, ADAMTS5, CD16, CXCR7 (ACKR3), glucocorticoid-induced TNFR-related protein (GITR), most preferably selected from: HER2, c-Met, VEGFR2, CXCR7, CD71 and EGFR1.

These cell-surface molecules are typically present on tumor cells with tumor cell specificity, at least to a certain extent. Tumor cell specificity makes these receptors suitable targets for the conjugates of the invention, and therefore the sdAb in the conjugate is capable of binding to such a cell-surface receptor. Tumor cells are commonly targeted by conjugates comprising a tumor-cell receptor targeting binding molecule such as an antibody or a binding fragment or domain thereof, and comprising an effector molecule to be delivered inside the target tumor cell, more specifically in the cytosol of said tumor cell. Since the saponins comprised by the conjugate of the invention are capable of stimulating the release and delivery of effector molecules in the cytosol of cells, such as the (tumor) cells targeted by the sdAb comprised by the conjugate of the invention, it is particularly suitable to select as the target (tumor) cell surface molecule for the sdAb a cell-surface receptor known for its suitability to serve as the target for e.g. ADCs and AOCs. The conjugates of the invention are therewith suitable for co-delivery of an effector molecule that is part of an ADC or an AOC together with the saponin comprised by the conjugate of the invention. Targeting a tumor cell specific receptor with the conjugate of the invention promotes endocytosis and delivery of the saponin as part of the conjugate into the target cell endosome and/or lysosome. When the tumor cell is also contacted with an ADC or an AOC, the effector molecule comprised by such ADC or AOC is co-delivered into the endosome or lysosome, and under influence of the saponin, the effector molecule is subsequently delivered into the cytosol of the target cell. As said before, the application of targeted saponin when part of the conjugate of the invention results in an about 100 times to 1000 times potentiation of the stimulatory effect of the saponin, when biological activity of the effector molecule is considered, compared to the application of free saponin lacking a cell-targeting moiety such as an sdAb or a whole antibody. Application of the small sdAb such as a camelid V_{H} in the conjugate of the invention prevents or slows down clearance of the conjugate of the invention from the circulation and from the body of a human subject to whom the conjugate was administered, when compared to clearance rates commonly observed for antibody based ADCs. In addition, due to the relatively small size of the sdAb, the risk for limiting or hampering the saponin activity inside a target cell due to the presence of the linked protein domain is limited, compared to the larger size of e.g. an antibody bound to the saponin. In general, the smaller the size of the molecule linked to the saponin, the smaller the risk for interference with the saponin activity inside cells due to the presence of the bound molecule, e.g. an antibody domain such as a V_{HH}. Moreover, the relative small size of the sdAbs leads to their rapid distribution in tissue, such as tumor tissue, allowing for improved reaching of target cells and therewith to improved (extent of) binding to the target cells, compared to the relatively large-sized IgGs commonly applied in e.g. ADCs, OACs. One of the many benefits of applying sdAbs in the conjugates of the invention (which also applies for applying sdAbs in e.g. ADCs and AOCs), is the absence of an Fc tail common to regular antibodies of e.g. the IgG type. Absence of an Fc tail in the sdAb in the conjugate of the invention prevents occurrence of Fcγ-Receptor mediated off-target effects such as undesired side effects relating to Fcγ-Receptor activation, when the conjugate is administered to a patient in need thereof. Absence of an Fc tail eliminates the risk of side effects generated by the binding of an Fc to cells of a patient to whom e.g. an antibody-based ADC is administered. The conjugates of the invention do not bear this risk for Fc-mediated undesired side effects, whereas the inventors now show that such conjugates are highly efficient and effective in potentiation of an effector molecule comprised by an ADC, when tumor cells are contacted with both the ADC (at relatively low dose; see also the Examples section) and the conjugate of the invention (at relatively low dose; see also the Examples section).

An embodiment is the conjugate of the invention, wherein the sdAb is a single sdAb or are at least two, preferably two sdAbs, wherein the sdAb(s) is/are selected from: an anti-CD71 sdAb, an anti-HER2 sdAb, an anti-CD20 sdAb, an anti-CA125 sdAb, an anti-EpCAM (17-1A) sdAb, an anti-EGFR sdAb, an anti-CD30 sdAb, an anti-CD33 sdAb, an anti-vascular integrin alpha-v beta-3 sdAb, an anti-CD52 sdAb, an anti-CD22 sdAb, an anti-CEA sdAb, an anti-CD44v6 sdAb, an anti-FAP sdAb, an anti-CD19 sdAb, an anti-CanAg sdAb, an anti-CD56 sdAb, an anti-CD38 sdAb, an anti-CA6 sdAb, an anti-IGF-1R sdAb, an anti-integrin sdAb, an anti-syndecan-1 sdAb, an anti-CD79b, an anti-c-Met sdAb, an anti-EGFR1 sdAb, an anti-VEGFR2 sdAb, an anti-CXCR7 sdAb, wherein the sdAb(s) is/are preferably V_{HH}(s), more preferably camelid V_{H}(s). Preferred are sdAbs originating from antibodies capable of binding to tumor-cell specific receptors, in particular Her2, CD71 and EGFR.

An embodiment is the conjugate of the invention, wherein the conjugate comprises an sdAb that can bind to HER2, CD71 or to EGFR, wherein the sdAb is preferably a V_{HH}, more preferably camelid V_{H}. The inventors established that these tumor-cell specific receptors can be efficiently and effectively targeted by conjugates of the invention. For example, anti-HER2 V_{HH}, anti-CD71 V_{HH} and anti-EGFR V_{HH} are suitable for coupling to at least one saponin, or at least one covalent saponin conjugate according to the invention.

An embodiment is the conjugate of the invention, wherein the conjugate comprises an sdAb for binding to HER2 selected from: sdAb produced by clone 11A4, clone 18C3, clone 22G12, clone 017, clone Q17-C-tag; or an sdAb for binding to EGFR and produced by clone anti-EGFR Q86-C-tag; or an sdAb for binding to CD71 and produced by clone anti-CD71 Q52-C-tag; or an sdAb for binding to HIVgp41 and produced by clone anti-HIVgp41 Q8-C-tag; or an sdAb encoded by a cDNA of any one of the SEQ ID NO: 1, 3, 5, 7, 9, 11, 13, 15, 17, 19, 21, 23, 25, 27, 29 and 31; or any one of the sdAbs with an amino-acid sequence of SEQ ID NO: 2, 4, 6, 8, 10, 12, 14, 16, 18, 20, 22, 24, 26, 28, 30, 32, 36-72, wherein optionally the conjugate further comprises an sdAb for binding to albumin, such as any one or more of sdAbs with an amino-acid sequence of SEQ ID NO: 33, 34 and 35. An embodiment is the conjugate of the invention, wherein the conjugate comprises an sdAb for binding to HER2 selected from: sdAb produced by clone 11A4, clone 18C3, clone 22G12, clone 017, clone Q17-C-tag; or an sdAb for binding to EGFR and produced by clone anti-EGFR Q86-C-tag; or an sdAb for binding to CD71 and produced by clone anti-CD71 Q52-C-tag; or an sdAb for binding to HIVgp41 and produced by clone anti-HIVgp41 Q8-C-tag; or an sdAb encoded by a cDNA of any one of the SEQ ID NO: 1, 3, 5, 7, 9, 11, 13, 15, 17, 19, 21, 23, 25, 27, 29 and 31; or any one of the sdAbs with an amino-acid sequence of SEQ ID NO: 2, 4, 6, 8, 10, 12, 14, 16, 18, 20, 22, 24, 26, 28, 30, 32, 36-72, or the biparatopic tandem of sdAbs 7D12 and 9G8 with an amino-acid sequence of SEQ ID NO: 74 or sdAb 7D12 with an amino-acid sequence of SEQ ID NO: 75 or sdAb 9G8 with an amino-acid sequence of SEQ ID NO: 76, wherein optionally the conjugate further comprises an sdAb for binding to albumin, such as any one or more of sdAbs with an amino-acid sequence of SEQ ID NO: 33, 34 and 35. For example, the conjugate of a saponin and at least one V_{HH} comprises the tandem of biparatopic sdAbs with the amino-acid sequence of SEQ ID NO: 74, or comprises one or more copies of 7D12 and/or one or more copies of 9G8. V_{HH}s suitable for incorporation in the conjugate of the invention are for example found in the single domain antibody database (Wilton, E. E. et al. (2018)), in patent applications US20160251440 (anti-CD123, anti-CEACAM), U.S. Pat. No. 9,683,045 (anti-c-Met), US20090252681 (anti-EGFR, anti-IGF-1R), U.S. Pat. No. 9,969,805 (anti-HER2), US20190023796A1 (anti-HER3), and in Kijanka et al. (2013), for anti-HER2 and in Mercier et al. (2019) for anti-HER2. The amino-acid sequences and/or the cDNA sequences of a series of suitable V_{HH}s is also provided here below for anti-HER2, anti-HER3, anti-CD123, anti-CEACAM, anti-c-Met, anti-EGFR, anti-IGF-1R, anti-PD-L1, anti-CTLA-4, anti-CD19, anti-HER1 and anti-VGFR2, as SEQ ID NOs 1-32 and 36-72, in view of their ability to bind to tumor-cell specific receptors.

An embodiment is the conjugate of the invention, wherein the conjugate is represented by the general structure of Molecule I:

sdAb1(-L1-Sᵤ₁)ₙ-[L2a-((-sdAb2)ₘ₁(-L3-Sᵤ₂)ₚ)_{q1} . . . L2i-((-sdAb9)ₘ₂(-L4-Sᵤ₃)ₚ)_{q2}]-(L5-sdAb10ᵣ(-L6-Sᵤ₄)ₜ)ᵥ (Molecule I),
wherein sdAb1, sdAb2 . . . sdAb9, sdAb10 are sdAbs sdAb1, sdAb2, sdAb3, sdAb4, sdAb5, sdAb6, sdAb7, sdAb8, sdAb9 and sdAb10, which sdAbs are (copies of) the same sdAb;
S is at least one saponin moiety;
L1, L3, L4 and L6 are each independently a covalent bond or a covalent linker linking the at least one saponin to the sdAb, wherein such linkers are the same or different;
L2a-L2i, and L5 are each independently a covalent bond or a covalent linker linking two consecutive sdAbs if more than one sdAb is present, wherein such linkers are the same or different;
n is an integer selected from 0-4, preferably n is 1, 2, 3 or 4;
m1, m2 each independently is an integer selected from 0-10, preferably m1 and/or m2 is/are 0, 1, 2, 3, 4, 5, 6, 7 or 8, wherein, if m1 or m2 is >1, consecutive sdAbs are covalently linked, preferably via a linker;
p is an integer selected from 0-4, preferably p is 1, 2, 3 or 4;
q1, q2 each independently is an integer selected from 0-10, preferably q1 and/or q2 is/are 0, 1, 2, 3, 4, 5, 6, 7 or 8;
r is an integer selected from 0-10, preferably r is 0, 1, 2, 3, 4, 5, 6, 7, 8 or 9;
t is an integer selected from 0-4, preferably t is 1, 2, 3 or 4;
u1, u2, u3, u4 are an integer, each independently selected from 0-100, preferably u1, u2, u3 and/or u4 is/are 0, 1, 2, 4, 16, 32 or 64, preferably u1, u2, u3 and u4 are the same; and
v is 0 or 1.

An embodiment is the conjugate of the invention, wherein L1 and L2a are linkers, u1 is >0, n is >0, m1 is any of 1-9, p is 0, q1 is 1, m2 is 0, u2 is 0, u3 is 0, q2 is 0, v is 0, r is 0, u4 is 0 and t is 0, or wherein L1, L2a, L5 and L6 are a linker, u1 is 0, n is 0, m1 is any of 1-9, u2 is 0, p is 0, q1 is 1, m2 is 0, u3 is 0, p is 0, q2 is 0, r is 1, u4 is >1, t is >1 and v is 1.

A second aspect of the invention relates to a pharmaceutical combination comprising:
a first pharmaceutical composition comprising the conjugate of the invention and optionally comprising a pharmaceutically acceptable excipient and/or a pharmaceutically acceptable diluent; and
a second pharmaceutical composition comprising an active pharmaceutical ingredient (API) such as selected from any one or more of: a drug molecule, an oligonucleotide such as an mRNA, an anti-sense oligonucleotide, a ligand-drug conjugate such as EGF-dianthin or EGF-saporin, an antibody-drug conjugate (ADC) such as OKT9 monoclonal anti-CD71 antibody, trastuzumab or cetuximab conjugated with saporin, dianthin or a BNA, a ligand-oligonucleotide conjugate such as an antibody-oligonucleotide conjugate (AOC) such as an antibody-BNA conjugate or an antibody-siRNA conjugate, wherein the antibody of the ADC or the AOC optionally comprises or consists of at least one sdAb, wherein the at least one sdAb comprised by the ADC or AOC is/are different from or the same as the sdAb of the conjugate of the invention, and optionally comprising a pharmaceutically acceptable excipient and/or a pharmaceutically acceptable diluent. Preferred API's are an ADC and an AOC, wherein such an ADC or AOC comprises preferably an antibody such as an IgG or an sdAb such as one or more V_{HH}'s.

A further aspect of the invention relates to a pharmaceutical composition comprising:
the conjugate of the invention;
one or more active pharmaceutical ingredient(s) (API(s)); and
optionally comprising a pharmaceutically acceptable excipient and/or a pharmaceutically acceptable diluent.

The API or APIs comprised by the pharmaceutical composition is/are for example an API such as selected from: a drug molecule, an oligonucleotide such as an mRNA, an ASO, a ligand-drug conjugate such as EGF-dianthin or EGF-saporin, an ADC such as one of OKT9 monoclonal anti-CD71 antibody, trastuzumab or cetuximab, conjugated with saporin, dianthin or a BNA, a ligand-oligonucleotide conjugate such as an AOC such as an antibody-BNA conjugate or an antibody-siRNA conjugate, wherein optionally the antibody of the ADC or the AOC comprises or consists of at least one sdAb, wherein the at least one sdAb comprised by the ADC or AOC is/are different from or the same as the sdAb of the conjugate of the invention. Preferred API's are an ADC and an AOC, wherein such an ADC or AOC comprises preferably an antibody such as an IgG or an sdAb such as one or more V_{HH}'s.

An embodiment is the pharmaceutical composition comprising the conjugate of the invention and one or more API(s) as part of a second conjugate such as an ADC or an AOC comprising an effector molecule, wherein the at least one effector molecule comprises or consists of at least one of: urease and Cre-recombinase, a proteinaceous toxin, a ribosome-inactivating protein, a protein toxin, a bacterial toxin, a plant toxin, more preferably selected from any one or more of a viral toxin such as apoptin; a bacterial toxin such as Shiga toxin, Shiga-like toxin, Pseudomonas aeruginosa exotoxin (PE) or exotoxin A of PE, full-length or truncated diphtheria toxin (DT), cholera toxin; a fungal toxin such as alpha-sarcin; a plant toxin including ribosome-inactivating proteins and the A chain of type 2 ribosome-inactivating proteins such as dianthin e.g. dianthin-30 or dianthin-32, saporin e.g. saporin-S3 or saporin-S6, bouganin or de-immunized derivative debouganin of bouganin, shiga-like toxin A, pokeweed antiviral protein, ricin, ricin A chain, modeccin, modeccin A chain, abrin, abrin A chain, volkensin, volkensin A chain, viscumin, viscumin A chain; or an animal or human toxin such as frog RNase, or granzyme B or human angiogenin, or any toxic fragment or toxic derivative thereof; preferably the protein toxin is dianthin and/or saporin.

Typically, the ADC or the AOC comprises an antibody for binding to any one of the tumor-cell receptors listed here above, i.e. CD71, CA125, EpCAM(17-1A), CD52, CEA, CD44v6, FAP, EGF-IR, integrin, syndecan-1, vascular integrin alpha-V beta-3, HER2, EGFR, CD20, CD22, Folate receptor 1, CD146, CD56, CD19, CD138, CD27L receptor, prostate specific membrane antigen (PSMA), CanAg, integrin-alphaV, CA6, CD33, mesothelin, Cripto, CD3, CD30, CD239, CD70, CD123, CD352, DLL3, CD25, ephrinA4, MUC-1, Trop2, CEACAM5, CEACAM6, HER3, CD74, PTK7, Notch3, FGF2, C4.4A, FLT3, CD38, FGFR3, CD7, PD-L1, CTLA-4, CD52, PDGFRA, VEGFR1, VEGFR2, c-Met (HGFR), EGFR1, RANKL, ADAMTS5, CD16, CXCR7 (ACKR3), glucocorticoid-induced TNFR-related protein (GITR), preferably selected from: HER2, c-Met, VEGFR2, CXCR7, CD71 and EGFR1. Examples of such antibodies are cetuximab, trastuzumab, OKT-9. Since the ADC or the AOC may target the same cell-surface molecule as targeted by the sdAb in the conjugate of the invention, or since the ADC or the AOC may target a second cell-surface molecule different from the cell-surface molecule targeted by the sdAb comprised by the conjugate of the invention, the ADC or the AOC can comprise a sdAb selected from: an sdAb for binding to HER2 selected from: sdAb produced by clone 11A4, clone 18C3, clone 22G12, clone Q17, clone Q17-C-tag; or an sdAb for binding to EGFR and produced by clone anti-EGFR Q86-C-tag; or an sdAb for binding to CD71 and produced by clone anti-CD71 Q52-C-tag; or an sdAb for binding to HIVgp41 and produced by clone anti-HIVgp41 Q8-C-tag; or an sdAb encoded by a cDNA of any one of the SEQ ID NO: 1, 3, 5, 7, 9, 11, 13, 15, 17, 19, 21, 23, 25, 27, 29 and 31; or any one of the sdAbs with an amino-acid sequence of SEQ ID NO: 2, 4, 6, 8, 10, 12, 14, 16, 18, 20, 22, 24, 26, 28, 30, 32, 36-72. The ADC or the AOC can comprise a sdAb selected from: an sdAb for binding to HER2 selected from: sdAb produced by clone 11A4, clone 18C3, clone 22G12, clone 017, clone Q17-C-tag; or an sdAb for binding to EGFR and produced by clone anti-EGFR Q86-C-tag; or an sdAb for binding to CD71 and produced by clone anti-CD71 Q52-C-tag; or an sdAb for binding to HIVgp41 and produced by clone anti-HIVgp41 Q8-C-tag; or an sdAb encoded by a cDNA of any one of the SEQ ID NO: 1, 3, 5, 7, 9, 11, 13, 15, 17, 19, 21, 23, 25, 27, 29 and 31; or any one of the sdAbs with an amino-acid sequence of SEQ ID NO: 2, 4, 6, 8, 10, 12, 14, 16, 18, 20, 22, 24, 26, 28, 30, 32, 36-72, or the biparatopic tandem of sdAbs with an amino-acid sequence of SEQ ID NO: 74 or one of the two sdAbs with an amino-acid sequence of SEQ ID NO: 74, wherein optionally the conjugate further comprises an sdAb for binding to albumin, such as any one or more of sdAbs with an amino-acid sequence of SEQ ID NO: 33, 34 and 35. V_{HH}s suitable for incorporation in ADCs or AOCs according to the invention are for example found in the single domain antibody database (Wilton, E. E. et al. (2018)), in patent applications US20160251440 (anti-CD123, anti-CEACAM), U.S. Pat. No. 9,683,045 (anti-c-Met), US20090252681 (anti-EGFR, anti-IGF-1R), U.S. Pat. No. 9,969,805 (anti-HER2), US20190023796A1 (anti-HER3), and in Kijanka et al. (2013), for anti-HER2 and in Mercier et al. (2019) for anti-HER2. The amino-acid sequences and/or the cDNA sequences of a series of suitable V_{HH}s is also provided here below for anti-HER2, anti-HER3, anti-CD123, anti-CEACAM, anti-c-Met, anti-EGFR, anti-IGF-1R, anti-PD-L1, anti-CTLA-4, anti-CD19, anti-HER1 and anti-VGFR2, as SEQ ID NOs 1-32 and 36-72, in view of their ability to bind to tumor-cell specific receptors. Examples of such V_{HH}s suitable for incorporation in ADCs or AOCs according to the invention are for example anti-HER2 V_{HH}, anti-CD71 V_{HH}, anti-EGFR V_{HH}, which V_{HH}s can for example be coupled to an BNA, siRNA, toxin such as a protein toxin, such as dianthin and saporin. See for example the exemplifying embodiments in FIGS. 5-7.

An effector moiety useful as part of an ADC or an AOC in the present invention preferably relies on late endosomal escape for exerting its effect. Some effector molecules, such as, e.g., a pseudomonas exotoxin, are rerouted to other organelles prior to the “late endosomal stage” and, thus, would normally not benefit from incorporation in an ADC for combining with the conjugate according to the present invention. However, such toxin may be adapted for use with the present invention, e.g., by deleting the signal peptide responsible for rerouting. In particular toxins that are highly toxic and would require only one molecule to escape the endosomes to kill a cell maybe modified to be less potent. It is preferred to use a toxin that kills a cell if at least 2, more preferably at least 5, more preferably at least 10, more preferably at least 20, more preferably at least 50, most preferably at least 100 toxin molecules escape the endosome (and enter the cytosol). It is further preferred that a second conjugate of the invention comprised by the pharmaceutical composition or comprised by the pharmaceutical combination of the invention, comprises a covalently conjugated functionalized scaffold, i.e. a scaffold such as an oligomeric or polymeric molecule or a tri-functional linker, comprising covalently bound effector moiety or moieties for targeting the scaffold comprising the bound effector moiety/moieties at a target cell such as a tumor cell or an auto-immune cell. Further, in order to reduce off-target toxicity, cell membrane non-permeable small molecule toxins are preferred effector molecules over cell membrane permeable toxins.

Preferably, the effector moiety comprised by the ADC or the AOC for combination with the conjugate of the invention, which effector moiety effect is enhanced by the saponins comprised by the conjugate of the invention, detaches from the second conjugate (i.e. an ADC, an AOC), e.g. detaches from the antibody, such as an sdAb, present in the second conjugate as the cell-surface molecule targeting moiety of the second conjugate, when endocytosed. This can be achieved by a cleavable bond that breaks, e.g. under acidic, reductive, enzymatic or light-induced conditions.

The inventors show that a tumor-cell targeting monoclonal antibody or an sdAb provided with covalently coupled antisense BNA such as BNA(HSP27) and combined with a tumor-cell targeting monoclonal antibody or an sdAb, which is the same or different, provided with covalently coupled saponin (i.e. the conjugate of the invention), that is contacted with tumor cells, both the BNA coupled to the cell-targeting ligand (i.e. the second conjugate) (via a cleavable bond) and the saponin coupled to the antibody (e.g. sdAb in the conjugate of the invention) (via a cleavable bond), is capable of silencing HSP27 in vivo in tumors, compared to control and compared to the AOC bearing the BNA in the absence of the conjugate of the invention comprising the saponin (SO1861). Administering an ADC together with a conjugate comprising saponin of the invention, or administering an antibody-oligonucleotide conjugate such as an antibody-BNA conjugate, together with a conjugate comprising saponin of the invention, thus endows the ADC or AOC with anti-tumor cell activity not seen with only the ADC or only the AOC, in the absence of the conjugate of the invention, at the same dose. The antisense BNA (HSP27) was a BNA with oligonucleic acid sequence according to Zhang et al. (2011) [YZhang, Z Qu, S Kim, V Shi, B Liao1, P Kraft, R Bandaru, Y Wu, LM Greenberger and ID Horak, Down-modulation of cancer targets using locked nucleic acid (LNA)-based antisense oligonucleotides without transfection, Gene Therapy (2011) 18, 326-333]. Noteworthy, to the best of the knowledge of the inventors, BNA is designed for application as a free nucleic acid. The inventors are now the first to demonstrate that the antisense BNA can be covalently coupled through a (non-)cleavable linker with a ligand or an antibody such as a mAb or an sdAb such as a V_{HH}, in a way that gene-silencing activity is retained in vitro and more importantly in vivo in the tumor cells of a tumor-bearing animal. This approach of providing BNA based AOCs opens new ways to administer targeted BNA to human (cancer) patients in need thereof.

It will be appreciated that when the sdAb in the conjugate of the invention and the antibody or sdAb in the ADC or the AOC target different cell-surface molecules such as different tumor-cell specific receptors, the two different targets are both present at the surface of the target cell in which the effector molecule comprised by the ADC or the AOC and the saponin comprised by the conjugate of the invention, should be co-delivered. When the ADC or the AOC and the conjugate of the invention target different (tumor) cell receptors, the advantage is that e.g. the risk for off-target effects on e.g. cells that have one of the two cell-surface molecules on their surface, is reduced, when doses of the ADC or the AOC and of the conjugate of the invention are contacted with a mixture of different cell types including the target cells, such as occurs when the combination of the ADC or the AOC and the conjugate of the invention are (co-)administered to a patient in need thereof. By administering a dose of the ADC or the AOC which is too low to deliver a therapeutically effective amount of the effector molecule inside target cells, when the ADC or the AOC binds to such target cells, in the absence of the targeted saponin in the form of the conjugate of the invention, entrance of cells that may express the cell-surface molecule to which the ADC or the AOC can bind, but that do not express the target for the sdAb in the conjugate of the invention, will accordingly not induce e.g. a cytotoxic effect when the ADC is considered. Only in the target cell bearing both receptors for binding of the ADC or the AOC and for binding of the conjugate of the invention (on the same cell surface), the dose of the effector molecule will be sufficient and enough for exerting its biological effect in the cytosol of said cells, once endosomal escape occurred under influence of the saponin that co-localizes inside said target cells expressing both receptors.

In some embodiments, the sdAb comprised by the conjugate of the invention and the ADC or the AOC target the same cell-surface molecule such as the same tumor-cell specific receptor. This way, presence of only a single tumor-cell specific receptor allows for co-delivery of both the ADC or the AOC and the conjugate of the invention to inside the target cell bearing such single type of target receptor. An advantage of such co-delivery inside target cells using a single cell-specific surface molecule such as a tumor-cell specific receptor, e.g. HER2, EGFR, CD71, is the possibility to target tumor cells which only express a single receptor which is specific enough for specific targeting of such a target tumor cell by the conjugate of the invention and an ADC or an AOC. Using such a single specific receptor makes it possible to co-deliver the targeted saponin and the targeted effector molecule inside the tumor cell, such that such difficult-to-treat tumor cells can still be effectively provided with an effective dose of the effector molecule.

An embodiment is the pharmaceutical combination of the invention or the pharmaceutical composition of the invention, wherein the antibody of the ADC or the AOC comprises or is a V_{HH}, preferably a camelid V_{H}.

An embodiment is the pharmaceutical combination of the invention or the pharmaceutical composition of the invention, wherein the conjugate of the invention comprises an sdAb that can bind to HER2, CD71 or EGFR, and/or wherein one or more active pharmaceutical ingredient(s) comprise(s) an sdAb that can bind to HER2, CD71 or EGFR, and/or wherein the ADC comprises dianthin or saporin.

A fourth aspect of the invention relates to the pharmaceutical combination of the invention, comprising the first pharmaceutical composition and comprising the second pharmaceutical composition, or to the pharmaceutical composition of the invention, for use as a medicament.

A fifth aspect of the invention relates to the pharmaceutical combination of the invention, comprising the first pharmaceutical composition and comprising the second pharmaceutical composition, or to the pharmaceutical composition of the invention, for use in the treatment or the prophylaxis of a cancer, an auto-immune disease, an infection such as a viral infection, an enzyme deficiency, a disorder or disease relating to an enzyme deficiency, a gene defect, a disorder or disease relating to a gene defect.

Surprisingly, the inventors found that a dose of an ADC which does not result in any tumor-cell killing is sufficient and enough for efficient tumor-cell killing when the ADC is contacted with the tumor cells in the presence of the conjugate of the invention. Examples are provided in FIGS. 2-4. For example, a dose of ADC anti-CD71-toxin such as OKT-9-saporin does not exert a toxic effect on tumor cells when contacted with the tumor cells. However, when the ADC is co-administered together with a conjugate of the invention, efficient tumor cell killing is achieved. The conjugate of the invention is for example anti-HER2 V_{HH}-SO1861, anti-CD71 V_{HH}-SO1861, anti-EGFR V_{HH}-SO1861. The ADC is for example an ADC comprising an antibody or V_{HH} capable of binding to HER2, CD71 or EGFR, and for example, the ADC comprises a protein toxin such as dianthin or saporin. As said before, by applying the conjugate of the invention, the saponin dose required to achieve an efficient biological activity of the effector molecule comprised by an ADC or an AOC inside a target cell, is about 100 to 1000 fold lower when the conjugate comprising the saponin is co-administered with the ADC or AOC to the target cells, compared to the dose of free saponin that is required when the ADC or the AOC is co-administered to the target cell together with the free saponin. Herewith, the conjugate of the invention potentiates an ADC or an AOC at a dose of the ADC or the AOC that would otherwise not be effective in tumor cells when it would be administered to a patient in need thereof in the absence of the targeted saponin, and in addition, herewith the conjugate of the invention is already sufficiently effective when potentiation of the effector molecule of an ADC or an AOC is considered, already at a relatively low dose i.e. at a dose at which a free saponin that is not provided with a tumor-cell targeting sdAb, is not sufficiently effective in effector molecule potentiation. In combination, the inventors provide for an improved method for treating a human patient in need of treatment with a therapeutically effective amount of an ADC or an AOC, since the ADC or the AOC is already effective at lower dose when co-administered to patients in the presence of the conjugate of the invention. One of the many benefits of the conjugates of the invention resides in the absence of an Fc tail in the sdAb part of the conjugates. Absence of the Fc tail prevents unwanted binding of conjugates to off-target patient cells bearing Fc receptors, which binding could otherwise result in side-effects if such an Fc tail would be present, such as seen for many conventional ADCs, AOCs. Antibody-based ADCs and AOCs comprising an Fc tail suffer from a decreased efficacy due to the undesired binding of such IgG based ADCs, AOCs to Fc receptors. As a result of Fc receptor binding, the effective dose of such IgG-based ADCs and AOCs is lowered. Therewith, absence of the Fc tail provides for several benefits in this regard. Off-target and undesired binding of the conjugate to Fc receptors cannot occur. Herewith, the conjugate of the invention has a lower effective dose when target receptor mediated endocytosis and delivery of the conjugate of the invention inside endosomes is considered, since no conjugate is ‘lost’ due to Fc receptor binding, a drawback seen with IgG-based conjugates. As a result, the therapeutic window of the conjugates of the invention is wider than the therapeutic window that would have been achieved when the sdAb is replaced by a conventional IgG comprising the Fc tail.

A sixth aspect of the invention relates to an in vitro or ex vivo method for transferring a molecule from outside a cell to inside said cell, preferably to the cytosol of said cell, comprising the steps of:
a) providing a cell which expresses a binding site for the sdAb comprised by the conjugate of the invention on its surface, preferably selected from a liver cell, an aberrant cell such as a virally infected cell, an auto-immune cell and a tumor cell;
b) providing the molecule for transferring into the cell provided in step a), wherein the molecule is any one or more of the API(s) of embodiments of the invention;
c) providing the conjugate of the invention;
d) contacting the cell of step a) in vitro or ex vivo with the molecule of step b) and the conjugate of step c),
   therewith establishing the transfer of the molecule from outside the cell into said cell, preferably into the cytosol of said cell.

Unless specifically indicated otherwise and in particular when relating to the endosomal escape mechanism of the saponin of the invention, whenever the word “endosome” or “endosomal escape” is used herein, it also includes the endolysosome and lysosome, and escape from the endolysosome and lysosome, respectively. After entering the cytosol, said substance might move to other cell units such as the nucleus.

In formal terms, a glycoside is any molecule in which a sugar group is bound through its anomeric carbon to another group via a glycosidic bond. Glycoside molecules, such as saponins, in the context of the invention are such molecules that are further able to enhance the effect of an effector moiety, without wishing to be bound by any theory, in particular by facilitating the endosomal escape of the effector moiety. Without wishing to be bound by any theory, the glycoside molecules (saponins of the invention, such as those exemplified herein and in the claims) interact with the membranes of compartments and vesicles of the endocytic and recycling pathway and make them leaky for said effector moieties resulting in augmented endosomal escape. With the term “the scaffold is able to augment endosomal escape of the effector moiety” is meant that the at least one saponin (glycoside molecule), which is coupled via a linker or directly to the cell-surface molecule targeting antibody such as an sdAb or via the polymeric or oligomeric structure of the scaffold (covalent saponin conjugate of the invention), is able to enhance endosomal escape of an effector moiety when both molecules are within an endosome, e.g. a late endosome, optionally and preferably after the at least one saponin is released from the conjugate of the invention such as from a linker or polymeric or oligomeric structure comprised by said conjugate, e.g., by cleavage of a cleavable bond between the at least one glycoside (saponin) and the conjugate (for example via a polymeric or oligomeric structure of a scaffold and/or via a linker). Even though a bond between the at least one saponin according to the invention and the cell-surface molecule targeting sdAb of the conjugate of the invention, optionally via a linker or a scaffold, may be a “stable bond”, that does not mean that such bond cannot be cleaved in the endosomes by, e.g., enzymes. For instance, the saponin, optionally together with a linker or a part of the oligomeric or polymeric structure of a scaffold, may be cleaved off from the remaining linker fragment or oligomeric or polymeric structure. It could, for instance be that a protease cuts a (proteinaceous) linker or proteinaceous polymeric structure, e.g., albumin, thereby releasing the at least one saponin. It is, however, preferred that the glycoside molecule (preferably saponin) is released in an active form, preferably in the original form that it had before it was (prepared to be) coupled to the cell-surface molecule targeting sdAb of the conjugate of the invention optionally via a linker and/or an oligomeric or polymeric scaffold (covalent saponin conjugate of the invention); thus the glycoside (saponin) has its natural structure after such cleavage or the glycoside (saponin) has (part of) a chemical group or linker bound thereto, after such cleavage, while glycoside biological activity (saponin biological activity), e.g. endosomal/lysosomal escape enhancing activity towards an effector moiety present in the same endosome or lysosome, is maintained or restored upon said cleavage of the bond between the glycoside (saponin) and the cell-surface molecule targeting antibody such as an sdAb, optionally comprising a linker and/or a scaffold of the invention. With regard to the present invention the term “stable” with respect to bonds between e.g. saponins and amino-acid residues of the cell-surface molecule targeting sdAb in the conjugate, a linker, a polymeric or oligomeric structures (of the scaffold, a.k.a. the covalent saponin conjugate of the invention), ligands, (monoclonal) immunoglobulins or binding domains or—fragments thereof, and/or effectors (effector moieties, effector molecules), is meant that the bond is not readily broken or at least not designed to be readily broken by, e.g., pH differences, salt concentrations, or UV-light, reductive conditions. With regard to the present invention the term “cleavable” with respect to bonds between e.g. saponins and the cell-surface molecule targeting sdAb, linkers, amino-acid residues, polymeric or oligomeric structures of the covalent saponin conjugate, ligands, antibodies and/or effectors, is meant that the bond is designed to be readily broken by, e.g., pH differences, salt concentrations, under reductive conditions, and the like. The skilled person is well aware of such cleavable bonds and how to prepare them.

Before the present invention one of the major hurdles of introducing ADCs and AOCs on the market was the small therapeutic window: a therapeutically effective dose of an ADC or an AOC is accompanied with (unacceptable) side effects, hampering development and implication in treatment of patients with the ADCs. By the application of the conjugate of the invention, i.e. the sdAb-saponin conjugate, in combination with an ADC or an AOC, it has now become possible to guide one or multiple glycoside molecules (saponin(s)) to a (target) cell, together with the ADC carrying a payload or together with a (monoclonal) antibody (sdAb) conjugated with an oligonucleotide such as a BNA according to the invention. In particular, it was previously not possible to specifically guide an effector moiety of an ADC or AOC or any other conjugate of a payload and a (proteinaceous) cell-surface molecule targeting molecule, and a (predefined, controllable) particular number or range of glycoside molecules (saponins) per effector moiety at the same time to the cytosol of cells, such as via the endocytic pathway of a cell. As said before, the ADC or the AOC and the conjugate of the invention can target the same cell-surface molecule or can target different cell surface molecules, wherein the two different cell-surface molecules are expressed at the surface of the same target cell such as a tumor cell or an auto-immune cell.

A solution provided for by the invention comprises the covalent binding of at least one saponin to the cell-surface molecule targeting molecule of the conjugate of the invention, i.e. an sdAb. A further solution provided for by the invention comprises (first) polymerizing the glycoside molecules (saponins) using an oligomeric or polymeric scaffold, and providing the cell-surface molecule targeting molecule comprised by the conjugate of the invention with a cluster of covalently bound saponins, enabling re-monomerization of the one or more saponins at the intracellular site where the mode of action of the saponin is desired, e.g. after endocytosis. “Polymerizes” in this context means the reversible and/or irreversible multiple conjugation of saponin molecules to the sdAb, either via linker, or directly or via a polymeric or oligomeric structure to form a scaffold (covalent saponin conjugate of the invention) or the reversible and/or irreversible multiple conjugation of (modified) saponins thereby forming a polymeric or oligomeric structure to form a scaffold (covalent saponin conjugate of the invention). “Re-monomerization” in this context means the cleavage of the saponins from the conjugate, from the linker linking the saponin(s) to the cell-surface molecule targeting sdAb of the conjugate or from the scaffold, for example after endocytosis, and regaining the (native) chemical state of the unbound saponins, which unbound saponins may or may not comprise additional chemical groups such as a chemical group for linking the saponin to a linker, an amino-acid residue of the conjugate or to the scaffold, and/or a (chemical) linker bound to a chemical group of the saponin such as an aldehyde group or carboxylic acid group. Due to the complex chemistry of the saponins for example the “polymerization” of saponins at a scaffold or other linking linker and their “re-monomerization” at a desired location such as intracellularly e.g. after endocytosis, was a challenging task. In particular, the chemical reactions used for providing the linkers and the scaffold comprising covalently linked glycosides for covalent binding to the conjugate, e.g. triterpenoid saponins (polymerization of the glycosides), normally occur in water-free organic solvents, but saponins and for example biocompatible polymers applied as a scaffold for bearing bound saponins, are water-soluble molecules. The chemical properties of the unmodified saponin further prohibited polymerization by itself and, one other possible solution, to bind multiple saponins (directly) to the sdAb was estimated not to be very promising, as an sdAb does typically not provide sufficient binding sites and because the coupling product would become quite heterogeneous and/or coupling biologically active molecules such as a saponin and an sdAb together bears the risk for influencing and hampering the activity of the saponin and/or the capacity of the sdAb to bind to the cell-surface molecule when both molecules are bound together in such saponin-comprising conjugate. Embodiments of the present invention solves at least one of these drawbacks.

A seventh aspect of the invention relates to a kit of parts, comprising the pharmaceutical combination of the invention, comprising the first pharmaceutical composition and comprising the second pharmaceutical composition, or comprising the pharmaceutical composition of the invention, or comprising the conjugate of the invention, and (optionally) instructions for use of said pharmaceutical combination according to the invention, or optionally instructions for use of said pharmaceutical composition according to the invention, or optionally instructions for use of said conjugate according to the invention.

Whether or not a conjugate of the invention comprising saponins, either or not further comprising one or more (cleavable) linkers and/or optionally a scaffold (covalent saponin conjugate of the invention), is able to disturb the acidic environment and inhibit the endosomal escape function of the at least one glycoside (saponin) can be easily determined with an assay as described in the examples section, and as known in the art. The inhibition is described as “fold amount increases of glycoside (saponin of the invention) necessary to induced 50% cell killing”. It is preferred that the scaffold or the conjugate of the invention does not lead to an increase that is at least the increase in glycoside molecules (saponins) necessary to obtain 50% cell killing observed when using Chloroquine as a positive control. Alternatively, and preferably, the conjugate comprising saponins, either or not further comprising one or more (cleavable) linkers and/or optionally a scaffold does not lead to an at least 4-fold increase of glycoside molecules to induce 50% cell killing, more preferably does not lead to an at least 2-fold increase. The fold increase is to be measured in assay, wherein Chloroquine, as a positive control, induces a 2-fold increase in glycoside amount, preferably saponin amount wherein the saponin is any one or more of the saponins of the invention (previous embodiments) to observe 50% cell killing.

As said before, the at least one saponin that is comprised by the conjugate according to the invention increases the efficacy of at least current and new effector moieties as defined in this invention. Potential side-effects will be decreased due to lowering of dosing of the effector moiety comprised by the second conjugate such as an ADC or an AOC, without lowering the efficacy. Therefore, the invention provides a conjugate according to the invention for use in medicine or for use as a medicament. An aspect of the invention relates to a pharmaceutical composition of the invention, for use as a medicament, wherein the pharmaceutical composition comprises the conjugate of the invention.

A number of preferred features can be formulated for endosomal escape enhancers comprised by the conjugate of the invention, i.e. a saponin of the invention: (1) they are preferably not toxic and do not invoke an immune response, (2) they preferably do not mediate the cytosolic uptake of the effector moiety into off-target cells, (3) their presence at the site of action is preferably synchronized with the presence of the effector moiety, (4) they are preferably biodegradable or excretable, and (5) they preferably do not substantially interfere with biological processes of the organism unrelated to the biological activity of the effector molecule with which the endosomal escape enhancer is combined with, e.g. interact with hormones. Examples of saponins of the invention that fulfill the before mentioned criteria, at least to some extent, are bidesmosidic triterpenes, preferably bidesmosidic triterpene saponins, such as SO1861, SA1641, QS-21, GE1741, and the further saponins according to the invention and listed throughout the specification, and as listed in Table A1.

Also provided is the use of a conjugate according to the invention for manufacturing a medicament. Especially cancer medicines, and in particular the classical chemotherapy medicaments, are notorious for their side effects. Because of targeting and synchronization in time and place of both the pharmaceutically active substance comprised by the second conjugate such as an ADC or an AOC, and the saponin comprised by the conjugate molecule of the invention, a conjugate according to the invention is especially valuable for use as a medicament or as a constituent of a medicament, in particular for use in a method of treating cancer, wherein the medicament is a single composition comprising an ADC, an AOC or an oligonucleotide such as a BNA or an siRNA and the conjugate of the invention, or is a therapeutic combination of a first pharmaceutical composition comprising an ADC, an AOC or an oligonucleotide such as a BNA or an siRNA, and a second pharmaceutical composition comprising the conjugate of the invention. The invention thus provides a conjugate according to the invention for use in a method of treating cancer. The invention also provides a conjugate according to the invention for use in a method of treating acquired or hereditary disorders, in particular monogenic deficiency disorders. The conjugate thus comprises the at least one saponin and an sdAb for targeting the conjugate at an aberrant target cell such as a tumor cell or an auto-immune cell, and the conjugate of the invention is combined with at least one effector moiety such as a BNA or an ADC or an AOC. Thus, an aspect of the invention relates to a conjugate according to the invention, wherein the conjugate comprises a covalently bound saponin and a cell-surface molecule binding antibody such as an sdAb, for use in a method for the treatment of a cancer or an auto-immune disease, the method further comprising the administration of an effector molecule to a subject such as a cancer patient in need thereof, wherein the effector molecule is provided as such or as part of an ADC or an AOC.

A further application of the conjugate of the invention in medicine is the use in a method for the substitution of intracellular enzymes in target cells that produce these enzymes in insufficient amount or insufficient functionality. The conjugate of the invention is for example administered to a patient in need thereof in combination with a molecule or further conjugate comprising the enzyme to be substituted or an oligonucleotide for gene therapy, for substitution of a target intracellular enzyme. The resulting disease might be hereditary or acquired. In most cases, only symptomatic treatment is possible and for a number of rare diseases, insufficient treatment options lead to a shortened life span of concerned patients. An example for such a disease is phenylketonuria, which is an inborn error of metabolism that results in decreased metabolism of the amino acid phenylalanine. The disease is characterized by mutations in the gene for the hepatic enzyme phenylalanine hydroxylase. Phenylketonuria is not curable to date. The incidence is approximately 1:10,000 with the highest known incidence in Turkey with 1:2,600. A cell-surface molecule targeting antibody comprised by the conjugate of the invention, preferably an sdAb such as a V_{HH}, with bound saponin and a second conjugate comprising the same or a different cell-surface binding molecule such as an antibody such as an sdAb and phenylalanine hydroxylase or a second conjugate comprising the same or a different cell-surface binding molecule such as an antibody such as an sdAb with a bound polynucleotide that encodes phenylalanine hydroxylase, can be used to target liver cells by use of a suitable specific antibody or sdAb in the conjugate of the invention and in the second conjugate according to the invention, and to substitute the defect enzyme in hepatocytes. This is one example of use of the conjugate of the invention comprising a saponin bound thereto and, in this example, a second conjugate comprising an effector molecule, such as the enzyme or the oligonucleotide bound thereto according to the invention, for substitution or gene therapy. In a preferred embodiment, a conjugate according to the invention for use in a method of gene therapy or substitution therapy is provided in combination of a second conjugate comprising a cell-surface binding molecule and an enzyme or a nucleic acid encoding for the enzyme.

With the conjugate of the invention it has now become possible to design and manufacture a two-component, non-viral clinically applicable gene delivery technology. For example, the conjugate of the invention allows for development of non-viral based combinations of conjugates for gene delivery technology, which enhances therapeutic efficacy with lower therapeutic dose thereby improving the health of patients. The conjugate of the invention, in particular when comprising a covalently bound cell-surface molecule targeting antibody such as a monoclonal antibody or sdAb for binding to a (tumor, auto-immune) cell-surface specific molecule, and combined with a second conjugate comprising an effector moiety such as an oligonucleotide for example a BNA, allows for overcoming a longstanding and major bottleneck in the field of gene delivery, namely efficient, safe and cost-effective transfer of gene therapeutic products across the endosomal membrane into the cytosol/nucleosol. Indeed, gene therapy is one of the most promising treatment options for future advanced therapies in a broad range of diseases. Successful gene delivery requires the recognition of target cells as well as cytosolic and nucleosolic uptake of the gene. One of the major problems in the field of non-viral gene therapy is the inefficient and insufficiently safe delivery of genetic material for therapeutic use in patients.

Thus, when applying the conjugate of the invention, comprising a cell-targeting cell-surface molecule targeting molecule such as a ligand or preferably an antibody (fragment, domain thereof, preferably sdAb) and combined with a second conjugate such as an AOC or an sdAb comprising conjugate comprising an oligonucleotide such as an antisense BNA, the inventors now made it possible to overcome a longstanding and major bottleneck in the field of gene delivery: safe transfer of gene therapeutic products across the endosomal membrane into the cytosol/nucleosol. The conjugate of the invention represents technology designed for allowing targeting of any addressable cell type with all known genetic agents and subsequent efficient cytosolic delivery of a gene, thereby ensuring better patient therapy not limited to inherited disorders, but also for cancer therapy and therefore of importance for large patient groups. The technology based on the conjugate of the invention may comprise a polymeric or oligomeric scaffold (covalent saponin conjugate of the invention) that serves as a carrier for endosomal escape enhancers (EEEs), such as the saponins as exemplified herein, and the saponins of the embodiments according to the invention, for the cell-surface molecule targeting molecule such as a targeting ligand or (monoclonal) (tumor-cell specific) antibody, or a fragment thereof, or preferably an sdAb such as a V_{HH}. A second conjugate then comprises a second cell-surface molecule binding molecule such as a receptor ligand, antibody such as an sdAb, and an effector moiety, here an effector gene such as an LNA or BNA. Use of the conjugate of the invention, e.g. comprising a cell-targeting antibody (fragment) or sdAb in combination with a second conjugate comprising the same or a different cell-surface targeting molecule and an oligonucleotide such as a BNA, has potential to bring any kind of biological macromolecules into the cytosol and the nucleus. Development of new targeting ligands, sdAbs and monoclonal (human, humanized) antibodies is under continuous investigation by numerous research groups and companies worldwide. The same for the oligonucleotides that are aimed for delivery in the cytosol of diseases cells such as cancer cells. The conjugate of the invention thus also presents as a molecular tool in which present and future targeting sdAbs and antibodies can be used by click chemistry, allowing for customized drug applications and for future developments in the field of tissue and cell targeting techniques, and the second conjugates according to the invention thus also present as a molecular tool in which present and future therapeutic oligonucleotides (as well as payloads such as protein toxins) are linked or can be linked to for example an sdAb by click chemistry, allowing for customized drug applications and for future developments in the field of tissue and cell targeting techniques. The conjugate of the invention can comprise antibodies and ligands as the cell-surface molecule targeting molecule, but an sdAb is preferred. The worldwide market of gene therapeutics is rapidly growing and is covering potential treatments for a wide range of disease areas such as, cancer, cardiovascular diseases, Parkinson's, Alzheimer, HIV and many rare (monogenetic) diseases. The current viral vector-based gene therapeutic technologies have significant challenges, such as safety, manufacturing logistics, and associated high costs. The conjugate of the invention allows for use in a technology platform which represents an alternative for a current viral gene delivery technology, when combined with a second conjugate comprising the gene to be delivered and a cell-surface targeting molecule such as an antibody such as an sdAb. Therefore, the conjugate of the invention is suitable for implementing in approaches for developing non-viral gene treatments for diseases such as cancers, cardiovascular diseases, Parkinson's disease, Alzheimer's disease, HIV infection and many rare (monogenetic) diseases. The conjugate of the invention is suitable for developing novel treatments for transforming the field of antibody-drug conjugates (ADCs) and oligonucleotide-based therapeutics by making non-viral vector based gene therapeutics such as based on targeted antisense BNA. The application of the conjugate of the invention, in particular in a covalent conjugate with an antibody such as an sdAb and a saponin, and combined with a second conjugate comprising a cell-surface molecule binding molecule such as an antibody and an oligonucleotide such as a BNA, is one of the many beneficial approaches made possible due to the present invention. For example, use of the conjugate of the invention now allows for exploitation of the endocytic pathway of mammalian cells. Endocytosis is exploited for the delivery of therapeutics, wherein the conjugate of the invention contributes to improved uptake and endosomal escape of e.g. siRNAs which are comprised by the second conjugate. The conjugate of the invention is suitably used together with small molecules that act as delivery enhancers for e.g. payloads, oligonucleotides. Herewith, the second conjugate of the invention bearing the covalently coupled oligonucleotide such as a BNA and bearing the covalently coupled cell targeting moiety such as a ligand and preferably an antibody (domain or fragment, preferably a V_{HH}) in combination with the conjugate of the invention, bearing the saponins of the invention and bearing the covalently coupled cell targeting moiety such as a ligand and preferably an antibody (domain or fragment, preferably a V_{HH}), provides a solution for the current problem seen with current endosomal escape enhancers and gene therapeutic product, relating to their application as two components comprising free non-targeted saponin, thus complicating therapeutic approval and clinical applicability, since such a conjugate of the invention encompassing the saponin, combined with a second conjugate comprising a gene product such as a BNA and a (tumor) cell targeting moiety such as a (monoclonal) antibody or sdAb. Thus the invention provides a non-viral gene delivery technology where endosomal escape enhancers (e.g. the glycosides of the embodiments of the invention and of the examples provided) and targeting ligand or antibody (according to e.g. the embodiments of the invention and the sdAbs exemplified here below in the Examples section) are comprised by the conjugate of the invention, and are combined with a second conjugate comprising a gene therapeutic product (oligonucleotides according to the invention such as a BNA) and a second cell-surface molecule targeting molecule such as the same or a different cell-surface targeting molecule as encompassed by the conjugate of the invention, wherein the second cell-surface molecule targeting molecule binds to the same or to a different cell surface molecule as the cell surface molecule targeting molecule of the conjugate of the invention. Such a conjugate of the invention thus provides therapeutic opportunities for current and future macromolecule drugs for a broad range of diseases and large patient groups. With the application of such a conjugate of the invention comprising at least one saponin and at least one specific cell-targeting moiety such as an immunoglobulin or sdAb, in combination with a second conjugate comprising at least one oligonucleotide and at least one specific cell-targeting moiety such as an immunoglobulin or sdAb, the problem is addressed which is apparent for current methods of applying non-cell-surface molecule targeted free endosomal escape enhancers (e.g. saponin not conjugated with a cell-surface molecule binding antibody) and gene therapeutic product separately, which current methods do not ensure that both compounds are at the same time at the site of interaction. This problem is now overcome by using the conjugate of the invention in combination with a second conjugate comprising the nucleic acid, wherein the conjugate of the invention and the second conjugate both comprise a cell-surface molecule targeting molecule such as an antibody such as an sdAb. That is to say, such a combination of a conjugate of the invention and a second conjugate comprising an oligonucleotide provides a non-viral gene delivery technology with increased synchronization (in time and place) of both compounds, i.e. the saponin and the gene product such as a BNA.

Gene therapies could help with hereditary, previously incurable diseases such as cystic fibrosis, chorea, Huntington's disease or hemophilia. However, currently some problems have not been overcome: for example, the therapeutic genes must precisely reach specific target cells in the body. On the other hand, the therapeutic genes should be absorbed by the targeted cells, but the therapeutic genes should not be destroyed. The current gene therapy approaches use viruses as a ferry for genes. However, these procedures involve considerable risks and cannot be transferred to the introduction of other biomolecules. An embodiment is the conjugate of the invention comprising (plant-derived) glycosides (e.g. any one of the saponins of the invention) for use a platform technology that allows not only delivery of genes when such genes are comprised by the second conjugate as the carrier molecule, but also allows for the delivery of different therapeutic biomolecules to be introduced into target cells. Therefore, the conjugate of the invention is used for developing treatments based on nucleic acids for cystic fibrosis, chorea, Huntington's disease or hemophilia. Herewith, with the conjugate of the invention, a new gene therapy strategy is available for improving the health of patients with genetic diseases, including those patients with cystic fibrosis, Huntington's disease, and hemophilia. As part of the invention, a non-viral gene delivery technology is developed that combines plant-derived endosomal escape enhancers (glycosides; i.e. the saponins of the invention) and a targeting ligand (i.e. an sdAb) that are comprised in a single conjugate of the invention, for combination with a second conjugate comprising a cell-surface molecule binding molecule and a gene therapeutic products. The resulting non-viral gene therapy based on the conjugate of the invention displays about 40 times increased delivery efficiency at a lower dosage of the second conjugate comprising the gene over currently available strategies. Herewith, the conjugate of the invention is for use in clinical applications such as for the repair or replacement of defective genes, like in cystic fibrosis patients, and for the targeted delivery of specific genes, for instance, to destroy cancer cells. In fact, the conjugate of the invention is suitable for application in treatment regimens for any disease caused by a genetic defect—such as cystic fibrosis, Huntington's disease and hemophilia and which are currently incurable. Gene therapy which makes use of the conjugate of the invention helps in overcoming two current problems: Firstly, it is possible with the conjugate of the invention to deliver the second conjugate which comprises therapeutic genes to specific target cells in the body; secondly, the therapeutic genes enter the interior of these cells, but are not destroyed, due to the presence of saponin(s) and a targeting moiety such as an antibody or an sdAb for binding a target cell (conjugate of the invention), and the presence of the oligonucleotide product and a targeting moiety such as an antibody or an sdAb for binding a target cell.

The present invention also provides a method of treating cancer, the method comprising administering a medicament comprising a conjugate according to the invention to a patient in need thereof in combination with an ADC or an AOC, preferably administering an effective dose of said medicament to a patient in need thereof, preferably a human cancer patient.

Considerations concerning forms suitable for administration are known in the art and include toxic effects, solubility, route of administration, and maintaining activity. For example, pharmacological compositions injected into the bloodstream should be soluble.

Suitable dosage forms, in part depend upon the use or the route of entry, for example transdermal or by injection. Such dosage forms should allow the compound to reach a target cell whether the target cell is present in a multicellular host. Other factors are known in the art, and include considerations such as toxicity and dosage form which retard the compound or composition from exerting its effect.

TABLE A1
Saponins displaying (late) endosomal/lysosomal escape enhancing activity,
and saponins comprising a structure reminiscent to such saponins displaying
(late) endosomal/lysosomal escape enhancing activity

| | | Carbohydrate substituent | Carbohydrate substituent |
|---|---|---|---|
| Saponin Name | Aglycone core | at the C-3beta-OH group | at the C-28-OH group |
| NP-005236 | 2alpha- | GlcA- | Glc/Gal- |
| | Hydroxyoleanolic | | |
| | acid | | |
| AMA-1 | 16alpha- | Glc- | Rha-(1→2)-[Xyl-(1→4)]-Rha- |
| | Hydroxyoleanolic | | |
| | acid | | |
| AMR | 16alpha- | Glc- | Rha-(1→2)-[Ara-(1→3)-Xyl-(1→4)]- |
| | Hydroxyoleanolic | | Rha- |
| | acid | | |
| alpha-Hederin | Hederagenin (23- | Rha-(1→2)-Ara- | — |
| | Hydroxyoleanolic | | |
| | acid) | | |
| NP-012672 | 16alpha,23- | Ara/Xyl-(1→4)- | Ara/Xyl- |
| | Dihydroxyoleanolic | Rha/Fuc-(1→2)- | |
| | acid | Glc/Gal-(1→2)- | |
| | | Rha/Fuc-(1→2)-GlcA- | |
| NP-017777 | Gypsogenin | Gal-(1→2)-[Xyl-(1→3)]- | Xyl-(1→4)-Rha-(1→2)-[R-(→4)]- |
| | | GlcA- | Fuc- (R═4E-Methoxycinnamic acid) |
| NP-017778 | Gypsogenin | Gal-(1→2)-[Xyl-(1→3)]- | Xyl-(1→4)-Rha-(1→2)-[R-(→4)]- |
| | | GlcA- | Fuc- (R═4Z-Methoxycinnamic acid) |
| NP-017774 | Gypsogenin | Gal-(1→2)-[Xyl-(1→3)]- | Xyl-(1→4)-[Gal-(1→3)]-Rha-(1→2)- |
| | | GlcA- | 4-OAc-Fuc- |
| NP-018110^{c}, | Gypsogenin | Gal-(1→2)-[Xyl-(1→3)]- | Xyl-(1→4)-[Glc-(1→3)]-Rha-(1→2)- |
| NP-017772^{d} | | GlcA- | 3,4-di-OAc-Fuc- |
| NP-018109 | Gypsogenin | Gal-(1→2)-[Xyl-(1→3)]- | Xyl-(1→4)-[Glc-(1→3)]-Rha-(1→2)- |
| | | GlcA- | [R-(→4)]-3-OAc-Fuc- (R═4E- |
| | | | Methoxycinnamic acid) |
| NP-017888 | Gypsogenin | Gal-(1→2)-[Xyl-(1→3)]- | Glc-(1→3)-Xyl-(1→4)-[Glc-(1→3)]- |
| | | GlcA- | Rha-(1→2)-4-OAc-Fuc- |
| NP-017889 | Gypsogenin | Gal-(1→2)-[Xyl-(1→3)]- | Glc-(1→3)-Xyl-(1→4)-Rha-(1→2)-4- |
| | | GlcA- | OAc-Fuc- |
| NP-018108 | Gypsogenin | Gal-(1→2)-[Xyl-(1→3)]- | Ara/Xyl-(1→3)-Ara/Xyl-(1→4)- |
| | | GlcA- | Rha/Fuc-(1→2)-[4-OAc-Rha/Fuc- |
| | | | (1→4)]-Rha/Fuc- |
| SA1641^{a}, | Gypsogenin | Gal-(1→2)-[Xyl-(1→3)]- | Xyl-(1→3)-Xyl-(1→4)-Rha-(1→2)- |
| AEX55^{b} | | GlcA- | [Qui-(1→4)]-Fuc- |
| NP-017674 | Quillaic acid | Gal-(1→2)-[Xyl-(1→3)]- | Api-(1→3)-Xyl-(1→4)-[Glc-(1→3)]- |
| | | GlcA- | Rha-(1→2)-Fuc- |
| NP-017810 | Quillaic acid | Gal-(1→2)-[Xyl-(1→3)]- | Xyl-(1→4)-[Gal-(1→3)]-Rha-(1→2)- |
| | | GlcA- | Fuc- |
| AG1 | Quillaic acid | Gal-(1→2)-[Xyl-(1→3)]- | Xyl-(1→4)-[Glc-(1→3)]-Rha-(1→2)- |
| | | GlcA- | Fuc- |
| NP-003881 | Quillaic acid | Gal-(1→2)-[Xyl-(1→3)]- | Ara/Xyl-(1→4)-Rha/Fuc-(1→4)- |
| | | GlcA- | [Glc/Gal-(1→2)]-Fuc- |
| NP-017676 | Quillaic acid | Gal-(1→2)-[Xyl-(1→3)]- | Api-(1→3)-Xyl-(1→4)-[Glc-(1→3)]- |
| | | GlcA- | Rha-(1→2)-[R-(→4)]-Fuc- |
| | | | (R═5-O-[5-O-Ara/Api-3,5- |
| | | | dihydroxy-6-methyl-octanoyl]-3,5- |
| | | | dihydroxy-6-methyl-octanoic acid) |
| NP-017677 | Quillaic acid | Gal-(1→2)-[Xyl-(1→3)]- | Api-(1→3)-Xyl-(1→4)-Rha-(1→2)- |
| | | GlcA- | [R-(→4)]-Fuc- |
| | | | (R═5-O-[5-O-Ara/Api-3,5- |
| | | | dihydroxy-6-methyl-octanoyl]-3,5- |
| | | | dihydroxy-6-methyl-octanoic acid) |
| NP-017706 | Quillaic acid | Gal-(1→2)-[Xyl-(1→3)]- | Api-(1→3)-Xyl-(1→4)-Rha-(1→2)- |
| | | GlcA- | [Rha-(1→3)]-4-OAc-Fuc- |
| NP-017705 | Quillaic acid | Gal-(1→2)-[Xyl-(1→3)]- | Api-(1→3)-Xyl-(1→4)-[Glc-(1→3)]- |
| | | GlcA- | Rha-(1→2)-[Rha-(1→3)]-4-OAc- |
| | | | Fuc- |
| NP-017773 | Quillaic acid | Gal-(1→2)-[Xyl-(1→3)]- | 6-OAc-Glc-(1→3)-Xyl-(1→4)-Rha- |
| | | GlcA- | (1→2)-[3-OAc-Rha-(1→3)]-Fuc- |
| NP-017775 | Quillaic acid | Gal-(1→2)-[Xyl-(1→3)]- | Glc-(1→3)-Xyl-(1→4)-Rha-(1→2)- |
| | | GlcA- | [3-OAc--Rha-(1→3)]-Fuc- |
| SA1657 | Quillaic acid | Gal-(1→2)-[Xyl-(1→3)]- | Xyl-(1→3)-Xyl-(1→4)-Rha-(1→2)- |
| | | GlcA- | [Qui-(1→4)]-Fuc- |
| AG2 | Quillaic acid | Gal-(1→2)-[Xyl-(1→3)]- | Glc-(1→3)-[Xyl-(1→4)]-Rha-(1→2)- |
| | | GlcA- | [Qui-(1→4)]-Fuc- |
| SO1861 | Quillaic acid | Gal-(1→2)-[Xyl-(1→3)]- | Glc-(1→3)-Xyl-(1→4)-Rha-(1→2)- |
| | | GlcA- | [Xyl-(1→3)-4-OAc-Qui-(1→4)]-Fuc- |
| GE1741 | Quillaic acid | Gal-(1→2)-[Xyl-(1→3)]- | Xyl-(1→3)-Xyl-(1→4)-Rha-(1→2)- |
| | | GlcA- | [3,4-di-OAc-Qui-(1→4)]-Fuc- |
| SO1542 | Quillaic acid | Gal-(1→2)-[Xyl-(1→3)]- | Glc-(1→3)-[Xyl-(1→4)]-Rha-(1→2)- |
| | | GlcA- | Fuc- |
| SO1584 | Quillaic acid | Gal-(1→2)-[Xyl-(1→3)]- | 6-OAc-Glc-(1→3)-[Xyl-(1→4)]-Rha- |
| | | GlcA- | (1→2)-Fuc- |
| SO1658 | Gypsogenin | Gal-(1→2)-[Xyl-(1→3)]- | Glc-(1→3)-[Xyl-(1→3)-Xyl-(1→4)]- |
| | | GlcA- | Rha-(1→2)-Fuc- |
| SO1674 | Quillaic acid | Gal-(1→2)-[Xyl-(1→3)]- | Glc-(1→3)-[Xyl-(1→3)-Xyl-(1→4)]- |
| | | GlcA- | Rha-(1→2)-Fuc- |
| SO1832 | Quillaic acid | Gal-(1→2)-[Xyl-(1→3)]- | Xyl-(1→3)-Xyl-(1→4)-Rha-(1→2)- |
| | | GlcA- | [Xyl-(1→3)-4-OAc-Qui-(1→4)]-Fuc- |
| QS-7 (also | Quillaic acid | Gal-(1→2)-[Xyl-(1→3)]- | Api/Xyl-(1→3)-Xyl-(1→4)-[Glc- |
| referred to as | | GlcA- | (1→3)]-Rha-(1→2)-[Rha-(1→3)]- |
| QS1861) | | | 4OAc-Fuc- |
| QS-7 api (also | Quillaic acid | Gal-(1→2)-[Xyl-(1→3)]- | Api-(1→3)-Xyl-(1→4)-[Glc-(1→3)]- |
| referred to as | | GlcA- | Rha-(1→2)-[Rha-(1→3)]-4OAc-Fuc- |
| QS1862) | | | |
| QS-17 | Quillaic acid | Gal-(1→2)-[Xyl-(1→3)]- | Api/Xyl-(1→3)-Xyl-(1→4)-[Glc- |
| | | GlcA- | (1→3)]-Rha-(1→2)-[R-(→4)]-Fuc- |
| | | | (R═5-O-[5-O-Rha-(1→2)-Ara/Api- |
| | | | 3,5-dihydroxy-6-methyl-octanoyl]- |
| | | | 3,5-dihydroxy-6-methyl-octanoic |
| | | | acid) |
| QS-18 | Quillaic acid | Gal-(1→2)-[Xyl-(1→3)]- | Api/Xyl-(1→3)-Xyl-(1→4)-[Glc- |
| | | GlcA- | (1→3)]-Rha-(1→2)-[R-(→4)]-Fuc- |
| | | | (R═5-O-[5-O-Ara/Api-3,5- |
| | | | dihydroxy-6-methyl-octanoyl]-3,5- |
| | | | dihydroxy-6-methyl-octanoic acid) |
| QS-21 A-apio | Quillaic acid | Gal-(1→2)-[Xyl-(1→3)]- | Api-(1→3)-Xyl-(1→4)-Rha-(1→2)- |
| | | GlcA- | [R-(→4)]-Fuc- |
| | | | (R═5-O-[5-O-Ara/Api-3,5- |
| | | | dihydroxy-6-methyl-octanoyl]-3,5- |
| | | | dihydroxy-6-methyl-octanoic acid) |
| QS-21 A-xylo | Quillaic acid | Gal-(1→2)-[Xyl-(1→3)]- | Xyl-(1→3)-Xyl-(1→4)-Rha-(1→2)- |
| | | GlcA- | [R-(→4)]-Fuc- |
| | | | (R═5-O-[5-O-Ara/Api-3,5- |
| | | | dihydroxy-6-methyl-octanoyl]-3,5- |
| | | | dihydroxy-6-methyl-octanoic acid) |
| QS-21 B-apio | Quillaic acid | Gal-(1→2)-[Xyl-(1→3)]- | Api-(1→3)-Xyl-(1→4)-Rha-(1→2)- |
| | | GlcA- | [R-(→3)]-Fuc- |
| | | | (R═5-O-[5-O-Ara/Api-3,5- |
| | | | dihydroxy-6-methyl-octanoyl]-3,5- |
| | | | dihydroxy-6-methyl-octanoic acid) |
| QS-21 B-xylo | Quillaic acid | Gal-(1→2)-[Xyl-(1→3)]- | Xyl-(1→3)-Xyl-(1→4)-Rha-(1→2)- |
| | | GlcA- | [R-(→3)]-Fuc- |
| | | | (R═5-O-[5-O-Ara/Api-3,5- |
| | | | dihydroxy-6-methyl-octanoyl]-3,5- |
| | | | dihydroxy-6-methyl-octanoic acid) |
| beta-Aescin | Protoaescigenin- | Glc-(1→2)-[Glc- | — |
| (described: | 21(2-methylbut-2- | (1→4)]-GlcA- | |
| Aescin Ia) | enoate)-22-acetat | | |
| Teaseed | 23-Oxo- | Glc-(1→2)-Ara-(1→3)- | — |
| saponin I | barringtogenol | [Gal-(1→2)]-GlcA- | |
| | C - 21,22-bis(2- | | |
| | methylbut-2- | | |
| | enoate) | | |
| Teaseedsaponin | 23-Oxo- | Xyl-(1→2)-Ara-(1→3)- | — |
| J | barringtogenol | [Gal-(1→2)]-GlcA- | |
| | C - 21,22-bis(2- | | |
| | methylbut-2- | | |
| | enoate) | | |
| Assamsaponin F | 23-Oxo- | Glc-(1→2)-Ara-(1→3)- | — |
| | barringtogenol | [Gal-(1→2)]-GlcA- | |
| | C - 21(2-methylbut- | | |
| | 2-enoate)-16,22- | | |
| | diacetat | | |
| Digitonin | Digitogenin | Glc-(1→3)-Gal-(1→2)- | — |
| | | [Xyl-(1→3)]-Glc-(1→4)- | |
| | | Gal- | |
| Primula acid 1 | 3,16,28- | Rha-(1→2)-Gal-(1→3)- | — |
| | Trihydroxyoleanan- | [Glc-(1→2)]-GlcA- | |
| | 12-en | | |
| AS64R | Gypsogenic acid | — | Glc-(1→3)-[Glc-(1→6)]-Gal- |
| | | Carbohydrate substituent | |
| | | at the C-23-OH group | |
| AS6.2 | Gypsogenic acid | Gal- | Glc-(1→3)-[Glc-(1→6)]-Gal- |
| Sapofectosid¹ | Quillaic acid | | |
| Agrostemmoside E | Quillaic acid | | |
| (AG1856, AG2.8)² | | | |

^{a, b}Different names refer to different isolates of the same structure
^{c, d}Different names refer to different isolates of the same structure
¹The structure of sapofectosid is provided in S. Sama et al, Sapofectosid - Ensuring non-toxic and effective DNA and RNA delivery, International Journal of Pharmaceutics, Volume 534, Issues 1-2, 20 Dec. 2017, Pages 195-205.
²The structure of Agrostemmoside E (also referred to as AG1856 or AG2.8) is given FIG. 4 of J. Clochard et al, A new acetylated triterpene saponin from Agrostemma githago L. modulates gene delivery efficiently and shows a high cellular tolerance, International Journal of Pharmaceutics, Volume 589, 15 Nov. 2020, 119822.

### EXAMPLES AND EXEMPLARY EMBODIMENTS

### Example 1. V_{HH}-SO1861+mAb-saporin (1T2C and 2T2C)

The 1 target 2-components system (1T2C) is the combination treatment of V_{HH}-SO1861 and mAb-protein toxin, where V_{HH} and mAb recognize and bind the same cell surface receptor (FIG. 1A). The 2 target 2-components system (2T2C) is the combination treatment of V_{HH}-SO1861 and mAb-protein toxin, where V_{HH} recognizes and binds a different cell surface receptor as the mAb (FIG. 1B). SO1861-EMCH was conjugated (labile) via the terminal cysteine residue (Cys) to antiHER2V_{HH}, with a DAR 1, (HER2V_{HH}-SO1861). HER2V_{HH}-SO1861 was titrated on a fixed concentration of 10 pM CD71mab-saporin (CD71 monoclonal antibody conjugated to the protein toxin, saporin, with a DAR4) or 50 pM trastuzumab-saporin (trastuzumab conjugated to the protein toxin, saporin, with a DAR4). Targeted protein toxin mediated cell killing on SK-BR-3 (HER2⁺⁺/CD71-9 and MDA-MB-468 (HER2⁻/CD71⁺) was determined. This revealed enhanced cell killing at low concentrations of HER2V_{HH}-SO1861 in SK-BR-3 for both combinations with 10 pM CD71mab-saporin or 50 pM trastuzumab-saporin (IC50=300 nM; FIG. 2A). Equivalent concentrations of HER2V_{HH}-SO1861 alone induced cell killing at high concentrations (IC50=4.000 nM), whereas equivalent concentrations of HER2V_{HH}, HER2V_{HH}+CD71 mab-saporin or HER2V_{HH}+trastuzumab-saporin could not induce cell killing activity (IC50>5000 nM; FIG. 2A). In MDA-MB-468 (HER2⁻/CD71⁺) the combination of HER2V_{HH}-SO1861+10 pM CD71mab-saporin revealed cell killing activity at high concentrations (IC50=2.000 nM; FIG. 2B), whereas the combination of HER2V_{HH}-SO1861+50 pM trastuzumab-saporin showed cell killing activity at much higher concentrations (IC50>5.000 nM; FIG. 2B). Equivalent concentrations of HER2V_{HH}, HER2V_{HH}+CD71mab-saporin or HER2V_{HH}+trastuzumab-saporin could not induce cell killing activity in MDA-MB-468 cells (IC50>5.000 nM; FIG. 2B).

All this shows that conjugation of SO1861-EMCH to a HER2 targeting V_{HH} enhances the endosomal escape and cytoplasmic delivery of a targeted protein toxin (targeting the same or different cell surface receptor) resulting in cell killing of HER2 expressing cells.

Next, trastuzumab-saporin or CD71 mab-saporin was titrated on a fixed concentration of 900 nM HER2V_{HH}-SO1861 and targeted protein toxin mediated cell killing on SK-BR-3 (HER2⁺⁺/CD71-9 and MDA-MB-468 (HER2⁻/CD71⁺) was determined. This revealed that 900 nM HER2V_{HH}-SO1861 in combination with low concentrations trastuzumab-saporin or CD71 mab-saporin induced already efficient cell killing of SK-BR-3 (IC50=0,0001 pM; FIG. 3A), whereas CD71 mab-saporin+900 nM HER2V_{HH} or trastuzumab-saporin+900 nM HER2V_{HH} could only induce cell killing at high concentrations (IC50=50 pM; IC50=400 pM resp; FIG. 3). In MDA-MB-468 cells (HER2⁻/CD71⁺) CD71mab-saporin+900 nM HER2V_{HH}-SO1861 showed cell killing at IC50=0.01 pM, whereas trastuzumab-saporin+900 nM HER2V_{HH}-SO1861 showed activity at IC50=2.000 pM. Trastuzumab-saporin+900 nM HER2V_{HH} or CD71 mab-saporin+900 nM HER2V_{HH} showed cell killing only at (IC50>10.000 pM and IC50=20 pM resp. FIG. 3B). All this shows that relatively low concentrations of trastuzumab-saporin or CD71mab-saporin can be effective and induce cell killing in combination with relatively low HER2V_{HH}-SO1861 (DAR1) concentrations in HER2⁺⁺/CD71⁺ expressing cells.

### Example 2. V_{HH}-SO1861+V_{HH}-dianthin (2T2C)

The 2 target 2-components system (2T2C) is the combination treatment of V_{HH}1-SO1861 and V_{HH}2-protein toxin, where each V_{HH} recognizes another cell surface receptor (FIG. 1C). SO1861-EMCH was conjugated to the terminal cysteine residues of the V_{HH} targeting HER2, producing HER2V_{HH}-SO1861 (DAR1). HER2V_{HH}-SO1861 was titrated on a fixed concentration of 50 pM CD71V_{HH}-dianthin and targeted protein toxin mediated cell killing on SK-BR-3 (HER2⁺⁺/CD71-9 and MDA-MB-468 (HER2-/CD71-9 was determined. This revealed enhanced cell killing at relatively low concentrations of VHHHER2-L-SO1861 (SK-BR-3: IC50=300 nM; FIG. 4A). Equivalent concentrations of HER2V_{HH}-SO1861 alone induced cell killing at high concentrations (IC50=4.000 nM), whereas equivalent concentrations of HER2V_{HH}, HER2V_{HH}+50 pM CD71V_{HH}-dianthin could not induce cell killing (IC50>5.000 nM; FIG. 4A). In MDA-MB-468 (HER2⁻/CD71⁺) the combination of HER2V_{HH}-SO1861+50 pM CD71V_{HH}-dianthin revealed cell killing activity at higher concentrations (IC50=600 nM; FIG. 4B), whereas equivalent concentrations of HER2V_{HH}, HER2V_{HH}-SO1861 or HER2V_{HH}+50 pM CD71V_{HH}-dianthin could not induce cell killing activity (IC50>5.000 nM; FIG. 4B).

Next, CD71V_{HH}-dianthin was titrated on a fixed concentration of 900 nM HER2V_{HH}-SO1861 and targeted protein toxin mediated cell killing on SK-BR-3 (HER2⁺⁺/CD71-9 and MDA-MB-468 (HER2-/CD71+) was determined. This revealed that 900 nM HER2V_{HH}-SO1861 in combination with low concentrations CD71V_{HH}-dianthin induced efficient cell killing of SK-BR-3 cells (IC50=0.05 pM; FIG. 5A), whereas CD71V_{HH} dianthin or CD71V_{HH}-dianthin+900 nM HER2V_{HH} could only induce cell killing at high concentrations (IC50>10.000 pM); FIG. 5A). Besides, CD71V_{HH}-dianthin was also titrated on a fixed concentration of 77 nM trastuzumab-SO1861 (DAR4) and this revealed also a strong enhancement in cell killing activity in SK-BR-3 (HER2⁺⁺/CD71-9 cells ((IC50<0,0001 pM). In MDA-MB-468 cells (HER2⁻/CD71⁺) CD71V_{HH}-dianthin+900 nM HER2V_{HH}-SO1861 showed cell killing only at much higher concentrations (IC50=10 pM, FIG. 5B), whereas CD71V_{HH}-dianthin, CD71V_{HH}-dianthin+900 nM HER2V_{HH} or CD71V_{HH}-dianthin+trastuzumab-SO1861 (DAR4) showed cell killing only at IC50=2.000 pM; FIG. 5B).

All this shows that relatively low concentrations of VHHCD71-dianthin can be effective and induce cell killing in combination with low VHHHER2-SO1861 conjugate concentrations in high HER2/CD71 expressing cells.

The combination according to the invention in MDA-MB-468 cells (HER2⁻/CD71⁺) did not reveal any cell killing activity. This shows that in the absence of sufficient receptor expression, effective intracellular delivered SO1861 concentrations are not reached (threshold) to induce endosomal escape and cytoplasmic delivery of the protein toxin.

### Example 3. V_{HH}-dianthin+mAb-SO1861 (1T2C and 2T2C)

The 1 target 2-components system (1T2C) is the combination treatment of mAb-SO1861 and V_{HH}-protein toxin, where mAb and V_{HH} recognize and bind the same cell surface receptor (FIG. 1E). The 2 target 2-components system (2T2C) is also the combination treatment of mAb-SO1861 and V_{HH}-protein toxin, where the mAb and V_{HH} recognize another cell surface receptor (FIG. 1D).

Dianthin-C(dianthin with a terminal cysteine) was conjugated to the terminal cysteine residues of the V_{HH} targeting HER2, V_{HH} targeting CD71 or V_{HH} targeting EGFR producing HER2V_{HH}-dianthin (DAR1), CD71V_{HH}-dianthin (DAR1) and EGFRV_{HH}-dianthin (DAR1).

CD71V_{HH}-dianthin, HER2V_{HH}-dianthin or EGFRV_{HH}-dianthin was titrated on a fixed concentration of cetuximab-SO1861 (DAR4) and targeted protein toxin mediated cell killing on A431 (EGFR⁺⁺/HER2^{+/-}/CD71+) and A2058 (EGFR⁻/HER2^{+/-}/CD71⁺) was determined. This revealed that very low concentrations CD71V_{HH}-dianthin in combination with 77 nM cetuximab-SO1861 induced efficient cell killing of A431 cells (IC50<0,0001 pM; FIG. 6A), whereas CD71V_{HH}-dianthin alone showed activity at IC50=2000 pM. The other two combinations EGFRV_{HH}-dianthin+77 nM cetuximab-SO1861 and HER2V_{HH}-dianthin+77 nM cetuximab-SO1861 showed efficient cell killing at respectively IC50=20 pM and IC50=50 pM, whereas EGFRV_{HH}-dianthin or HER2V_{HH}-dianthin alone could not induce efficient cell killing in A431 cells (IC50>10.000 pM; FIG. 6A). In A2058 cells (EGFR⁻/HER2^{+/-}/CD71+), CD71V_{HH}-dianthin and CD71V_{HH}-dianthin+77 nM cetuximab-SO1861 showed cell killing activity at respectively, IC50=3.000 and IC50=1.000 pM, whereas all other treatments or combinations showed no cell killing up to IC50=10.000 pM V_{HH}-toxin in A2058 cells (FIG. 6B).

This shows that cetuximab-SO1861 (DAR4) can efficiently induce endosomal escape of three different V_{HH}-dianthin conjugates, thereby inducing enhanced cell killing in A431 cells.

Next, CD71V_{HH}-dianthin, HER2V_{HH}-dianthin or EGFRV_{HH}-dianthin was titrated on a fixed concentration of trastuzumab-SO1861 (DAR4) and targeted protein toxin mediated cell killing on SK-BR-3 (HER2⁺⁺/EGFR⁻/CD71⁺) and MDA-MB-468 cells (HER2⁻/EGFR⁺⁺/CD71⁺) was determined. This revealed that very low concentrations CD71V_{HH}-dianthin in combination with 77 nM trastuzumab-SO1861 induced efficient cell killing of SK-BR-3 cells (IC50<0,0001 pM; FIG. 7A), whereas CD71 V_{HH}-dianthin alone showed activity at IC50=10.000 pM. The other two combinations EGFRV_{HH}-dianthin+77 nM trastuzumab-SO1861 and HER2V_{HH}-dianthin+77 nM trastuzumab-SO1861 showed efficient cell killing at respectively IC50=400 pM and IC50=6 pM, whereas EGFRV_{HH}-dianthin or HER2V_{HH}-dianthin alone could not induce efficient cell killing in SK-BR-3 cells (IC50>10.000 pM; FIG. 7A). In MDA-MB-468 cells (HER2⁻/EGFR⁺⁺/CD71⁺), CD71V_{HH}-dianthin and CD71V_{HH}-dianthin+77 nM cetuximab-SO1861 showed cell killing activity at respectively, IC50=3000 and IC50=2000 pM, whereas all other treatments or combinations showed no cell killing up to IC50=10.000 pM V_{HH}-dianthin in MDA-MB-468 cells (FIG. 7B). This shows that trastuzumab-SO1861 (DAR4) can efficiently induce endosomal escape of three different V_{HH}-dianthin conjugates, thereby inducing enhanced cell killing in SK-BR-3 cells.

Materials and Methods

Materials

SO1861 was isolated and purified by Analyticon Discovery GmbH from raw plant extract obtained from Saponaria officinalis. V_{HH} were purchased from QVQ, Utrecht, The Netherlands (HER2V_{HH}: clone name: Q17c; CD71V_{HH}: clone name: Q52c EGFRV_{HH}: clone name: Q86c). Trastuzumab (Tras, Herceptin®, Roche), Cetuximab (Cet, Erbitux®, Merck KGaA) were purchased from the pharmacy (Charite, Berlin). CD71 monoclonal antibody was purchased from BioCell (Okt9, #BE0023). Custom trastuzumab-saporin and antiCD71 mab-saporin conjugate was produced and purchased from Advanced Targeting Systems (San Diego, Calif.). Dianthin-Cys (Dia-Cys, Dianthin mutant with a single C-terminal cysteine was produced by Proteogenix, France.

Tris(2-carboxyethyl)phosphine hydrochloride (TCEP, 98%, Sigma-Aldrich), 5,5-Dithiobis(2-nitrobenzoic acid) (DTNB, Ellman's reagent, 99%, Sigma-Aldrich), Zeba™ Spin Desalting Columns (2 mL, Thermo-Fisher), NuPAGE™ 4-12% Bis-Tris Protein Gels (Thermo-Fisher), NuPAGE™ MES SDS Running Buffer (Thermo-Fisher), Novex™ Sharp Pre-stained Protein Standard (Thermo-Fisher), PageBlue™ Protein Staining Solution (Thermo-Fischer), Pierce™ BCA Protein Assay Kit (Thermo-Fisher), N-Ethylmaleimide (NEM, 98%, Sigma-Aldrich), 1,4-Dithiothreitol (DTT, 98%, Sigma-Aldrich), Sephadex G25 (GE Healthcare), Sephadex G50 M (GE Healthcare), Superdex 200P (GE Healthcare), Isopropyl alcohol (IPA, 99.6%, VWR), Tris(hydroxymethyl)aminomethane (Tris, 99%, Sigma-Aldrich), Tris(hydroxymethyl)aminomethane hydrochloride (Tris·HCL, Sigma-Aldrich), L-Histidine (99%, Sigma-Aldrich), D-(+)-Trehalose dehydrate (99%, Sigma-Aldrich), Polyethylene glycol sorbitan monolaurate (TWEEN 20, Sigma-Aldrich), Dulbecco's Phosphate-Buffered Saline (DPBS, Thermo-Fisher), Guanidine hydrochloride (99%, Sigma-Aldrich), Ethylenediaminetetraacetic acid disodium salt dihydrate (EDTA-Naz, 99%, Sigma-Aldrich), sterile filters 0.2 μm and 0.45 μm (Sartorius), Succinimidyl 4-(N-maleimidomethyl)cyclohexane-1-carboxylate (SMCC, Thermo-Fisher), Vivaspin T4 and T15 concentrator (Sartorius), Superdex 200PG (GE Healthcare), Tetra(ethylene glycol) succinimidyl 3-(2-pyridyldithio)propionate (PEG4-SPDP, Thermo-Fisher), HSP27 BNA disulfide oligonucleotide (Biosynthesis), [0-(7-Azabenzotriazol-1-yl)-N,N,N,N-tetramethyluronium-hexafluorphosphat] (HATU, 97%, Sigma-Aldrich), Dimethyl sulfoxide (DMSO, 99%, Sigma-Aldrich), N-(2-Aminoethyl)maleimide trifluoroacetate salt (AEM, 98%, Sigma-Aldrich), L-Cysteine (98.5%, Sigma-Aldrich), deionized water (DI) was freshly taken from Ultrapure Lab Water Systems (MilliQ, Merck), Nickel-nitrilotriacetic acid agarose (Ni-NTA agarose, Protino), Glycine (99.5%, VWR), 5,5-Dithiobis(2-nitrobenzoic acid (Ellman's reagent, DTNB, 98%, Sigma-Aldrich), S-Acetylmercaptosuccinic anhydride Fluorescein (SAMSA reagent, Invitrogen) Sodium bicarbonate (99.7%, Sigma-Aldrich), Sodium carbonate (99.9%, Sigma-Aldrich), PD MiniTrap desalting columns with Sephadex G-25 resin (GE Healthcare), PD10 G25 desalting column (GE Healthcare), Zeba Spin Desalting Columns in 0.5, 2, 5, and 10 mL (Thermo-Fisher), Vivaspin Centrifugal Filters T4 10 kDa MWCO, T4 100 kDa MWCO, and T15 (Sartorius), Biosep s3000 aSEC column (Phenomenex), Vivacell Ultrafiltration Units 10 and 30 kDa MWCO (Sartorius), Nalgene Rapid-Flow filter (Thermo-Fisher),

Methods

SO1861-EMCH Synthesis

To SO1861 (121 mg, 0.065 mmol) and EMCH·TFA (110 mg, 0.325 mmol) was added methanol (extra dry, 3.00 mL) and TFA (0.020 mL, 0.260 mmol). The reaction mixture stirred at room temperature. After 1.5 hours the reaction mixture was subjected to preparative MP-LC. ¹ Fractions corresponding to the product were immediately pooled together, frozen and lyophilized overnight to give the title compound (120 mg, 90%) as a white fluffy solid. Purity based on LC-MS 96%.
LRMS (m/z): 2069 [M−1]¹⁻
LC-MS r.t. (min): 1.08⁴

Cell Viability Assay

After treatment the cells were incubated for 72 hr at 37° C. before the cell viability was determined by a MTS-assay, performed according to the manufacturer's instruction (CellTiter 96® AQueous One Solution Cell Proliferation Assay, Promega). Briefly, the MTS solution was diluted 20× in DMEM without phenol red (PAN-Biotech GmbH) supplemented with 10% FBS. The cells were washed once with 200 μL/PBS well, after which 100 μL diluted MTS solution was added/well. The plate was incubated for approximately 20-30 minutes at 37° C. Subsequently, the OD at 492 nm was measured on a Thermo Scientific Multiskan FC plate reader (Thermo Scientific). For quantification the background signal of ‘medium only’ wells was subtracted from all other wells, before the cell viability percentage of treated/untreated cells was calculated, by dividing the background corrected signal of treated wells over the background corrected signal of the untreated wells (×100).

FACS Analysis

Cells were seeded in DMEM (PAN-Biotech GmbH) supplemented with 10% fetal calf serum (PAN-Biotech GmbH) and 1% penicillin/streptomycin (PAN-Biotech GmbH), at 500,000 c/plate in 10 cm dishes and incubated for 48 hrs (5% CO₂, 37° C.), until a confluency of 90% was reached. Next, the cells were trypsinized (TrypIE Express, Gibco Thermo Scientific) to single cells. 0.75×10⁶ Cells were transferred to a 15 mL falcon tube and centrifuged (1,400 rpm, 3 min). The supernatant was discarded while leaving the cell pellet submerged. The pellet was dissociated by gentle tapping the falcon tube on a vortex shaker and the cells were washed with 4 mL cold PBS (Mg²⁺ and Ca²⁺ free, 2% FBS). After washing the cells were resuspended in 3 mL cold PBS (Mg²⁺ and Ca²⁺ free, 2% FBS) and divided equally over 3 round bottom FACS tubes (1 mL/tube). The cells were centrifuged again and resuspended in 200 μL cold PBS (Mg²⁺ and Ca²⁺ free, 2% FBS) or 200 μL antibody solution; containing 5 μL antibody in 195 μL cold PBS (Mg²⁺ and Ca²⁺ free, 2% FBS). APC Mouse IgG1, κ APC anti-human EGFR (#352906, Biolegend) was used to stain the EGFR receptor. PE anti-human HER2 APC anti-human CD340 (erbB2/HER-2) (#324408 Biolegend) was used to stain the HER2 receptor, PE Mouse IgG2a, K Isotype Ctrl FC (#400212, Biolegend) was used as its matched isotype control. PE anti-human CD71 (#334106, Biolegend) was used to stain the CD71 receptor, PE Mouse IgG2a, K Isotype Ctrl FC (#400212, Biolegend) was used as its matched isotype control. Samples were incubated for 30 min at 4° C. on a tube roller mixer. Afterwards, the cells were washed 3× with cold PBS (Mg²⁺ and Ca²⁺ free, 2% FBS) and fixated for 20 min at room temperature using a 2% PFA solution in PBS. Cells were washed 2× with cold PBS, and resuspended in 250-350 μL cold PBS for FACS analysis. Samples were analyzed with a BD FACSCanto II flow cytometry system (BD Biosciences) and FlowJo software. Results of the analyses of the cell-surface expression of EGFR, HER2 and CD71 on the various cells is summarized in Table A2.

TABLE A2
Cell surface expression levels of EGFR,
HER2 and CD71 of various cells

| | EGFR | HER2 | CD71 |
|---|---|---|---|
| | expression | expression | expression |
| Cell line | level (MFI) | level (MFI) | level (MFI) |
| MDA-MB-468 | 1656 | 1 | 186 |
| A431 | 1593 | 10 | 322 |
| SK-BR-3 | 28 | 1162 | 331 |
| A2058 | 1 | 5 | 59 |

Procedure for the Conjugation of V_{HH}-SO1861

To an aliquot of V_{HH} was added an aliquot of freshly prepared TCEP solution (10.0 mg/ml), the mixture vortexed briefly then incubated for 30 minutes at 20° C. with roller-mixing. After incubation, the resulting V_{HH}-SH was purified by gel filtration using zeba spin desalting column into TBS pH 7.5. To the resulting V_{HH}-SH was added freshly prepared SPT-EMCH solution the mixture vortexed briefly then incubated overnight at 20° C.

After incubation, an aliquot of V_{HH}-SO1861 mixture was removed and characterised by Ellman's assay to ascertain SO1861 incorporation. The conjugate was purified by 1.6×35 cm Superdex 200PG column eluting with DPBS pH 7.5 to give purified V_{HH}-SO1861. The aliquot was filtered to 0.2 μm, concentrated and normalised to 1.0 mg/ml to afford V_{HH}-SO1861.

Procedure for the conjugation of V_{HH}-Dianthin Dianthin-Cys was concentrated by ultrafiltration using a vivaspin T15 10 KDa MWCO centrifugal filter and buffer exchanged into TBS pH 7.5. To the concentrated Dianthin-Cys was added an aliquot of freshly prepared TCEP solution (10.0 mg/ml), the mixture vortexed briefly then incubated for 60 minutes at 20° C. with roller-mixing. After incubation, the resulting Dianthin-SH was purified by gel filtration using a zeba spin desalting column then repeated centrifugal-wash cycles using a vivaspin T15 10 KDa MWCO centrifugal filter into TBS pH 7.5. The resulting Dianthin-SH was reacted with freshly prepared DTME solution (10 mg/ml) in DMSO, the mixture vortexed briefly then incubated for 60 minutes at 20° C. After, the Dianthin-DTME was obtained following purification by gel filtration using a zeba spin desalting column into TBS pH 7.5. The Dianthin-DTME was stored at 20° C. until conjugated. At the same time, an aliquot of V_{HH} was concentrated by ultrafiltration using a vivaspin T15 10 KDa MWCO centrifugal filter and buffer exchanged into TBS pH 7.5. To the concentrated V_{HH} was added an aliquot of freshly prepared TCEP solution (10.0 mg/ml), the mixture vortexed briefly then incubated for 60 minutes at 37° C. with roller-mixing. After incubation, the resulting V_{HH} was purified by gel filtration using a zeba spin desalting column then repeated centrifugal-wash cycles using a vivaspin T4 5 KDa MWCO centrifugal filter into TBS pH 7.5. An aliquot of the resulting V_{HH}-SH was reacted with Dianthin-DTME, the mixture vortexed briefly then incubated overnight at 20° C. After, the reaction mixture was concentrated using a vivaspin T4 10 KDa MWCO centrifuge tube and purified by gel filtration using a 1.6×35 cm Superdex 200PG column eluting into DPBS pH 7.5.

Antibody-(L-SO1861)⁴

Trastuzumab, Cetuximab, are referred hereafter as “Ab”. Ab was conjugated to the saponin SO1861-EMCH via Michael-type thiol-ene conjugation reaction at DARs of 1, 2, 3, 4, 5, and 6. The SO1861-EMCH molecule obtains a labile (L) hydrazone bond between its structure and its maleimide function generating a labile bond between the saponin and Ab. The procedure is exemplary described for Trastuzumab-(L-SO1861)₄:

To a solution of Cetuximab (40 mg, 8.0 ml) was added 10 μl/ml each of Tris concentrate (127 mg/ml, 1.05 M), Tris·HCl concentrate (623 mg/ml, 3.95 M) and EDTA-Naz concentrate (95 mg/ml, 0.26 M) to give a 50 mM TBS, 2.5 mM EDTA buffer pH 7.5.

To Cetuximab divided into four portions (each of 9.73 mg, 4.864 mg/ml, 65 nmol) was added an aliquot of freshly prepared TCEP solution (0.5-2.0 mg/ml, 1.15-7.02 mole equivalents, 75-455 nmol), the mixtures vortexed briefly then incubated for 300 minutes at 20° C. with roller-mixing. After incubation (prior to addition of SO1861-EMCH), a ca. 1 mg (0.210 ml) aliquot of Ab-SH was removed from each mixture and purified by gel filtration using a zeba spin desalting column into TBS pH 7.5. These aliquots were characterized by UV-vis analysis and Ellman's assay (thiol to Ab ratio=2.0, 4.2, 5.9 and 6.8 respectively). To each of the bulk Ab-SH was added an aliquot of freshly prepared SO1861-EMCH solution (2 mg/ml, 1.3 mole equivalents per ‘thiol’, 0.15-0.61 μmol, 0.16-0.63 ml), the mixtures vortexed briefly then incubated for 120 minutes at 20° C. Besides each conjugation reaction, two aliquots of desalted Ab-SH (0.25 mg, 1.67 nmol) were reacted with NEM (1.3 mole equivalents per ‘thiol’, 4.3-17.4 nmol, 2.2-8.7 μl of a 0.25 mg/ml solution) or TBS pH 7.5 buffer (2.2-8.7 μl) for 120 minutes at 20° C., as positive and negative controls, respectively. After incubation (prior to addition of NEM), a 0.200 ml aliquot of Ab-SO1861-EMCH mixture was removed and purified by gel filtration using zeba spin desalting column into TBS pH 7.5. This aliquot was characterized by UV-vis and alongside positive and negative controls were characterized by Ellman's assay to obtain SO1861-EMCH incorporations. To the bulk Ab-SO1861-EMCH mixture was added an aliquot of freshly prepared NEM solution (2.5 mg/ml, 2.5-10 mole equivalents, 0.15-0.58 μmol) and the mixtures purified by zeba spin desalting columns eluting with DPBS pH 7.5 to give purified Cetuximab-(L-SO1861) conjugates. The products were normalized to 2.5 mg/ml and filtered to 0.2 μm prior to dispensing for biological evaluation. The reaction conditions and results for Trastuzumab-L-SO1861 conjugates and the reaction conditions and results for Cetuximab-L-SO1861 conjugates are summarized in Table A3 and Table A4.

TABLE A3
Summarized reaction conditions and results
for Trastuzumab-L-SO1861 conjugates

| | | TCEP | | | | |
|---|---|---|---|---|---|---|
| | | feed | | | Purity by | |
| | | mole | SO1861- | Ob- | analyt- | |
| | | equiv- | EMCH | tained | ical SEC | Yield |
| Batch | Ab feed | alents | feed | DAR | (%) | (%) |
| Tras-(L- | 9.91 mg | 3.83 | 0.46 μmol | 4.0 | 98.4 | 81 |
| SO1861)₄ | 66 nmol | | | | | |

TABLE A4
Summarized reaction conditions and results
for Cetuximab-L-SO1861 conjugates

| | | TCEP | | | | |
|---|---|---|---|---|---|---|
| | | feed | | | Purity by | |
| | | mole | SO1861- | Ob- | analyt- | |
| | | equiv- | EMCH | tained | ical SEC | Yield |
| Batch | Ab feed | alents | feed | DAR | (%) | (%) |
| Cet-(L- | 9.73 mg | 4.19 | 0.46 μmol | 4.1 | 99.0 | 77 |
| SO1861)₄ | 65 nmol | | | | | |

Summary of a Number of Embodiments Relating to Antibodies and Receptor Ligands Covalently Bound to a Protein Toxin or to a Saponin
mAb: trastuzumab (HER2) or cetuximab (EGFR)
Ligand: EGF
Protein toxin: Ribosome inactivating protein, saporin or dianthin

endosomal escape enhancing conjugates of saponin with a ligand:

mAb-SO1861 Endosomal Escape Enhancing Conjugates

| | |
|---|---|
| contain a cleavable hydrazone linker | |
| Trastuzumab-SO1861 | DAR 4.0 |
| Cetuximab -SO1861 | DAR 3.7; |

the endosomal escape enhancing conjugates of saponin with a ligand combined in in vitro or in vivo test models, with:

mAb/Ligand-Protein Toxin Conjugates

| | |
|---|---|
| contain a non-cleavable chemical linker | |
| or are recombinant fusion proteins | |
| Trastuzumab-Saporin | DAR 3.0 |
| Cetuximab-Saporin | DAR 2.6 |
| Trastuzumab-Dianthin | DAR 1.0 |
| EGF-Dianthin (fusion protein) | DAR 1.0 |
| IgG-Saporin | DAR 2.2 |

For Examples 4-9:

Materials:

Trastuzumab and cetuximab were purchased from the pharmacy (Charite, Berlin). SO1861 was isolated and purified by Analyticon Discovery GmbH from raw plant extract obtained from Saponaria officinalis L.

Methods

SO1861-EMCH Synthesis

SO1861 was from Saponaria officinalis L (Analyticon Discovery GmbH), and was coupled to EMCH according to conventional steps known in the art.

Conjugation of SO1861 to Antibodies

Custom production of trastuzumab-SO1861 and cetuximab-SO1861 was performed by FleetBioprocessing (UK). SO1861-EMCH was conjugated to cysteines of the antibody.

Conjugation of Saporin to Trastuzumab and Cetuximab

Custom trastuzumab-saporin and cetuximab-saporin conjugates were produced and purchased from Advanced Targeting Systems (San Diego, Calif.). IgG-saporin and saporin was purchased from Advanced

Targeting Systems

FACS Analyses

FACS analysis was performed on a BD FACSCanto II, data analysis with FlowJo V10 software, FACS antibodies were: 1) Isotype: APC Mouse IgG1, K Isotype Ctrl (FC) (400122, Biolegend). EGFR: APC anti-human EGFR (352906, Biolegend) HER2: APC anti-human CD340 (erbB2/HER-2) (324408, Biolegend).

Dianthin Production

Dianthin was expressed in a bacterium culture and the protein was purified following conventional cell culturing and protein purification steps known in the art.

Conjugation of Antibody to Dianthin

Conjugation of antibody and dianthin was according to common procedures known in the art.

Cell Culture

Cells were cultured in DMEM (PAN-Biotech GmbH) supplemented with 10% fetal bovine serum (FBS) (PAN-Biotech GmbH) at 37° C. and 5% CO2.

Cell Viability Assay

Cells were seeded in a 96 well plate at 5.000-10.000 c/w in 100 μL/well and incubated overnight at 37° C. The next day 10× concentrated treatment-mix samples were prepared in PBS, which contain antibody-conjugated SO1861 (i.e. a ‘binding molecule’ or an ‘endosomal escape enhancing conjugate’ of the invention) and targeted-toxin (i.e. a ‘binding molecule’) both at 10× final concentration. The media was removed from the cell culture plate and replaced by 180 μL culture media, followed by the addition of 20 μL treatment-mix/well. For control, 10× treatment-mix samples were prepared that contained the corresponding concentrations of only antibody-conjugated SO1861, only antibody, only SO1861, only targeted-toxin, or PBS without compound as vehicle control. In the case that endosomal acidification inhibitors (chloroquine (Sigma Aldrich) or bafilomycin A1 (Enzo Life Sciences)) were used, the cell culture media in step 1 of treatment was replaced by 180 μL media containing 1 μM chloroquine or 0.2 μM bafilomycin A1. The plate was incubated for 1 hour at 37° C., before the 10× treatment-mix samples were added. The remaining incubation and treatment steps were performed according to standard procedures known in the field.

After treatment the cells were incubated for 72 hr at 37° C. before the cell viability was determined by a MTS-assay, performed according to the manufacturer's instruction (CellTiter 96® AQueous One Solution Cell Proliferation Assay, Promega). Briefly, the MTS solution was diluted 20× in DMEM without phenol red (PAN-Biotech GmbH) supplemented with 10% FBS (PAN-Biotech GmbH). The cells were washed once with 200 μL PBS per well, after which 100 μL diluted MTS solution was added per well. The plate was incubated for approximately 20-30 minutes at 37° C. Subsequently, the optical density at 492 nm was measured on a Thermo Scientific Multiskan FC plate reader (Thermo Scientific). For quantification the background signal of ‘medium only’ wells was subtracted from all other wells, before the ratio of untreated/treated cells was calculated, by dividing the background corrected signal of untreated wells over the background corrected signal of the treated wells.

### Results

### Example 4. 1 Target 2-Component System

1 target 2-components system is the combination treatment of mAb1-protein toxin and mAb1-SO1861 (see FIG. 1A, E), whereas the 2 target 2-component system is the combination of mAb1-protein toxin and mAb2-SO1861 or mAb2-protein toxin+mAb1-SO1861 (FIG. 1B-D).

Cetuximab-SO1861 (monoclonal antibody recognizing and binding EGFR, conjugated to the saponin molecule, SO1861; an endosomal escape enhancing conjugate) was titrated (calculated on concentration SO1861) on a fixed concentration of 10 pM cetuximab-saporin (monoclonal antibody recognizing and binding EGFR, conjugated to the protein toxin, saporin) and cell killing on high EGFR expressing cells was determined. High EGFR expressing cells (A431or CaSKi) showed efficient cell killing when 10 pM cetuximab-saporin was combined with high concentrations of non-targeted unconjugated SO1861 (A431: [SO1861] IC50=600 nM and Caski: [SO1861] IC50=700 nM; FIG. 8A, 8B; Table A6). However, when cetuximab-saporin was combined with cetuximab-SO1861 strong cell killing was induced already at low concentrations of SO1861 (A431: [SO1861] IC50=5 nM and Caski [SO1861] IC50=8 nM; FIG. 8A, 8B; Table A6). This shows that targeted conjugated SO1861 is more effective in inducing endosomal escape compared to non-targeted unconjugated SO1861. Next, cetuximab-saporin was titrated on a fixed concentration of 300 nM cetuximab-SO1861 and targeted protein toxin mediated cell killing on EGFR expressing cells was determined. High EGFR expressing cells (A431 or CaSKi) show cell killing only at high cetuximab-saporin concentrations in combination with non-targeted unconjugated 300 nM SO1861 (A431: [toxin] IC50=40 pM; CaSki: [toxin] IC50=40 pM; FIG. 8C, 8D; Table A7) whereas 300 nM cetuximab-SO1861 in combination with low cetuximab-saporin concentrations already induced efficient cell killing (A431: [toxin] IC50=0.4 pM; CaSKi: [toxin] IC50=2 pM; FIGS. 8C and 8D; Table A7). Highest cell killing efficiency is achieved when high concentrations of non-targeted unconjugated SO1861 (1500 nM) is combined with low concentrations of cetuximab-saporin (A431: [toxin] IC50=0.03 pM; CaSki: [toxin] IC50=0.02 pM; FIG. 8C, 8D; Table A7). All this shows that when conjugated to cetuximab, relatively low concentrations of SO1861 efficiently can kill high EGFR expressing cells in combination with relatively low concentrations of cetuximab-saporin. High concentrations (1500 nM) of non-targeted unconjugated SO1861 in combination with low concentrations of cetuximab-saporin is still most effective since receptor competition does not play a role for SO1861 to enter the cell, since in the 1-target 2-component system both conjugates compete for the same EGFR receptor. The receptor competition principle is also clearly shown in the cetuximab-toxin titration treatments without (A431: [toxin] IC50=40 pM; Caski: [toxin] IC50=40 pM) or with 75 nM cetuximab (A431: [toxin] IC50=1000 pM; Caski: from IC50=1000 pM; FIG. 8C, 8D).

Next, cetuximab-SO1861 was titrated (calculated on concentration SO1861) on a fixed concentration of 10 pM cetuximab-saporin and cell killing on low/no EGFR expressing cells was determined. Low EGFR expressing cells (HeLa) showed only cell killing when 10 pM cetuximab-saporin was combined with high concentrations of non-targeted unconjugated SO1861, whereas A2058 cells that do not express EGFR (A2058) were not sensitive at all (HeLa: [SO1861] IC50=1000 nM; A2058: [SO1861]IC>1000 nM; FIG. 9A, 9B; Table A6). The combination of 10 pM cetuximab-saporin with increasing concentrations of cetuximab-SO1861 did not induce any significant cell killing in both cell lines (HeLa: [SO1861] IC50=1000 nM; A2058: [SO1861] IC50>1000 nM; FIG. 9A, 9B; Table A6). This shows that in the absence of sufficient receptor expression, effective intracellular SO1861 concentrations are not reached (threshold) to induce endosomal protein toxin escape and toxin-mediated cell killing. Next, cetuximab-saporin was titrated on a fixed concentration of 300 nM cetuximab-SO1861 and targeted protein toxin mediated cell killing on low/no EGFR expressing cells was determined. Low EGFR expressing cells (HeLa) show cell killing only at very high cetuximab-saporin concentrations in combination with 278 nM cetuximab-SO1861 or 300 nM unconjugated SO1861, whereas A2058 cells (EGFR) are not sensitive at any of the tested concentrations (HeLa: [toxin] IC50=60 pM; A2058: [toxin] IC50>10.000 pM; FIG. 9C, 9D; Table A7). High concentrations of unconjugated SO1861 (1500 nM) in combination with low concentrations of cetuximab-saporin in low EGFR expressing cells (HeLa) show efficient cell killing, whereas in A2058 cells only at very high cetuximab-saporin concentrations in combination with 1500 nM SO1861 non-targeted, a-specific cell killing is induced (Hela: [toxin] IC50=0.03 pM; A2058: [toxin] IC50=20 pM; FIG. 9C, 9D; Table A6). All this shows that cells with low or no EGFR receptor expression are not susceptible for the combination of cetuximab-SO1861+cetuximab-saporin, due to a lack of sufficient EGFR receptor that facilitates the entry of sufficient SO1861 and toxin within the cell.

Trastuzumab-SO1861 (monoclonal antibody recognizing and binding HER2, conjugated to the saponin molecule, SO1861; an endosomal escape enhancing conjugate according to the invention) was titrated (calculated on concentration SO1861) on a fixed concentration of 50 pM trastuzumab-saporin (monoclonal antibody recognizing and binding HER2, conjugated to the protein toxin, saporin) and cell killing on high HER2 expressing cells was determined. High HER2 expressing cells (SKBR3) showed efficient cell killing when 50 pM trastuzumab-saporin was combined with high concentrations of non-targeted unconjugated SO1861 (SKBR3; FIG. 10A, 10B; Table A6). However, when trastuzumab-saporin was combined with trastuzumab-SO1861 strong cell killing was induced already at low concentrations of SO1861 (SKBR3; FIG. 10A, 10B; Table A6). This shows that targeted conjugated SO1861 is more effective in inducing endosomal escape compared to non-targeted unconjugated SO1861. Next, trastuzumab-saporin was titrated on a fixed concentration of 50 nM trastuzumab-SO1861 and targeted protein toxin mediated cell killing on HER2 expressing cells was determined. High HER2 expressing cells (SKBR3 or BT474) show cell killing only at high trastuzumab-saporin concentrations in combination with non-targeted unconjugated 10 nM SO1861 (Table A7) whereas 10 nM trastuzumab-SO1861 in combination with low trastuzumab-saporin concentrations already induced efficient cell killing (Table A7). Highest cell killing efficiency is achieved when high concentrations of non-targeted unconjugated SO1861 (1500 nM) is combined with low concentrations of trastuzumab-saporin (Table A7). All this shows that when conjugated to trastuzumab, low concentrations of SO1861 efficiently can kill high HER2 expressing cells in combination with relatively low concentrations of trastuzumab-saporin. High concentrations (1500 nM) of non-targeted unconjugated SO1861 in combination with low concentrations of trastuzumab-saporin is still most effective, since receptor competition does not play a role for SO1861 to enter the cell, since in the 1-target 2-component system both conjugates compete for the same EGFR receptor. The receptor competition principle is also clearly shown in the trastuzumab-toxin titration treatments without or with 2.5 nM trastuzumab (SKBR3: [toxin] IC50=1000 nM).

Next, trastuzumab-SO1861 was titrated (calculated on concentration SO1861) on a fixed concentration of 50 pM trastuzumab-saporin and cell killing on low/no EGFR expressing cells was determined. Low EGFR expressing cells (JIMT1; A431) showed only cell killing when 50 pM trastuzumab-saporin was combined with high concentrations of non-targeted unconjugated SO1861 (JIMT1: [SO1861] IC50>1000 nM; A431: [SO1861] IC50>1000 nM; FIG. 11A, 11B; Table A6). The combination of 50 pM trastuzumab-saporin with increasing concentrations of trastuzumab-SO1861 did not induce any significant cell killing in both cell lines (JIMT1: [SO1861]IC>1000 nM; A431: [SO1861] IC50>1000 nM; FIG. 11A, 11B; Table A6). This shows that in the absence of sufficient receptor expression, effective intracellular SO1861 concentrations are not reached (threshold) to induce endosomal protein toxin escape and toxin-mediated cell killing. Next, trastuzumab-saporin was titrated on a fixed concentration of 10 nM trastuzumab-SO1861 and targeted protein toxin mediated cell killing on low/no HER2 expressing cells was determined. Low HER2 expressing cells (JIMT1; A431) show no significant cell killing at high trastuzumab-saporin concentrations in combination with 10 nM trastuzumab-SO1861 (JIMT-1: [toxin] IC50>10.000 pM; A431: [toxin] IC50>10.000 pM; FIG. 110, 11D; Table A7). High concentrations of unconjugated SO1861 (1500 nM) in combination with low concentrations of trastuzumab-saporin in low HER2 expressing cells (show efficient cell killing (JIMT1: [toxin] IC50=0.1 pM; A431: [toxin] IC50=0.8 pM; FIG. 11C, 11D; Table A5). All this shows that cells with low HER2 receptor expression are not susceptible for the combination of trastuzumab-SO1861+trastuzumab-saporin, due to a lack of sufficient HER2 receptor that facilitates the entry of sufficient SO1861 and toxin within the cell.

### Example 5. 2 Target 2-Component System

Cetuximab-SO1861 was titrated (calculated on concentration SO1861) on a fixed concentration of 50 pM trastuzumab-saporin and cell killing on high EGFR/low HER2 expressing cells was determined. A431 and CaSki cells showed efficient cell killing when 50 pM trastuzumab-saporin was combined with high concentrations of non-targeted unconjugated SO1861 (A431 and Caski: [SO1861] IC50=1000 nM; FIG. 12A, 12B; Table A6). However, when trastuzumab-saporin was combined with cetuximab-SO1861 strong cell killing was induced already at low concentrations of SO1861 (A431: [SO1861] IC50=12 nM and Caski [SO1861] IC50=40 nM; FIG. 12A, 12B; Table A6). This shows that targeted conjugated SO1861 is more effective in inducing endosomal escape compared to non-targeted unconjugated SO1861. Next, trastuzumab-saporin was titrated on a fixed concentration of 300 nM cetuximab-SO1861 and targeted protein toxin mediated cell killing was determined on high EGFR/low HER2 expressing cells (A431 and CaSki) No effective cell killing was observed with high trastuzumab-saporin concentrations in combination with non-targeted unconjugated 300 nM SO1861 (A431 and Caski: [toxin] IC50>10.000 pM; FIG. 12C, 12D; Table A7) whereas 300 nM cetuximab-SO1861 in combination with low trastuzumab-saporin concentrations already induced efficient cell killing (A431: [toxin] IC50=3 pM; Caski: [toxin] IC50=1 pM; FIGS. 12C and 12D; Table A7). In A431 cells, comparable cell killing efficiency is achieved when high concentrations (1500 nM) of non-targeted unconjugated SO1861 is combined with low concentrations of trastuzumab-saporin (A431: [toxin] IC50=1 pM; see FIG. 12C; Table A7). In Caski cells the response was slightly stronger compared to the combination of cetuximab-SO1861 and Trastuzumab-saporin, due to the fact that the EGFR expression in these cells is significantly lower compared to A431 and thus targeted delivery of SO1861 to Caski cells is less sufficient (Caski: [toxin] IC50=0.2 pM see FIG. 12D; Table A7). All this shows that when conjugated to cetuximab, low concentrations of SO1861 efficiently can kill high EGFR expressing cells in combination with relatively low concentrations of trastuzumab-saporin. High concentrations (1500 nM) of non-targeted unconjugated SO1861 in combination with low concentrations of trastuzumab-saporin has comparable activity, since receptor competition does not play a role for SO1861 to enter the cell, since in the 2-target 2-component system both conjugates are delivered via different receptors, SO1861 via EGFR and toxin via HER2 receptor.

Next, cetuximab-SO1861 was titrated (calculated on concentration SO1861) on a fixed concentration of 50 pM trastuzumab-saporin and cell killing on low/no EGFR/HER2 expressing cells was determined. Low EGFR/HER2 expressing cells showed only cell killing when 50 pM trastuzumab-saporin was combined with high concentrations of non-targeted unconjugated SO1861 (HeLa: [SO1861] IC50>1000 nM; A2058: [SO1861] IC50>1000 nM; FIG. 13A, 13B; Table A6). The combination of 50 pM trastuzumab-saporin with increasing concentrations of cetuximab-SO1861 only showed significant cell killing at high concentrations of cetuximab-SO1861 in both cell lines (HeLa: [SO1861] IC50>1000 nM; A2058: [SO1861]IC>1000 nM; FIG. 13A, 13B; Table A6). This shows that in the absence of sufficient receptor expression, effective intracellular SO1861 concentrations are not reached (threshold) to induce endosomal protein toxin escape and toxin-mediated cell killing. Next, trastuzumab-saporin was titrated on a fixed concentration of cetuximab-SO1861 and targeted protein toxin mediated cell killing on low/no EGFR/HER2 expressing cells was determined. Low/no EGFR/HER2 expressing cells (HeLa and A2058) show no significant cell killing at high trastuzumab-saporin concentrations in combination with 278 nM cetuximab-SO1861 (HeLa: [toxin] IC50>10.000 pM; A2058: [toxin] IC50>10.000 pM; FIG. 13C, 13D; Table A7). High concentrations of unconjugated SO1861 (1500 nM) in combination with low concentrations of trastuzumab-saporin show efficient cell killing (HeLa: [toxin] IC50=0.4 pM; A2058: [toxin] IC50=0.5 pM; FIG. 13C, 13D; Table A5). All this shows that cells with low/no EGFR/low HER2 expression are not susceptible for the combination of cetuximab-SO1861+trastuzumab-saporin, due to a lack of sufficient EGFR receptor that facilitates the entry of sufficient SO1861 to ensure efficient cytoplasmic delivery of the toxin within the cell.

Next, Trastuzumab-SO1861 was titrated (calculated on concentration SO1861) on a fixed concentration of 1.5 pM EGF-dianthin and cell killing on high HER2/low EGFR expressing cells was determined. SKBR3 showed efficient cell killing when 1.5 pM EGF-dianthin was combined with high concentrations of non-targeted unconjugated SO1861 (SKBR3: [SO1861] IC50=800 nM; FIG. 14A; Table A6). However, when EGF-dianthin was combined with trastuzumab-SO1861 strong cell killing was induced already at low concentrations of conjugated SO1861 (SKBR3: [SO1861] IC50=2 nM; FIG. 14A; Table A5). This shows that targeted conjugated SO1861 is more effective in inducing endosomal escape compared to non-targeted unconjugated SO1861. Next, EGF-dianthin was titrated on a fixed concentration of trastuzumab-SO1861 and targeted protein toxin mediated cell killing was determined on SKBR3. No effective cell killing was observed with high EGF-dianthin concentrations in combination with non-targeted unconjugated 10 nM SO1861 (SKBR3 (shown) and BT474 (not shown): [toxin] IC50>10.000 pM; FIG. 14B; Table A7) whereas 9.4 nM trastuzumab-SO1861 in combination with low EGF-dianthin concentrations already induced efficient cell killing (SKBR3: [toxin] IC50=3 pM (shown); BT474: [toxin] IC50=1 pM (not shown); FIG. 14B; Table A7). Comparable cell killing efficiency is achieved when high concentrations (1075 nM) of non-targeted unconjugated SO1861 is combined with low concentrations of EGF-dianthin; FIG. 14B; Table A7). All this shows that when conjugated to trastuzumab, low concentrations of SO1861 efficiently can kill high HER2 expressing cells in combination with relatively low concentrations of EGF-dianthin. High concentrations (1500 nM) of non-targeted unconjugated SO1861 in combination with low concentrations of EGF-dianthin has comparable activity, since receptor competition does not play a role for SO1861 to enter the cell, since in the 2-target 2-component system both conjugates are delivered via different receptors, SO1861 via HER2 and toxin via EGFR receptor.

Next, Trastuzumab-SO1861 was titrated (calculated on concentration SO1861) on a fixed concentration of 5 pM cetuximab-saporin and cell killing on low HER2, low/high EGFR expressing cells was determined showed cell killing when 5 pM cetuximab-saporin was combined with high concentrations of non-targeted unconjugated SO1861 (JIMT-1: [SO1861] IC50>1000 nM; A431: [SO1861] IC50>1000 nM; FIG. 15A, 22B; Table A6). The combination of 5 pM cetuximab-saporin with increasing concentrations of trastuzumab-SO1861 showed cell killing only at high concentrations of cetuximab-SO1861 in both cell lines (JIMT-1: [SO1861] IC50>1000 nM; A431: [SO1861] IC50>1000 nM; FIG. 15A, 15B; Table A6). This shows that in the absence of sufficient receptor expression, effective intracellular SO1861 concentrations are not reached (threshold) to induce endosomal protein toxin escape and toxin-mediated cell killing. Next, cetuximab-saporin was titrated on a fixed concentration of trastuzumab-SO1861 and targeted protein toxin mediated cell killing on JIMT-1 and A431 was determined. Cell killing was observed only at high cetuximab-saporin concentrations in combination with 10 nM trastuzumab-SO1861 (JIMT-1: [toxin] IC50>90 pM; A431: [toxin] IC50>20 pM; FIG. 15C, 15D; Table A7). High concentrations of unconjugated SO1861 (1500 nM) in combination with low concentrations of cetuximab-saporin show efficient cell killing (JIMT-1: [toxin] IC50=0.02 pM; A431: [toxin] IC50=0.03 pM; FIG. 15C, 15D; Table A5). All this shows that cells with low HER2, low/high EGFR expression are not susceptible for the combination of trastuzumab-SO1861+cetuximab-saporin, due to a lack of sufficient HER2 receptor that facilitates the entry of sufficient SO1861 to ensure efficient cytoplasmic delivery of the toxin within the cell. Even a very high EGFR expression in A431 cells did not result in efficient cell killing by cetuximab-saporin, since the threshold of SO1861 was not reached due to a lack of HER2 receptors that could facilitate the uptake of SO1861 via trastuzumab-SO1861.

### Example 6

The 2 targeted 2 component system results in cell killing of very low target expressing cells. In A431 cells T-DM1 kills cells at nanomolar concentrations, whereas the targeted 2 component system can efficiently kill cells at picomolar concentrations (7000 fold decrease in toxin concentration) (FIG. 16)

### Example 7. Mechanism of Action

When endosomal acidification is blocked the targeted 2 component system is not active, due to the fact that SPT001 (a plant-derived saponin, SO1861) is only active at low endosomal pH.

### Example 8

Endosomal acidification inhibitors block the targeted 2-component system activity showing that SO1861 function is reduced when acidification of endosomes is blocked.

### Example 9

FIG. 17A-E displays the relative cell viability when trastuzumab (FIG. 17A), cetuximab (FIG. 17B) or T-DM1 (FIG. 17C), free toxins saporin (FIG. 17D) and dianthin (FIG. 17D), saporin coupled to a non-cell binding IgG (FIG. 17D), and saporin coupled to a non-cell binding IgG combined with free saponin SO1861 (FIG. 17E) are contacted with the indicated cell lines SK-BR-3, JIMT-1, MDA-MB-468, A431, CaSki, HeLa, A2058, BT-474.

Trastuzumab and cetuximab do not or hardly influence cell viability when exposed to most of the cell lines, with some effect on cell viability when trastuzumab is exposed to SK-BR-3 cells at relatively high dose, and with some effect on cell viability when cetuximab is exposed to MDA-MB-468 cells at relatively high dose.

TDM-1, or ado-trastuzumab emtansine, is a targeted therapy approved by the U.S. Food and Drug Administration to treat: HER2-positive metastatic breast cancer that has previously been treated with Herceptin (chemical name: trastuzumab) and taxane chemotherapy; early-stage HER2-positive breast cancer after surgery if residual disease was found after neoadjuvant (before surgery) treatment with Herceptin and taxane chemotherapy. The TDM-1 is a combination of Herceptin and the chemotherapy medicine emtansine. FIG. 17C shows that the TDM-1 results in decreased cell viability for all cell lines tested.

The free toxins saporin and dianthin and the toxin saporin coupled to a control IgG with no affinity for any of the cell surface molecules on the cell lines tested, do not or hardly have any influence on cell viability over a wide range of concentrations toxin tested, up to 10000 pM.

When the toxin saporin is coupled to a non-cell binding IgG, combining the conjugate with the free saponin SO1861 results in an IgG-saporin dose dependent decrease of the relative cell viability (FIG. 17E).

TABLE A5
summary of IC50 values for mAb, toxin, ligan toxin or mAb-toxin monotherapy +/− SO1861.

| | EGFR | HER2 | | | | Saporin (pM) | | IgG-saporin (pM) | |
|---|---|---|---|---|---|---|---|---|---|
| | expression | expression | | | | | 1500 | | 1500 |
| Cell | level | level | Trastuzumab | Cetuximab | T-DM1 | No | nM | No | nM |
| line | (MFI) | (MFI) | (nM) | (nM) | (nM) | SO1861 | SO1861 | SO1861 | SO1861 |
| MDA- | 1656 | 1 | >3.000 | >3.000 * | 60 | >10.000 | 20 | >10.000 | 30 |
| MB-468 | | | | | | | | | |
| A431 | 1593 | 10 | >3.000 | >3.000 | 80 | >10.000 | n.d. | >10.000 | 90 |
| CaSki | 481 | 12 | >3.000 | >3.000 | 40 | >10.000 | 15 | >10.000 | 60 |
| SK-BR-3 | 28 | 1162 | >3.000 ** | >3.000 | 1 | >10.000 | n.d. | >10.000 | 100 |
| JIMT-1 | 58 | 74 | >3.000 | >3.000 | 10 | >10.000 | 30 | >10.000 | 90 |
| HeLa | 91 | 7 | >3.000 | >3.000 | 40 | >10.000 | 30 | >10.000 | 200 |
| A2058 | 1 | 5 | >3.000 | >3.000 | 20 | >10.000 | n.d. | >10.000 | 90 |

| | Trastuzumab- | | Trastuzumab- | | Cetuximab- | | EGF-Dianthin | |
|---|---|---|---|---|---|---|---|---|
| | Saporin (pM) | | Dianthin (pM) | | Saporin (pM) | | (pM) | |
| | | 1500 | | 1500 | | 1500 | | 1500 |
| Cell | No | nM | No | nM | No | nM | No | nM |
| line | SO1861 | SO1861 | SO1861 | SO1861 | SO1861 | SO1861 | SO1861 | SO1861 |
| MDA- | >10.000 | 25 | >10.000 | 200 | 180 | 0.06 | 240 | 0.09 |
| MB-468 | | | | | | | | |
| A431 | >10.000 | 0.8 | >10.000 | 60 | 40 | 0.03 | 700 | 0.03 |
| CaSki | >10.000 | 0.2 | >10.000 | 5 | 40 | 0.02 | 2000 | 0.03 |
| SK-BR-3 | 4500 | 0.05 | >10.000 | 1 | n.d. | n.d. | >10.000 | 0.5 |
| JIMT-1 | >10.000 | 0.1 | >10.000 | 40 | 300 | 0.02 | >10.000 | 0.07 |
| HeLa | >10.000 | 0.4 | >10.000 | 18 | 60 | 0.03 | 3800 | 0.04 |
| A2058 | >10.000 | 0.5 | >10.000 | 5 | >10.000 | 20 | >10.000 | 30 |

Legend to Table A5:
* MDA-MB-468 cells show a 20-25% reduction in cell viability at all Cetuximab [ ] above 5 nM;
** SK-BR-3 cells show a 20% reduction in cell viability at 1 nM Trastuzumab and 30-35% reduction for all Trastuzumab [ ] above 1 nM

TABLE A6
Data summary of IC50 value of untargeted SO1861, targeted 2-
component system mAb-SO1861 titration with fixed [mAb-toxin].

| | | | | | | | 1-target 2-component | |
|---|---|---|---|---|---|---|---|---|
| | | | | | | | Both SO1861 and | |
| | | | | | | | toxin to EGFR | |
| | | | Untargeted SO1861 | | | | 10 pM | |
| | EGFR | HER2 | SO1861 + | SO1861 + | SO1861 + | SO1861 + | Cetuximab- | 10 pM |
| | expres- | expres- | 50 pM | 50 pM | 10 pM | 10 pM | Saporin + | Dianthin:EGF + |
| | sion | sion | Trastuzumab- | Trastuzumab- | Cetuximab- | EGF − | Cetuximab- | Cetuximab- |
| Cell | level | level | saporin | dianthin | saporin | dianthin | SO1861 | SO1861 |
| line | (MFI) | (MFI) | (IC50, nM) | (IC50, nM) | (IC50, nM) | (IC50, nM) | (IC50, nM) | (IC50, nM) |
| MDA- | 1656 | 1 | >1.000 | >1.000 | >1.000 | >1.000 | 3 | 6 |
| MB-468 | | | | | | | | |
| A431 | 1593 | 10 | >1.000 | >1.000 | 600 | >1.000 | 5 | 8 |
| CaSki | 481 | 12 | >1.000 | >1.000 | 700 | >1.000 | 5-10 | 10 |
| SK-BR-3 | 28 | 1162 | 700 | n.d. | 800 | 650 | >1.000 | >1.000 |
| JIMT-1 | 58 | 74 | >1.000 | >1.000 | >1.000 | >1.000 | >1.000 | >1.000 |
| HeLa | 91 | 7 | >1.000 | >1.000 | >1.000 | >1.000 | >1.000 | >1.000 |
| A2058 | 1 | 5 | >1.000 | >1.000 | >1.000 | >1.000 | >1.000 | >1.000 |

| | 1-target 2-component | | 2-target 2-component | | |
|---|---|---|---|---|---|
| | Both SO1861 and | | SO1861 to EGFR; | SO1861 to HER2; | |
| | toxin to HER2 | | toxin to HER2 | toxin to EGFR | |
| | 50 pM | 50 pM | 50 pM | 5 pM | |
| | Trastuzumab- | Trastuzumab- | Trastuzumab- | Cetuximab- | 1.5 pM |
| | Saporin + | Dianthin + | Saporin + | Saporin + | Dianthin:EGF + |
| | Trastuzumab- | Trastuzumab- | Cetuximab- | Trastuzumab- | Trastuzumab- |
| Cell | SO1861 | SO1861 | SO1861 | SO1861 | SO1861 |
| line | (IC50, nM) | (IC50, nM) | (IC50, nM) | (IC50, nM) | (IC50, nM) |
| MDA- | >1.000 | >1.000 | 18 | >1.000 | >1.000 |
| MB-468 | | | | | |
| A431 | >1.000 | >1.000 | 12 | >1.000 | >1.000 |
| CaSki | >1.000 | >1.000 | 40 | >1.000 | >1.000 |
| SK-BR-3 | 2* | 3* | >1.000 | n.d. | 3 |
| JIMT-1 | >1.000 | >1.000 | >1.000 | >1.000 | >1.000 |
| HeLa | >1.000 | >1.000 | >1.000 | >1.000 | >1.000 |
| A2058 | >1.000 | >1.000 | >1.000 | >1.000 | >1.000 |

TABLE A7
Data summary of IC50 values for the targeted 2-component system, mAb-toxin titration
with fixed [mAb-SO1861]. IC50 for each value is calculated as a percentage relative
to the component(s) with constant concentration for that treatment (100%)

| | | | 1-target 2-component | | | |
|---|---|---|---|---|---|---|
| | | | Both SO1861 and | | Both SO1861 and | |
| | | | toxin to EGFR | | toxin to HER2 | |
| | | | 278 nM | 278 nM | 300 nM | 300 nM |
| | EGFR | HER2 | Cetuximab- | Cetuximab- | Trastuzumab- | Trastuzumab- |
| | expres- | expres- | SO1861 + | SO1861 + | SO1861 + | SO1861 + |
| | sion | sion | Cetuximab- | EGF- | Trastuzumab- | Trastuzumab- |
| Cell | level | level | Saporin | Dianthin | Saporin | Dianthin |
| line | (MFI) | (MFI) | (IC50, pM) | (IC50, pM) | (IC50, pM) | (IC50, pM) |
| MDA- | 1656 | 1 | 0.5 | 1 | >10.000 | >10.000 |
| MB-468 | | | | | | |
| A431 | 1593 | 10 | 0.4 | 0.4 | >10.000 | >10.000 |
| CaSki | 481 | 12 | 2 | 1 | >10.000 | >10.000 |
| SK-BR-3 | 28 | 1162 | n.d. | n.d. | 20 | n.d. |
| JIMT-1 | 58 | 74 | >10.000 | >10.000 | >10.000 | >10.000 |
| HeLa | 91 | 7 | 6.000 | >10.000 | >10.000 | >10.000 |
| A2058 | 1 | 5 | >10.000 | >10.000 | >1.0000 | >10.000 |

| | 2-target 2-component | | | |
|---|---|---|---|---|
| | SO1861 to EGFR; | | SO1861 to HER2; | |
| | toxin to HER2 | | toxin to EGFR | |
| | 278 nM | 1388 nM | 300 nM | 300 nM |
| | Cetuximab- | Cetuximab- | Trastuzumab- | Trastuzumab- |
| | SO1861 + | SO1861 + | SO1861 + | SO1861 + |
| | Trastuzumab- | Trastuzumab- | Cetuximab- | EGF- |
| Cell | Saporin | Dianthin | Saporin | Dianthin |
| line | (IC50, pM) | (IC50, pM) | (IC50, pM) | (IC50, pM) |
| MDA- | 55 | 500 | 100 | 750 |
| MB-468 | | | | |
| A431 | 3 | 30 | 20 | 2.300 |
| CaSki | 1 | 25 | 14 | 1.600 |
| SK-BR-3 | n.d. | n.d. | n.d. | 2 |
| JIMT-1 | 3.000 | >10.000 | 90 | 4.000 |
| HeLa | >10.000 | 10.000 | 50 | 4.000 |
| A2058 | >10.000 | >10.000 | >10000 | >10.000 |

### Example 10—Treating a Mammalian Tumor-Bearing Animal with a Conjugate of the Invention in Combination with an ADC Results in Survival and Tumor Regression

Female Balb/c nude mice were injected subcutaneously with a suspension of human A431 tumor cells. Under the skin of the mice, a human epidermal carcinoma developed in the xenograft animal tumor model. After injection of the tumor cells, the xenograft tumor was allowed to develop to a size of approximately 170-180 mm³. The A431 tumor cells have the following characteristics: high EGFR expressors, medium CD71 expressors, low HER2 expressors.

In Table 6A, the results of the treatment of control mice and tumor-bearing mice are presented. Tumor-bearing mice were treated with the indicated antibodies directed to either human Her2/neu, human EGFR, or human CD71, which are cell-surface receptors on the xenograft tumor. Cetuximab was covalently conjugated with saponin SO1861. The SO1861 was first provided with the linker EMCH (N-ε-maleimidocaproic acid hydrazide), which EMCH is a maleimide-and-hydrazide crosslinker for covalently conjugating sulfhydryls (reduced cysteines of the antibody)) to carbonyls (aldehyde or ketones; here the carbonyl of the aldehyde at position C-23 of the saponin). The saponin-EMCH was covalently coupled to reduced cysteines of the Cetuximab, forming a covalent thio-ether bond between the EMCH and the cysteine side chain. The ADCs trastuzumab-saporin (covalent conjugate) and anti-CD71 mAb (OKT-9, IgG)-saporin (covalent conjugate) were tested for their tumor-attacking efficacy in the mice, measured as tumor volume in time after start of the treatment with the ADCs. The dose of the ADCs was sub-optimal in the tumor model. That is to say, from previous experiments, it was established at which sub-optimal dose of the ADCs no tumor-regression or arrest of tumor growth would be observable.

TABLE 6A
RESULTS OF TREATING A MAMMALIAN TUMOR-BEARING ANIMAL
WITH A CONJUGATE OF THE INVENTION IN COMBINATION WITH
AN ADC RESULTS IN SURVIVAL AND TUMOR REGRESSION

| | | | tumor size (volume in mm³ or ‘+’ |
|---|---|---|---|
| | | | for growth, ‘−’ for regression, |
| Treatment | Patient/healthy | | and ‘stable’ for growth nor |
| group | animal | treatment | regression) |
| 1 | xenograft | vehicle | 2000 mm³ (death/euthanasia) |
| 2 | xenograft | Trastuzumab-saporin | 2000 mm³ (death/euthanasia) |
| 3 | xenograft | Anti-CD71 mAb OKT-9 - | 2000 mm³ (death/euthanasia) |
| | | saporin (covalent | |
| | | conjugate) | |
| 4 | xenograft | Cetuximab-SO1861 | 2000 mm³ (death/euthanasia) |
| | | (covalent conjugate) | |
| 5 | xenograft | Cetuximab | >170 mm³, but <2000 mm³ |
| | | | (death/euthanasia) |
| 6 | xenograft | Trastuzumab-saporin | Tumor regression from 180 mm³ |
| | | (covalent conjugate) + | at the start of treatment back to |
| | | Cetuximab-SO1861 | 80 mm³ (survival) |
| | | (covalent conjugate) | |
| 7 | xenograft | Anti-CD71 mAb OKT-9 - | Tumor regression from 180 mm³ |
| | | saporin (covalent | at the start of treatment back to |
| | | conjugate) + Cetuximab- | 40 mm³ (survival) |
| | | SO1861 (covalent | |
| | | conjugate) | |

These results demonstrate that the combination therapy of an ADC at a dose which is ineffective when treatment of tumor-bearing mice with the ADC alone is considered (tumor growths, death of the mice is not prevented (euthanasia)), with a conjugate of the invention consisting of a tumor-cell specific receptor targeting antibody covalently bound to a saponin, i.e. SO1861, the covalent conjugate administered to the mice suffering from cancer, at a non-effective dose when administered alone (tumor growths, death of the mice is not prevented (euthanasia)), provides an efficient and efficacious treatment regimen, expressed as tumors in regression and prolonged survival of the treated animals (beyond the duration of the experiment). The sub-optimal dose of ADC combined with a covalently bound saponin-comprising conjugate of the invention which has no anti-tumor activity when administered alone, thus provide for an effective treatment option for cancer patients, wherein a relative low dose of the ADC is efficacious. A lower dose of ADC bears the promise of less risk for adverse events, or even no side effects at all. In addition, the stimulatory effect of the saponin-bearing conjugate when the efficacy of the ADC is considered, shows that ADCs which previously have proven to lack efficacy when tumor patient treatment is concerned, may gain renewed attention and value, since ADC efficacy is improved in combination therapy setting, as the current example demonstrated. Reference is made to ADCs known in the art which were previously investigated in the human clinical setting, but then were for some ADCs retracted from further clinical investigation. Especially the ADCs for which clinical development was terminated due to observed lack of efficacy and/or due to occurrence of unacceptable adverse event are ADCs which may gain renewed value for cancer patients when combined with a covalently bound saponin-comprising conjugate, such as the cetuximab-saponin tested.

### Example 11—Saponins Mixture of Quillaja saponaria Comprising QS-21, with Endosomal/Lysosomal Escape Enhancing Activity

Scheme Q displays the common molecular structure of a series of QS-21 saponins (in part adapted from: Conrado Pedebos, Laercio Pol-Fachin, Ramon Pons, Cilaine V. Teixeira Hugo Verli, Atomic Model and Micelle Dynamics of QS-21 Saponin, Molecules 2014, 19, 3744-3760; four isoforms, wherein each of the depicted glycans can be bound as the R group). A mixture of water-soluble saponins obtained from Quillaja saponaria (Sigma-Aldrich, product No. S4521; Roth, Item No. 6857; InvivoGen, product ‘Quil-A’) may be applied in an endosomal/lysosomal escape enhancing conjugate, composition and combination of the invention, based on endosomal/lysosomal escape enhancing properties of at least one individual saponin present in the mixture, e.g. QS-21, or based on a combination of two or more of the saponins comprised by the mixture, such as QS-21 and QS-7.

The inventors demonstrated that the mixture of saponins from Quillaja saponaria at 2.5 microgram/ml dose was capable of enhancing endosomal escape of dianthin, as tested with mammalian tumor cells in a cell-based bioassay. The effector molecule exposed to the cells was dianthin covalently coupled to the ligand EGF: EGF-dianthin. Cells tested were tumor cell lines HeLa for free saponins, and A431, MDA-MB-468, CaSki and A2058 for testing the saponins when covalently coupled to cetuximab.

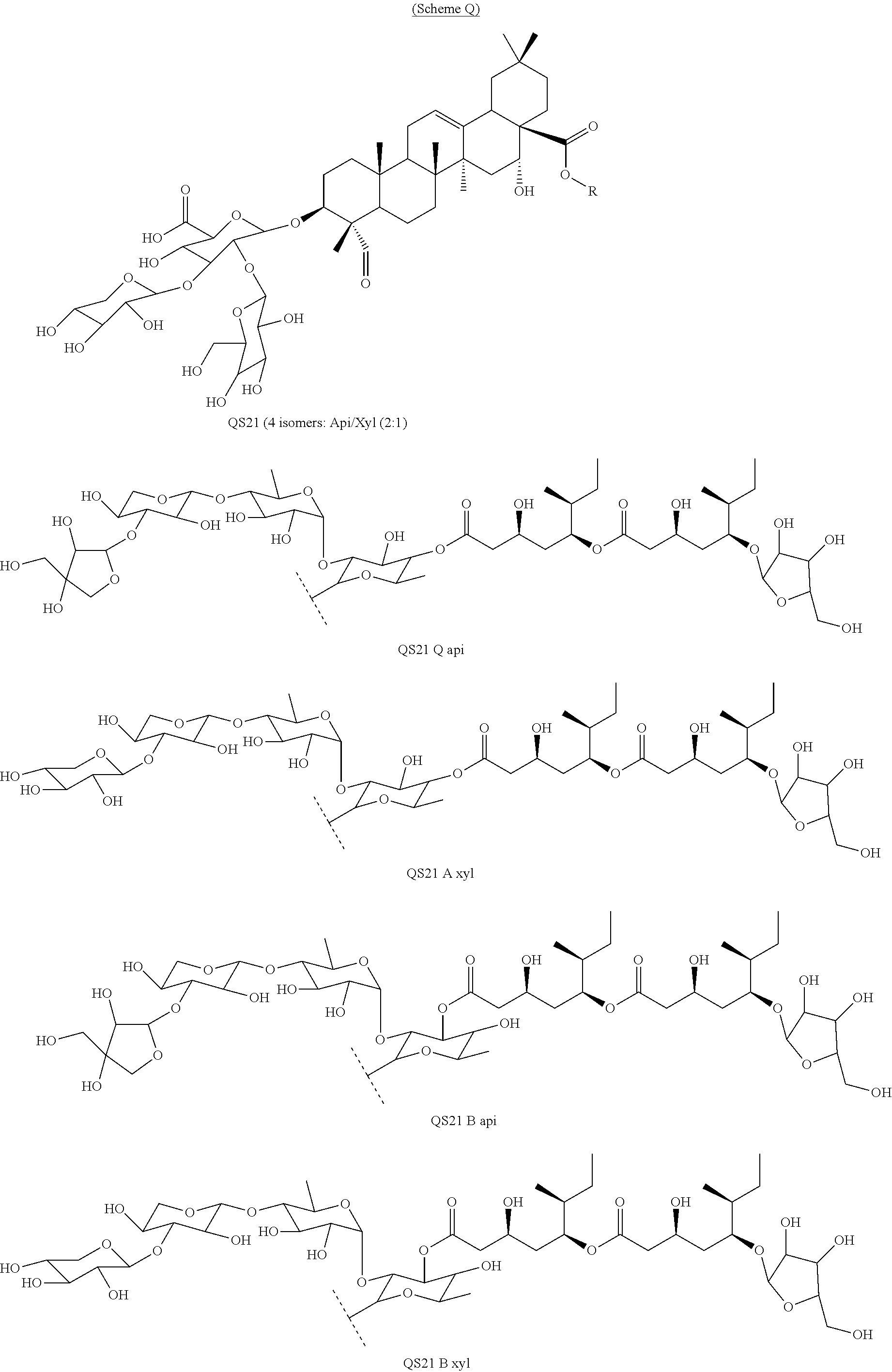

### Example 12

The 1 target 2-components system (1T2C) is the combination treatment of mAb1-protein toxin and mAb1-SO1861, as illustrated in FIG. 1A, E. SO1861-EMCH was conjugated via cysteine residues (Cys) and HSP27BNA oligo was conjugated via lysine residues to cetuximab (monoclonal antibody recognizing and binding human EGFR), both with a DAR 4 resulting in the production of 2 conjugates: cetuximab-(Cys-L-SO1861)⁴ and cetuximab-(Lys-L-HSP27BNA)⁴. The combination of cetuximab-(Cys-L-SO1861)⁴ (intraperitoneal administration, (i.p.)) and cetuximab-(Lys-L-HSP27BNA)⁴ (intravenous administration, (i.v.)) was tested in a A431 xenograph mouse tumor model for EGFR tumor targeted gene silencing activity. Dosings started at day 12 when tumors reached ˜150 mm³ in size and tumor samples were collected at 72 h after the first dosing and analysed for HSP27 gene expression compared to control gene mRNA expression levels (reference genes). This revealed that 1 dosing of 50 mg/kg cetuximab-(Cys-L-SO1861)⁴+25 mg/kg cetuximab-(Lys-L-HSP27BNA)⁴ resulted in a 50% reduction in HSP27 gene expression in the A431 tumors compared to single dosing of cetuximab-(Cys-L-SO1861)⁴ or cetuximab-(Lys-L-HSP27BNA)⁴ mono therapies (FIG. 18). Compared to the vehicle control tumors, a reduction of 40% HSP27 gene silencing was observed. This shows and enables that the combination of cetuximab-conjugated SO1861+cetuximab-conjugated HSP27BNA oligo, according to the 1T2C invention, induces efficient targeted delivery of a therapeutic antisense oligo nucleotide in the cytoplasm of solid tumor cells, thereby inducing tumor targeted gene silencing, in vivo.

Next, SO1861-EMCH was conjugated via cysteine residues (Cys) to trastuzumab (monoclonal antibody recognizing and binding human HER2), with a DAR 4 resulting in the production of trastuzumab-(Cys-L-SO1861)⁴. The combination of trastuzumab-(Cys-L-SO1861)⁴ and trastuzumab-saporin (trastuzumab protein toxin conjugate) was tested in a mouse tumor model (patient derived xenograph tumor model, PDX) with high HER2 expression levels and resistant for trastuzumab mono therapy. The combination, according to the 1T2C invention of 40 mg/kg trastuzumab-(Cys-L-SO1861)⁴ (intraperitoneal administration, (i.p.))+0.03 (Dayl, 8)/0.02 (Day 15, 22, 30, 36, 43) mg/kg trastuzumab-saporin (intravenous administration, (i.v.)) revealed strong tumor growth inhibition compared to the vehicle control and the 40 mg/kg trastuzumab-(Cys-L-SO1861)⁴ or 0.03/0.02 mg/kg trastuzumab-saporin mono therapies (FIG. 19). Besides, in tumor bearing mice that were treated with a lower dosing combination (40 mg/kg trastuzumab-(Cys-L-SO1861)⁴+0.01 mg/kg trastuzumab-saporin) no tumor growth inhibiting activity was observed (FIG. 26). This shows and enables that the 1T2C combination of trastuzumab conjugated SO1861+trastuzumab conjugated protein toxin induces efficient targeted delivery of a therapeutic protein toxin in the cytoplasm of solid tumor cells, thereby inducing tumor cell death and tumor growth inhibition, in vivo.

### Example 13. 2 Target 2-Component System (In Vivo)

The 2 target 2-components system (2T2C) is the combination treatment of mAb1-SO1861 and mAb2-protein toxin, (FIG. 1B-D). SO1861-EMCH was conjugated via cysteine residues (Cys) to cetuximab (monoclonal antibody recognizing and binding human EGFR), with a DAR 4 resulting in the production of: cetuximab-(Cys-L-SO1861)⁴. The combination of cetuximab-(Cys-L-SO1861)⁴ and trastuzumab-saporin or CD71mab-saporin was tested in a A431 (EGFR⁺⁺/HER2^{+/-}/CD71⁺) xenograph ‘nude’ mouse tumor model for EGFR tumor targeted cell killing as illustrated in FIG. 1B-D. Dose escalation was performed to determine the therapeutic efficacy (Day 9: 0.3 mg/kg trastuzumab-saporin or 0.1 mg/kg CD71mab-saporin+5 mg/kg cetuximab-(Cys-L-SO1861)⁴; Day 14, 18: 0.1 mg/kg trastuzumab-saporin or 0.05 mg/kg CD71 mab-saporin+5 mg/kg cetuximab-(Cys-L-SO1861)⁴; Day 21: 0.05 mg/kg trastuzumab-saporin or 0.05 mg/kg CD71 mab-saporin+15 mg/kg cetuximab-(Cys-L-SO1861)⁴; Day 28: 0.02 mg/kg trastuzumab-saporin or 0.02 mg/kg CD71mab-saporin+15 mg/kg cetuximab-(Cys-L-SO1861)⁴ trastuzumab-saporin/cetuximab-SO1861. Controls were on the same dosing scheme respectively, only cetuximab (i. v.) was given 25 mg/kg every treatment day). At day 32 (dashed vertical line), day 35 and day 39 the inventors started the combination, according to the 2T2C invention of 25 mg/kg cetuximab-(Cys-L-SO1861)⁴ (intraperitoneal injection (i.p.)+0.02 mg/kg trastuzumab-saporin or 0.02 CD71 mab-saporin (intravenous administration, (i.v.)) and this revealed strong tumor regression for both 2T2C combination groups compared to the vehicle control, 25 mg/kg cetuximab-(Cys-L-SO1861)⁴ or 0.02 mg/kg trastuzumab-saporin/CD71mab-saporin mono therapies (FIG. 20). The 2T2C system even outcompetes cetuximab, the clinically used monoclonal antibody against EGFR. Next the inventors performed the same experiment but then the test was started with 25 mg/kg cetuximab-(Cys-L-SO1861)⁴ (intraperitoneal injection (i.p.)+0.03 mg/kg trastuzumab-saporin or 0.03 CD71mab-saporin (intravenous administration, (i.v.)) treatment with a dosing at day 9 and 14 and thereafter 1 dosing per week. The 2T2C system according to the invention showed tumor regression in all mice and even in 1 mice in both 2T2C groups, complete tumor eradication (tumor volume=0 mm3) (FIG. 21). Also here the controls showed a strong increased in tumor volume whereas the positive control for this A431 mice model, cetuximab showed only tumor growth inhibition, but no regression (FIG. 21). This shows and enables the 2T2C system approach, of cetuximab conjugated SO1861+trastuzumab conjugated protein toxin or CD71mab conjugated protein toxin inducing highly efficient targeted delivery of a therapeutic protein toxin in the cytoplasm of solid tumors of tumor bearing mice, in vivo, thereby inducing even full tumor eradication in some mice and strong tumor regression in others even in large size tumors (2000 mm³).

### Example 14. 2 Target 2-Component System (In Vitro)

Results

The 2 target 2-components system (2T2C) is the combination treatment of mAb1-SO1861 and mAb2-protein toxin, (FIG. 1B-D). SO1861-EMCH was conjugated via cysteine residues (Cys) to cetuximab (monoclonal antibody recognizing and binding human EGFR), with a DAR 3,7 (cetuximab-(Cys-L-SO1861)^{3,7}). Cetuximab-(Cys-L-SO1861)^{3,7} was titrated on a fixed concentration of 50 pM trastuzumab-saporin (trastuzumab, conjugated to the protein toxin, saporin) and targeted protein toxin mediated cell killing on EGFR/HER2 expressing cells (A431, EGFR⁺⁺/HER2^{+/-}; CaSKi, EGFR⁺/HER2^{+/-}) was determined as illustrated in FIG. 1B-D. This revealed strong cell killing at low concentrations of cetuximab-(Cys-L-SO1861)^{3,7} (A431: IC50=3 nM and CaSKi IC50=10 nM; FIG. 22A, 22B) whereas equivalent concentrations cetuximab, cetuximab-(Cys-L-SO1861)^{3,7} or cetuximab+50 pM trastuzumab-saporin could not induce any cell killing activity in EGFR/HER2 expressing cells. This shows that relatively low concentrations of cetuximab-SO1861 conjugate efficiently enhances endosomal escape of the trastuzumab conjugated protein toxin (at non-effective concentrations), thereby inducing efficient cell killing of high EG FR/low HER2 expressing cells.

Next, trastuzumab-saporin was titrated on a fixed concentration of 75 nM cetuximab-(Cys-L-SO1861)^{3,7} and targeted protein toxin mediated cell killing on EGFR/HER2 expressing cells was determined. This revealed that 75 nM cetuximab-(Cys-L-SO1861)^{3,7} in combination with low concentrations trastuzumab-saporin induced already efficient cell killing in EGFR/HER2 expressing cells (A431: IC50=5 pM; and CaSKi: IC50=1 pM; FIGS. 22C and 22D), whereas trastuzumab-saporin alone or trastuzumab-saporin+75 nM cetuximab did not show significant cell killing activity (IC50>10.000 pM) in both cell lines (FIG. 22C, 22D). All this shows that relatively low concentrations of trastuzumab-saporin can be effective and induce cell killing in combination with low cetuximab-SO1861 conjugate concentrations in high EGFR/low HER2 expressing cells.

Next, cetuximab-(Cys-L-SO1861)^{3,7} was titrated on a fixed concentration of 50 pM trastuzumab-saporin and targeted protein toxin-mediated cell killing on HeLa (EGFR^{+/-}/HER2^{+/-}) or A2058 (EGFR⁻/HER2^{+/-}) was determined as illustrated in FIG. 1B-D. Both HeLa (EGFR^{+/-}/HER2^{+/-}) and A2058 (EGFR⁻/HER2^{+/-}) cells do not show cell killing at low concentrations of cetuximab-(Cys-L-SO1861)^{3,7+50} pM trastuzumab-saporin (HeLa: IC50=400 nM; A2058: IC50>400 nM; FIG. 230A, 23B). This shows that in the absence of sufficient receptor expression, effective intracellular delivered SO1861 concentrations are not reached (threshold) to induce endosomal escape and cytoplasmic delivery of the protein toxin. Next, trastuzumab-saporin was titrated on a fixed concentration of 75 nM cetuximab-(Cys-L-SO1861)^{3,7} and targeted protein toxin mediated cell killing on HeLa (EGFR^{+/-}/HER2^{+/-}) or A2058 (EGFR⁻/HER2^{+/-}) was determined. Both HeLa (EGFR^{+/-}/HER2^{+/-}) and A2058 (EGFR⁻/HER2^{+/-}) cells showed no cell killing activity (HeLa: IC50>10.000 pM; A2058: IC50>10.000 pM; FIG. 23C, 23D). All this shows that cells with low or no EGFR receptor expression are not susceptible for the combination of cetuximab-(Cys-L-SO1861)^{3,7}+trastuzumab-saporin, due to a lack of sufficient EGFR receptor that facilitates the antibody-mediated delivery of sufficient SO1861 (threshold) to ensure endosomal escape of the toxin within the cytoplasm of the cell.

Next, SO1861-EMCH was conjugated via cysteine residues (Cys) to trastuzumab (monoclonal antibody recognizing and binding human HER2), with a DAR 4 (trastuzumab-(Cys-L-SO1861)⁴). Trastuzumab-(Cys-L-SO1861)⁴ was titrated on a fixed concentration of 1.5 pM EGFdianthin (EGFR targeted ligand toxin fusion protein) and targeted protein toxin mediated cell killing on HER2/EGFR expressing cells (SK-BR-3: HER2⁺⁺/EGFR^{+/-}) was determined. This revealed strong cell killing at low concentrations of trastuzumab-(Cys-L-SO1861)⁴+1.5 pM EGFdianthin (SK-BR-3: IC50=1 nM; FIG. 24A) whereas equivalent concentrations trastuzumab, trastuzumab-(Cys-L-SO1861)⁴ or trastuzumab+1.5 pM EGFdianthin could not induce any cell killing activity in HER2⁺⁺/EGFR^{+/-} expressing cells. This shows that trastuzumab conjugated SO1861 efficiently enhances endosomal escape of the EGF fusion protein toxin (at non-effective concentrations), thereby inducing cell killing of high HER2/low EGFR expressing cells.

Next, EGFdianthin was titrated on a fixed concentration of 2.5 nM trastuzumab-(Cys-L-SO1861)⁴ and targeted protein toxin mediated cell killing on SK-BR-3 (HER2⁺⁺/EGFR^{+/-}) expressing cells was determined. This revealed that 2.5 nM trastuzumab-(Cys-L-SO1861)⁴ in combination with low concentrations EGFdianthin induced already efficient cell killing in HER2/EGFR expressing cells (SK-BR-3: IC50=1 pM) (FIG. 24B), whereas EGFdianthin alone or EGFdianthin+2.5 nM trastuzumab showed no cell killing activity (IC50>10.000 pM) (FIG. 24B). All this shows that relatively low concentrations of EGFdianthin can be effective and induce cell killing only in combination with low trastuzumab-(Cys-L-SO1861)⁴ concentrations in high HER2/low EGFR expressing cells.

Next, trastuzumab-(Cys-L-SO1861)⁴ was titrated on a fixed concentration of 1.5 pM EGFdianthin and targeted protein toxin mediated cell killing on JIMT-1 (HER2^{+/-}/EGFR^{+/-}) or MDA-MB-468: HER2⁻/EGFR⁺⁺) was determined. Both cell lines were not sensitive for any combination of trastuzumab-(Cys-L-SO1861)⁴+1.5 pM EGFdianthin (JIMT-1: IC50>1000 nM; MDA-MB-468: IC50>1000 nM; FIG. 25A, 25B). This shows that in the absence of sufficient HER2 receptor expression, effective intracellular delivered SO1861 concentrations are not reached (threshold) to induce endosomal escape and cytoplasmic delivery of the protein toxin.

Next, EGFdianthin was titrated on a fixed concentration of 2.5 nM trastuzumab-(Cys-L-SO1861)⁴ and targeted protein toxin mediated cell killing on JIMT-1 (HER2^{+/-}/EGFR^{+/-}) or MDA-MB-468 (HER2⁻/EGFR⁺⁺) was determined. Both cell lines showed cell killing at high EGFdianthin concentrations with or without 2.5 nM trastuzumab-(Cys-L-SO1861)⁴ (JIMT-1: IC50=10.000 pM; MDA-MB-468: IC50=200 pM FIG. 25C, 25D).

All this shows that cells with low or no HER2 receptor expression are not susceptible for the combination of trastuzumab-(Cys-L-SO1861)^{3,7}+1.5 pM EGFdianthin, due to a lack of sufficient HER2 receptor that facilitates the antibody-mediated delivery of sufficient SO1861 (threshold) to ensure endosomal escape of the toxin within the cytoplasm of the cell.

Next, SO1861-EMCH was conjugated via cysteine residues (Cys) to trastuzumab (monoclonal antibody recognizing and binding human HER2), with a DAR 4, (trastuzumab-(Cys-L-SO1861)⁴). Trastuzumab-(Cys-L-SO1861)⁴ was titrated on a fixed concentration of 5 pM cetuximab-saporin (EGFR targeting antibody-protein toxin conjugate) and targeted protein toxin mediated cell killing on HER2/EGFR expressing cells (SK-BR-3: HER2⁺⁺/EGFR^{+/-}) was determined as illustrated in FIG. 1B-D. This revealed strong cell killing at low concentrations of trastuzumab-(Cys-L-SO1861)⁴+5 pM cetuximab-saporin (SK-BR-3: IC50=1 nM; FIG. 26A) whereas equivalent concentrations trastuzumab, trastuzumab-(Cys-L-SO1861)⁴ or trastuzumab+5 pM cetuximab-saporin could not induce any cell killing activity in HER2⁺⁺/EGFR^{+/-} expressing cells. This shows that trastuzumab conjugated SO1861 efficiently enhances endosomal escape of the cetuximab conjugated protein toxin (at non-effective concentrations), thereby inducing cell killing of HER2⁺⁺/EGFR^{+/-} expressing cells.

Next, cetuximab-saporin was titrated on a fixed concentration of 2.5 nM trastuzumab-(Cys-L-SO1861)⁴ and 75 nM trastuzumab-(Cys-L-SO1861)⁴ and targeted protein toxin mediated cell killing on HER2/EGFR expressing cells (SK-BR-3: HER2⁺⁺/EGFR^{+/-}) was determined. This revealed that 2.5 nM trastuzumab-(Cys-L-SO1861)⁴ in combination with low concentrations cetuximab-saporin induced already efficient cell killing in SK-BR-3 cells (SK-BR-3: IC50=1 pM; FIG. 26B), whereas cetuximab-saporin alone or cetuximab-saporin+2.5 nM trastuzumab showed cell killing only at high concentrations trastuzumab-saporin (SK-BR-3: IC50>4000 pM; FIG. 26B). All this shows that relatively low concentrations of cetuximab-saporin can be effective and induce cell killing only in combination with low trastuzumab-(Cys-L-SO1861)⁴ concentrations in HER2⁺⁺/EGFR^{+/-} expressing cells.

Next, trastuzumab-(Cys-L-SO1861)⁴ was titrated on a fixed concentration of 5 pM cetuximab-saporin and targeted protein toxin mediated cell killing on JIMT-1 (HER2^{+/-}/EGFR^{+/-}) and MDA-MB-468 (HER2⁻/EGFR⁺⁺) cells was determined. Both cell lines were not sensitive for the combination of trastuzumab-(Cys-L-SO1861)⁴+5 pM cetuximab-saporin (JIMT-1: IC50>1000 nM; MDA-MB-468: IC50>1000 nM; FIG. 27A, 27B). This shows that in the absence of sufficient HER2 receptor expression, effective intracellular delivered SO1861 concentrations are not reached (threshold) to induce endosomal escape and cytoplasmic delivery of the protein toxin.

Next, cetuximab-saporin was titrated on a fixed concentration of 2.5 nM trastuzumab-(Cys-L-SO1861)⁴ and targeted protein toxin mediated cell killing on JIMT-1 (HER2^{+/-}/EGFR^{+/-}) and MDA-MB-468 (HER2⁻/EGFR⁺⁺) cells was determined. Both cell lines showed cell killing at similar cetuximab-saporin concentrations with or without 2.5 nM trastuzumab-(Cys-L-SO1861)⁴ (JIMT-1: IC50=80 pM; MDA-MB-468: IC50=100 pM; FIG. 27C, 27D).

All this shows that cells with low or no HER2 receptor expression are not susceptible for the combination of trastuzumab-(Cys-L-SO1861)⁴+cetuximab-saporin, due to a lack of sufficient HER2 receptor that facilitates the antibody-mediated delivery of sufficient SO1861 (threshold) to ensure endosomal escape of the toxin within the cytoplasm of the cell.

### Example 14. BivalentV_{HH}EGFR-dianthin+mAb-SO1861 (1T2C and 2T2C)

Results

The 1 target 2-components system (1T2C) is the combination treatment of mAb-SO1861 and V_{HH}-protein toxin, where mAb and V_{HH} recognize and bind the same cell surface receptor (FIG. 1E). The 2 target 2-components system (2T2C) is also the combination treatment of mAb-SO1861 and V_{HH}-protein toxin, where the mAb and V_{HH} recognize another cell surface receptor (FIG. 1D).

BivalentV_{HH}EGFR-dianthin (SEQ ID NO: 73) was produced as recombinant fusion protein comprising the bivalentV_{HH}EGFR with amino-acid sequence as depicted as SEQ ID NO: 74. Cetuximab-SO1861 (DAR4) was titrated on a fixed (non-effective) concentration of 50 pM bivalentV_{HH}EGFR-dianthin and targeted protein toxin mediated cell killing on A431 (EGFR⁺⁺/HER2^{+/-}), MDA-MB-468 (EGFR⁺⁺/HER2^{+/-}), SK-BR-3 (HER2⁺⁺/EGFR⁺) and A2058 (EGFR⁻/HER2^{+/-}) was determined. This revealed that very low concentrations cetuximab-SO1861 in combination with 50 pM bivalentV_{HH}EG FR-dianthin induced efficient cell killing of MDA-MB-468 cells (IC50=0.5 nM; FIG. 28) and A431 (IC50=4 nM; FIG. 28) whereas on SK-BR-3 or A2058 cells (low or no EGFR expression) activity was detected only with very high concentrations of cetuximab-SO1861 (IC50>100 nM; FIG. 28).

This shows that low concentrations of cetuximab-SO1861 (DAR4) can efficiently induce endosomal escape of a bivalentV_{HH}EGFR-dianthin fusion protein only in high EGFR expressing cells, thereby inducing enhanced cell killing.

Next, trastuzumab-SO1861 (DAR4) was titrated on a fixed (non-effective) concentration of 50 pM bivalentV_{HH}EGFR-dianthin and targeted protein toxin mediated cell killing on SK-BR-3 (HER2⁺⁺/EGFR⁻), A431 (EGFR⁺⁺/HER2^{+/-}), A2058 (EGFR⁻/HER2^{+/-}) and MDA-MB-468 cells (HER2-/EGFR⁺⁺) was determined. This revealed that very low concentrations trastuzumab-SO1861 in combination with 50 pM bivalentV_{HH}EGFR-dianthin induced efficient cell killing of SK-BR-3 cells (IC50=0.3 nM; FIG. 28), whereas the combination showed only at very high concentratiosn trastuzumab-SO1861 cell killing activity in A431 (low HER2), A2058 (low HER2) and MDA-MB-468 (no HER2) cells (IC50>100 nM; FIG. 28) that express low levels of HER2 (A431, A2058) or lack HER2 expression (MDA-MB-468). This shows that low concentrations of trastuzumab-SO1861 (DAR4) can efficiently induce endosomal escape of a bivalentV_{HH}EGFR-dianthin only in high HER2 expressing cells, thereby inducing enhanced cell killing.

Materials and Methods

Materials

SO1861 was isolated and purified by Analyticon Discovery GmbH from raw plant extract obtained from Saponaria officinalis. Trastuzumab (Tras, Herceptin®, Roche), Cetuximab (Cet, Erbitux®, Merck KGaA) were purchased from the pharmacy (Charite, Berlin). Bivalent-VHHEGFR-dianthin fusion was produced as recombinant fusion protein in E. coli according to standard procedures (Genscript) and has the amino-acid sequence as depicted as SEQ ID NO: 74.

Tris(2-carboxyethyl)phosphine hydrochloride (TCEP, 98%, Sigma-Aldrich), 5,5-Dithiobis(2-nitrobenzoic acid) (DTNB, Ellman's reagent, 99%, Sigma-Aldrich), Zeba™ Spin Desalting Columns (2 mL, Thermo-Fisher), NuPAGE™ 4-12% Bis-Tris Protein Gels (Thermo-Fisher), NuPAGE™ MES SDS Running Buffer (Thermo-Fisher), Novex™ Sharp Pre-stained Protein Standard (Thermo-Fisher), PageBlue™ Protein Staining Solution (Thermo-Fischer), Pierce™ BCA Protein Assay Kit (Thermo-Fisher), N-Ethylmaleimide (NEM, 98%, Sigma-Aldrich), 1,4-Dithiothreitol (DTT, 98%, Sigma-Aldrich), Sephadex G25 (GE Healthcare), Sephadex G50 M (GE Healthcare), Superdex 200P (GE Healthcare), Isopropyl alcohol (IPA, 99.6%, VWR), Tris(hydroxymethyl)aminomethane (Tris, 99%, Sigma-Aldrich), Tris(hydroxymethyl)aminomethane hydrochloride (Tris·HCL, Sigma-Aldrich), L-Histidine (99%, Sigma-Aldrich), D-(+)-Trehalose dehydrate (99%, Sigma-Aldrich), Polyethylene glycol sorbitan monolaurate (TWEEN 20, Sigma-Aldrich), Dulbecco's Phosphate-Buffered Saline (DPBS, Thermo-Fisher), Guanidine hydrochloride (99%, Sigma-Aldrich), Ethylenediaminetetraacetic acid disodium salt dihydrate (EDTA-Naz, 99%, Sigma-Aldrich), sterile filters 0.2 μm and 0.45 μm (Sartorius), Vivaspin T4 and T15 concentrator (Sartorius), Superdex 200PG (GE Healthcare), Tetra(ethylene glycol), Dimethyl sulfoxide (DMSO, 99%, Sigma-Aldrich), N-(2-Aminoethyl)maleimide trifluoroacetate salt (AEM, 98%, Sigma-Aldrich), L-Cysteine (98.5%, Sigma-Aldrich), deionized water (DI) was freshly taken from Ultrapure Lab Water Systems (MilliQ, Merck), Nickel-nitrilotriacetic acid agarose (Ni-NTA agarose, Protino), Glycine (99.5%, VWR), 5,5-Dithiobis(2-nitrobenzoic acid (Ellman's reagent, DTNB, 98%, Sigma-Aldrich), S-Acetylmercaptosuccinic anhydride Fluorescein (SAMSA reagent, Invitrogen) Sodium bicarbonate (99.7%, Sigma-Aldrich), Sodium carbonate (99.9%, Sigma-Aldrich), PD MiniTrap desalting columns with Sephadex G-25 resin (GE Healthcare), PD10 G25 desalting column (GE Healthcare), Zeba Spin Desalting Columns in 0.5, 2, 5, and 10 mL (Thermo-Fisher), Vivaspin Centrifugal Filters T4 10 kDa MWCO, T4 100 kDa MWCO, and T15 (Sartorius), Biosep s3000 aSEC column (Phenomenex), Vivacell Ultrafiltration Units 10 and 30 kDa MWCO (Sartorius), Nalgene Rapid-Flow filter (Thermo-Fisher),

Methods

Cell Viability Assay

After treatment the cells were incubated for 72 hr at 37° C. before the cell viability was determined by a MTS-assay, performed according to the manufacturer's instruction (CellTiter 96® AQueous One Solution Cell Proliferation Assay, Promega). Briefly, the MTS solution was diluted 20× in DMEM without phenol red (PAN-Biotech GmbH) supplemented with 10% FBS. The cells were washed once with 200 μL/PBS well, after which 100 μL diluted MTS solution was added/well. The plate was incubated for approximately 20-30 minutes at 37° C. Subsequently, the OD at 492 nm was measured on a Thermo Scientific Multiskan FC plate reader (Thermo Scientific). For quantification the background signal of ‘medium only’ wells was subtracted from all other wells, before the cell viability percentage of treated/untreated cells was calculated, by dividing the background corrected signal of treated wells over the background corrected signal of the untreated wells (×100).

FACS Analysis

Cells were seeded in DMEM (PAN-Biotech GmbH) supplemented with 10% fetal calf serum (PAN-Biotech GmbH) and 1% penicillin/streptomycin (PAN-Biotech GmbH), at 500,000 c/plate in 10 cm dishes and incubated for 48 hrs (5% CO₂, 37° C.), until a confluency of 90% was reached. Next, the cells were trypsinized (TrypIE Express, Gibco Thermo Scientific) to single cells. 0.75×10⁶ Cells were transferred to a 15 mL falcon tube and centrifuged (1,400 rpm, 3 min). The supernatant was discarded while leaving the cell pellet submerged. The pellet was dissociated by gentle tapping the falcon tube on a vortex shaker and the cells were washed with 4 mL cold PBS (Mg²⁺ and Ca²⁺ free, 2% FBS). After washing the cells were resuspended in 3 mL cold PBS (Mg²⁺ and Ca²⁺ free, 2% FBS) and divided equally over 3 round bottom FACS tubes (1 mL/tube). The cells were centrifuged again and resuspended in 200 μL cold PBS (Mg²⁺ and Ca²⁺ free, 2% FBS) or 200 μL antibody solution; containing 5 μL antibody in 195 μL cold PBS (Mg²⁺ and Ca²⁺ free, 2% FBS). APC Mouse IgG1, K APC anti-human EGFR (#352906, Biolegend) was used to stain the EGFR receptor. PE anti-human HER2 APC anti-human CD340 (erbB2/HER-2) (#324408 Biolegend) was used to stain the HER2 receptor, PE Mouse IgG2a, K Isotype Ctrl FC (#400212, Biolegend) was used as its matched isotype control. PE anti-human CD71 (#334106, Biolegend) was used to stain the CD71 receptor, PE Mouse IgG2a, K Isotype Ctrl FC (#400212, Biolegend) was used as its matched isotype control. Samples were incubated for 30 min at 4° C. on a tube roller mixer. Afterwards, the cells were washed 3× with cold PBS (Mg²⁺ and Ca²⁺ free, 2% FBS) and fixated for 20 min at room temperature using a 2% PFA solution in PBS. Cells were washed 2× with cold PBS and resuspended in 250-350 μL cold PBS for FACS analysis. Samples were analyzed with a BD FACSCanto II flow cytometry system (BD Biosciences) and FlowJo software. See Table A2 for the results.

SO1861-EMCH Synthesis

To SO1861 (121 mg, 0.065 mmol) and EMCH·TFA (110 mg, 0.325 mmol) was added methanol (extra dry, 3.00 mL) and TFA (0.020 mL, 0.260 mmol). The reaction mixture stirred at room temperature. After 1.5 hours the reaction mixture was subjected to preparative MP-LC. ¹ Fractions corresponding to the product were immediately pooled together, frozen and lyophilized overnight to give the title compound (120 mg, 90%) as a white fluffy solid. Purity based on LC-MS 96%.

LRMS (m/z): 2069 [M−1]¹⁻

LC-MS r.t. (min): 1.084

Antibody-(L-SO1861)⁴

Trastuzumab, Cetuximab, are referred hereafter as “Ab”. Ab was conjugated to the saponin SO18161-EMCH via Michael-type thiol-ene conjugation reaction at DARs of 1, 2, 3, 4, 5, and 6. The SO1861-EMCH molecule obtains a labil (L) hydrazone bond between its structure and its maleimide function generating a labil bond between the saponin and Ab. The procedure is exemplary described for Trastuzumab-(L-SO1861)₄:

To a solution of Cetuximab (40 mg, 8.0 ml) was added 10 μl/ml each of Tris concentrate (127 mg/ml, 1.05 M), Tris·HCl concentrate (623 mg/ml, 3.95 M) and EDTA-Na₂ concentrate (95 mg/ml, 0.26M) to give a 50 mM TBS, 2.5 mM EDTA buffer pH 7.5.

To Cetuximab divided into four portions (each of 9.73 mg, 4.864 mg/ml, 65 nmol) was added an aliquot of freshly prepared TCEP solution (0.5-2.0 mg/ml, 1.15-7.02 mole equivalents, 75-455 nmol), the mixtures vortexed briefly then incubated for 300 minutes at 20° C. with roller-mixing. After incubation (prior to addition of SO1861-EMCH), a ca. 1 mg (0.210 ml) aliquot of Ab-SH was removed from each mixture and purified by gel filtration using a zeba spin desalting column into TBS pH 7.5. These aliquots were characterized by UV-vis analysis and Ellman's assay (thiol to Ab ratio=2.0, 4.2, 5.9 and 6.8 respectively). To each of the bulk Ab-SH was added an aliquot of freshly prepared SO1861-EMCH solution (2 mg/ml, 1.3 mole equivalents per ‘thiol’, 0.15-0.61 μmol, 0.16-0.63 ml), the mixtures vortexed briefly then incubated for 120 minutes at 20° C. Besides each conjugation reaction, two aliquots of desalted Ab-SH (0.25 mg, 1.67 nmol) were reacted with NEM (1.3 mole equivalents per ‘thiol’, 4.3-17.4 nmol, 2.2-8.7 μl of a 0.25 mg/ml solution) or TBS pH 7.5 buffer (2.2-8.7 μl) for 120 minutes at 20° C., as positive and negative controls, respectively. After incubation (prior to addition of NEM), a 0.200 ml aliquot of Ab-SO1861-EMCH mixture was removed and purified by gel filtration using zeba spin desalting column into TBS pH 7.5. This aliquot was characterized by UV-vis and alongside positive and negative controls were characterized by Ellman's assay to obtain SO1861-EMCH incorporations. To the bulk Ab-SO1861-EMCH mixture was added an aliquot of freshly prepared NEM solution (2.5 mg/ml, 2.5-10 mole equivalents, 0.15-0.58 μmol) and the mixtures purified by zeba spin desalting columns eluting with DPBS pH 7.5 to give purified Cetuximab-(L-SO1861) conjugates. The products were normalized to 2.5 mg/ml and filtered to 0.2 μm prior to dispensing for biological evaluation. See Table A3 and Table A4.

### LITERATURE REFERENCES

Wilton, E. E. et al. (2018) “sdAb-DB: The Single Domain Antibody Database”, ACS Synthetic Biology 7(11): 2480-2484. DOI: 10.1021/acssynbio.8b00407
Marta Kijanka & Frank-Jan Warnders & Mohamed El Khattabi & Marjolijn Lub-de Hooge & Gooitzen M. van Dam & Vasilis Ntziachristos & Liesbeth de Vries & Sabrina Oliveira & Paul M. P. van Bergen en Henegouwen, “Rapid optical imaging of human breast tumour xenografts using anti-HER2 V_{HH}s site-directly conjugated to IRDye 800CW for image-guided surgery”, Eur J Nucl Med Mol Imaging (2013) 40:1718-1729 DOI 10.1007/s00259-013-2471-2
Karen Mercier, Raimond Heukers and Chiraz Frydman, “Surface Plasmon Resonance imaging (SPRi)—Production of a single domain antibody Q17c directed against recombinant HER2 protein and its binding study by Surface Plasmon Resonance imaging technology”, Horiba Application Note Pharmaceuticals SPRi 42, 2019

SEQ ID NOS
SEQ ID NO: 1: Amino-acid coding DNA sequence of Anti-HER2 sdAb 2Rb17c from camelid
gaagttcagctgcaggaatctggtggtggtctggttcagccgggtggttctctgcgtctgtcttgcgcggcgtctggtttcatcttctctaacgacgcg
atgacctgggttcgtcaggcgccgggtaaaggtctggaatgggtttcttctatcaactggtctggtacccacaccaactacgcggactctgttaaa
ggtcgtttcaccatctctcgtgacaacgcgaaacgtaccctgtacctgcagatgaactctctgaaagacgaagacaccgcgctgtactactgcg
ttaccggttacggtgttaccaaaaccccgaccggtcagggtacccaggttaccgtttcttctcaccaccaccaccaccactctccgtctaccccgc
cgaccccgtctccgtctaccccgccgtgc
SEQ ID NO: 2: Amino-acid sequence of Anti-HER2 sdAb 2Rb17c from camelid
EVQLQESGGGLVQPGGSLRLSCAASGFIFSNDAMTWVRQAPGKGLEWVSSINWSGTHTNYADSVK
GRFTISRDNAKRTLYLQMNSLKDEDTALYYCVTGYGVTKTPTGQGTQVTVSSHHHHHHSPSTPPTPS
PSTPPC
SEQ ID NO: 3: Amino-acid coding DNA sequence of Anti-HER2 sdAb NB2 from Camelus dromedarius
atggaagttcagctggttgaatctggtggtggtctggttcaggcgggtggttctctgcgtctgtcttgcgcggcgtctggtatcaccttctctatcaac
accatgggttggtaccgtcaggcgccgggtaaacagcgtgaactggttgcgctgatctcttctatcggtgacacctactacgcggactctgttaaag
gtcgtttcaccatctctcgtgacaacgcgaaaaacaccgtttacctgcagatgaactctctgaaaccggaagacaccgcggtttactactgcaa
acgtttccgtaccgcggcgcagggtaccgactactggggtcagggtacccaggttaccgtttcttctcaccaccaccaccaccac
SEQ ID NO: 4: Amino-acid sequence of Anti-HER2 sdAb NB2 from Camelus dromedarius
MEVQLVESGGGLVQAGGSLRLSCAASGITFSINTMGWYRQAPGKQRELVALISSIGDTYYADSVKGR
FTISRDNAKNTVYLQMNSLKPEDTAVYYCKRFRTAAQGTDYWGQGTQVTVSSHHHHHH
SEQ ID NO: 5: Amino-acid coding DNA sequence of Anti-HER2 sdAb pcNB2, a synthetic construct
atggaagttcagctggttgaaaaaggtggtggtcgtgttcaggcgggtggttctctgcgtctgcgttgcgcggcgtctggtatcaccttctctatcaa
caccatgggttggtaccgtcaggcgccgggtaaacagcgtgaactggttgcgctgatctcttctatcggtgacacctactacgcggactctgttaa
aggtcgtttccgtatccgtcgtgacaacgcgaaaaacaccgtttacctgcgtatgcgtcgtctgaaaccggaagacaccgcggtttactactgca
aacgtttccgtaccgcggcgcagggtaccgactactggggtcagggtacccgtgttaccgtttctaaacaccaccaccaccaccac
SEQ ID NO: 6: Amino-acid sequence of Anti-HER2 sdAb pcNB2, a synthetic construct
MEVQLVEKGGGRVQAGGSLRLRCAASGITFSINTMGWYRQAPGKQRELVALISSIGDTYYADSVKGR
FRIRRDNAKNTVYLRMRRLKPEDTAVYYCKRFRTAAQGTDYWGQGTRVTVSKHHHHHH
SEQ ID NO: 7: amino-acid coding DNA sequence of Anti-HER1 sdAb 7D12 from camelid
gcggcgcaggttaaactggaagaatctggtggtggttctgttcagaccggtggttctctgcgtctgacctgcgcggcgtctggtcgtacctctcgttc
ttacggtatgggttggttccgtcaggcgccgggtaaagaacgtgaattcgtttctggtatctcttggcgtggtgactctaccggttacgcggactctgt
taaaggtcgtttcaccatctctcgtgacaacgcgaaaaacaccgttgacctgcagatgaactctctgaaaccggaagacaccgcgatctacta
ctgcgcggcggcggcgggttctgcgtggtacggtaccctgtacgaatacgactactggggtcagggtacccaggttaccgtttcttct
SEQ ID NO: 8: amino-acid sequence of Anti-HER1 sdAb 7D12 from camelid
AAQVKLEESGGGSVQTGGSLRLTCAASGRTSRSYGMGWFRQAPGKEREFVSGISWRGDSTGYADS
VKGRFTISRDNAKNTVDLQMNSLKPEDTAIYYCAAAAGSAWYGTLYEYDYWGQGTQVTVSS
SEQ ID NO: 9: amino-acid coding DNA sequence of Anti-HER1 sdAb 9G8 from camelid
gaagttcagctggttgaatctggtggtggtctggttcaggcgggtggttctctgcgtctgtcttgcgcggcgtctggtcgtaccttctcttcttacgcg
atgggttggttccgtcaggcgccgggtaaagaacgtgaattcgttgttgcgatcaactggtcttctggttctacctactacgcggactctgttaaaggt
cgtttcaccatctctcgtgacaacgcgaaaaacaccatgtacctgcagatgaactctctgaaaccggaagacaccgcggtttactactgcgcgg
cgggttaccagatcaactctggtaactacaacttcaaagactacgaatacgactactggggtcagggtacccaggttaccgtttcttct
SEQ ID NO: 10: amino-acid sequence of Anti-HER1 sdAb 9G8 from camelid
EVQLVESGGGLVQAGGSLRLSCAASGRTFSSYAMGWFRQAPGKEREFVVAINWSSGSTYYADSVK
GRFTISRDNAKNTMYLQMNSLKPEDTAVYYCAAGYQINSGNYNFKDYEYDYWGQGTQVTVSS
SEQ ID NO: 11: Amino-acid coding DNA sequence of Anti-VGFR2 sdAb NTV1, a synthetic construct
atggcgcaggttcagctgctggaatctggtggtggtctggttcagccgggtggttctctgcgtctgtcttgcgcggcgtctggttactctgttatcaac
gacttcatgacctgggttcgtcaggcgccgggtaaaggtctggaatgggtttcttctatctctgttgcggacggttctacctactacgcggactctgtt
aaaggtcgtttcaccatctctcgtgacaactctaaaaacaccctgtacctgcagatgaactctctgcgtgcggaagacaccgcggtttactactg
cgcggcgcgtgttggtggtcgtgacctgggttggccgtacgaactggactactggggtcagggtaccctggttaccgtttcttct
SEQ ID NO: 12: Amino-acid sequence of Anti-VGFR2 sdAb NTV1, a synthetic construct
MAQVQLLESGGGLVQPGGSLRLSCAASGYSVINDFMTWVRQAPGKGLEWVSSISVADGSTYYADSV
KGRFTISRDNSKNTLYLQMNSLRAEDTAVYYCAARVGGRDLGWPYELDYWGQGTLVTVSS
SEQ ID NO: 13: Amino-acid coding DNA sequence of Anti-VGFR2 sdAb NTV2, a synthetic construct
atggcgcaggttcagctgctggaatctggtggtggtctggttcagccgggtggttctctgcgtctgtcttgcgcggcgtctggtttcaaaatcaccaa
caaaaccatggcgtgggttcgtcaggcgccgggtaaaggtctggaatgggtttcttctatcggttcttcttctggttctacctactacgcggactctgt
taaaggtcgtttcaccatctctcgtgacaactctaaaaacaccctgtacctgcagatgaactctctgcgtgcggaagacaccgcggtttactactg
cgcgcgtcgtaaaggtaaccgtctgggtccggcggcgctgcgttcttggggtcagggtaccctggttaccgtttcttct
SEQ ID NO: 14: Amino-acid sequence of Anti-VGFR2 sdAb NTV2, a synthetic construct
MAQVQLLESGGGLVQPGGSLRLSCAASGFKITNKTMAWVRQAPGKGLEWVSSIGSSSGSTYYADSV
KGRFTISRDNSKNTLYLQMNSLRAEDTAVYYCARRKGNRLGPAALRSWGQGTLVTVSS
SEQ ID NO: 15: Amino-acid coding DNA sequence of Anti-VGFR2 sdAb NTV3, a synthetic construct
atggcgcaggttcagctgctggaatctggtggtggtctggttcagccgggtggttctctgcgtctgtcttgcgcggcgtctggtgttcgtgttaactac
aaatctatgtcttgggttcgtcaggcgccgggtaaaggtctggaatgggtttctaccatcacctctcgtaacggttctacctactacgcggactctgtt
aaaggtcgtttcaccatctctcgtgacaactctaaaaacaccctgtacctgcagatgaactctctgcgtgcggaagacaccgcggtttactactg
cgcgaccggtcgtgcgcaccacgcgccggttcgttactggggtcagggtaccctggttaccgtttcttct
SEQ ID NO: 16: Amino-acid sequence of Anti-VGFR2 sdAb NTV3, a synthetic construct
MAQVQLLESGGGLVQPGGSLRLSCAASGVRVNYKSMSWVRQAPGKGLEWVSTITSRNGSTYYADS
VKGRFTISRDNSKNTLYLQMNSLRAEDTAVYYCATGRAHHAPVRYWGQGTLVTVSS
SEQ ID NO: 17: Amino-acid coding DNA sequence of Anti-VGFR2 sdAb NTV4, a synthetic construct
atggcgcaggttcagctgctggaatctggtggtggtctggttcagccgggtggttctctgcgtctgtcttgcgcggcgtctggtgttaccatcaccga
cgaagacatgacccgtgttcgtcaggcgccgggtaaaggtctggaatgggtttcttctatcctgaacaccggtggttctacctactacgcggactc
tgttaaaggtcgtttcaccatctctcgtgacaactctaaaaacaccctgtacctgcagatgaactctctgcgtgcggaagacaccgcggtttacta
ctgcgcggcggttcacgaaaaagcggcggacatgaacttctggggtcagggtaccctggttaccgtttcttct
SEQ ID NO: 18: Amino-acid sequence of Anti-VGFR2 sdAb NTV4, a synthetic construct
AQVQLLESGGGLVQPGGSLRLSCAASGVTITDEDMTRVRQAPGKGLEWVSSILNTGGSTYYADSVK
GRFTISRDNSKNTLYLQMNSLRAEDTAVYYCAAVHEKAADMNFWGQGTLVTVSS
SEQ ID NO: 19: amino-acid coding DNA sequence of Anti-human CD19 sdAb SRB-85 from Bactrian camel
gaagttcagctgctggaatctggtggtggtctggttcagccgggtggttctctgcgttcttgcgaagcgtctggtttcaacgcgatgacctctatcga
ctcttggaccgacgcggttaaaggttgggttcgtcagccgccgggtaaaggtctggaatgggtttctcgtttcgcgatctctcaggacaacgcga
aaaacaccgtttacctgcagatgaactctctgaaaccggaagacaccgcgatgtactactgcgcgctgtctaaatgctacacccgtgtttacga
ctactggggtcagggtacccaggttaccgtttcttctggt
SEQ ID NO: 20: amino-acid sequence of Anti-human CD19 sdAb SRB-85 from Bactrian camel
EVQLLESGGGLVQPGGSLRSCEASGFNAMTSIDSWTDAVKGWVRQPPGKGLEWVSRFAISQDNAK
NTVYLQMNSLKPEDTAMYYCALSKCYTRVYDYWGQGTQVTVSS
SEQ ID NO: 21: amino-acid coding DNA sequence of Anti-human CD19 sdAb SRB-37 from Bactrian camel
gaagttcagctgcaggaatctggtggtggtctggttcagccgggtggttctctgcgtctgtcttgcgcggcgtctggtttcatctacatggttggtatc
aaaaccgaacgtgacggtgttaaaggttgggttcgtcaggcgccgggtaaaggtctggaatggctgtctcgtttcaccatcccgcgtgacaacgc
gaaaaacaccctgtacctgcagatgaacaacctgaaatctgaagacaccgcgctgtactactgcgcgaccgaagaaaacgactggggtca
gggtacccaggttaccgtttcttctggt
SEQ ID NO: 22: amino-acid sequence of Anti-human CD19 sdAb SRB-37 from Bactrian camel
EVQLQESGGGLVQPGGSLRLSCAASGFIYMVGIKTERDGVKGWVRQAPGKGLEWLSRFTIPRDNAK
NTLYLQMNNLKSEDTALYYCATEENDWGQGTQVTVSS
SEQ ID NO: 23: Amino-acid coding DNA sequence of Anti-CTLA-4 sdAb NB16 from Camelus dromedarius
caggttcagctgcaggaatctggtggtggttctgttcaggcgggtggttctctgcgtctgtcttgcaccgcgtctggtttcggtgttgacggtaccgac
atgggttggtaccgtcaggcgccgggtaacgaatgcgaactggtttcttctatctcttctatcggtatcggttactactctgaatctgttaaaggtcgt
ttcaccatctctcgtgacaacgcgaaaaacaccgtttacctgcagatgaactctctgcgtccggacgacaccgcggtttactactgcggtcgtcgtt
ggatcggttaccgttgcggtaactggggtcgtggtacccaggttaccgtttcttct
SEQ ID NO: 24: Amino-acid sequence of Anti-CTLA-4 sdAb NB16 from Camelus dromedarius
QVQLQESGGGSVQAGGSLRLSCTASGFGVDGTDMGWYRQAPGNECELVSSISSIGIGYYSESVKGR
FTISRDNAKNTVYLQMNSLRPDDTAVYYCGRRWIGYRCGNWGRGTQVTVSS
SEQ ID NO: 25: Amino-acid coding DNA sequence of Anti-CTLA-4 sdAb NB36 from Camelus dromedarius
caggttcagctgcaggaatctggtggtggttctgttcaggcgggtggttctctgcgtctgtcttgcaccggttctcgttacacctacaccatgggttgg
ttccgtcaggcgccgggtaaagaacgtgaaggtgttgttgcgatcaccgcgttcggttctccgttctacgcggactctgttaaaggtcgtttcaccat
ctctcgtgacaacgcgaacaacaccatcttcctgcagatgaactctctgaaaccggaagactctgcgatgtactactgcgcggcgcgtggttctt
ctggtacctcttacaaatggaacgaatacggttcttacaactactggggtcagggtacccaggttaccgtttcttct
SEQ ID NO: 26: Amino-acid sequence of Anti-CTLA-4 sdAb NB36 from Camelus dromedarius
QVQLQESGGGSVQAGGSLRLSCTGSRYTYTMGWFRQAPGKEREGVVAITAFGSPFYADSVKGRFTI
SRDNANNTIFLQMNSLKPEDSAMYYCAARGSSGTSYKWNEYGSYNYWGQGTQVTVSS
SEQ ID NO: 27: Amino-acid coding DNA sequence of Anti-CTLA-4 sdAb NB91 from Camelus dromedarius
caggttcagctgcaggaatctggtggtggttctgttcaggcgggtggttctctgcgtctgtcttgcgcggcgtctaaatacacctcttgcatgggttgg
ttccgtcaggcgccgggtaaagaacgtgaagttgttgcgcacatcgactctggtccgcgtaccctgtacgcggactctgttaaaggtcgtttcacc
atctctaaagacaacgcgaaaaacaccctgtacctggaaatgtctaccctgaaaccggacgacaccgcgatgtactactgcgcggcgggtc
cgatgtactctggttcttgcaactacaactactggggtcagggtacccaggttaccgtttcttct
SEQ ID NO: 28: Amino-acid sequence of Anti-CTLA-4 sdAb NB91 from Camelus dromedarius
QVQLQESGGGSVQAGGSLRLSCAASKYTSCMGWFRQAPGKEREVVAHIDSGPRTLYADSVKGRFTI
SKDNAKNTLYLEMSTLKPDDTAMYYCAAGPMYSGSCNYNYWGQGTQVTVSS
SEQ ID NO: 29: Amino-acid coding DNA sequence of Anti-human PD-L1 sdAb A1 from Camelus dromedarius
Caggttcagctgcaggaatctggtggtggtctggttcagccgggtggttctctgcgtctgtcttgcgcggcgtctggtttcaccctggactactacgc
gatcggttggttccgtcaggcgccgggtaaagaacgtgaaggtgtttcttgcatctcttcttctgacggttctacctactacgcggactctgttaaag
gtcgtttcaccatctctcgtgacaacgcgaaaaacaccgtttacctgcagatgtcttctctgaaaccggaagacaccgcggtttactactgcggta
tctctggttcttgcctgctggaagactacggtatggactactggggtaaaggtacccaggttaccgtttcttct
SEQ ID NO: 30: Amino-acid sequence of Anti-human PD-L1 sdAb A1 from Camelus dromedarius
QVQLQESGGGLVQPGGSLRLSCAASGFTLDYYAIGWFRQAPGKEREGVSCISSSDGSTYYADSVKG
RFTISRDNAKNTVYLQMSSLKPEDTAVYYCGISGSCLLEDYGMDYWGKGTQVTVSS
SEQ ID NO: 31: Amino-acid coding DNA sequence of Anti-human PD-L1 sdAb B1 from Camelus dromedarius
caggttcagctgcaggaatctggtggtggtctggttcacccgggtggttctctgcgtctgtcttgcgcggcgtctggtttctctctggacaactacgcg
atcggttggttccgtcaggcgccgggtaaagaacgtgaaggtgtttcttgcatctcttctggttctgaaggtcgtcgttactacgcggacttcgttaaa
ggtcgtttcaccatctctcgtgacaacgcgaaaaacaccgcgttcctgcagatgaactctctgaaaccggaagacaccgcggactactactgc
gcgaccgttggtttctgctcttctcagtacggtatggaattcgttggtgactactggggtcagggtacccaggttaccgtttcttct
SEQ ID NO: 32: Amino-acid sequence of Anti-human PD-L1 sdAb B1 from Camelus dromedaries
QVQLQESGGGLVHPGGSLRLSCAASGFSLDNYAIGWFRQAPGKEREGVSCISSGSEGRRYYADFVK
GRFTISRDNAKNTAFLQMNSLKPEDTADYYCATVGFCSSQYGMEFVGDYWGQGTQVTVSS
SEQ ID NO: 33: Amino-acid sequence of Anti-mouse serum albumin sdAb MSA21 (organism: artificial
sequence)
QVQLQESGGGLVQPGGSLRLSCEASGFTFSRFGMTWVRQAPGKGVEWVSGISSLGDSTLYADSVK
GRFTISRDNAKNTLYLQMNSLKPEDTAVYYCTIGGSLNPGGQGTQVTVSS
SEQ ID NO: 34: Amino-acid sequence of Anti-human serum albumin sdAb Alb-1 (organism: artificial
sequence)
AVQLVESGGGLVQPGNSLRLSCAASGFTFRSFGMSWVRQAPGKEPEWVSSISGSGSDTLYADSVKG
RFTISRDNAKTTLYLQMNSLKPEDTAVYYCTIGGSLSRSSQGTQVTVSS
SEQ ID NO: 35: Amino-acid sequence of Anti-human serum albumin sdAb Alb23 (Humanized, optimized
Alb1) (organism: artificial sequence)
EVQLLESGGGLVQPGGSLRLSCAASGFTFRSFGMSWVRQAPGKGPEWVSSISGSGSDTLYADSVK
GRFTISRDNSKTLYLQMNSLRPEDTAVYYCTIGGSLSRSSQGTLVTVSS
SEQ ID NO: 36: Amino-acid sequence of Anti-EGFR VHH 7A5 (organism: artificial; recombinant peptide)
EVQLVESGGGLVQAGGSLRLSCAASDRTFSSNNMGWFRQAPGKEREFVAAIGWGGLETHYSDSVK
GRFTISRDNAKNTVYLQMNSLKPEDTARYYCAVSSTRTVIYTLPRMYNYWGQGTQVTVSS
SEQ ID NO: 37: Amino-acid sequence of Anti-EGFR VHH 7D12 (organism: artificial; recombinant peptide)
QVKLEESGGGSVQTGGSLRLTCAASGRTSRSYGMGWFRQAPGKEREFVSGISWRGDSTGYADSVK
GRFTISRDNAKNTVDLQMNSLKPEDTAIYYCAAAAGSAWYGTLYEYDYWGQGTQVTVSS
SEQ ID NO: 38: Amino-acid sequence of Anti-EGFR VHH 7C12 (organism: artificial; recombinant peptide)
AVQLVESGGGSVQAGGSLRLTCAASGRTSRSYGMGWFRQAPGKEREFVSGISWRGDSTGYADSVK
GRFTISRDNAKNTVDLQMNSLKPEDTAIYYCAAAAGSTWYGTLYEYDYWGQGTQVTVSS
SEQ ID NO: 39: Amino-acid sequence of Anti-insulin-like growth factor 1 receptor VHH 4B11 (organism:
artificial; recombinant peptide)
EVQLVESGGGLVQPGGSLRLSCAASGSIFTFNAMGWYRQAPGKQRELVAVIISGGSTHYVDSVKGRF
TISRDNAKKMVYLQMNSLKPEDTAVYYCNVKKFGDYWGQGTQVTVSS
SEQ ID NO: 40: Amino-acid sequence of Anti-insulin-like growth factor 1 receptor VHH 3G7 (organism:
artificial; recombinant peptide)
DVQLVESGGGLVQAGGSLRLSCAASESISTINVMAWYRQAPGKQRELVAEITRSGRTNYVDSVKGRF
TISRDNAKNTMYLQMNSLNLEDTAVYYCRTIDGSWREYWGQGTQVTVSS
SEQ ID NO: 41: Amino-acid sequence of Anti-insulin-like growth factor 1 receptor VHH 2C7 (organism:
artificial; recombinant peptide)
QVKLEESGGGLVQPGGSLRLSCVASGRTFSNYAIVIGWFRQAPGQEREFVAAINWNSRSTYYADSVK
GRFTISRLNARNTVYLQMNRLKPEDTAVYDCAASHDSDYGGTNANLYDYWGQGTQVTVSS
SEQ ID NO: 42: Amino-acid sequence of Anti-insulin-like growth factor 1 receptor VHH 1C7 (organism:
artificial; recombinant peptide)
QVKLEESGGGLVQAGGSLRLSCVASGRTFSRTANAWFRQAPGKEREFVATITWNSGTTRYADSVKG
RFFISKDSAKNTIYLEMNSLEPEDTAVYYCAATAAAVITPTRGYYNYWGQGTQVTVSS
SEQ ID NO: 43: Amino-acid sequence of Anti-CEACAM VHH NbCEA5 (organism: artificial; recombinant
peptide)
EVQLVESGGGSVQAGGSLRLSCAASGDTYGSYWMGWFRQAPGKEREGVAAINRGGGYTVYADSV
KGRFTISRDTAKNTVYLQMNSLRPDDTADYYCAASGVLGGLHEDWFNYWGQGTLVTVSS
SEQ ID NO: 44: Amino-acid sequence of Anti-CEACAM VHH CEA5 (organism: artificial; recombinant
peptide)
EVQLVESGGGSVQAGGSLRLSCAASGDTYGSYWMGWFRQAPGQEREAVAAINRGGGYTVYADSV
KGRFTISRDNAKNTLYLQMNSLRPDDTADYYCAASGVLGGLHEDWFNYWGQGTLVTVSS
SEQ ID NO: 45: Amino-acid sequence of Anti-CD123 VHH 57A07 (organism: artificial; recombinant
peptide)
EVQLVESGGGLVQAGGSLRLSCAASGSIFSGNVMGWYRRQAPGKEREWVAAIASGGSIYYRDSVKG
RFTISRDNAKNTVYLQMNSLKPEDTAVYYCNSHPPTLPYWGLGTQVTVSS
SEQ ID NO: 46: Amino-acid sequence of Anti-CD123 VHH 57B04 (organism: artificial; recombinant
peptide)
EVqLVESGGGLVQPGGSLRLSCAASGINFRFNSMGWWRRRAPGKEREWVAAITSGDITNYRDSVRG
RFTISRDNVKNTVYLQMNTLKLEDTAVYYCNTFPPIADYWGLGTQVTVSS
SEQ ID NO: 47: Amino-acid sequence of Anti-CD123 VHH 51D09 (organism: artificial; recombinant
peptide)
EVQLVESGGGLVQPGGSLRLSCAASGSIFSGNTMGWYRQAPGKQRELVAAISSGGSTDYADSVKGR
FTISRDNSKNTVYLQMNSLRPEDTAVYYCNAAILLYRLYGYEEGDYWGLGTLVTVSS
SEQ ID NO: 48: Amino-acid sequence of Anti-CD123 VHH 55C05 (organism: artificial; recombinant
peptide)
EVQLVESGGGLVPAGDSLRLSCVASGRSLNTYTMGWFRQAPGKECEEVAAINWNGVYRDYADSAK
GRETASRDNAMNTVFLQMNSLKPEDTAVYFCATATQGWDRHTEPSDFGSWGLGTQVTVSS
SEQ ID NO: 49: Amino-acid sequence of Anti-CD123 VHH 50F07 (organism: artificial; recombinant
peptide)
EVQLVESGGGLVQPGGSLRLSCTGSGSTFSINAMGWYRQAPGKQRELVAAITSGGRTNYADSVKGR
FTISRDNSKNTVYLQMNSLRPEDTAVYYCNARISAGTAFWLWSDYEYWGLGTLVTVSS
SEQ ID NO: 50: Amino-acid sequence of Anti-CD123 VHH 55F03 (organism: artificial; recombinant
peptide)
EVQLVESGGGLVQAGGPLRLSCAASGRTFSSYVMGWFRQAPGKEREFVAAIYWSNGKTQYTDSVK
GRFTISGDNAKNTVYLQMNSLNPEDTAVYYCVADKDETGFRTLPIAYDYWGLGTQVTVSS
SEQ ID NO: 51: Amino-acid sequence of Anti-CD123 VHH 55A01 (organism: artificial; recombinant
peptide)
EVQLVESGGGSVQAGGSLRLSCTTSGRALNMYVMGWFRQAPGNEREFVAATSSSGGSTSYPDSVK
GRFTISRDNAKNTVYLQMNSLKPEDTAAYRCAASPYVSTPTMNILEEYRYWGLGTQVTVSS
SEQ ID NO: 52: Amino-acid sequence of Anti-c-MET VHH 04E09 (organism: artificial sequence)
EVQLVESGGGLVQPGGSLRLSCAASGFILDYYAIGWFRQAPGKEREGVLCIDASDDITYYADSVKGRF
TISRDNAKNTVYLQMNSLKPEDTGVYYCATPIGLSSSCLLEYDYDYWGQGTLVTVSS
SEQ ID NO: 53: Amino-acid sequence of Anti-c-MET VHH 06B08 (organism: artificial sequence)
EVQLVESGGGLVQAGGSLRLSCAASGRTISRYTMGWFRQAPGKEREFVAAISWSGDNTNYADSVKG
RFTISRPNTKNTMYLQMNSLKPEDTAVYYCAADYRSGSYYQASEWTRPSGYDYWGQGTLVTVSS
SEQ ID NO: 54: Amino-acid sequence of Anti-c-MET VHH 06C12 (organism: artificial sequence)
EVQLVESGGGLVQPGGSLRLSCAASGFSLDYFAIGWFRQAPGKEREEISCISNSDGSTYYANSVKGR
FTISIDNAKNTVYLQMTSLKPEDTAVYYCATPVGLGPFCKTTNDYDYSGQGTLVTVSS
SEQ ID NO: 55: Amino-acid sequence of Anti-c-MET VHH 06F10 (organism: artificial sequence)
EVQLVESGGGLVQPGGSLRLSCAASGFTLDYYAINWFRQAPGKEREGVSCISGGDGSTYYADSVKG
RFTISRDNAKNTVYLQMNSLKPEDTAVYYCATALGLSSSCHGDGYDYWGQGTLVTVSS
SEQ ID NO: 56: Amino-acid sequence of Anti-Her3 VHH 21F6 (organism: artificial sequence)
EVQLVESGGGLVQAGGSLRLSCAASGRTYYLNAMGWFRQGPGKDREFVAAIDWSDGNKDYADSVK
GRFTISRDNAKNTVYLQMNSLKPEDTAVYYCAADTPPWGPMIYIESYDSWGQGTLVTVSS
SEQ ID NO: 57: Amino-acid sequence of Anti-Her3 VHH 4C7 (organism: artificial sequence)
EVQLVESGGGLVQAGGSLRLSCAASGFTFSSYPMSWVRQAPGKGPAWVSTVSPGGITTSYADSVKG
RFTISRDNAKNTLYLQMNSLKPEDTAVYYCLRDLNNRGQGTLVTVSS
SEQ ID NO: 58: Amino-acid sequence of Anti-Her3 VHH 17B5 (organism: artificial sequence)
EVQLVESGGGLVQPGGSLRLSCAASGSIGGLNAMAWYRQAPGKERELVAGIFGVGSTRYADSVKGR
FTISRDIAKNTVFLQMNSLNSEDTAVYYCRMSSVTRGSSDYWGQGTQVTVSS
SEQ ID NO: 59: Amino-acid sequence of Anti-Her3 VHH 18G11 (organism: artificial sequence)
EVQLVESGGGLVQPGGSLRLSCAASGTLFKINAMGWYRQAPGKRRELVALITSSDTTDYAESVEGRF
TISRDNTWNAVYLQMNSLKPEDTAVYYCHSDHYSMGVPEKRVIMYGQGTQVTVSS
SEQ ID NO: 60: Amino-acid sequence of Anti-Her3 VHH 34C7 (organism: artificial sequence)
EVQLVESGGGLVQPGGSLGLSCVASGSIFRINAMAWYRQAPGKQRELVAEITAGGSTNYADSVKGRF
TISVDNAWNTLYLQMNSLKVEDTAVYYCNLDHYTTWDRRSAYWGQGTQVTVSS
SEQ ID NO: 61: Amino-acid sequence of Anti-Her2 VHH 47D5 (organism: llama)
KVQLVESGGGLVQPGGSLRLSCAASGSIFGFNDMAWYRQAPGKQRELVALISRVGVTSSADSVKGR
FTISRVNAKDTVYLQMNSLKPEDTAVYYCYMDQRLDGSTLAYWGQGTQVTVSS
SEQ ID NO: 62: Amino-acid sequence of Anti-Her2 VHH 2D3 (organism: llama)
EVQLVESGGSLVQPGGSLRLSCAASGFTFDDYAMSWVRQVPGKGLEWVSSINWSGTHTDYADSVK
GRFTISRNNANNTLYLQMNSLKSEDTAVYYCAKNWRDAGTTWFEKSGSAGQGTQVTVSS
SEQ ID NO: 63: Amino-acid sequence of Anti-Her2 VHH 5F7 (organism: llama)
EVQLVESGGGLVQPGGSLRLSCAASGFTFSINTMGWYRQAPGKQRELVALISSIGDTYYADSVKGRF
TISRDNAKNTVYLQMNSLKPEDTAVYYCKRFRTAAQGTDYWGQGTQVTVSS
SEQ ID NO: 64: Amino-acid sequence of Anti-Her2 VHH 13D11 (organism: llama)
EVQLVESGGGLVHPGGSLRLSCVGSGFSLDDYGMTWVRRAPGKGLEWVSSINWSGTHTDYADSVK
GRFTISRDNAKNTLFLQMNSLNPEDTAVYYCGQGWKIVPTNPRGHGTQVTVSS
SEQ ID NO: 65: Amino-acid sequence of Anti-Her2 VHH 2B4 (organism: llama)
EVQLVESGGGLVQPGGSLRLSCVGSGFSLDDYAMTWVRQAPGKGLEWVSSINWSGTHTDYADSVK
GRFTISRDNAKNTLFLQMNSLSPEDTAVYYCNQGWKIRPTIPMGHGTQVTVSS
SEQ ID NO: 66: Amino-acid sequence of Anti-Her2 VHH 2G2 (organism: llama)
EVQLVESGGGLVQPGGSLRLSCVASGFSLDDYGMTWVRQAPGKGLEWVSSINWSGTHTDYTDPVK
GRFTISRDNAKNTLFLQMNNLTPEDTAVYYCNRGWKIVPTDLGGHGTQVTVSS
SEQ ID NO: 67: Amino-acid sequence of Anti-Her2 VHH 13G11 (organism: llama)
EVQLVESGGGLVQAGGSLRLSCAASGRTFISNYAMGWFRQAPGKEREFVATINWSGSHSDYADSVK
GRFTISRDNAKNTVYLQMNNLKSEDTAVYYCAPGWGTAPLSTSVYWGQGTQVTVSS
SEQ ID NO: 68: Amino-acid sequence of Anti-Her2 VHH 12E33 (organism: llama)
EVQLVESGGGMVQAGGSLRLSCAASGLTLSNYGMGWFRQAPGKEREFVSSINWSGTHTYDADFVK
GRFIISRDNAKNTVYLQINSLKPEDTAVYYCAAGGWGTGRYNYWGQGTQVTVSS
SEQ ID NO: 69: Amino-acid sequence of Anti-Her2 VHH 13F21 (organism: llama)
EVQLVESGGGLVQSGGSLRLSCVASGTIVSINATSWYRQAPGNQRELVATIIGDGRTHYADSVKDRFT
ISRDAAANLVYLQMNSLKPSDTAIYSCNANGIESYGWGNRHFNYWTVGTQVTVSS
SEQ ID NO: 70: Amino-acid sequence of Anti-Her2 VHH 11A101 (organism: llama)
EVQLVESGGGLVQAGGSLRLSCAASGRTFNAMGWFRQAPGKEREFVAAISRSPGVTYYADSVKGRF
TTSRDNAKNTVYLQMNDLKPEDTAVYYCAADFYLATLAHEYDYWGQGTQVTVSS
SEQ ID NO: 71: Amino-acid sequence of Anti-Her2 VHH 11A22 (organism: llama)
EVQLVESGGGLVQAGGSLRLSCAASGRTFSSYAMAWFRQAPGTEREFIAGIRWSDGSTYYADSVKG
RFTISRDNAKNTVYLQMNSLKPEDTAVYYCAADFYVSTLAHEYDYWGQGTQVTVSS
SEQ ID NO: 72: Amino-acid sequence of Anti-Her2 VHH 12D44 (organism: llama)
KVQLVESGGGLVQAGGSLRLSCAASGRTFSSYAMAWFRQAPGTEREFIAGIRWSDGSTYYADSVKG
RFTISRANAKNTVYLQMNGLKPEDTAVYYCAADFYVSTLAHEYDYWGQGTQVTVSS
SEQ ID NO: 73: Amino acid sequence of bivalent VHHEGFR-dianthin-Cys
QVKLEESGGGSVQTGGSLRLTCAASGRTSRSYGMGWFRQAPGKEREFVSGISWRGDSTGYADSVK
GRFTISRDNAKNTVDLQMNSLKPEDTAIYYCAAAAGSAWYGTLYEYDYWGQGTQVTVSSGGGGSGG
GGSEVQLVESGGGLVQAGGSLRLSCAASGRTFSSYAMGWFRQAPGKEREFVVAINWSSGSTYYAD
SVKGRFTISRDNAKNTMYLQMNSLKPEDTAVYYCAAGYQINSGNYNFKDYEYDYWGQGTQVTVSSG
GGGSGGGGSAAATAYTLNLANPSASQYSSFLDQIRNNVRDTSLIYGGTDVAVIGAPSTTDKFLRLNFQ
GPRGTVSLGLRRENLYVVAYLAMDNANVNRAYYFKNQITSAELTALFPEVVVANQKQLEYGEDYQAIE
KNAKITTGDQSRKELGLGINLLITMIDGVNKKVRVVKDEARFLLIAIQMTAEAARFRYIQNLVTKNFPNKF
DSENKVIQFQVSWSKISTAIFGDCKNGVFNKDYDFGFGKVRQAKDLQMGLLKYLGRPKGGGGSGGG
GSHRWCCPGCCKTFGGGGSHHHHHHHHHHHRWCCPGCCKTF
SEQ ID NO: 74: Amino acid sequence of bivalent VHHEGFR-Cys
QVKLEESGGGSVQTGGSLRLTCAASGRTSRSYGMGWFRQAPGKEREFVSGISWRGDSTGYADSVK
GRFTISRDNAKNTVDLQMNSLKPEDTAIYYCAAAAGSAWYGTLYEYDYWGQGTQVTVSSGGGGSGG
GGSEVQLVESGGGLVQAGGSLRLSCAASGRTFSSYAMGWFRQAPGKEREFVVAINWSSGSTYYAD
SEQ ID NO: 75: Amino acid sequence of VHH 7D12
QVKLEESGGGSVQTGGSLRLTCAASGRTSRSYGMGWFRQAPGKEREFVSGISWRGDSTGYADSVK
GRFTISRDNAKNTVDLQMNSLKPEDTAIYYCAAAAGSAWYGTLYEYDYWGQGTQVTVSS
SEQ ID NO: 76: Amino acid sequence of VHH 9G8
EVQLVESGGGLVQAGGSLRLSCAASGRTFSSYAMGWFRQAPGKEREFVVAINWSSGSTYYADSVK
GRFTISRDNAKNTMYLQMNSLKPEDTAVYYCAAGYQINSGNYNFKDYEYDYWGQGTQVTVSS

1. Conjugate for transferring a saponin from outside a cell into said cell, comprising a single-domain antibody (sdAb), capable of binding to said cell, covalently bound to at least one saponin, directly or via a linker, wherein the at least one saponin is a monodesmosidic triterpene glycoside or a bidesmosidic triterpene glycoside.

2. Conjugate of claim 1, wherein the sdAb is a V_{H} domain derived from a heavy chain of an antibody, preferably of immunoglobulin G origin, preferably of human origin, a V_{L} domain derived from a light chain of an antibody, preferably of immunoglobulin G origin, preferably of human origin, a V_{HH} domain such as derived from a heavy-chain only antibody (HCAb) such as from Camelidae origin or Ig-NAR origin such as a variable heavy chain new antigen receptor (V_{NAR}) domain, preferably the HCAb is from Camelidae origin, preferably the sdAb is a V_{HH} domain derived from an HCAb from Camelidae origin (camelid V_{H}) such as derived from an HCAb from camel, lama, alpaca, dromedary, vicuna, guanaco and Bactrian camel.

3. Conjugate of claim 1 or 2, wherein the conjugate comprises at least two sdAbs with a single first sdAb covalently linked to the at least one saponin, or with two or more sdAbs linked to at least one saponin, or with all of the at least two sdAbs linked to at least one saponin.

4. Conjugate of any one of the claims 1-3, wherein the sdAb comprises at least two sdAbs, which are the same sdAbs, preferably two-eight sdAbs, more preferably two-four sdAbs.

5. Conjugate of any one of the claims 1-4, comprising one-eight sdAbs, capable of binding to the same binding site on a cell-surface molecule of the cell, wherein the at least one saponin is linked to a single first sdAb of the one-eight sdAbs or wherein the at least one saponin is linked to two or more of the sdAbs, if present.

6. Conjugate of any one of the claims 1-5, comprising at least two sdAbs which are biparatopic, preferably comprising two sdAbs which are biparatopic.

7. Conjugate of any one of the claims 1-6, wherein the conjugate comprises 1-100 saponin moieties of the at least one saponin, preferably 2-64 saponin moieties, more preferably 4-32 saponin moieties, most preferably 8-16 saponin moieties, or any number therein between.

8. Conjugate of any one of the claims 1-7, wherein the at least one saponin comprises an aglycone core structure selected from any one of:
2alpha-hydroxy oleanolic acid;
16alpha-hydroxy oleanolic acid;
hederagenin (23-hydroxy oleanolic acid);
16alpha,23-dihydroxy oleanolic acid;
gypsogenin;
quillaic acid;
protoaescigenin-21(2-methyl but-2-enoate)-22-acetate;
23-oxo-barringtogenol C-21,22-bis(2-methylbut-2-enoate);
23-oxo-barringtogenol C-21(2-methylbut-2-enoate)-16,22-diacetate;
digitogenin;
3,16,28-trihydroxy oleanan-12-en; and
gypsogenic acid,
preferably, the at least one saponin comprises an aglycone core structure selected from quillaic acid and gypsogenin, more preferably the aglycone core structure of the at least one saponin is quillaic acid.

9. Conjugate of any one of the claims 1-8, wherein the at least one saponin comprises a first saccharide chain, which is bound to the 03 atom or the 028 atom of the aglycone core structure of the at least one saponin, preferably to the 03 atom, and/or wherein the at least one saponin comprises a second saccharide chain, which is bound to the 028 atom of the aglycone core structure of the at least one saponin, preferably, the at least one saponin comprises the first and second saccharide chain.

10. Conjugate of any one of the claims 1-9, wherein the at least one saponin comprises a first saccharide chain selected from:
GlcA-,
Glc-,
Gal-,
Rha-(1→2)-Ara-,
Gal-(1→2)-[Xyl-(1→3)]-GlcA-,
Glc-(1→2)-[Glc-(1→4)]-GlcA-,
Glc-(1→2)-Ara-(1→3)-[Gal-(1→2)]-GlcA-,
Xyl-(1→2)-Ara-(1→3)-[Gal-(1→2)]-GlcA-,
Glc-(1→3)-Gal-(1→2)-[Xyl-(1→3)]-Glc-(1→4)-Gal-,
Rha-(1→2)-Gal-(1→3)-[Glc-(1→2)]-GlcA-,
Ara-(1-4)-Rha-(1→2)-Glc-(1→2)-Rha-(1→2)-GlcA-,
Ara-(1-4)-Fuc-(1→2)-Glc-(1→2)-Rha-(1→2)-GlcA-,
Ara-(1-4)-Rha-(1→2)-Gal-(1→2)-Rha-(1→2)-GlcA-,
Ara-(1-4)-Fuc-(1→2)-Gal-(1→2)-Rha-(1→2)-GlcA-,
Ara-(1-4)-Rha-(1→2)-Glc-(1→2)-Fuc-(1→2)-GlcA-,
Ara-(1-4)-Fuc-(1→2)-Glc-(1→2)-Fuc-(1→2)-GlcA-,
Ara-(1→4)-Rha-(1→2)-Gal-(1→2)-Fuc-(1→2)-GlcA-,
Ara-(1-4)-Fuc-(1→2)-Gal-(1→2)-Fuc-(1→2)-GlcA-,
Xyl-(1-4)-Rha-(1→2)-Glc-(1→2)-Rha-(1→2)-GlcA-,
Xyl-(1-4)-Fuc-(1→2)-Glc-(1→2)-Rha-(1→2)-GlcA-,
Xyl-(1→4)-Rha-(1→2)-Gal-(1→2)-Rha-(1→2)-GlcA-,
Xyl-(1→4)-Fuc-(1→2)-Gal-(1→2)-Rha-(1→2)-GlcA-,
Xyl-(1-4)-Rha-(1→2)-Glc-(1→2)-Fuc-(1→2)-GlcA-,
Xyl-(1→4)-Fuc-(1→2)-Glc-(1→2)-Fuc-(1→2)-GlcA-,
Xyl-(1→4)-Rha-(1→2)-Gal-(1→2)-Fuc-(1→2)-GlcA-, and
Xyl-(1→4)-Fuc-(1→2)-Gal-(1→2)-Fuc-(1→2)-GlcA-,
and/or wherein the at least one saponin comprises a second saccharide chain is selected from:
Glc-,
Gal-,
Rha-(1→2)-[Xyl-(1-4)]-Rha-,
Rha-(1→2)-[Ara-(1→3)-Xyl-(1→4)]-Rha-,
Ara-,
Xyl-,
Xyl-(1→4)-Rha-(1→2)-[R1-(→4)]-Fuc- wherein R1 is 4E-Methoxycinnamic acid,
Xyl-(1→4)-Rha-(1→2)-[R2-(→4)]-Fuc- wherein R2 is 4Z-Methoxycinnamic acid,
Xyl-(1→4)-[Gal-(1→3)]-Rha-(1→2)-4-OAc-Fuc-,
Xyl-(1-4)-[Glc-(1→3)]-Rha-(1→2)-3,4-d i-OAc-Fuc-,
Xyl-(1→4)-[Glc-(1→3)]-Rha-(1→2)-[R3-(→4)]-3-OAc-Fuc- wherein R3 is 4E-Methoxycinnamic acid,
Glc-(1→3)-Xyl-(1→4)-[Glc-(1→3)]-Rha-(1→2)-4-OAc-Fuc-,
Glc-(1→3)-Xyl-(1-4)-Rha-(1→2)-4-OAc-Fuc-,
(Ara- or Xyl-)(1→3)-(Ara- or Xyl-)(1-4)-(Rha- or Fuc-)(1→2)-[4-OAc-(Rha- or Fuc-)(1→4)]-(Rha- or Fuc-),
Xyl-(1→3)-Xyl-(1→4)-Rha-(1→2)-[Qui-(1→4)]-Fuc-,
Api-(1→3)-Xyl-(1→4)-[Glc-(1→3)]-Rha-(1→2)-Fuc-,
Xyl-(1-4)-[Gal-(1→3)]-Rha-(1→2)-Fuc-,
Xyl-(1-4)-[Glc-(1→3)]-Rha-(1→2)-Fuc-,
Ara/Xyl-(1-4)-Rha/Fuc-(1-4)-[Glc/Gal-(1→2)]-Fuc-,
Api-(1→3)-Xyl-(1→4)-[Glc-(1→3)]-Rha-(1→2)-[R4-(→4)]-Fuc- wherein R4 is 5-O-[5-O-Ara/Api-3,5-dihydroxy-6-methyl-octanoyl]-3,5-dihydroxy-6-methyl-octanoic acid),
Api-(1→3)-Xyl-(1→4)-Rha-(1→2)-[R5-(→4)]-Fuc- wherein R5 is 5-O-[5-O-Ara/Api-3,5-dihydroxy-6-methyl-octanoyl]-3,5-dihydroxy-6-methyl-octanoic acid),
Api-(1→3)-Xyl-(1→4)-Rha-(1→2)-[Rha-(1→3)]-4-OAc-Fuc-,
Api-(1→3)-Xyl-(1→4)-[Glc-(1→3)]-Rha-(1→2)-[Rha-(1→3)]-4-OAc-Fuc-,
6-OAc-Glc-(1→3)-Xyl-(1-4)-Rha-(1→2)-[3-OAc-Rha-(1→3)]-Fuc-,
Glc-(1→3)-Xyl-(1-4)-Rha-(1→2)-[3-OAc-Rha-(1→3)]-Fuc-,
Xyl-(1→3)-Xyl-(1→4)-Rha-(1→2)-[Qui-(1→4)]-Fuc-,
Glc-(1→3)-[Xyl-(1→4)]-Rha-(1→2)-[Qui-(1-4)]-Fuc-,
Glc-(1→3)-Xyl-(1-4)-Rha-(1→2)-[Xyl-(1→3)-4-OAc-Qui-(1-4)]-Fuc-,
Xyl-(1→3)-Xyl-(1→4)-Rha-(1→2)-[3,4-di-OAc-Qui-(1→4)]-Fuc-,
Glc-(1→3)-[Xyl-(1→4)]-Rha-(1→2)-Fuc-,
6-OAc-Glc-(1→3)-[Xyl-(1→4)]-Rha-(1→2)-Fuc-,
Glc-(1→3)-[Xyl-(1→3)-Xyl-(1→4)]-Rha-(1→2)-Fuc-,
Xyl-(1→3)-Xyl-(1-4)-Rha-(1→2)-[Xyl-(1→3)-4-OAc-Qui-(1-4)]-Fuc-,
Api/Xyl-(1→3)-Xyl-(1→4)-[Glc-(1→3)]-Rha-(1→2)-[Rha-(1→3)]-40Ac-Fuc-,
Api-(1→3)-Xyl-(1→4)-[Glc-(1→3)]-Rha-(1→2)-[Rha-(1→3)]-40Ac-Fuc-,
Api/Xyl-(1→3)-Xyl-(1→4)-[Glc-(1→3)]-Rha-(1→2)-[R6-(→4)]-Fuc- wherein R6 is 5-O-[5-O-Rha-(1→2)-Ara/Api-3,5-dihydroxy-6-methyl-octanoyl]-3,5-dihydroxy-6-methyl-octanoic acid),
Api/Xyl-(1→3)-Xyl-(1→4)-[Glc-(1→3)]-Rha-(1→2)-[R7-(→4)]-Fuc- wherein R7 is 5-O-[5-O-Ara/Api-3,5-dihydroxy-6-methyl-octanoyl]-3,5-dihydroxy-6-methyl-octanoic acid),
Api/Xyl-(1→3)-Xyl-(1→4)-[Glc-(1→3)]-Rha-(1→2)-[R8-(→4)]-Fuc- wherein R8 is 5-O-[5-O-Ara/Api-3,5-dihydroxy-6-methyl-octanoyl]-3,5-dihydroxy-6-methyl-octanoic acid),
Api-(1→3)-Xyl-(1→4)-Rha-(1→2)-[R9-(→4)]-Fuc- wherein R9 is 5-O-[5-O-Ara/Api-3,5-dihydroxy-6-methyl-octanoyl]-3,5-dihydroxy-6-methyl-octanoic acid),
Xyl-(1→3)-Xyl-(1-4)-Rha-(1→2)-[R10-(→4)]-Fuc- wherein R10 is 5-O-[5-O-Ara/Api-3,5-dihydroxy-6-methyl-octanoyl]-3,5-dihydroxy-6-methyl-octanoic acid),
Api-(1→3)-Xyl-(1→4)-Rha-(1→2)-[R11-(→3)]-Fuc- wherein R11 is 5-O-[5-O-Ara/Api-3,5-dihydroxy-6-methyl-octanoyl]-3,5-dihydroxy-6-methyl-octanoic acid),
Xyl-(1→3)-Xyl-(1→4)-Rha-(1→2)-[R12-(→3)]-Fuc- wherein R12 is 5-O-[5-O-Ara/Api-3,5-dihydroxy-6-methyl-octanoyl]-3,5-dihydroxy-6-methyl-octanoic acid) and
Glc-(1→3)-[Glc-(1→6)]-Gal-,
preferably the at least one saponin comprises such a selected first saccharide chain and such a selected second saccharide chain.

11. Conjugate of any one of the claims 1-10, wherein the at least one saponin is selected from: 3-O-beta-D-galactopyranosyl-(1→2)-[beta-D-xylopyranosyl-(1→3)]-beta-D-glucuronopyranosyl quillaic acid 28-O-beta-D-glucopyranosyl-(1→3)-beta-D-xylopyranosyl-(1→4)-alpha-L-rhamnopyranosyl-(1→2)-[beta-D-xylopyranosyl-(1→3)-40Ac-beta-D-quinovopyranosyl-(1→4)]-beta-D-fucopyranoside, Quillaja bark saponin, dipsacoside B, saikosaponin A, saikosaponin D, macranthoidin A, esculentoside A, phytolaccagenin, aescinate, AS6.2, NP-005236, AMA-1, AMR, alpha-Hederin, NP-012672, NP-017777, NP-017778, NP-017774, NP-018110, NP-017772, NP-018109, NP-017888, NP-017889, NP-018108, SA1641, AE X55, NP-017674, NP-017810, AG1, NP-003881, NP-017676, NP-017677, NP-017706, NP-017705, NP-017773, NP-017775, SA1657, AG2, SO1861, SO1862, SO1904, GE1741, SO1542, SO1584, SO1658, SO1674, SO1832, QS-7, QS1861, QS-7 api, QS1862, QS-17, QS-18, QS-21 A-apio, QS-21 A-xylo, QS-21 B-apio, QS-21 B-xylo, beta-Aescin, Aescin Ia, Teaseed saponin I, Teaseedsapon in J, Assamsapon in F, Digitonin, Primula acid 1 and AS64R, and/or a functional derivative thereof, and/or a stereoisomer thereof, and/or any combination thereof, the functional derivative optionally without an aldehyde group in the aglycone core structure of the at least one saponin, and/or without a glucuronic acid moiety comprising a carboxyl group in a first saccharide chain of the at least one saponin, and/or without an acetyl group in a second saccharide chain of the at least one saponin, preferably the at least one saponin is selected from: QS-21, GE1741, SA1641 and SO1861, and/or a functional derivative thereof, the functional derivative preferably without an aldehyde group in the aglycone core structure of the at least one saponin, and/or without a glucuronic acid moiety comprising a carboxyl group in a first saccharide chain of the at least one saponin, and/or without an acetyl group in a second saccharide chain of the at least one saponin.

12. Conjugate of any one of the claims 1-11, wherein the at least one saponin is selected from: SO1861, SA1657, GE1741, SA1641, QS-21, QS-21A, QS-21 A-api, QS-21 A-xyl, QS-21B, QS-21 B-api, QS-21 B-xyl, QS-7-xyl, QS-7-api, QS-17-api, QS-17-xyl, QS1861, QS1862, Quillajasaponin, Saponinum album, QS-18, Quil-A, Gyp1, gypsoside A, AG1, AG2, SO1542, SO1584, SO1658, SO1674, SO1832, SO1862, SO1904, and/or a stereoisomer thereof, and/or a functional derivative thereof, and/or any combination thereof, optionally the functional derivative without an aldehyde group in the aglycone core structure of the at least one saponin, and/or without a glucuronic acid moiety comprising a carboxyl group in a first saccharide chain of the at least one saponin, and/or without an acetyl group in a second saccharide chain of the at least one saponin, preferably the at least one saponin is selected from: SO1861, GE1741, SA1641 and QS-21, preferably a functional derivative thereof without an aldehyde group in the aglycone core structure of the at least one saponin, and/or without a glucuronic acid moiety comprising a carboxyl group in a first saccharide chain of the at least one saponin, and/or without an acetyl group in a second saccharide chain of the at least one saponin.

13. Conjugate of any one of the claims 1-12, wherein the at least one saponin comprises an aglycone core structure comprising an aldehyde group, and/or comprises a first saccharide chain comprising a glucuronic acid moiety comprising a carboxyl group, and/or comprises a second saccharide chain comprising an acetyl group, preferably, the at least one saponin comprises an aglycone core structure comprising an aldehyde group, a first saccharide chain comprising a glucuronic acid moiety comprising a carboxyl group and a second saccharide chain comprising an acetyl group, more preferably, the at least one saponin comprises one or two of: an aglycone core structure comprising an aldehyde group, a first saccharide chain comprising a glucuronic acid moiety comprising a carboxyl group, a second saccharide chain comprising an acetyl group.

14. Conjugate of claim 11 or 12, wherein the at least one saponin is a saponin functional derivative comprising none of an aglycone core structure comprising an aldehyde group, a first saccharide chain comprising a glucuronic acid moiety comprising a carboxyl group and a second saccharide chain comprising an acetyl group.

15. Conjugate of any one of the claims 1-14, wherein the at least one saponin is covalently bound to the sdAb via a linker, preferably a cleavable linker.

16. Conjugate of claim 15, wherein the cleavable linker is subject to cleavage under acidic conditions, reductive conditions, enzymatic conditions and/or light-induced conditions, and preferably the cleavable linker comprises a cleavable bond selected from a hydrazone bond and a hydrazide bond subject to cleavage under acidic conditions, and/or a bond susceptible to proteolysis, for example proteolysis by Cathepsin B, and/or a bond susceptible for cleavage under reductive conditions such as a disulfide bond.

17. Conjugate of claim 15 or 16, wherein the cleavable linker is subject to cleavage in vivo under acidic conditions such as for example present in endosomes and/or lysosomes of mammalian cells, preferably human cells, preferably the cleavable linker is subject to cleavage in vivo at pH 4.0-6.5, and more preferably at pH 5.5.

18. Conjugate of any one of the claims 1-17, wherein the at least one saponin is part of a covalent saponin conjugate comprising an oligomeric molecule or a polymeric molecule to which the saponin is covalently bound, and wherein the sdAb is also covalently bound to the same oligomeric molecule or polymeric molecule as to which the saponin is bound.

19. Conjugate of claim 18, wherein 1-8 of the oligomeric molecules or polymeric molecules comprising the saponin(s) is/are covalently bound to the sdAb, or 2-4 of the oligomeric molecules or polymeric molecules comprising the saponin(s), wherein the oligomeric molecule or the polymeric molecule of the covalent saponin conjugate is optionally a dendron such as a G2 dendron, G3 dendron, G4 dendron or G5 dendron with 4, 8, 16 and 32 binding sites for covalently binding 4, 8, 16 or 32 saponin moieties, respectively, wherein 1-32 saponin moieties, preferably 2, 3, 4, 5, 6, 8, 10, 16, 32 saponin moieties, or any number of saponin moieties therein between, such as 7, 9, 12 saponin moieties, are covalently bound to the oligomeric molecule or to the polymeric molecule of the covalent saponin conjugate.

20. Conjugate of claim 18 or 19, wherein the at least one saponin is covalently bound to the oligomeric molecule or to the polymeric molecule of the covalent saponin conjugate via any one or more of an imine bond, a hydrazone bond, a hydrazide bond, an oxime bond, a 1,3-dioxolane bond, a disulfide bond, a thio-ether bond, an amide bond, a peptide bond or an ester bond, preferably via a linker.

21. Conjugate of claim 20, wherein the at least one saponin comprises an aldehyde function in position C₂₃ of the aglycone core structure of the at least one saponin and optionally a glucuronic acid function in a first saccharide chain at the C₃beta-OH group of the aglycone core structure of the at least one saponin, which aldehyde function is involved in the covalent bonding to the oligomeric molecule or to the polymeric molecule of the covalent saponin conjugate, and/or, if present, the glucuronic acid function is involved in the covalent bonding of the at least one saponin to the oligomeric molecule or to the polymeric molecule of the covalent saponin conjugate, the bonding of the at least one saponin either via a direct covalent bond, or via a linker.

22. Conjugate of claim 21, wherein the aldehyde function in position C₂₃ of the aglycone core structure of the at least one saponin is covalently bound to a linker, preferably to linker N-ε-maleimidocaproic acid hydrazide (EMCH), which linker is covalently bound via a thio-ether bond to a sulfhydryl group in the oligomeric molecule or in the polymeric molecule of the covalent saponin conjugate, such as a sulfhydryl group of a cysteine.

23. Conjugate of claim 21 or 22, wherein the glucuronic acid function in the first saccharide chain at the C₃beta-OH group of the aglycone core structure of the at least one saponin is covalently bound to a linker such as linker 1-[Bis(dimethylamino)methylene]-1H-1,2,3-triazolo[4,5-b]pyridinium 3-oxid hexafluorophosphate (HATU), which linker is covalently bound via an amide bond to an amine group in the oligomeric molecule or in the polymeric molecule of the covalent saponin conjugate, such as an amine group of a lysine or an N-terminus of a protein.

24. Conjugate of any one of the claims 20-23, wherein the polymeric molecule or the oligomeric molecule of the covalent saponin conjugate is bound to the sdAb, preferably to an amino-acid residue of the sdAb, involving a click chemistry group on the polymeric molecule or the oligomeric molecule of the covalent saponin conjugate, the click chemistry group preferably selected from a tetrazine, an azide, an alkene or an alkyne, or a cyclic derivative of these groups, more preferably the click chemistry group is an azide.

25. Conjugate of any one of the claims 18-24, wherein the polymeric molecule or the oligomeric molecule of the covalent saponin conjugate comprises a polymeric structure and/or an oligomeric structure selected from: a linear polymer, a branched polymer and/or a cyclic polymer, an oligomer, a dendrimer, a dendron, a dendronized polymer, a dendronized oligomer, a DNA, a polypeptide, a poly-lysine, a poly-ethylene glycol, an oligo-ethylene glycol (OEG), such as OEG₃, OEG₄ and OEG₅, or an assembly of these polymeric structures and/or oligomeric structures which assembly is preferably built up by covalent cross-linking, preferably the polymeric molecule or the oligomeric molecule of the covalent saponin conjugate is a dendron such as a poly-amidoamine (PAMAM) dendrimer.

26. Conjugate of any one of the claims 1-25, wherein the sdAb is a single sdAb or are at least two, preferably two sdAbs, wherein the sdAb(s) can bind to a tumor-cell surface molecule of the cell, preferably a tumor-cell surface receptor such as a tumor-cell specific receptor, more preferably a receptor of the cell selected from any one or more of: CD71, CA125, EpCAM(17-1A), CD52, CEA, CD44v6, FAP, EGF-IR, integrin, syndecan-1, vascular integrin alpha-V beta-3, HER2, EGFR, CD20, CD22, Folate receptor 1, CD146, CD56, CD19, CD138, CD27L receptor, prostate specific membrane antigen (PSMA), CanAg, integrin-alphaV, CA6, CD33, mesothelin, Cripto, CD3, CD30, CD239, CD70, CD123, CD352, DLL3, CD25, ephrinA4, MUC-1, Trop2, CEACAM5, CEACAM6, HER3, CD74, PTK7, Notch3, FGF2, C4.4A, FLT3, CD38, FGFR3, CD7, PD-L1, CTLA-4, CD52, PDGFRA, VEGFR1, VEGFR2, c-Met (HGFR), EGFR1, RANKL, ADAMTS5, CD16, CXCR7 (ACKR3), glucocorticoid-induced TNFR-related protein (GITR), most preferably selected from: HER2, c-Met, VEGFR2, CXCR7, CD71 and EGFR1.

27. Conjugate of any one of the claims 1-26, wherein the sdAb is a single sdAb or are at least two, preferably two sdAbs, wherein the sdAb(s) is/are selected from: an anti-CD71 sdAb, an anti-HER2 sdAb, an anti-CD20 sdAb, an anti-CA125 sdAb, an anti-EpCAM (17-1A) sdAb, an anti-EGFR sdAb, an anti-CD30 sdAb, an anti-CD33 sdAb, an anti-vascular integrin alpha-v beta-3 sdAb, an anti-CD52 sdAb, an anti-CD22 sdAb, an anti-CEA sdAb, an anti-CD44v6 sdAb, an anti-FAP sdAb, an anti-CD19 sdAb, an anti-CanAg sdAb, an anti-CD56 sdAb, an anti-CD38 sdAb, an anti-CA6 sdAb, an anti-IGF-1R sdAb, an anti-integrin sdAb, an anti-syndecan-1 sdAb, an anti-CD79b, an anti-c-Met sdAb, an anti-EGFR1 sdAb, an anti-VEGFR2 sdAb, an anti-CXCR7 sdAb, wherein the sdAb(s) is/are preferably V_{HH}(s), more preferably camelid V_{H}(s).

28. Conjugate of any one of the claims 1-27, wherein the conjugate comprises an sdAb that can bind to HER2, CD71 or to EGFR, wherein the sdAb is preferably a V_{HH}, more preferably camelid V_{H}.

29. Conjugate of any one of the claims 1-28, wherein the conjugate comprises an sdAb for binding to HER2 selected from: sdAb produced by clone 11A4, clone 18C3, clone 22G12, clone 017, clone Q17-C-tag; or an sdAb for binding to EGFR and produced by clone anti-EGFR Q86-C-tag; or an sdAb for binding to CD71 and produced by clone anti-CD71 Q52-C-tag; or an sdAb for binding to HIVgp41 and produced by clone anti-HIVgp41 Q8-C-tag; or an sdAb encoded by a cDNA of any one of the SEQ ID NO: 1, 3, 5, 7, 9, 11, 13, 15, 17, 19, 21, 23, 25, 27, 29 and 31; or any one of the sdAbs with an amino-acid sequence of SEQ ID NO: 2, 4, 6, 8, 10, 12, 14, 16, 18, 20, 22, 24, 26, 28, 30, 32, 36-72, or the biparatopic tandem of sdAbs 7D12 and 9G8 with an amino-acid sequence of SEQ ID NO: 74 or sdAb 7D12 with an amino-acid sequence of SEQ ID NO: 75 or sdAb 9G8 with an amino-acid sequence of SEQ ID NO: 76, wherein optionally the conjugate further comprises an sdAb for binding to albumin, such as any one or more of sdAbs with an amino-acid sequence of SEQ ID NO: 33, 34 and 35.

30. Conjugate of any one of the claims 1-29, wherein the conjugate is represented by the general structure of Molecule I:
sdAb1(-L1-Sᵤ₁)ₙ-[L2a-((-sdAb2)ₘ₁(-L3-Sᵤ₂)ₚ)_{q1} . . . L2i-((-sdAb9)ₘ₂(-L4-Sᵤ₃)ₚ)_{q2}]-(L5-sdAb10ᵣ(-L6-Sᵤ₄)ₜ)ᵥ (Molecule I),
wherein sdAb1, sdAb2 . . . sdAb9, sdAb10 are sdAbs sdAb1, sdAb2, sdAb3, sdAb4, sdAb5, sdAb6, sdAb7, sdAb8, sdAb9 and sdAb10, which sdAbs are the same sdAb;
S is at least one saponin moiety;
L1, L3, L4 and L6 are each independently a covalent bond or a covalent linker linking the at least one saponin to the sdAb, wherein such linkers are the same or different;
L2a-L2i, and L5 are each independently a covalent bond or a covalent linker linking two consecutive sdAbs if more than one sdAb is present, wherein such linkers are the same or different;
n is an integer selected from 0-4, preferably n is 1, 2, 3 or 4;
m1, m2 each independently is an integer selected from 0-10, preferably m1 and/or m2 is/are 0, 1, 2, 3, 4, 5, 6, 7 or 8, wherein, if m1 or m2 is >1, consecutive sdAbs are covalently linked, preferably via a linker;
p is an integer selected from 0-4, preferably p is 1, 2, 3 or 4;
q1, q2 each independently is an integer selected from 0-10, preferably q1 and/or q2 is/are 0, 1, 2, 3, 4, 5, 6, 7 or 8;
r is an integer selected from 0-10, preferably r is 0, 1, 2, 3, 4, 5, 6, 7, 8 or 9;
t is an integer selected from 0-4, preferably t is 1, 2, 3 or 4;
u1, u2, u3, u4 are an integer, each independently selected from 0-100, preferably u1, u2, u3 and/or
u4 is/are 0, 1, 2, 4, 16, 32 or 64, preferably u1, u2, u3 and u4 are the same; and
v is 0 or 1.

31. Conjugate of claim 30, wherein L1 and L2a are linkers, u1 is >0, n is >0, m1 is any of 1-9, p is 0, q1 is 1, m2 is 0, u2 is 0, u3 is 0, q2 is 0, v is 0, r is 0, u4 is 0 and t is 0, or wherein L1, L2a, L5 and L6 are a linker, u1 is 0, n is 0, m1 is any of 1-9, u2 is 0, p is 0, q1 is 1, m2 is 0, u3 is 0, p is 0, q2 is 0, r is 1, u4 is >1, t is >1 and v is 1.

32. Pharmaceutical combination comprising:
a first pharmaceutical composition comprising the conjugate of any one of the claims 1-31 and optionally comprising a pharmaceutically acceptable excipient and/or a pharmaceutically acceptable diluent; and
a second pharmaceutical composition comprising an active pharmaceutical ingredient such as selected from any one or more of: a drug molecule, an oligonucleotide such as an mRNA, an anti-sense oligonucleotide, a ligand-drug conjugate such as EGF-dianthin or EGF-saporin, an antibody-drug conjugate (ADC) such as OKT9 monoclonal anti-CD71 antibody, trastuzumab or cetuximab conjugated with saporin, dianthin or a BNA, a ligand-oligonucleotide conjugate such as an antibody-oligonucleotide conjugate (AOC) such as an antibody-BNA conjugate or an antibody-siRNA conjugate, wherein the antibody of the ADC or the AOC optionally comprises or consists of at least one sdAb, wherein the at least one sdAb comprised by the ADC or AOC is/are different from or the same as the sdAb of the conjugate of any one of the claims 1-31, and optionally comprising a pharmaceutically acceptable excipient and/or a pharmaceutically acceptable diluent.

33. Pharmaceutical composition comprising:
the conjugate of any one of the claims 1-31;
one or more active pharmaceutical ingredients, such as selected from: a drug molecule, an oligonucleotide such as an mRNA, an ASO, a ligand-drug conjugate such as EGF-dianthin or EGF-saporin, an ADC such as OKT9 monoclonal anti-CD71 antibody, trastuzumab or cetuximab conjugated with saporin, dianthin or a BNA, a ligand-oligonucleotide conjugate such as an AOC such as an antibody-BNA conjugate or an antibody-siRNA conjugate, wherein optionally the antibody of the ADC or the AOC comprises or consists of at least one sdAb, wherein the at least one sdAb comprised by the ADC or AOC is/are different from or the same as the sdAb of the conjugate of any one of the claims 1-31; and
optionally comprising a pharmaceutically acceptable excipient and/or a pharmaceutically acceptable diluent.

34. Pharmaceutical combination of claim 32 or pharmaceutical composition of claim 33, wherein the antibody of the ADC or the AOC comprises or is a V_{HH}, preferably a camelid V_{H}.

35. Pharmaceutical combination of claim 32 or 34 or pharmaceutical composition of claim 33-34, wherein the conjugate of any of the claims 1-31 comprises an sdAb that can bind to HER2, CD71 or EGFR, and/or wherein one or more active pharmaceutical ingredients comprise(s) an sdAb that can bind to HER2, CD71 or EGFR, and/or wherein the ADC comprises dianthin or saporin.

36. Pharmaceutical combination of any one of the claim 32 or 34-35 or pharmaceutical composition of any one of the claims 33-35, for use as a medicament.

37. Pharmaceutical combination of any one of the claim 32 or 34-35 or pharmaceutical composition of any one of the claims 32-34, for use in the treatment or the prophylaxis of a cancer, an auto-immune disease, an infection such as a viral infection, an enzyme deficiency, a disorder or disease relating to an enzyme deficiency, a gene defect, a disorder or disease relating to a gene defect.

38. In vitro or ex vivo method for transferring a molecule from outside a cell to inside said cell, preferably to the cytosol of said cell, comprising the steps of:
a) providing a cell which expresses a binding site for the sdAb according to any one of the claims 1-31 on its surface, preferably selected from a liver cell, an aberrant cell such as a virally infected cell, an auto-immune cell and a tumor cell;
b) providing the molecule for transferring into the cell provided in step a), wherein the molecule is any one or more of the active pharmaceutical ingredient(s) of any one of the claims 32-35;
c) providing the conjugate of any one of the claims 1-31;
d) contacting the cell of step a) in vitro or ex vivo with the molecule of step b) and the conjugate of step c),
therewith establishing the transfer of the molecule from outside the cell into said cell, preferably into the cytosol of said cell.

39. Kit of parts, comprising the pharmaceutical combination of any one of the claim 32 or 34-35 or the pharmaceutical composition of any one of the claims 33-35, or the conjugate of any one of the claims 1-31, and instructions for use of said pharmaceutical combination according to any one of the claim 32 or 34-38, for use of said pharmaceutical composition according to any one of the claims 33-38, or for use of said conjugate according to claim 38.
